SEQUENCE LISTING (585 sequences)

SEQ ID NO: 1 - protein, 879 aa, Homo sapiens
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 2 - protein, 731 aa, Homo sapiens
PSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 3 - protein, 611 aa, Homo sapiens
VATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 4 - protein, 485 aa, homo sapien
SPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 5 - protein, 343 aa, Homo sapiens
KPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 6 - protein, 217 aa, Homo sapiens
VRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 7 - protein, 66 aa, homo sapiens
SKPVFITVKMDVLNAFKYPLLIGVGLSTVIGLLSCLIGYCSSHWCCKKEVQETRRERRRLMSMEMD

SEQ ID NO: 8 - protein, 21 aa, Homo sapiens
MGRLASRPLLLALLSLALCRG

SEQ ID NO: 9 - protein, 385 aa, Homo sapiens
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 10 - protein, 247 aa, Homo sapiens
PGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 11 - protein, 168 aa, Homo sapiens
HGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 12 - protein, 66 aa, homo sapiens
SHLAALWPFLGIVAEVLVLVTIIFIYEKRRKPEDVLDDDDAGSAPLKSSGQHQNDKGKNVRQRNSS

SEQ ID NO: 13 - protein, 18 aa, homo sapiens
MAAALFVLLGFALLGTHG

SEQ ID NO: 14 - protein, 613 aa, homo sapiens
MGALRPTLLPPSLPLLLLLMLGMGCWAREVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 15 - protein, 456 aa, homo sapiens
APPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 16 - protein, 320 aa, Homo sapiens
AHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 17 - protein, 179 aa, homo sapiens
REEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 18 - protein, 24 aa, Homo sapiens
VALVTGATVLGTITCCFMKRLRKR

SEQ ID NO: 19 - protein, 27 aa, Homo sapiens
MGALRPTLLPPSLPLLLLLMLGMGCWA

SEQ ID NO: 20 - protein, 1195 aa, homo sapiens
MKCFFPVLSCLAVLGVVSAQRQVTVQEGPLYRTEGSHITIWCNVSGYQGPSEQNFQWSIYLPSSPEREVQIVSTMDSSFPYAIYTQRVRGGKIFIERVQGNSTLLHITDLQARDAGEYECHTPSTDKQYFGSYSAKMNLVVIPDSLQTTAMPQTLHRVEQDPLELTCEVASETIQHSHLSVAWLRQKVGEKPVEVISLSRDFMLHSSSEYAQRQSLGEVRLDKLGRTTFRLTIFHLQPSDQGEFYCEAAEWIQDPDGSWYAMTRKRSEGAVVNVQPTDKEFTVRLETEKRLHTVGEPVEFRCILEAQNVPDRYFAVSWAFNSSLIATMGPNAVPVLNSEFAHREARGQLKVAKESDSVFVLKIYHLRQEDSGKYNCRVTEREKTVTGEFIDKESKRPKNIPIIVLPLKSSISVEVASNASVILEGEDLRFSCSVRTAGRPQGRFSVIWQLVDRQNRRSNIMWLDRDGTVQPGSSYWERSSFGGVQMEQVQPNSFSLGIFNSRKEDEGQYECHVTEWVRAVDGEWQIVGERRASTPISITALEMGFAVTAISRTPGVTYSDSFDLQCIIKPHYPAWVPVSVTWRFQPVGTVEFHDLVTFTRDGGVQWGDRSSSFRTRTAIEKAESSNNVRLSISRASDTEAGKYQCVAELWRKNYNNTWTRLAERTSNLLEIRVLQPVTKLQVSKSKRTLTLVENKPIQLNCSVKSQTSQNSHFAVLWYVHKPSDADGKLILKTTHNSAFEYGTYAEEEGLRARLQFERHVSGGLFSLTVQRAEVSDSGSYYCHVEEWLLSPNYAWYKLAEEVSGRTEVTVKQPDSRLRLSQAQGNLSVLETRQVQLECVVLNRTSITSQLMVEWFVWKPNHPERETVARLSRDATFHYGEQAAKNNLKGRLHLESPSPGVYRLFIQNVAVQDSGTYSCHVEEWLPSPSGMWYKRAEDTAGQTALTVMRPDASLQVDTVVPNATVSEKAAFQLDCSIVSRSSQDSRFAVAWYSLRTKAGGKRSSPGLEEQEEEREEEEEEEEDDDDDDPTERTALLSVGPDAVFGPEGSPWEGRLRFQRLSPVLYRLTVLQASPQDTGNYSCHVEEWLPSPQKEWYRLTEEESAPIGIRVLDTSPTLQSIICSNDALFYFVFFYPFPIFGILIITILLVRFKSRNSSKNSDGKNGVPLLWIKEPHLNYSPTCLEPPVLSIHPGAID

SEQ ID NO: 21 - protein, 798 aa, homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 22 - protein, 1032 aa, homo sapiens [sequence omitted]

SEQ ID NO: 23 - protein, 660 aa, homo sapiens
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTETGFHHVSQADIEFLTSIDPTASASGSAGITGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYA

SEQ ID NO: 24 - protein, 1023 aa, homo sapiens
MGKGVGRDKYEPAAVSEQGDKKGKKGKKDRDMDELKKEVSMDDHKLSLDELHRKYGTDLSRGLTSARAAEILARDGPNALTPPPTTPEWIKFCRQLFGGFSMLLWIGAILCFLAYSIQAATEEEPQNDNLYLGVVLSAVVIITGCFSYYQEAKSSKIMESFKNMVPQQALVIRNGEKMSINAEEVVVGDLVEVKGGDRIPADLRIISANGCKVDNSSLTGESEPQTRSPDFTNENPLETRNIAFFSTNCVEGTARGIVVYTGDRTVMGRIATLASGLEGGQTPIAAEIEHFIHIITGVAVFLGVSFFILSLILEYTWLEAVIFLIGIIVANVPEGLLATVTVCLTLTAKRMARKNCLVKNLEAVETLGSTSTICSDKTGTLTQNRMTVAHMWFDNQIHEADTTENQSGVSFDKTSATWLALSRIAGLCNRAVFQANQENLPILKRAVAGDASESALLKCIELCCGSVKEMRERYAKIVEIPFNSTNKYQLSIHKNPNTSEPQHLLVMKGAPERILDRCSSILLHGKEQPLDEELKDAFQNAYLELGGLGERVLGFCHLFLPDEQFPEGFQFDTDDVNFPIDNLCFVGLISMIDPPRAAVPDAVGKCRSAGIKVIMVTGDHPITAKAIAKGVGIISEGNETVEDIAARLNIPVSQVNPRDAKACVVHGSDLKDMTSEQLDDILKYHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLIFIIANIPLPLGTVTILCIDLGTDMVPAISLAYEQAESDIMKRQPRNPKTDKLVNERLISMAYGQIGMIQALGGFFTYFVILAENGFLPIHLLGLRVDWDDRWINDVEDSYGQQWTYEQRKIVEFTCHTAFFVSIVVVQWADLVICKTRRNSVFQQGMKNKILIFGLFEETALAAFLSYCPGMGVALRMYPLKPTWWFCAFPYSLLIFVYDEVRKLIIRRRPGGWVEKETYY

SEQ ID NO: 25 - protein, 1020 aa, homo sapiens
MGRGAGREYSPAATTAENGGGKKKQKEKELDELKKEVAMDDHKLSLDELGRKYQVDLSKGLTNQRAQDVLARDGPNALTPPPTTPEWVKFCRQLFGGFSILLWIGAILCFLAYGIQAAMEDEPSNDNLYLGVVLAAVVIVTGCFSYYQEAKSSKIMDSFKNMVPQQALVIREGEKMQINAEEVVVGDLVEVKGGDRVPADLRIISSHGCKVDNSSLTGESEPQTRSPEFTHENPLETRNICFFSTNCVEGTARGIVIATGDRTVMGRIATLASGLEVGRTPIAMEIEHFIQLITGVAVFLGVSFFVLSLILGYSWLEAVIFLIGIIVANVPEGLLATVTVCLTLTAKRMARKNCLVKNLEAVETLGSTSTICSDKTGTLTQNRMTVAHMWFDNQIHEADTTEDQSGATFDKRSPTWTALSRIAGLCNRAVFKAGQENISVSKRDTAGDASESALLKCIELSCGSVRKMRDRNPKVAEIPFNSTNKYQLSIHEREDSPQSHVLVMKGAPERILDRCSTILVQGKEIPLDKEMQDAFQNAYMELGGLGERVLGFCQLNLPSGKFPRGFKFDTDELNFPTEKLCFVGLMSMIDPPRAAVPDAVGKCRSAGIKVIMVTGDHPITAKAIAKGVGIISEGNETVEDIAARLNIPMSQVNPREAKACVVHGSDLKDMTSEQLDEILKNHTEIVFARTSPQQKLIIVEGCQRQGAIVAVTGDGVNDSPALKKADIGIAMGISGSDVSKQAADMILLDDNFASIVTGVEEGRLIFDNLKKSIAYTLTSNIPEITPFLLFIIANIPLPLGTVTILCIDLGTDMVPAISLAYEAAESDIMKRQPRNSQTDKLVNERLISMAYGQIGMIQALGGFFTYFVILAENGFLPSRLLGIRLDWDDRTMNDLEDSYGQEWTYEQRKVVEFTCHTAFFASIVVVQWADLIICKTRRNSVFQQGMKNKILIFGLLEETALAAFLSYCPGMGVALRMYPLKVTWWFCAFPYSLLIFIYDEVRKLILRRYPGGWVEKETYY

SEQ ID NO: 26 - protein, 1026 aa, homo sapiens
MGSGGSDSYRIATSQDKKDDKDSPKKNKGKERRDLDDLKKEVAMTEHKMSVEEVCRKYNTDCVQGLTHSKAQEILARDGPNALTPPPTTPEWVKFCRQLFGGFSILLWIGAILCFLAYGIQAGTEDDPSGDNLYLGIVLAAVVIITGCFSYYQEAKSSKIMESFKNMVPQQALVIREGEKMQVNAEEVVVGDLVEIKGGDRVPADLRIISAHGCKVDNSSLTGESEPQTRSPDCTHDNPLETRNITFFSTNCVEGTARGVVVATGDRTVMGRIATLASGLEVGKTPIAIEIEHFIQLITGVAVFLGVSFFILSLILGYTWLEAVIFLIGIIVANVPEGLLATVTVCLTLTAKRMARKNCLVKNLEAVETLGSTSTICSDKTGTLTQNRMTVAHMWFDNQIHEADTTEDQSGTSFDKSSHTWVALSHIAGLCNRAVFKGGQDNIPVLKRDVAGDASESALLKCIELSSGSVKLMRERNKKVAEIPFNSTNKYQLSIHETEDPNDNRYLLVMKGAPERILDRCSTILLQGKEQPLDEEMKEAFQNAYLELGGLGERVLGFCHYYLPEEQFPKGFAFDCDDVNFTTDNLCFVGLMSMIDPPRAAVPDAVGKCRSAGIKVIMVTGDHPITAKAIAKGVGIISEGNETVEDIAARLNIPVSQVNPRDAKACVIHGTDLKDFTSEQIDEILQNHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLLFIMANIPLPLGTITILCIDLGTDMVPAISLAYEAAESDIMKRQPRNPRTDKLVNERLISMAYGQIGMIQALGGFFSYFVILAENGFLPGNLVGIRLNWDDRTVNDLEDSYGQQWTYEQRKVVEFTCHTAFFVSIVVVQWADLIICKTRRNSVFQQGMKNKILIFGLFEETALAAFLSYCPGMDVALRMYPLKPSWWFCAFPYSFLIFVYDEIRKLILRRNPGGWVEKETYY

SEQ ID NO: 27 - protein, 1029 aa, Homo sapiens
MGLWGKKGTVAPHDQSPRRRPKKGLIKKKMVKREKQKRNMEELKKEVVMDDHKLTLEELSTKYSVDLTKGHSHQRAKEILTRGGPNTVTPPPTTPEWVKFCKQLFGGFSLLLWTGAILCFVAYSIQIYFNEEPTKDNLYLSIVLSVVVIVTGCFSYYQEAKSSKIMESFKNMVPQQALVIRGGEKMQINVQEVVLGDLVEIKGGDRVPADLRLISAQGCKVDNSSLTGESEPQSRSPDFTHENPLETRNICFFSTNCVEGTARGIVIATGDSTVMGRIASLTSGLAVGQTPIAAEIEHFIHLITVVAVFLGVTFFALSLLLGYGWLEAIIFLIGIIVANVPEGLLATVTVCLTLTAKRMARKNCLVKNLEAVETLGSTSTICSDKTGTLTQNRMTVAHMWFDMTVYEADTTEEQTGKTFTKSSDTWFMLARIAGLCNRADFKANQEILPIAKRATTGDASESALLKFIEQSYSSVAEMREKNPKVAEIPFNSTNKYQMSIHLREDSSQTHVLMMKGAPERILEFCSTFLLNGQEYSMNDEMKEAFQNAYLELGGLGERVLGFCFLNLPSSFSKGFPFNTDEINFPMDNLCFVGLISMIDPPRAAVPDAVSKCRSAGIKVIMVTGDHPITAKAIAKGVGIISEGTETAEEVAARLKIPISKVDASAAKAIVVHGAELKDIQSKQLDQILQNHPEIVFARTSPQQKLIIVEGCQRLGAVVAVTGDGVNDSPALKKADIGIAMGISGSDVSKQAADMILLDDNFASIVTGVEEGRLIFDNLKKSIMYTLTSNIPEITPFLMFIILGIPLPLGTITILCIDLGTDMVPAISLAYESAESDIMKRLPRNPKTDNLVNHRLIGMAYGQIGMIQALAGFFTYFVILAENGFRPVDLLGIRLHWEDKYLNDLEDSYGQQWTYEQRKVVEFTCQTAFFVTIVVVQWADLIISKTRRNSLFQQGMRNKVLIFGILEETLLAAFLSYTPGMDVALRMYPLKITWWLCAIPYSILIFVYDEIRKLLIRQHPDGWVERETYY

SEQ ID NO: 28 - protein, 279 aa, homo sapiens
MTKNEKKSLNQSLAEWKLFIYNPTTGEFLGRTAKSWGLILLFYLVFYGFLAALFSFTMWVMLQTLNDEVPKYRDQIPSPGLMVFPKPVTALEYTFSRSDPTSYAGYIEDLKKFLKPYTLEEQKNLTVCPDGALFEQKGPVYVACQFPISLLQACSGMNDPDFGYSQGNPCILVKMNRIIGLKPEGVPRIDCVSKNEDIPNVAVYPHNGMIDLKYFPYYGKKLHVGYLQPLVAVQVSFAPNNTGKEVTVECKIDGSANLKSQDDRDKFLGRVMFKITARA

SEQ ID NO: 29 - protein, 1258 aa, Homo sapiens
MGDMANNSVAYSGVKNSLKEANHDGDFGITLAELRALMELRSTDALRKIQESYGDVYGICTKLKTSPNEGLSGNPADLERREAVFGKNFIPPKKPKTFLQLVWEALQDVTLIILEIAAIVSLGLSFYQPPEGDNALCGEVSVGEEEGEGETGWIEGAAILLSVVCVVLVTAFNDWSKEKQFRGLQSRIEQEQKFTVIRGGQVIQIPVADITVGDIAQVKYGDLLPADGILIQGNDLKIDESSLTGESDHVKKSLDKDPLLLSGTHVMEGSGRMVVTAVGVNSQTGIIFTLLGAGGEEEEKKDEKKKEKKNKKQDGAIENRNKAKAQDGAAMEMQPLKSEEGGDGDEKDKKKANLPKKEKSVLQGKLTKLAVQIGKAGLLMSAITVIILVLYFVIDTFWVQKRPWLAECTPIYIQYFVKFFIIGVTVLVVAVPEGLPLAVTISLAYSVKKMMKDNNLVRHLDACETMGNATAICSDKTGTLTMNRMTVVQAYINEKHYKKVPEPEAIPPNILSYLVTGISVNCAYTSKILPPEKEGGLPRHVGNKTECALLGLLLDLKRDYQDVRNEIPEEALYKVYTFNSVRKSMSTVLKNSDGSYRIFSKGASEIILKKCFKILSANGEAKVFRPRDRDDIVKTVIEPMASEGLRTICLAFRDFPAGEPEPEWDNENDIVTGLTCIAVVGIEDPVRPEVPDAIKKCQRAGITVRMVTGDNINTARAIATKCGILHPGEDFLCLEGKDFNRRIRNEKGEIEQERIDKIWPKLRVLARSSPTDKHTLVKGIIDSTVSDQRQVVAVTGDGTNDGPALKKADVGFAMGIAGTDVAKEASDIILTDDNFTSIVKAVMWGRNVYDSISKFLQFQLTVNVVAVIVAFTGACITQDSPLKAVQMLWVNLIMDTLASLALATEPPTESLLLRKPYGRNKPLISRTMMKNILGHAFYQLVVVFTLLFAGEKFFDIDSGRNAPLHAPPSEHYTIVFNTFVLMQLFNEINARKIHGERNVFEGIFNNAIFCTIVLGTFVVQIIIVQFGGKPFSCSELSIEQWLWSIFLGMGTLLWGQLISTIPTSRLKFLKEAGHGTQKEEIPEEELAEDVEEIDHAERELRRGQILWFRGLNRIQTQMDVVNAFQSGSSIQGALRRQPSIASQHHDVTNISTPTHIRVVNAFRSSLYEGLEKPESRSSIHNFMTHPEFRIEDSEPHIPLIDDTDAEDDAPTKRNSSPPPSPNKNNNAVDSGIHLTIEMNKSATSSSPGSPLHSLETSL

SEQ ID NO: 30 - protein, 1272 aa, Homo sapiens
MGDMTNSDFYSKNQRNESSHGGEFGCTMEELRSLMELRGTEAVVKIKETYGDTEAICRRLKTSPVEGLPGTAPDLEKRKQIFGQNFIPPKKPKTFLQLVWEALQDVTLIILEIAAIISLGLSFYHPPGEGNEGCATAQGGAEDEGEAEAGWIEGAAILLSVICVVLVTAFNDWSKEKQFRGLQSRIEQEQKFTVVRAGQVVQIPVAEIVVGDIAQVKYGDLLPADGLFIQGNDLKIDESSLTGESDQVRKSVDKDPMLLSGTHVMEGSGRMLVTAVGVNSQTGIIFTLLGAGGEEEEKKDKKGVKKGDGLQLPAADGAAASNAADSANASLVNGKMQDGNVDASQSKAKQQDGAAAMEMQPLKSAEGGDADDRKKASMHKKEKSVLQGKLTKLAVQIGKAGLVMSAITVIILVLYFTVDTFVVNKKPWLPECTPVYVQYFVKFFIIGVTVLVVAVPEGLPLAVTISLAYSVKKMMKDNNLVRHLDACETMGNATAICSDKTGTLTTNRMTVVQAYVGDVHYKEIPDPSSINTKTMELLINAIAINSAYTTKILPPEKEGALPRQVGNKTECGLLGFVLDLKQDYEPVRSQMPEEKLYKVYTFNSVRKSMSTVIKLPDESFRMYSKGASEIVLKKCCKILNGAGEPRVFRPRDRDEMVKKVIEPMACDGLRTICVAYRDFPSSPEPDWDNENDILNELTCICVVGIEDPVRPEVPEAIRKCQRAGITVRMVTGDNINTARAIAIKCGIIHPGEDFLCLEGKEFNRRIRNEKGEIEQERIDKIWPKLRVLARSSPTDKHTLVKGIIDSTHTEQRQVVAVTGDGTNDGPALKKADVGFAMGIAGTDVAKEASDIILTDDNFSSIVKAVMWGRNVYDSISKFLQFQLTVNVVAVIVAFTGACITQDSPLKAVQMLWVNLIMDTFASLALATEPPTETLLLRKPYGRNKPLISRTMMKNILGHAVYQLALIFTLLFVGEKMFQIDSGRNAPLHSPPSEHYTIIFNTFVMMQLFNEINARKIHGERNVFDGIFRNPIFCTIVLGTFAIQIVIVQFGGKPFSCSPLQLDQWMWCIFIGLGELVWGQVIATIPTSRLKFLKEAGRLTQKEEIPEEELNEDVEEIDHAERELRRGQILWFRGLNRIQTQIEVVNTFKSGASFQGALRRQSSVTSQSQDIRVVKAFRSSLYEGLEKPESRTSIHNFMAHPEFRIEDSQPHIPLIDDTDLEEDAALKQNSSPPSSLNKNNSAIDSGINLTTDTSKSATSSSPGSPIHSLETSL

SEQ ID NO: 31 - protein, 1241 aa, Homo sapiens
MTNPSDRVLPANSMAESREGDFGCTVMELRKLMELRSRDALTQINVHYGGVQNLCSRLKTSPVEGLSGNPADLEKRRQVFGHNVIPPKKPKTFLELVWEALQDVTLIILEIAAIISLVLSFYRPAGEENELCGQVATTPEDENEAQAGWIEGAAILFSVIIVVLVTAFNDWSKEKQFRGLQCRIEQEQKFSIIRNGQLIQLPVAEIVVGDIAQVKYGDLLPADGILIQGNDLKIDESSLTGESDHVKKSLDKDPMLLSGTHVMEGSGRMVVTAVGVNSQTGIILTLLGVNEDDEGEKKKKGKKQGVPENRNKAKTQDGVALEIQPLNSQEGIDNEEKDKKAVKVPKKEKSVLQGKLTRLAVQIGKAGLLMSALTVFILILYFVIDNFVINRRPWLPECTPIYIQYFVKFFIIGITVLVVAVPEGLPLAVTISLAYSVKKMMKDNNLVRHLDACETMGNATAICSDKTGTLTMNRMTVVQAYIGGIHYRQIPSPDVFLPKVLDLIVNGISINSAYTSKILPPEKEGGLPRQVGNKTECALLGFVTDLKQDYQAVRNEVPEEKLYKVYTFNSVRKSMSTVIRNPNGGFRMYSKGASEIILRKCNRILDRKGEAVPFKNKDRDDMVRTVIEPMACDGLRTICIAYRDFDDTEPSWDNENEILTELTCIAVVGIEDPVRPEVPDAIAKCKQAGITVRMVTGDNINTARAIATKCGILTPGDDFLCLEGKEFNRLIRNEKGEVEQEKLDKIWPKLRVLARSSPTDKHTLVKGIIDSTVGEHRQVVAVTGDGTNDGPALKKADVGFAMGIAGTDVAKEASDIILTDDNFTSIVKAVMWGRNVYDSISKFLQFQLTVNVVAVIVAFTGACITQDSPLKAVQMLWVNLIMDTFASLALATEPPTESLLKRRPYGRNKPLISRTMMKNILGHAFYQLIVIFILVFAGEKFFDIDSGRKAPLHSPPSQHYTIVFNTFVLMQLFNEINSRKIHGEKNVFSGIYRNIIFCSVVLGTFICQIFIVEFGGKPFSCTSLSLSQWLWCLFIGIGELLWGQFISAIPTRSLKFLKEAGHGTTKEEITKDAEGLDEIDHAEMELRRGQILWFRGLNRIQTQIDVINTFQTGASFKGVLRRQNMGQHLDVKLVPSSSYIKVVKAFHSSLHESIQKPYNQKSIHSFMTHPEFAIEEELPRTPLLDEEEEENPDKASKFGTRVLLLDGEVTPYANTNNNAVDCNQVQLPQSDSSLQSLETSV

SEQ ID NO: 32 - protein, 1241 aa, homo sapiens; identical to SEQ ID NO: 31

SEQ ID NO: 33 - protein, 193 aa, homo sapiens
GPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 34 - protein, 1021 aa, homo sapiens
MAGISYVASFFLLLTKLSIGQREVTVQKGPLFRAEGYPVSIGCNVTGHQGPSEQHFQWSVYLPTNPTQEVQIISTKDAAFSYAVYTQRVRSGDVYVERVQGNSVLLHISKLQMKDAGEYECHTPNTDEKYYGSYSAKTNLIVIPDTLSATMSSQTLGKEEGEPLALTCEASKATAQHTHLSVTWYLTQDGGGSQATEIISLSKDFILVPGPLYTERFAASDVQLNKLGPTTFRLSIERLQSSDQGQLFCEATEWIQDPDETWMFITKKQTDQTTLRIQPAVKDFQVNITADSLFAEGKPLELVCLVVSSGRDPQLQGIWFFNGTEIAHIDAGGVLGLKNDYKERASQGELQVSKLGPKAFSLKIFSLGPEDEGAYRCVVAEVMKTRTGSWQVLQRKQSPDSHVHLRKPAARSVVMSTKNKQQVVWEGETLAFLCKAGGAESPLSVSWWHIPRDQTQPEFVAGMGQDGIVQLGASYGVPSYHGNTRLEKMDWATFQLEITFTAITDSGTYECRVSEKSRNQARDLSWTQKISVTVKSLESSLQVSLMSRQPQVMLTNTFDLSCVVRAGYSDLKVPLTVTWQFQPASSHIFHQLIRITHNGTIEWGNFLSRFQKKTKVSQSLFRSQLLVHDATEEETGVYQCEVEVYDRNSLYNNRPPRASAISHPLRIAVTLPESKLKVNSRSQVQELSINSNTDIECSILSRSNGNLQLAIIWYFSPVSTNASWLKILEMDQTNVIKTGDEFHTPQRKQKFHTEKVSQDLFQLHILNVEDSDRGKYHCAVEEWLLSTNGTWHKLGEKKSGLTELKLKPTGSKVRVSKVYWTENVTEHREVAIRCSLESVGSSATLYSVMWYWNRENSGSKLLVHLQHDGLLEYGEEGLRRHLHCYRSSSTDFVLKLHQVEMEDAGMYWCRVAEWQLHGHPSKWINQASDESQRMVLTVLPSEPTLPSRICSSAPLLYFLFICPFVLLLLLLISLLCLYWKARKLSTLRSNTRKEKALWVDLKEAGGVTTNRREDEEEDEGN

SEQ ID NO: 35 - protein, 20 aa, homo sapiens
MAGISYVASFFLLLTKLSIG

SEQ ID NO: 36 - DNA, 20 bp, Artificial Sequence
  Synthetic  Oligonucleotide
cgttggcagtccgccttaac

SEQ ID NO: 37 - DNA, 20 bp, Artificial Sequence
  Synthetic  oligonucleotide
catagtcactgacgttgcag

SEQ ID NO: 38 - DNA, 20 bp, Artificial Sequence
  Synthetic  oligonucleotide
ttgtggagcttgcaagcacc

SEQ ID NO: 39 - DNA, 20 bp, Artificial Sequence
  Synthetic  oligonucleotide
gttctttatgtggagctcca

SEQ ID NO: 40 - DNA, 20 bp, Artificial Sequence
  Synthetic  oligonucleotide
tatcccttgctgatcggcgt

SEQ ID NO: 41 - DNA, 20 bp, Artificial Sequence
  Synthetic  oligonucleotide
gctgcagtacccgatgagac

SEQ ID NO: 42 - protein, 38 aa, Artificial Sequence
  Synthetic Polypeptide
EHSAGGGGSDYKDDDDKGGGGSLSNPIEIDFQTSGPIF

SEQ ID NO: 43 - protein, 34 aa, Artificial Sequence
  Synthetic Polypeptide
EHSAGGGGSDYKDDDDKGGGGSIEIDFQTSGPIF

SEQ ID NO: 44 - protein, 30 aa, Artificial Sequence
  Synthetic Polypeptide
EHSAGGGGSDYKDDDDKGGGGSFQTSGPIF

SEQ ID NO: 45 - protein, 26 aa, Artificial Sequence
  Synthetic Polypeptide
EHSAGGGGSDYKDDDDKGGGGSGPIF

SEQ ID NO: 46 - protein, 42 aa, Artificial Sequence
  Synthetic Polypeptide
FITVKMDTLDPRSFLLRNPNDKYEPFWEDEEKNESGSDKTHT

SEQ ID NO: 47 - protein, 332 aa, Homo sapiens
MGAQFSKTAAKGEAAAERPGEAAVASSPSKANGQENGHVKVNGDASPAAAESGAKEELQANGSAPAADKEEPAAAGSGAASPSAAEKGEPAAAAAPEAGASPVEKEAPAEGEAAEPGSPTAAEGEAASAASSTSSPKAEDGATPSPSNETPKKKKKRFSFKKSFKLSGFSFKKNKKEAGEGGEAEAPAAEGGKDEAAGGAAAAAAEAGAASGEQAAAPGEEAAAGEEGAAGGDPQEAKPQEAAVAPEKPPASDETKAAEEPSKVEEKKAEEAGASAAACEAPSAAGPGAPPEQEAAPAEEPAAAAASSACAAPSQEAQPECSPEAPPAEAAE

SEQ ID NO: 48 - protein, 195 aa, Homo sapiens
MGSQSSKAPRGDVTAEEAAGASPAKANGQENGHVKSNGDLSPKGEGESPPVNGTDEAAGATGDAIEPAPPSQGAEAKGEVPPKETPKKKKKFSFKKPFKLSGLSFKRNRKEGGGDSSASSPTEEEQEQGEIGACSDEGTAQEGKAAATPESQEPQAKGAEASAASEEEAGPQATEPSTPSGPESGPTPASAEQNE

SEQ ID NO: 49 - protein, 227 aa, Homo sapiens
MGGKLSKKKKGYNVNDEKAKEKDKKAEGAATEEEGTPKESEPQAAAEPAEAKEGKEKPDQDAEGKAEEKEGEKDAAAAKEEAPKAEPEKTEGAAEAKAEPPKAPEQEQAAPGPAAGGEAPKAAEAAAAPAESAAPAAGEEPSKEEGEPKKTEAPAAPAAQETKSDGAPASDSKPGSSEAAPSSKETPAATEAPSSTPKAQGPAASAEEPKPVEAPAANSDQTVTVKE

SEQ ID NO: 50 - protein, 30 aa, Artificial Sequence
  Description of Artificial Sequence: Synthetic peptide
MGGKLSKKKKGYNVNDEKAKEKDKKAEGAA

SEQ ID NO: 51 - protein, 27 aa, Artificial Sequence
  Description of Artificial Sequence: Synthetic peptide
MGGKLSKKKKGYNVNDEKAKEKDKKAE

SEQ ID NO: 52 - protein, 24 aa, Artificial Sequence
  Description of Artificial Sequence: Synthetic peptide
MGGKLSKKKKGYNVNDEKAKEKDK

SEQ ID NO: 53 - protein, 21 aa, Artificial Sequence
  Description of Artificial Sequence: Synthetic peptide
MGGKLSKKKKGYNVNDEKAKE

SEQ ID NO: 54 - protein, 18 aa, Artificial Sequence
  Description of Artificial Sequence: Synthetic peptide
MGGKLSKKKKGYNVNDEK

SEQ ID NO: 55 - protein, 15 aa, Artificial Sequence
  Description of Artificial Sequence: Synthetic peptide
MGGKLSKKKKGYNVN

SEQ ID NO: 56 - protein, 12 aa, Artificial Sequence
  Description of Artificial Sequence: Synthetic peptide
MGGKLSKKKKGY

SEQ ID NO: 57 - protein, 11 aa, Artificial Sequence
  Description of Artificial Sequence: Synthetic peptide
MGGKLSKKKKG

SEQ ID NO: 58 - protein, 10 aa, Artificial Sequence
  Description of Artificial Sequence: Synthetic peptide
MGGKLSKKKK

SEQ ID NO: 59 - protein, 9 aa, Artificial Sequence
  Description of Artificial Sequence: Synthetic peptide
MGGKLSKKK

SEQ ID NO: 60 - protein, 8 aa, Artificial Sequence
  Description of Artificial Sequence: Synthetic peptide
MGGKLSKK

SEQ ID NO: 61 - protein, 7 aa, Artificial Sequence
  Description of Artificial Sequence: Synthetic peptide
MGGKLSK

SEQ ID NO: 62 - protein, 8 aa, Artificial Sequence
  Description of Artificial Sequence: Synthetic peptide
MGGKLAKK

SEQ ID NO: 63 - protein, 8 aa, Artificial Sequence
  Description of Artificial Sequence: Synthetic peptide
MGGKFSKK

SEQ ID NO: 64 - protein, 8 aa, Artificial Sequence
  Description of Artificial Sequence: Synthetic peptide
MGGKFAKK

SEQ ID NO: 65 - protein, 8 aa, Artificial Sequence
  Description of Artificial Sequence: Synthetic peptide
MGGKSSKK

SEQ ID NO: 66 - protein, 8 aa, Artificial Sequence
  Description of Artificial Sequence: Synthetic peptide
MGGKSAKK

SEQ ID NO: 67 - protein, 8 aa, Artificial Sequence
  Description of Artificial Sequence: Synthetic peptide
MGGKQSKK

SEQ ID NO: 68 - protein, 8 aa, Artificial Sequence
  Description of Artificial Sequence: Synthetic peptide
MGGKQAKK

SEQ ID NO: 69 - protein, 8 aa, Artificial Sequence
  Description of Artificial Sequence: Synthetic peptide
MGGQLSKK

SEQ ID NO: 70 - protein, 8 aa, Artificial Sequence
  Description of Artificial Sequence: Synthetic peptide
MGGQLAKK

SEQ ID NO: 71 - protein, 8 aa, Artificial Sequence
  Description of Artificial Sequence: Synthetic peptide
MGGQFSKK

SEQ ID NO: 72 - protein, 8 aa, Artificial Sequence
  Description of Artificial Sequence: Synthetic peptide
MGGQFAKK

SEQ ID NO: 73 - protein, 8 aa, Artificial Sequence
  Description of Artificial Sequence: Synthetic peptide
MGGQSSKK

SEQ ID NO: 74 - protein, 8 aa, Artificial Sequence
  Description of Artificial Sequence: Synthetic peptide
MGGQSAKK

SEQ ID NO: 75 - protein, 8 aa, Artificial Sequence
  Description of Artificial Sequence: Synthetic peptide
MGGQQSKK

SEQ ID NO: 76 - protein, 8 aa, Artificial Sequence
  Description of Artificial Sequence: Synthetic peptide
MGGQQAKK

SEQ ID NO: 77 - protein, 8 aa, Artificial Sequence
  Description of Artificial Sequence: Synthetic peptide
MGAKLSKK

SEQ ID NO: 78 - protein, 8 aa, Artificial Sequence
  Description of Artificial Sequence: Synthetic peptide
MGAKLAKK

SEQ ID NO: 79 - protein, 8 aa, Artificial Sequence
  Description of Artificial Sequence: Synthetic peptide
MGAKFSKK

SEQ ID NO: 80 - protein, 8 aa, Artificial Sequence
  Description of Artificial Sequence: Synthetic peptide
MGAKFAKK

SEQ ID NO: 81 - protein, 8 aa, Artificial Sequence
  Description of Artificial Sequence: Synthetic peptide
MGAKSSKK

SEQ ID NO: 82 - protein, 8 aa, Artificial Sequence
  Description of Artificial Sequence: Synthetic peptide
MGAKSAKK

SEQ ID NO: 83 - protein, 8 aa, Artificial Sequence
  Description of Artificial Sequence: Synthetic peptide
MGAKQSKK

SEQ ID NO: 84 - protein, 8 aa, Artificial Sequence
  Description of Artificial Sequence: Synthetic peptide
MGAKQAKK

SEQ ID NO: 85 - protein, 8 aa, Artificial Sequence
  Description of Artificial Sequence: Synthetic peptide
MGAQLSKK

SEQ ID NO: 86 - protein, 8 aa, Artificial Sequence
  Description of Artificial Sequence: Synthetic peptide
MGAQLAKK

SEQ ID NO: 87 - protein, 8 aa, Artificial Sequence
  Description of Artificial Sequence: Synthetic peptide
MGAQFSKK

SEQ ID NO: 88 - protein, 8 aa, Artificial Sequence
  Description of Artificial Sequence: Synthetic peptide
MGAQFAKK

SEQ ID NO: 89 - protein, 8 aa, Artificial Sequence
  Description of Artificial Sequence: Synthetic peptide
MGAQSSKK

SEQ ID NO: 90 - protein, 8 aa, Artificial Sequence
  Description of Artificial Sequence: Synthetic peptide
MGAQSAKK

SEQ ID NO: 91 - protein, 8 aa, Artificial Sequence
  Description of Artificial Sequence: Synthetic peptide
MGAQQSKK

SEQ ID NO: 92 - protein, 8 aa, Artificial Sequence
  Description of Artificial Sequence: Synthetic peptide
MGAQQAKK

SEQ ID NO: 93 - protein, 8 aa, Artificial Sequence
  Description of Artificial Sequence: Synthetic peptide
MGSKLSKK

SEQ ID NO: 94 - protein, 8 aa, Artificial Sequence
  Description of Artificial Sequence: Synthetic peptide
MGSKLAKK

SEQ ID NO: 95 - protein, 8 aa, Artificial Sequence
  Description of Artificial Sequence: Synthetic peptide
MGSKFSKK

SEQ ID NO: 96 - protein, 8 aa, Artificial Sequence
  Description of Artificial Sequence: Synthetic peptide
MGSKFAKK

SEQ ID NO: 97 - protein, 8 aa, Artificial Sequence
  Description of Artificial Sequence: Synthetic peptide
MGSKSSKK

SEQ ID NO: 98 - protein, 8 aa, Artificial Sequence
  Description of Artificial Sequence: Synthetic peptide
MGSKSAKK

SEQ ID NO: 99 - protein, 8 aa, Artificial Sequence
  Description of Artificial Sequence: Synthetic peptide
MGSKQSKK

SEQ ID NO: 100 - protein, 8 aa, Artificial Sequence
  Description of Artificial Sequence: Synthetic peptide
MGSKQAKK

SEQ ID NO: 101 - protein, 8 aa, Artificial Sequence
  Description of Artificial Sequence: Synthetic peptide
MGSQLSKK

SEQ ID NO: 102 - protein, 8 aa, Artificial Sequence
  Description of Artificial Sequence: Synthetic peptide
MGSQLAKK

SEQ ID NO: 103 - protein, 8 aa, Artificial Sequence
  Description of Artificial Sequence: Synthetic peptide
MGSQFSKK

SEQ ID NO: 104 - protein, 8 aa, Artificial Sequence
  Description of Artificial Sequence: Synthetic peptide
MGSQFAKK

SEQ ID NO: 105 - protein, 8 aa, Artificial Sequence
  Description of Artificial Sequence: Synthetic peptide
MGSQSSKK

SEQ ID NO: 106 - protein, 8 aa, Artificial Sequence
  Description of Artificial Sequence: Synthetic peptide
MGSQSAKK

SEQ ID NO: 107 - protein, 8 aa, Artificial Sequence
  Description of Artificial Sequence: Synthetic peptide
MGSQQSKK

SEQ ID NO: 108 - protein, 8 aa, Artificial Sequence
  Description of Artificial Sequence: Synthetic peptide
MGSQQAKK

SEQ ID NO: 109 - protein, 7 aa, Artificial Sequence
  Description of Artificial Sequence: Synthetic peptide
MGGKLAK

SEQ ID NO: 110 - protein, 7 aa, Artificial Sequence
  Description of Artificial Sequence: Synthetic peptide
MGGKFSK

SEQ ID NO: 111 - protein, 7 aa, Artificial Sequence
  Description of Artificial Sequence: Synthetic peptide
MGGKFAK

SEQ ID NO: 112 - protein, 7 aa, Artificial Sequence
  Description of Artificial Sequence: Synthetic peptide
MGGKSSK

SEQ ID NO: 113 - protein, 7 aa, Artificial Sequence
  Description of Artificial Sequence: Synthetic peptide
MGGKSAK

SEQ ID NO: 114 - protein, 7 aa, Artificial Sequence
  Description of Artificial Sequence: Synthetic peptide
MGGKQSK

SEQ ID NO: 115 - protein, 7 aa, Artificial Sequence
  Description of Artificial Sequence: Synthetic peptide
MGGKQAK

SEQ ID NO: 116 - protein, 7 aa, Artificial Sequence
  Description of Artificial Sequence: Synthetic peptide
MGGQLSK

SEQ ID NO: 117 - protein, 7 aa, Artificial Sequence
  Description of Artificial Sequence: Synthetic peptide
MGGQLAK

SEQ ID NO: 118 - protein, 7 aa, Artificial Sequence
  Description of Artificial Sequence: Synthetic peptide
MGGQFSK

SEQ ID NO: 119 - protein, 7 aa, Artificial Sequence
  Description of Artificial Sequence: Synthetic peptide
MGGQFAK

SEQ ID NO: 120 - protein, 7 aa, Artificial Sequence
  Description of Artificial Sequence: Synthetic peptide
MGGQSSK

SEQ ID NO: 121 - protein, 7 aa, Artificial Sequence
  Description of Artificial Sequence: Synthetic peptide
MGGQSAK

SEQ ID NO: 122 - protein, 7 aa, Artificial Sequence
  Description of Artificial Sequence: Synthetic peptide
MGGQQSK

SEQ ID NO: 123 - protein, 7 aa, Artificial Sequence
  Description of Artificial Sequence: Synthetic peptide
MGGQQAK

SEQ ID NO: 124 - protein, 7 aa, Artificial Sequence
  Description of Artificial Sequence: Synthetic peptide
MGAKLSK

SEQ ID NO: 125 - protein, 7 aa, Artificial Sequence
  Description of Artificial Sequence: Synthetic peptide
MGAKLAK

SEQ ID NO: 126 - protein, 7 aa, Artificial Sequence
  Description of Artificial Sequence: Synthetic peptide
MGAKFSK

SEQ ID NO: 127 - protein, 7 aa, Artificial Sequence
  Description of Artificial Sequence: Synthetic peptide
MGAKFAK

SEQ ID NO: 128 - protein, 7 aa, Artificial Sequence
  Description of Artificial Sequence: Synthetic peptide
MGAKSSK

SEQ ID NO: 129 - protein, 7 aa, Artificial Sequence
  Description of Artificial Sequence: Synthetic peptide
MGAKSAK

SEQ ID NO: 130 - protein, 7 aa, Artificial Sequence
  Description of Artificial Sequence: Synthetic peptide
MGAKQSK

SEQ ID NO: 131 - protein, 7 aa, Artificial Sequence
  Description of Artificial Sequence: Synthetic peptide
MGAKQAK

SEQ ID NO: 132 - protein, 7 aa, Artificial Sequence
  Description of Artificial Sequence: Synthetic peptide
MGAQLSK

SEQ ID NO: 133 - protein, 7 aa, Artificial Sequence
  Description of Artificial Sequence: Synthetic peptide
MGAQLAK

SEQ ID NO: 134 - protein, 7 aa, Artificial Sequence
  Description of Artificial Sequence: Synthetic peptide
MGAQFSK

SEQ ID NO: 135 - protein, 7 aa, Artificial Sequence
  Description of Artificial Sequence: Synthetic peptide
MGAQFAK

SEQ ID NO: 136 - protein, 7 aa, Artificial Sequence
  Description of Artificial Sequence: Synthetic peptide
MGAQSSK

SEQ ID NO: 137 - protein, 7 aa, Artificial Sequence
  Description of Artificial Sequence: Synthetic peptide
MGAQSAK

SEQ ID NO: 138 - protein, 7 aa, Artificial Sequence
  Description of Artificial Sequence: Synthetic peptide
MGAQQSK

SEQ ID NO: 139 - protein, 7 aa, Artificial Sequence
  Description of Artificial Sequence: Synthetic peptide
MGAQQAK

SEQ ID NO: 140 - protein, 7 aa, Artificial Sequence
  Description of Artificial Sequence: Synthetic peptide
MGSKLSK

SEQ ID NO: 141 - protein, 7 aa, Artificial Sequence
  Description of Artificial Sequence: Synthetic peptide
MGSKLAK

SEQ ID NO: 142 - protein, 7 aa, Artificial Sequence
  Description of Artificial Sequence: Synthetic peptide
MGSKFSK

SEQ ID NO: 143 - protein, 7 aa, Artificial Sequence
  Description of Artificial Sequence: Synthetic peptide
MGSKFAK

SEQ ID NO: 144 - protein, 7 aa, Artificial Sequence
  Description of Artificial Sequence: Synthetic peptide
MGSKSSK

SEQ ID NO: 145 - protein, 7 aa, Artificial Sequence
  Description of Artificial Sequence: Synthetic peptide
MGSKSAK

SEQ ID NO: 146 - protein, 7 aa, Artificial Sequence
  Description of Artificial Sequence: Synthetic peptide
MGSKQSK

SEQ ID NO: 147 - protein, 7 aa, Artificial Sequence
  Description of Artificial Sequence: Synthetic peptide
MGSKQAK

SEQ ID NO: 148 - protein, 7 aa, Artificial Sequence
  Description of Artificial Sequence: Synthetic peptide
MGSQLSK

SEQ ID NO: 149 - protein, 7 aa, Artificial Sequence
  Description of Artificial Sequence: Synthetic peptide
MGSQLAK

SEQ ID NO: 150 - protein, 7 aa, Artificial Sequence
  Description of Artificial Sequence: Synthetic peptide
MGSQFSK

SEQ ID NO: 151 - protein, 7 aa, Artificial Sequence
  Description of Artificial Sequence: Synthetic peptide
MGSQFAK

SEQ ID NO: 152 - protein, 7 aa, Artificial Sequence
  Description of Artificial Sequence: Synthetic peptide
MGSQSSK

SEQ ID NO: 153 - protein, 7 aa, Artificial Sequence
  Description of Artificial Sequence: Synthetic peptide
MGSQSAK

SEQ ID NO: 154 - protein, 7 aa, Artificial Sequence
  Description of Artificial Sequence: Synthetic peptide
MGSQQSK

SEQ ID NO: 155 - protein, 7 aa, Artificial Sequence
  Description of Artificial Sequence: Synthetic peptide
MGSQQAK

SEQ ID NO: 156 - protein, 9 aa, Artificial Sequence
  Description of Artificial Sequence: Synthetic peptide
MGAKLSKKK

SEQ ID NO: 157 - protein, 167 aa, Artificial Sequence
  Description of Artificial Sequence: Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 158 - protein, 167 aa, Artificial Sequence
  Description of Artificial Sequence: Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQKRKYTIKVEVPKGAWRSYLNMELTIPIFATNSDCELIVKAMQGLLKDGNPIPSAIAANSGIY

SEQ ID NO: 159 - protein, 296 aa, Artificial Sequence
  Description of Artificial Sequence: Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSGGSGGSGGSGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 160 - protein, 296 aa, Artificial Sequence
  Description of Artificial Sequence: Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQKRKYTIKVEVPKGAWRSYLNMELTIPIFATNSDCELIVKAMQGLLKDGNPIPSAIAANSGIYGSGGSGGSGGSGMASNFTQFVLVDNGGTGDVTVAPSNFANGIAEWISSNSRSQAYKVTCSVRQSSAQKRKYTIKVEVPKGAWRSYLNMELTIPIFATNSDCELIVKAMQGLLKDGNPIPSAIAANSGIY

SEQ ID NO: 161 - RNA, 680 nt, Artificial Sequence
  Description of Artificial Sequence: Synthetic polynucleotide
augaagcccaccgagaacaacgaagacuucaacaucguggccguggccagcaacuucgcgaccacggaucucgaugcugaccgcgggaaguugcccggcaagaagcugccgcuggaggugcucaaagaguuggaagccaaugcccggaaagcuggcugcaccaggggcugucugaucugccugucccacaucaagugcacgcccaagaugaagaaguucaucccaggacgcugccacaccuacgaaggcgacaaagaguccgcacagggcggcauaggcgaggcgaucgucgacauuccugagauuccuggguucaaggacuuggagcccuuggagcaguucaucgcacaggucgaucuguguguggacugcacaacuggcugccucaaagggcuugccaacgugcaguguucugaccugcucaagaaguggcugccgcaacgcugugcgaccuuugccagcaagauccagggccagguggacaagaucaagggggccgguggugacuaaggauccaucgauaagcuucaucgaaacaugaggaucacccauaucugcagucgacaucgaaacaugaggaucacccaugucugcagucgacaucgaaacaugaggaucacccaugucugcagucgacaucgaaacaugaggaucacccaugucugcagucgacaucgaaa

SEQ ID NO: 162 - protein, 9 aa, Artificial Sequence
  Description of Artificial Sequence: Synthetic peptide
  MOD_RES (3)..(3): Any Naturally Occuring Amino Acid
MGXKLSKKK

SEQ ID NO: 163 - protein, 9 aa, Artificial Sequence
  Synthetic peptide
  MISC_FEATURE (3)..(3): Xaa can be any naturally occurring amino acid
MGXKLSKKK

SEQ ID NO: 164 - protein, 8 aa, Artificial Sequence
  Description of Artificial Sequence: Synthetic peptide
  MOD_RES (3)..(3): Gly, Ala, or Ser
  MOD_RES (4)..(4): Lys or Gln
  MOD_RES (5)..(5): Leu, Phe, Ser, or Gln
  MOD_RES (6)..(6): Ser or Ala
  MOD_RES (6)..(6): Ser or Ala, See specification as filed for detailed description of substitutions and preferred embodiments
MGXXXXKK

SEQ ID NO: 165 - DNA, 20 bp, Artificial Sequence
  Description of Artificial Sequence: Synthetic primer
tggaggtgctcaaagagttg

SEQ ID NO: 166 - DNA, 17 bp, Artificial Sequence
  Description of Artificial Sequence: Synthetic primer
ttgggcgtgcacttgat

SEQ ID NO: 167 - DNA, 13 bp, Artificial Sequence
  Description of Artificial Sequence: Synthetic probe
gggcattggcttc

SEQ ID NO: 168 - protein, 60 aa, Artificial Sequence
  Description of Artificial Sequence: Synthetic polypeptide
MGGKLSKKKKGYNVNDEKAKEKDKKAEGAASAGGGGSDYKDDDDKGGGGSVSKGEELFTG

SEQ ID NO: 169 - protein, 60 aa, Artificial Sequence
  Description of Artificial Sequence: Synthetic polypeptide
MAGKLSKKKKGYNVNDEKAKEKDKKAEGAASAGGGGSDYKDDDDKGGGGSVSKGEELFTG

SEQ ID NO: 170 - protein, 60 aa, Artificial Sequence
  Description of Artificial Sequence: Synthetic polypeptide
MGAKLSKKKKGYNVNDEKAKEKDKKAEGAASAGGGGSDYKDDDDKGGGGSVSKGEELFTG

SEQ ID NO: 171 - protein, 60 aa, Artificial Sequence
  Description of Artificial Sequence: Synthetic polypeptide
MAAKLSKKKKGYNVNDEKAKEKDKKAEGAASAGGGGSDYKDDDDKGGGGSVSKGEELFTG

SEQ ID NO: 172 - protein, 57 aa, Artificial Sequence
  Description of Artificial Sequence: Synthetic polypeptide
MGGKLSKKKKGYNVNDEKAKEKDKKAESAGGGGSDYKDDDDKGGGGSVSKGEELFTG

SEQ ID NO: 173 - protein, 54 aa, Artificial Sequence
  Description of Artificial Sequence: Synthetic polypeptide
MGGKLSKKKKGYNVNDEKAKEKDKSAGGGGSDYKDDDDKGGGGSVSKGEELFTG

SEQ ID NO: 174 - protein, 51 aa, Artificial Sequence
  Description of Artificial Sequence: Synthetic polypeptide
MGGKLSKKKKGYNVNDEKAKESAGGGGSDYKDDDDKGGGGSVSKGEELFTG

SEQ ID NO: 175 - protein, 48 aa, Artificial Sequence
  Description of Artificial Sequence: Synthetic polypeptide
MGGKLSKKKKGYNVNDEKSAGGGGSDYKDDDDKGGGGSVSKGEELFTG

SEQ ID NO: 176 - protein, 45 aa, Artificial Sequence
  Description of Artificial Sequence: Synthetic polypeptide
MGGKLSKKKKGYNVNSAGGGGSDYKDDDDKGGGGSVSKGEELFTG

SEQ ID NO: 177 - protein, 42 aa, Artificial Sequence
  Description of Artificial Sequence: Synthetic polypeptide
MGGKLSKKKKGYSAGGGGSDYKDDDDKGGGGSVSKGEELFTG

SEQ ID NO: 178 - protein, 39 aa, Artificial Sequence
  Description of Artificial Sequence: Synthetic polypeptide
MGGKLSKKKSAGGGGSDYKDDDDKGGGGSVSKGEELFTG

SEQ ID NO: 179 - protein, 36 aa, Artificial Sequence
  Description of Artificial Sequence: Synthetic polypeptide
MGGKLSSAGGGGSDYKDDDDKGGGGSVSKGEELFTG

SEQ ID NO: 180 - protein, 33 aa, Artificial Sequence
  Description of Artificial Sequence: Synthetic polypeptide
MGGSAGGGGSDYKDDDDKGGGGSVSKGEELFTG

SEQ ID NO: 181 - protein, 54 aa, Artificial Sequence
  Description of Artificial Sequence: Synthetic polypeptide
MGGKLSKKKKGYNVNDEKAKEKDKKAEGAASAGGGGSDYKDDDDKGGGGSVSKG

SEQ ID NO: 182 - protein, 36 aa, Artificial Sequence
  Description of Artificial Sequence: Synthetic polypeptide
MGGKLSKKKKGYSAGGGGSDYKDDDDKGGGGSVSKG

SEQ ID NO: 183 - protein, 35 aa, Artificial Sequence
  Description of Artificial Sequence: Synthetic polypeptide
MGGKLSKKKKGSAGGGGSDYKDDDDKGGGGSVSKG

SEQ ID NO: 184 - protein, 34 aa, Artificial Sequence
  Description of Artificial Sequence: Synthetic polypeptide
MGGKLSKKKKSAGGGGSDYKDDDDKGGGGSVSKG

SEQ ID NO: 185 - protein, 33 aa, Artificial Sequence
  Description of Artificial Sequence: Synthetic polypeptide
MGGKLSKKKSAGGGGSDYKDDDDKGGGGSVSKG

SEQ ID NO: 186 - protein, 32 aa, Artificial Sequence
  Description of Artificial Sequence: Synthetic polypeptide
MGGKLSKKSAGGGGSDYKDDDDKGGGGSVSKG

SEQ ID NO: 187 - protein, 31 aa, Artificial Sequence
  Description of Artificial Sequence: Synthetic polypeptide
MGGKLSKSAGGGGSDYKDDDDKGGGGSVSKG

SEQ ID NO: 188 - protein, 30 aa, Artificial Sequence
  Description of Artificial Sequence: Synthetic polypeptide
MGGKLSSAGGGGSDYKDDDDKGGGGSVSKG

SEQ ID NO: 189 - protein, 30 aa, Artificial Sequence
  Description of Artificial Sequence: Synthetic polypeptide
MGGKLDKKKKGYNVNDEKAKEKDKKAEGAA

SEQ ID NO: 190 - protein, 30 aa, Artificial Sequence
  Description of Artificial Sequence: Synthetic polypeptide
MGGKLAKKKKGYNVNDEKAKEKDKKAEGAA

SEQ ID NO: 191 - protein, 30 aa, Artificial Sequence
  Description of Artificial Sequence: Synthetic polypeptide
MGGKQSKKKKGYNVNDEKAKEKDKKAEGAA

SEQ ID NO: 192 - protein, 30 aa, Artificial Sequence
  Description of Artificial Sequence: Synthetic polypeptide
MGAKKKKKRFSFKKSFKLSGFSFKKNKKEA

SEQ ID NO: 193 - protein, 30 aa, Artificial Sequence
  Description of Artificial Sequence: Synthetic polypeptide
MAAKKKKKRFSFKKSFKLSGFSFKKNKKEA

SEQ ID NO: 194 - protein, 30 aa, Artificial Sequence
  Description of Artificial Sequence: Synthetic polypeptide
MGAKKSKKRFSFKKSFKLSGFSFKKNKKEA

SEQ ID NO: 195 - protein, 30 aa, Artificial Sequence
  Description of Artificial Sequence: Synthetic polypeptide
MGAKKAKKRFSFKKPFKLSGFSFKKNKKEA

SEQ ID NO: 196 - protein, 153 aa, Artificial Sequence
  Description of Artificial Sequence: Synthetic polypeptide
MGGKLSKKKKSAGGSGGSTSGSGDYKDDDDKGSGFEMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 197 - protein, 4 aa, Artificial Sequence
  Peptide Linker
GGGG

SEQ ID NO: 198 - protein, 7 aa, Artificial Sequence
  Peptide Linker
SGGSGGS

SEQ ID NO: 199 - protein, 15 aa, Artificial Sequence
  Peptide Linker
GGSGGSGGSGGSGGG

SEQ ID NO: 200 - protein, 16 aa, Artificial Sequence
  Peptide Linker
GGSGGSGGGGSGGGGS

SEQ ID NO: 201 - protein, 18 aa, Artificial Sequence
  Peptide Linker
GGSGGSGGSGGSGGSGGS

SEQ ID NO: 202 - protein, 15 aa, Artificial Sequence
  Peptide Linker
GGGGSGGGGSGGGGS

SEQ ID NO: 203 - protein, 400 aa, Artificial Sequence
  Peptide Linker
  MISC_FEATURE (1)..(400): This sequence may encompass 1 - 100 “GGGS” repeating units.
GGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 204 - protein, 800 aa, Artificial Sequence
  Peptide Linker
  MISC_FEATURE (1)..(300): This sequence may encompass 1-100 “GGS” repeating units
  MISC_FEATURE (301)..(800): This sequence may encompass 1-100 “GGGGS” repeating units.
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 205 - protein, 4 aa, Artificial Sequence
  Effector Domain Sequence
KKKK

SEQ ID NO: 206 - protein, 5 aa, Artificial Sequence
  Effector Domain Sequence
KKKKK

SEQ ID NO: 207 - protein, 4 aa, Artificial Sequence
  Effector Domain Sequence
RRRR

SEQ ID NO: 208 - protein, 5 aa, Artificial Sequence
  Effector Domain Sequence
RRRRR

SEQ ID NO: 209 - protein, 4 aa, Artificial Sequence
  Effector Domain Sequence
  misc_feature (1)..(4): Xaa can be Lys or Arg
XXXX

SEQ ID NO: 210 - protein, 5 aa, Artificial Sequence
  Effector Domain Sequence
  misc_feature (1)..(5): Xaa can be Lys or Arg
XXXXX

SEQ ID NO: 211 - protein, 7 aa, Artificial Sequence
  N Terminus Domain Sequence
GGKLSKK

SEQ ID NO: 212 - protein, 7 aa, Artificial Sequence
  N Terminus Domain Sequence
GAKLSKK

SEQ ID NO: 213 - protein, 7 aa, Artificial Sequence
  N Terminus Domain Sequence
GGKQSKK

SEQ ID NO: 214 - protein, 7 aa, Artificial Sequence
  N Terminus Domain Sequence
GGKLAKK

SEQ ID NO: 215 - protein, 6 aa, Artificial Sequence
  N Terminus Domain Sequence
GGKLSK

SEQ ID NO: 216 - protein, 6 aa, Artificial Sequence
  N Terminus Domain Sequence
GAKLSK

SEQ ID NO: 217 - protein, 6 aa, Artificial Sequence
  N Terminus Domain Sequence
GGKQSK

SEQ ID NO: 218 - protein, 6 aa, Artificial Sequence
  N Terminus Domain Sequence
GGKLAK

SEQ ID NO: 219 - protein, 4 aa, Artificial Sequence
  N Terminus Domain Sequence
KKKG

SEQ ID NO: 220 - protein, 5 aa, Artificial Sequence
  N Terminus Domain Sequence
KKKGY

SEQ ID NO: 221 - protein, 6 aa, Artificial Sequence
  N Terminus Domain Sequence
KKKGYN

SEQ ID NO: 222 - protein, 7 aa, Artificial Sequence
  N Terminus Domain Sequence
KKKGYNV

SEQ ID NO: 223 - protein, 8 aa, Artificial Sequence
  N Terminus Domain Sequence
KKKGYNVN

SEQ ID NO: 224 - protein, 6 aa, Artificial Sequence
  N Terminus Domain Sequence
KKKGYS

SEQ ID NO: 225 - protein, 6 aa, Artificial Sequence
  N Terminus Domain Sequence
KKKGYG

SEQ ID NO: 226 - protein, 7 aa, Artificial Sequence
  N Terminus Domain Sequence
KKKGYGG

SEQ ID NO: 227 - protein, 5 aa, Artificial Sequence
  N Terminus Domain Sequence
KKKGS

SEQ ID NO: 228 - protein, 6 aa, Artificial Sequence
  N Terminus Domain Sequence
KKKGSG

SEQ ID NO: 229 - protein, 7 aa, Artificial Sequence
  N Terminus Domain Sequence
KKKGSGS

SEQ ID NO: 230 - protein, 4 aa, Artificial Sequence
  N Terminus Domain Sequence
KKKS

SEQ ID NO: 231 - protein, 5 aa, Artificial Sequence
  N Terminus Domain Sequence
KKKSG

SEQ ID NO: 232 - protein, 6 aa, Artificial Sequence
  N Terminus Domain Sequence
KKKSGG

SEQ ID NO: 233 - protein, 7 aa, Artificial Sequence
  N Terminus Domain Sequence
KKKSGGS

SEQ ID NO: 234 - protein, 8 aa, Artificial Sequence
  N Terminus Domain Sequence
KKKSGGSG

SEQ ID NO: 235 - protein, 8 aa, Artificial Sequence
  N Terminus Domain Sequence
KKSGGSGG

SEQ ID NO: 236 - protein, 10 aa, Artificial Sequence
  N Terminus Domain Sequence
KKKSGGSGGS

SEQ ID NO: 237 - protein, 8 aa, Artificial Sequence
  N Terminus Domain Sequence
KRFSFKKS

SEQ ID NO: 238 - protein, 8 aa, Artificial Sequence
  N Terminus Domain Sequence
GGKLSKKK

SEQ ID NO: 239 - protein, 8 aa, Artificial Sequence
  N Terminus Domain Sequence
GGKLSKKS

SEQ ID NO: 240 - protein, 8 aa, Artificial Sequence
  N Terminus Domain Sequence
GAKLSKKK

SEQ ID NO: 241 - protein, 8 aa, Artificial Sequence
  N Terminus Domain Sequence
GAKLSKKS

SEQ ID NO: 242 - protein, 8 aa, Artificial Sequence
  N Terminus Domain Sequence
GGKQSKKK

SEQ ID NO: 243 - protein, 8 aa, Artificial Sequence
  N Terminus Domain Sequence
GGKQSKKS

SEQ ID NO: 244 - protein, 8 aa, Artificial Sequence
  N Terminus Domain Sequence
GGKLAKKK

SEQ ID NO: 245 - protein, 8 aa, Artificial Sequence
  N Terminus Domain Sequence
GGKLAKKS

SEQ ID NO: 246 - protein, 14 aa, Artificial Sequence
  Scaffold Y Sequence
GGKLSKKKKGYNVN

SEQ ID NO: 247 - protein, 14 aa, Artificial Sequence
  Scaffold Y Sequence
GAKLSKKKKGYNVN

SEQ ID NO: 248 - protein, 14 aa, Artificial Sequence
  Scaffold Y Sequence
GGKQSKKKKGYNVN

SEQ ID NO: 249 - protein, 14 aa, Artificial Sequence
  Scaffold Y Sequence
GGKLAKKKKGYNVN

SEQ ID NO: 250 - protein, 14 aa, Artificial Sequence
  Scaffold Y Sequence
GGKLSKKKKGYSGG

SEQ ID NO: 251 - protein, 14 aa, Artificial Sequence
  Scaffold Y Sequence
GGKLSKKKKGSGGS

SEQ ID NO: 252 - protein, 14 aa, Artificial Sequence
  Scaffold Y Sequence
GGKLSKKKKSGGSG

SEQ ID NO: 253 - protein, 14 aa, Artificial Sequence
  Scaffold Y Sequence
GGKLSKKKSGGSGG

SEQ ID NO: 254 - protein, 14 aa, Artificial Sequence
  Scaffold Y Sequence
GGKLSKKSGGSGGS

SEQ ID NO: 255 - protein, 14 aa, Artificial Sequence
  Scaffold Y Sequence
GGKLSKSGGSGGSV

SEQ ID NO: 256 - protein, 14 aa, Artificial Sequence
  Scaffold Y Sequence
GAKKSKKRFSFKKS

SEQ ID NO: 257 - protein, 29 aa, Artificial Sequence
  Scaffold Y Sequence
GGKLSKKKKGYNVNDEKAKEKDKKAEGAA

SEQ ID NO: 258 - protein, 28 aa, Artificial Sequence
  Scaffold Y Sequence
GGKLSKKKKGYNVNDEKAKEKDKKAEGA

SEQ ID NO: 259 - protein, 27 aa, Artificial Sequence
  Scaffold Y Sequence
GGKLSKKKKGYNVNDEKAKEKDKKAEG

SEQ ID NO: 260 - protein, 26 aa, Artificial Sequence
  Scaffold Y Sequence
GGKLSKKKKGYNVNDEKAKEKDKKAE

SEQ ID NO: 261 - protein, 25 aa, Artificial Sequence
  Scaffold Y Sequence
GGKLSKKKKGYNVNDEKAKEKDKKA

SEQ ID NO: 262 - protein, 24 aa, Artificial Sequence
  Scaffold Y Sequence
GGKLSKKKKGYNVNDEKAKEKDKK

SEQ ID NO: 263 - protein, 23 aa, Artificial Sequence
  Scaffold Y Sequence
GGKLSKKKKGYNVNDEKAKEKDK

SEQ ID NO: 264 - protein, 22 aa, Artificial Sequence
  Scaffold Y Sequence
GGKLSKKKKGYNVNDEKAKEKD

SEQ ID NO: 265 - protein, 21 aa, Artificial Sequence
  Scaffold Y Sequence
GGKLSKKKKGYNVNDEKAKEK

SEQ ID NO: 266 - protein, 20 aa, Artificial Sequence
  Scaffold Y Sequence
GGKLSKKKKGYNVNDEKAKE

SEQ ID NO: 267 - protein, 19 aa, Artificial Sequence
  Scaffold Y Sequence
GGKLSKKKKGYNVNDEKAK

SEQ ID NO: 268 - protein, 18 aa, Artificial Sequence
  Scaffold Y Sequence
GGKLSKKKKGYNVNDEKA

SEQ ID NO: 269 - protein, 17 aa, Artificial Sequence
  Scaffold Y Sequence
GGKLSKKKKGYNVNDEK

SEQ ID NO: 270 - protein, 16 aa, Artificial Sequence
  Scaffold Y Sequence
GGKLSKKKKGYNVNDE

SEQ ID NO: 271 - protein, 15 aa, Artificial Sequence
  Scaffold Y Sequence
GGKLSKKKKGYNVND

SEQ ID NO: 272 - protein, 13 aa, Artificial Sequence
  Scaffold Y Sequence
GGKLSKKKKGYNV

SEQ ID NO: 273 - protein, 12 aa, Artificial Sequence
  Scaffold Y Sequence
GGKLSKKKKGYN

SEQ ID NO: 274 - protein, 11 aa, Artificial Sequence
  Scaffold Y Sequence
GGKLSKKKKGY

SEQ ID NO: 275 - protein, 10 aa, Artificial Sequence
  Scaffold Y Sequence
GGKLSKKKKG

SEQ ID NO: 276 - protein, 9 aa, Artificial Sequence
  Scaffold Y Sequence
GGKLSKKKK

SEQ ID NO: 277 - protein, 28 aa, Artificial Sequence
  Scaffold Y Sequence
GAKKSKKRFSFKKSFKLSGFSFKKNKKE

SEQ ID NO: 278 - protein, 27 aa, Artificial Sequence
  Scaffold Y Sequence
GAKKSKKRFSFKKSFKLSGFSFKKNKK

SEQ ID NO: 279 - protein, 26 aa, Artificial Sequence
  Scaffold Y Sequence
GAKKSKKRFSFKKSFKLSGFSFKKNK

SEQ ID NO: 280 - protein, 25 aa, Artificial Sequence
  Scaffold Y Sequence
GAKKSKKRFSFKKSFKLSGFSFKKN

SEQ ID NO: 281 - protein, 24 aa, Artificial Sequence
  Scaffold Y Sequence
GAKKSKKRFSFKKSFKLSGFSFKK

SEQ ID NO: 282 - protein, 23 aa, Artificial Sequence
  Scaffold Y Sequence
GAKKSKKRFSFKKSFKLSGFSFK

SEQ ID NO: 283 - protein, 22 aa, Artificial Sequence
  Scaffold Y Sequence
GAKKSKKRFSFKKSFKLSGFSF

SEQ ID NO: 284 - protein, 21 aa, Artificial Sequence
  Scaffold Y Sequence
GAKKSKKRFSFKKSFKLSGFS

SEQ ID NO: 285 - protein, 20 aa, Artificial Sequence
  Scaffold Y Sequence
GAKKSKKRFSFKKSFKLSGF

SEQ ID NO: 286 - protein, 19 aa, Artificial Sequence
  Scaffold Y Sequence
GAKKSKKRFSFKKSFKLSG

SEQ ID NO: 287 - protein, 18 aa, Artificial Sequence
  Scaffold Y Sequence
GAKKSKKRFSFKKSFKLS

SEQ ID NO: 288 - protein, 17 aa, Artificial Sequence
  Scaffold Y Sequence
GAKKSKKRFSFKKSFKL

SEQ ID NO: 289 - protein, 16 aa, Artificial Sequence
  Scaffold Y Sequence
GAKKSKKRFSFKKSFK

SEQ ID NO: 290 - protein, 15 aa, Artificial Sequence
  Scaffold Y Sequence
GAKKSKKRFSFKKSF

SEQ ID NO: 291 - skipped

SEQ ID NO: 292 - protein, 13 aa, Artificial Sequence
  Scaffold Y Sequence
GAKKSKKRFSFKK

SEQ ID NO: 293 - protein, 12 aa, Artificial Sequence
  Scaffold Y Sequence
GAKKSKKRFSFK

SEQ ID NO: 294 - protein, 11 aa, Artificial Sequence
  Scaffold Y Sequence
GAKKSKKRFSF

SEQ ID NO: 295 - protein, 10 aa, Artificial Sequence
  Scaffold Y Sequence
GAKKSKKRFS

SEQ ID NO: 296 - protein, 9 aa, Artificial Sequence
  Scaffold Y Sequence
GAKKSKKRF

SEQ ID NO: 297 - protein, 8 aa, Artificial Sequence
  Scaffold Y Sequence
GAKKSKKR

SEQ ID NO: 298 - protein, 7 aa, Artificial Sequence
  Scaffold Y Sequence
GAKKSKK

SEQ ID NO: 299 - protein, 29 aa, Artificial Sequence
  Scaffold Y Sequence
GAKKSKKRFSFKKSFKLSGFSFKKNKKEA

SEQ ID NO: 300 - protein, 29 aa, Artificial Sequence
  Scaffold Y Sequence
GAKKAKKRFSFKKSFKLSGFSFKKNKKEA

SEQ ID NO: 301 - protein, 28 aa, Artificial Sequence
  Scaffold Y Sequence
GAQESKKKKKKRFSFKKSFKLSGFSFKK

SEQ ID NO: 302 - protein, 27 aa, Artificial Sequence
  Scaffold Y Sequence
GAQESKKKKKKRFSFKKSFKLSGFSFK

SEQ ID NO: 303 - protein, 26 aa, Artificial Sequence
  Scaffold Y Sequence
GAQESKKKKKKRFSFKKSFKLSGFSF

SEQ ID NO: 304 - protein, 25 aa, Artificial Sequence
  Scaffold Y Sequence
GAQESKKKKKKRFSFKKSFKLSGFS

SEQ ID NO: 305 - protein, 24 aa, Artificial Sequence
  Scaffold Y Sequence
GAQESKKKKKKRFSFKKSFKLSGF

SEQ ID NO: 306 - protein, 23 aa, Artificial Sequence
  Scaffold Y Sequence
GAQESKKKKKKRFSFKKSFKLSG

SEQ ID NO: 307 - protein, 22 aa, Artificial Sequence
  Scaffold Y Sequence
GAQESKKKKKKRFSFKKSFKLS

SEQ ID NO: 308 - protein, 21 aa, Artificial Sequence
  Scaffold Y Sequence
GAQESKKKKKKRFSFKKSFKL

SEQ ID NO: 309 - protein, 20 aa, Artificial Sequence
  Scaffold Y Sequence
GAQESKKKKKKRFSFKKSFK

SEQ ID NO: 310 - protein, 19 aa, Artificial Sequence
  Scaffold Y Sequence
GAQESKKKKKKRFSFKKSF

SEQ ID NO: 311 - protein, 18 aa, Artificial Sequence
  Scaffold Y Sequence
GAQESKKKKKKRFSFKKS

SEQ ID NO: 312 - protein, 17 aa, Artificial Sequence
  Scaffold Y Sequence
GAQESKKKKKKRFSFKK

SEQ ID NO: 313 - protein, 16 aa, Artificial Sequence
  Scaffold Y Sequence
GAQESKKKKKKRFSFK

SEQ ID NO: 314 - protein, 15 aa, Artificial Sequence
  Scaffold Y Sequence
GAQESKKKKKKRFSF

SEQ ID NO: 315 - protein, 14 aa, Artificial Sequence
  Scaffold Y Sequence
GAQESKKKKKKRFS

SEQ ID NO: 316 - protein, 13 aa, Artificial Sequence
  Scaffold Y Sequence
GAQESKKKKKKRF

SEQ ID NO: 317 - protein, 12 aa, Artificial Sequence
  Scaffold Y Sequence
GAQESKKKKKKR

SEQ ID NO: 318 - protein, 11 aa, Artificial Sequence
  Scaffold Y Sequence
GAQESKKKKKK

SEQ ID NO: 319 - protein, 10 aa, Artificial Sequence
  Scaffold Y Sequence
GAQESKKKKK

SEQ ID NO: 320 - protein, 9 aa, Artificial Sequence
  Scaffold Y Sequence
GAQESKKKK

SEQ ID NO: 321 - protein, 8 aa, Artificial Sequence
  Scaffold Y Sequence
GAQESKKK

SEQ ID NO: 322 - protein, 7 aa, Artificial Sequence
  Scaffold Y Sequence
GAQESKK

SEQ ID NO: 323 - protein, 30 aa, Artificial Sequence
  Scaffold Y Sequence
GSQSSKKKKKKFSFKKPFKLSGLSFKRNRK

SEQ ID NO: 324 - protein, 29 aa, Artificial Sequence
  Scaffold Y Sequence
GSQSSKKKKKKFSFKKPFKLSGLSFKRNR

SEQ ID NO: 325 - protein, 28 aa, Artificial Sequence
  Scaffold Y Sequence
GSQSSKKKKKKFSFKKPFKLSGLSFKRN

SEQ ID NO: 326 - protein, 27 aa, Artificial Sequence
  Scaffold Y Sequence
GSQSSKKKKKKFSFKKPFKLSGLSFKR

SEQ ID NO: 327 - protein, 26 aa, Artificial Sequence
  Scaffold Y Sequence
GSQSSKKKKKKFSFKKPFKLSGLSFK

SEQ ID NO: 328 - protein, 25 aa, Artificial Sequence
  Scaffold Y Sequence
GSQSSKKKKKKFSFKKPFKLSGLSF

SEQ ID NO: 329 - protein, 24 aa, Artificial Sequence
  Scaffold Y Sequence
GSQSSKKKKKKFSFKKPFKLSGLS

SEQ ID NO: 330 - protein, 23 aa, Artificial Sequence
  Scaffold Y Sequence
GSQSSKKKKKKFSFKKPFKLSGL

SEQ ID NO: 331 - protein, 22 aa, Artificial Sequence
  Scaffold Y Sequence
GSQSSKKKKKKFSFKKPFKLSG

SEQ ID NO: 332 - protein, 21 aa, Artificial Sequence
  Scaffold Y Sequence
GSQSSKKKKKKFSFKKPFKLS

SEQ ID NO: 333 - protein, 20 aa, Artificial Sequence
  Scaffold Y Sequence
GSQSSKKKKKKFSFKKPFKL

SEQ ID NO: 334 - protein, 19 aa, Artificial Sequence
  Scaffold Y Sequence
GSQSSKKKKKKFSFKKPFK

SEQ ID NO: 335 - protein, 18 aa, Artificial Sequence
  Scaffold Y Sequence
GSQSSKKKKKKFSFKKPF

SEQ ID NO: 336 - protein, 17 aa, Artificial Sequence
  Scaffold Y Sequence
GSQSSKKKKKKFSFKKP

SEQ ID NO: 337 - protein, 16 aa, Artificial Sequence
  Scaffold Y Sequence
GSQSSKKKKKKFSFKK

SEQ ID NO: 338 - protein, 15 aa, Artificial Sequence
  Scaffold Y Sequence
GSQSSKKKKKKFSFK

SEQ ID NO: 339 - protein, 14 aa, Artificial Sequence
  Scaffold Y Sequence
GSQSSKKKKKKFSF

SEQ ID NO: 340 - protein, 13 aa, Artificial Sequence
  Scaffold Y Sequence
GSQSSKKKKKKFS

SEQ ID NO: 341 - protein, 12 aa, Artificial Sequence
  Scaffold Y Sequence
GSQSSKKKKKKF

SEQ ID NO: 342 - protein, 11 aa, Artificial Sequence
  Scaffold Y Sequence
GSQSSKKKKKK

SEQ ID NO: 343 - protein, 10 aa, Artificial Sequence
  Scaffold Y Sequence
GSQSSKKKKK

SEQ ID NO: 344 - protein, 9 aa, Artificial Sequence
  Scaffold Y Sequence
GSQSSKKKK

SEQ ID NO: 345 - protein, 8 aa, Artificial Sequence
  Scaffold Y Sequence
GSQSSKKK

SEQ ID NO: 346 - protein, 7 aa, Artificial Sequence
  Scaffold Y Sequence
GSQSSKK

SEQ ID NO: 347 - protein, 28 aa, Artificial Sequence
  Scaffold Y Sequence
GAKKAKKRFSFKKSFKLSGFSFKKNKKE

SEQ ID NO: 348 - protein, 27 aa, Artificial Sequence
  Scaffold Y Sequence
GAKKAKKRFSFKKSFKLSGFSFKKNKK

SEQ ID NO: 349 - protein, 26 aa, Artificial Sequence
  Scaffold Y Sequence
GAKKAKKRFSFKKSFKLSGFSFKKNK

SEQ ID NO: 350 - protein, 25 aa, Artificial Sequence
  Scaffold Y Sequence
GAKKAKKRFSFKKSFKLSGFSFKKN

SEQ ID NO: 351 - protein, 24 aa, Artificial Sequence
  Scaffold Y Sequence
GAKKAKKRFSFKKSFKLSGFSFKK

SEQ ID NO: 352 - protein, 23 aa, Artificial Sequence
  Scaffold Y Sequence
GAKKAKKRFSFKKSFKLSGFSFK

SEQ ID NO: 353 - protein, 22 aa, Artificial Sequence
  Scaffold Y Sequence
GAKKAKKRFSFKKSFKLSGFSF

SEQ ID NO: 354 - protein, 21 aa, Artificial Sequence
  Scaffold Y Sequence
GAKKAKKRFSFKKSFKLSGFS

SEQ ID NO: 355 - protein, 20 aa, Artificial Sequence
  Scaffold Y Sequence
GAKKAKKRFSFKKSFKLSGF

SEQ ID NO: 356 - protein, 19 aa, Artificial Sequence
  Scaffold Y Sequence
GAKKAKKRFSFKKSFKLSG

SEQ ID NO: 357 - protein, 18 aa, Artificial Sequence
  Scaffold Y Sequence
GAKKAKKRFSFKKSFKLS

SEQ ID NO: 358 - protein, 17 aa, Artificial Sequence
  Scaffold Y Sequence
GAKKAKKRFSFKKSFKL

SEQ ID NO: 359 - protein, 16 aa, Artificial Sequence
  Scaffold Y Sequence
GAKKAKKRFSFKKSFK

SEQ ID NO: 360 - protein, 15 aa, Artificial Sequence
  Scaffold Y Sequence
GAKKAKKRFSFKKSF

SEQ ID NO: 361 - protein, 14 aa, Artificial Sequence
  Scaffold Y Sequence
GAKKAKKRFSFKKS

SEQ ID NO: 362 - protein, 13 aa, Artificial Sequence
  Scaffold Y Sequence
GAKKAKKRFSFKK

SEQ ID NO: 363 - protein, 12 aa, Artificial Sequence
  Scaffold Y Sequence
GAKKAKKRFSFK

SEQ ID NO: 364 - protein, 11 aa, Artificial Sequence
  Scaffold Y Sequence
GAKKAKKRFSF

SEQ ID NO: 365 - protein, 10 aa, Artificial Sequence
  Scaffold Y Sequence
GAKKAKKRFS

SEQ ID NO: 366 - protein, 9 aa, Artificial Sequence
  Scaffold Y Sequence
GAKKAKKRF

SEQ ID NO: 367 - protein, 8 aa, Artificial Sequence
  Scaffold Y Sequence
GAKKAKKR

SEQ ID NO: 368 - protein, 7 aa, Artificial Sequence
  Scaffold Y Sequence
GAKKAKK

SEQ ID NO: 369 - protein, 94 aa, Artificial Sequence
  ESAT6 Protein Sequence
MTEQQWNFAGIEAAASAIQGNVTSIHSLDEGKQSLTKLAAAWGGSGSEAYQGVQQKWDATATELNNALQNLARTISEAGQAMASTEGNVTGMFA

SEQ ID NO: 370 - protein, 96 aa, Artificial Sequence
  TB10.4 Protein Sequence
MSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGG

SEQ ID NO: 371 - protein, 8 aa, Artificial Sequence
  Scaffold Y Sequence
  misc_feature (2)..(2): Xaa can be any naturally occurring amino acid
GXKLSKKK

SEQ ID NO: 372 - skipped

SEQ ID NO: 373 - protein, 400 aa, Artificial Sequence
  Linker
  MISC_FEATURE (1)..(400): This sequence may encompass 1-100 “GGGG” repeating units.
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 374 - protein, 10100 aa, Artificial Sequence
  Linker
  misc_Feature (1)..(10100): This sequence may encompass 1-100 “G100-S” repeating units, which then may repeat 1-100 times.
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGSGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGS

SEQ ID NO: 375 - protein, 250 aa, Artificial Sequence
  Linker
  MISC_FEATURE (1)..(250): This sequence may encompass 1-50 “G-S” repeating units, where G may repeat 1 to 4 times and S may be present or absent.
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 376 - protein, 2 aa, Artificial Sequence
  Linker
GN

SEQ ID NO: 377 - protein, 200 aa, Artificial Sequence
  Synthetic
  MISC_FEATURE (1)..(200): This sequence may encompass 1-100 “GA” repeating units
GAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGAGA

SEQ ID NO: 378 - protein, 300 aa, Artificial Sequence
  Linker
  misc_feature (1)..(300): This sequence may encompass 1-100 “GGS” repeating units
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 379 - protein, 42 aa, Artificial Sequence [sequence omitted]

SEQ ID NO: 380 - protein, 6 aa, Artificial Sequence
  human amyloid-B protein fragment
AEFRHD

SEQ ID NO: 381 - protein, 40 aa, Artificial Sequence
  PolyGA
  misc_feature (1)..(40): This sequence may encompass 10-20 “GA” repeating units
GAGAGAGAGAGAGAGAGAGAGAGAGAGAGAGAGAGAGAGA

SEQ ID NO: 382 - protein, 13 aa, Artificial Sequence
  PADRE peptide
AKFVAAWTLKAAA

SEQ ID NO: 383 - protein, 14 aa, Artificial Sequence
  Tetanus fragment
QYIKANSKFIGITE

SEQ ID NO: 384 - protein, 15 aa, Artificial Sequence
  Diphtheria toxin fragment
QSIALSSLMVAQAIP

SEQ ID NO: 385 - protein, 119 aa, artificial sequence
  brain derived neurotrophic factor protein
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 386 - protein, 120 aa, artificial sequence
  nerve growth factor protein
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 387 - protein, 119 aa, artificial sequence [sequence omitted]

SEQ ID NO: 388 - protein, 130 aa, artificial sequence
  neurotrophin-4 protein
GVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 389 - protein, 29 aa, artificial sequence
  RVG peptide
YTIWMPENPRPGTPCDIFTNSRGKRASNG

SEQ ID NO: 390 - protein, 14 aa, artificial sequence
  cyclized TAxI peptide
SACQSQSQMRCGGG

SEQ ID NO: 391 - protein, 7 aa, artificial sequence
  TAxI transport peptide #1
QSQSQMR

SEQ ID NO: 392 - protein, 7 aa, artificial sequence
  TAxI transport peptide #2
ASGAQAR

SEQ ID NO: 393 - protein, 14 aa, artificial sequence
  TAxI transport peptide #3
TSTAPHLRLRLTSR

SEQ ID NO: 394 - protein, 7 aa, artificial sequence
  nuclear localizing signal #1
PPKKRKV

SEQ ID NO: 395 - protein, 6 aa, artificial sequence
  nuclear localizing signal #2
PKKRKV

SEQ ID NO: 396 - protein, 19 aa, artificial sequence
  Angiopep-2
TFFYGGSRGKRNNFKTEEY

SEQ ID NO: 397 - protein, 39 aa, artificial sequence
  ApoB (3371 3409)
SSVIDALQYKLEGTTRLTRKRGLKLATALSLSNKFVEGS

SEQ ID NO: 398 - protein, 18 aa, artificial sequence
  ApoE (159 167)2
LRKLRKRLLLRKLRKRLL

SEQ ID NO: 399 - protein, 8 aa, artificial sequence
  Peptide-22
CMPRLRGC

SEQ ID NO: 400 - protein, 12 aa, artificial sequence
  THR
THRPPMWSPVWP

SEQ ID NO: 401 - protein, 12 aa, artificial sequence
  THR retro-enantio
PWVPSWMPPRHT

SEQ ID NO: 402 - protein, 9 aa, artificial sequence
  CRT
CRTIGPSVC

SEQ ID NO: 403 - protein, 30 aa, artificial sequence
  Leptin30
YQQILTSMPSRNVIQISNDLENLRDLLHVL

SEQ ID NO: 404 - protein, 29 aa, artificial sequence
  RVG29
YTIWMPENPRPGTPCDIFTNSRGKRASNG

SEQ ID NO: 405 - protein, 16 aa, artificial sequence
  CDX
GREIRTGRAERWSEKF

SEQ ID NO: 406 - protein, 18 aa, artificial sequence
  Apamin
CNCKAPETALCARRCQQH

SEQ ID NO: 407 - protein, 8 aa, artificial sequence
  MiniAp-4
KAPETALD

SEQ ID NO: 408 - protein, 2 aa, artificial sequence
  GSH
CG

SEQ ID NO: 409 - protein, 13 aa, artificial sequence
  G23
HLNILSTLWKYRC

SEQ ID NO: 410 - protein, 6 aa, artificial sequence
  g7
GFGFLS

SEQ ID NO: 411 - protein, 12 aa, artificial sequence
  TGN
TGNYKALHPHNG

SEQ ID NO: 412 - protein, 11 aa, artificial sequence
  TAT (47 57)
YGRKKRRQRRR

SEQ ID NO: 413 - protein, 18 aa, artificial sequence
  SynB1
RGGRLSYSRRRFSTSTGR

SEQ ID NO: 414 - protein, 21 aa, artificial sequence
  Diketopiperazines
GNYTCEVTELTREGETIIELK

SEQ ID NO: 415 - protein, 731 aa, artificial sequence; identical to SEQ ID NO: 2
  PhPro

SEQ ID NO: 416 - protein, 29 aa, artificial sequence
  Linker
GGSSGSGSGSGGGGSGGGGTGTSSSGTGT

SEQ ID NO: 417 - protein, 1255 aa, artificial sequence
  Spike Protein
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 418 - protein, 76 aa, artificial sequence
  Envelope Protein
MYSFVSEETGTLIVNSVLLFLAFVVFLLVTLAILTALRLCAYCCNIVNVSLVKPTVYVYSRVKNLNSSEGVPDLLV

SEQ ID NO: 419 - protein, 221 aa, artificial sequence
  Membrane Protein
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 420 - protein, 9 aa, artificial sequence
  T Cell Epitope
LLLNCLWSV

SEQ ID NO: 421 - protein, 9 aa, artificial sequence
  T Cell Epitope
FKDGIYFAA

SEQ ID NO: 422 - protein, 9 aa, artificial sequence
  T Cell Epitope
LITGRLAAL

SEQ ID NO: 423 - protein, 9 aa, artificial sequence
  T Cell Epitope
LITGRLQSL

SEQ ID NO: 424 - protein, 9 aa, artificial sequence
  T Cell Epitope
ISVVLIFVV

SEQ ID NO: 425 - protein, 9 aa, artificial sequence
  T Cell Epitope
FIAGLIAIV

SEQ ID NO: 426 - protein, 9 aa, artificial sequence
  T Cell Epitope
GILGFVFTL

SEQ ID NO: 427 - protein, 9 aa, artificial sequence
  T Cell Epitope
VVFLHVTYV

SEQ ID NO: 428 - protein, 9 aa, artificial sequence
  T Cell Epitope
RLQSLQTYV

SEQ ID NO: 429 - protein, 9 aa, artificial sequence
  T Cell Epitope
VLNDILSRL

SEQ ID NO: 430 - protein, 9 aa, artificial sequence
  T Cell Epitope
VLYENQKQI

SEQ ID NO: 431 - protein, 9 aa, artificial sequence
  T Cell Epitope
NLNESLIDL

SEQ ID NO: 432 - protein, 9 aa, artificial sequence
  T Cell Epitope
LLLQYGSFC

SEQ ID NO: 433 - protein, 9 aa, artificial sequence
  T Cell Epitope
KLPDDFMGC

SEQ ID NO: 434 - protein, 9 aa, artificial sequence
  T Cell Epitope
ILPDPLKPT

SEQ ID NO: 435 - protein, 9 aa, artificial sequence
  T Cell Epitope
MIFDNAFNC

SEQ ID NO: 436 - protein, 9 aa, artificial sequence
  T Cell Epitope
FIFLLFLTL

SEQ ID NO: 437 - protein, 9 aa, artificial sequence
  T Cell Epitope
VLYQDVNCT

SEQ ID NO: 438 - protein, 9 aa, artificial sequence
  T Cell Epitope
IITTDNTFV

SEQ ID NO: 439 - protein, 9 aa, artificial sequence
  T Cell Epitope
RLNEVAKNL

SEQ ID NO: 440 - protein, 9 aa, artificial sequence
  T Cell Epitope
LLTDDMIAA

SEQ ID NO: 441 - protein, 9 aa, artificial sequence
  T Cell Epitope
LLQYGSFCT

SEQ ID NO: 442 - protein, 9 aa, artificial sequence
  T Cell Epitope
ILLCCMTSC

SEQ ID NO: 443 - protein, 9 aa, artificial sequence
  T Cell Epitope
LLFNKVTLA

SEQ ID NO: 444 - protein, 9 aa, artificial sequence
  T Cell Epitope
TMNNKSQSV

SEQ ID NO: 445 - protein, 9 aa, artificial sequence
  T Cell Epitope
ALNTLVKQL

SEQ ID NO: 446 - protein, 9 aa, artificial sequence
  T Cell Epitope
ELCDNPFFA

SEQ ID NO: 447 - protein, 9 aa, artificial sequence
  T Cell Epitope
FPRGQGVPI

SEQ ID NO: 448 - protein, 9 aa, artificial sequence
  T Cell Epitope
RLNQLESKV

SEQ ID NO: 449 - protein, 8 aa, artificial sequence
  T Cell Epitope
ILLNKHID

SEQ ID NO: 450 - protein, 18 aa, artificial sequence
  T Cell Epitope
AFFGMSRIGMEVTPSGTW

SEQ ID NO: 451 - protein, 10 aa, artificial sequence
  T Cell Epitope
MEVTPSGTWL

SEQ ID NO: 452 - protein, 9 aa, artificial sequence
  T Cell Epitope
GMSRIGMEV

SEQ ID NO: 453 - protein, 9 aa, artificial sequence
  T Cell Epitope
ILLNKHIDA

SEQ ID NO: 454 - protein, 9 aa, artificial sequence
  T Cell Epitope
ALNTPKDHI

SEQ ID NO: 455 - protein, 17 aa, artificial sequence
  T Cell Epitope
IRQGTDYKHWPQIAQFA

SEQ ID NO: 456 - protein, 17 aa, artificial sequence
  T Cell Epitope
KHWPQIAQFAPSASAFF

SEQ ID NO: 457 - protein, 9 aa, artificial sequence
  T Cell Epitope
LALLLLDRL

SEQ ID NO: 458 - protein, 9 aa, artificial sequence
  T Cell Epitope
LLLDRLNQL

SEQ ID NO: 459 - protein, 18 aa, artificial sequence
  T Cell Epitope
LLNKHIDAYKTFPPTEPK

SEQ ID NO: 460 - protein, 9 aa, artificial sequence
  T Cell Epitope
LQLPQGTTL

SEQ ID NO: 461 - protein, 15 aa, artificial sequence
  T Cell Epitope
AQFAPSASAFFGMSR

SEQ ID NO: 462 - protein, 18 aa, artificial sequence
  T Cell Epitope
AQFAPSASAFFGMSRIGM

SEQ ID NO: 463 - protein, 15 aa, artificial sequence
  T Cell Epitope
RRPQGLPNNTASWFT

SEQ ID NO: 464 - protein, 16 aa, artificial sequence
  T Cell Epitope
YKTFPPTEPKKDKKKK

SEQ ID NO: 465 - protein, 16 aa, artificial sequence
  T Cell Epitope
GAALQIPFAMQMAYRF

SEQ ID NO: 466 - protein, 16 aa, artificial sequence
  T Cell Epitope
MAYRFNGIGVTQNVLY

SEQ ID NO: 467 - protein, 18 aa, artificial sequence
  T Cell Epitope
QLIRAAEIRASANLAATK

SEQ ID NO: 468 - protein, 9 aa, artificial sequence
  T Cell Epitope
LLLLDRLNQ

SEQ ID NO: 469 - protein, 10 aa, artificial sequence
  T Cell Epitope
LLLLDRLNQL

SEQ ID NO: 470 - protein, 17 aa, artificial sequence
  T Cell Epitope
QALNTLVKQLSSNFGAI

SEQ ID NO: 471 - protein, 9 aa, artificial sequence
  T Cell Epitope
AQFAPSASA

SEQ ID NO: 472 - protein, 9 aa, artificial sequence
  T Cell Epitope
VLNDILSRL

SEQ ID NO: 473 - protein, 9 aa, artificial sequence
  T Cell Epitope
TTLPKGFYA

SEQ ID NO: 474 - protein, 10 aa, artificial sequence
  T Cell Epitope
GLIAIVMVTI

SEQ ID NO: 475 - protein, 8 aa, artificial sequence
  T Cell Epitope
LQYGSFCT

SEQ ID NO: 476 - protein, 9 aa, artificial sequence
  T Cell Epitope
RLDKVEAEV

SEQ ID NO: 477 - protein, 10 aa, artificial sequence
  T Cell Epitope
VLQLPQGTTL

SEQ ID NO: 478 - protein, 10 aa, artificial sequence
  T Cell Epitope
GYQPYRVVVL

SEQ ID NO: 479 - protein, 9 aa, artificial sequence
  T Cell Epitope
PYRVVVLSF

SEQ ID NO: 480 - protein, 9 aa, artificial sequence
  T Cell Epitope
LSPRWYFYY

SEQ ID NO: 481 - protein, 10 aa, artificial sequence
  T Cell Epitope
DSFKEELDKY

SEQ ID NO: 482 - protein, 10 aa, artificial sequence
  T Cell Epitope
LIDLQELGKY

SEQ ID NO: 483 - protein, 9 aa, artificial sequence
  T Cell Epitope
GTTLPKGFY

SEQ ID NO: 484 - protein, 10 aa, artificial sequence
  T Cell Epitope
VTPSGTWLTY

SEQ ID NO: 485 - protein, 9 aa, artificial sequence
  T Cell Epitope
GSFCTQLNR

SEQ ID NO: 486 - protein, 9 aa, artificial sequence
  T Cell Epitope
GVVFLHVTY

SEQ ID NO: 487 - protein, 9 aa, artificial sequence
  T Cell Epitope
AQALNTLVK

SEQ ID NO: 488 - protein, 9 aa, artificial sequence
  T Cell Epitope
MTSCCSCLK

SEQ ID NO: 489 - protein, 9 aa, artificial sequence
  T Cell Epitope
ASANLAATK

SEQ ID NO: 490 - protein, 10 aa, artificial sequence
  T Cell Epitope
SLIDLQELGK

SEQ ID NO: 491 - protein, 9 aa, artificial sequence
  T Cell Epitope
SVLNDILSR

SEQ ID NO: 492 - protein, 10 aa, artificial sequence
  T Cell Epitope
TQNVLYENQK

SEQ ID NO: 493 - protein, 10 aa, artificial sequence
  T Cell Epitope
CMTSCCSCLK

SEQ ID NO: 494 - protein, 10 aa, artificial sequence
  T Cell Epitope
VQIDRLITGR

SEQ ID NO: 495 - protein, 9 aa, artificial sequence
  T Cell Epitope
KTFPPTEPK

SEQ ID NO: 496 - protein, 10 aa, artificial sequence
  T Cell Epitope
KTFPPTEPKK

SEQ ID NO: 497 - protein, 9 aa, artificial sequence
  T Cell Epitope
ASAFFGMSR

SEQ ID NO: 498 - protein, 10 aa, artificial sequence
  T Cell Epitope
ATEGALNTPK

SEQ ID NO: 499 - protein, 10 aa, artificial sequence
  T Cell Epitope
QLPQGTTLPK

SEQ ID NO: 500 - protein, 9 aa, artificial sequence
  T Cell Epitope
QQQGQTVTK

SEQ ID NO: 501 - protein, 10 aa, artificial sequence
  T Cell Epitope
QQQQGQTVTK

SEQ ID NO: 502 - protein, 10 aa, artificial sequence
  T Cell Epitope
SASAFFGMSR

SEQ ID NO: 503 - protein, 10 aa, artificial sequence
  T Cell Epitope
SQASSRSSSR

SEQ ID NO: 504 - protein, 9 aa, artificial sequence
  T Cell Epitope
TPSGTWLTY

SEQ ID NO: 505 - protein, 10 aa, artificial sequence
  T Cell Epitope
FPNITNLCPF

SEQ ID NO: 506 - protein, 10 aa, artificial sequence
  T Cell Epitope
APHGVVFLHV

SEQ ID NO: 507 - protein, 10 aa, artificial sequence
  T Cell Epitope
APSASAFFGM

SEQ ID NO: 508 - protein, 15 aa, artificial sequence
  T Cell Epitope
GAALQIPFAMQMAYR

SEQ ID NO: 509 - protein, 15 aa, artificial sequence
  T Cell Epitope
GWTFGAGAALQIPFA

SEQ ID NO: 510 - protein, 15 aa, artificial sequence
  T Cell Epitope
IDRLITGRLQSLQTY

SEQ ID NO: 511 - protein, 15 aa, artificial sequence
  T Cell Epitope
ISGINASVVNIQKEI

SEQ ID NO: 512 - protein, 15 aa, artificial sequence
  T Cell Epitope
LDKYFKNHTSPDVDL

SEQ ID NO: 513 - protein, 15 aa, artificial sequence
  T Cell Epitope
LGDISGINASVVNIQ

SEQ ID NO: 514 - protein, 15 aa, artificial sequence
  T Cell Epitope
LGFIAGLIAIVMVTI

SEQ ID NO: 515 - protein, 15 aa, artificial sequence
  T Cell Epitope
LNTLVKQLSSNFGAI

SEQ ID NO: 516 - protein, 15 aa, artificial sequence
  T Cell Epitope
LQDVVNQNAQALNTL

SEQ ID NO: 517 - protein, 15 aa, artificial sequence
  T Cell Epitope
LQSLQTYVTQQLIRA

SEQ ID NO: 518 - protein, 15 aa, artificial sequence
  T Cell Epitope
LQTYVTQQLIRAAEI

SEQ ID NO: 519 - protein, 15 aa, artificial sequence
  T Cell Epitope
AQKFNGLTVLPPLLT

SEQ ID NO: 520 - protein, 15 aa, artificial sequence
  T Cell Epitope
PCSFGGVSVITPGTN

SEQ ID NO: 521 - protein, 15 aa, artificial sequence
  T Cell Epitope
QIPFAMQMAYRFNGI

SEQ ID NO: 522 - protein, 15 aa, artificial sequence
  T Cell Epitope
QQLIRAAEIRASANL

SEQ ID NO: 523 - protein, 15 aa, artificial sequence
  T Cell Epitope
QTYVTQQLIRAAEIR

SEQ ID NO: 524 - protein, 15 aa, artificial sequence
  T Cell Epitope
AYRFNGIGVTQNVLY

SEQ ID NO: 525 - protein, 15 aa, artificial sequence
  T Cell Epitope
SSNFGAISSVLNDIL

SEQ ID NO: 526 - protein, 15 aa, artificial sequence
  T Cell Epitope
TGRLQSLQTYVTQQL

SEQ ID NO: 527 - protein, 15 aa, artificial sequence
  T Cell Epitope
WLGFIAGLIAIVMVT

SEQ ID NO: 528 - protein, 15 aa, artificial sequence
  T Cell Epitope
CVNFNFNGLTGTGVL

SEQ ID NO: 529 - protein, 15 aa, artificial sequence
  T Cell Epitope
DKYFKNHTSPDVDLG

SEQ ID NO: 530 - protein, 15 aa, artificial sequence
  T Cell Epitope
IDAYKTFPPTEPKKD

SEQ ID NO: 531 - protein, 15 aa, artificial sequence
  T Cell Epitope
MSRIGMEVTPSGTWL

SEQ ID NO: 532 - protein, 15 aa, artificial sequence
  T Cell Epitope
NKHIDAYKTFPPTEP

SEQ ID NO: 533 - protein, 15 aa, artificial sequence
  T Cell Epitope
VLQLPQGTTLPKGFY

SEQ ID NO: 534 - protein, 9 aa, artificial sequence
  T Cell Epitope
LQIPFAMQM

SEQ ID NO: 535 - protein, 9 aa, artificial sequence
  T Cell Epitope
RVDFCGKGY

SEQ ID NO: 536 - protein, 10 aa, artificial sequence
  T Cell Epitope
YEQYIKWPWY

SEQ ID NO: 537 - protein, 9 aa, artificial sequence
  T Cell Epitope
GRLQSLQTY

SEQ ID NO: 538 - protein, 9 aa, artificial sequence
  T Cell Epitope
VRFPNITNL

SEQ ID NO: 539 - protein, 17 aa, artificial sequence
  T Cell Epitope
DVVNQNAQALNTLVKQL

SEQ ID NO: 540 - protein, 17 aa, artificial sequence
  T Cell Epitope
EAEVQIDRLITGRLQSL

SEQ ID NO: 541 - protein, 28 aa, artificial sequence
  T Cell Epitope
EIDRLNEVAKNLNESLIDLQELGKYEQY

SEQ ID NO: 542 - protein, 17 aa, artificial sequence
  T Cell Epitope
EVAKNLNESLIDLQELG

SEQ ID NO: 543 - protein, 17 aa, artificial sequence
  T Cell Epitope
GAALQIPFAMQMAYRFN

SEQ ID NO: 544 - protein, 8 aa, artificial sequence
  T Cell Epitope
GAGICASY

SEQ ID NO: 545 - protein, 17 aa, artificial sequence
  T Cell Epitope
AISSVLNDILSRLDKVE

SEQ ID NO: 546 - protein, 8 aa, artificial sequence
  T Cell Epitope
GSFCTQLN

SEQ ID NO: 547 - protein, 17 aa, artificial sequence
  T Cell Epitope
ILSRLDKVEAEVQIDRL

SEQ ID NO: 548 - protein, 8 aa, artificial sequence
  T Cell Epitope
KGIYQTSN

SEQ ID NO: 549 - protein, 8 aa, artificial sequence
  T Cell Epitope
AMQMAYRF

SEQ ID NO: 550 - protein, 17 aa, artificial sequence
  T Cell Epitope
KNHTSPDVDLGDISGIN

SEQ ID NO: 551 - protein, 17 aa, artificial sequence
  T Cell Epitope
MAYRFNGIGVTQNVLYE

SEQ ID NO: 552 - protein, 17 aa, artificial sequence
  T Cell Epitope
AATKMSECVLGQSKRVD

SEQ ID NO: 553 - protein, 17 aa, artificial sequence
  T Cell Epitope
PFAMQMAYRFNGIGVTQ

SEQ ID NO: 554 - protein, 17 aa, artificial sequence
  T Cell Epitope
QLIRAAEIRASANLAAT

SEQ ID NO: 555 - protein, 6 aa, artificial sequence
  T Cell Epitope
QQFGRD

SEQ ID NO: 556 - protein, 17 aa, artificial sequence
  T Cell Epitope
RASANLAATKMSECVLG

SEQ ID NO: 557 - protein, 17 aa, artificial sequence
  T Cell Epitope
RLITGRLQSLQTYVTQQ

SEQ ID NO: 558 - protein, 17 aa, artificial sequence
  T Cell Epitope
SLQTYVTQQLIRAAEIR

SEQ ID NO: 559 - protein, 17 aa, artificial sequence
  T Cell Epitope
DLGDISGINASVVNIQK

SEQ ID NO: 560 - protein, 17 aa, artificial sequence
  T Cell Epitope
FFGMSRIGMEVTPSGTW

SEQ ID NO: 561 - protein, 18 aa, artificial sequence
  T Cell Epitope
GLPNNTASWFTALTQHGK

SEQ ID NO: 562 - protein, 6 aa, artificial sequence
  T Cell Epitope
GTTLPK

SEQ ID NO: 563 - protein, 20 aa, artificial sequence
  T Cell Epitope
KHIDAYKTFPPTEPKKDKKK

SEQ ID NO: 564 - protein, 19 aa, artificial sequence
  T Cell Epitope
YNVTQAFGRRGPEQTQGNF

SEQ ID NO: 565 - protein, 15 aa, artificial sequence
  T Cell Epitope
KTFPPTEPKKDKKKK

SEQ ID NO: 566 - protein, 6 aa, artificial sequence
  T Cell Epitope
LLPAAD

SEQ ID NO: 567 - protein, 17 aa, artificial sequence
  T Cell Epitope
LNKHIDAYKTFPPTEPK

SEQ ID NO: 568 - protein, 10 aa, artificial sequence
  T Cell Epitope
LPQGTTLPKG

SEQ ID NO: 569 - protein, 8 aa, artificial sequence
  T Cell Epitope
LPQRQKKQ

SEQ ID NO: 570 - protein, 18 aa, artificial sequence
  T Cell Epitope
PKGFYAEGSRGGSQASSR

SEQ ID NO: 571 - protein, 17 aa, artificial sequence
  T Cell Epitope
QFAPSASAFFGMSRIGM

SEQ ID NO: 572 - protein, 8 aa, artificial sequence
  T Cell Epitope
QGTDYKHW

SEQ ID NO: 573 - protein, 15 aa, artificial sequence
  T Cell Epitope
QLPQGTTLPKGFYAE

SEQ ID NO: 574 - protein, 18 aa, artificial sequence
  T Cell Epitope
QLPQGTTLPKGFYAEGSR

SEQ ID NO: 575 - protein, 22 aa, artificial sequence
  T Cell Epitope
QLPQGTTLPKGFYAEGSRGGSQ

SEQ ID NO: 576 - protein, 8 aa, artificial sequence
  T Cell Epitope
TFPPTEPK

SEQ ID NO: 577 - protein, 8 aa, artificial sequence
  T Cell Epitope
SQASSRSS

SEQ ID NO: 578 - protein, 16 aa, artificial sequence
  T Cell Epitope
SRGGSQASSRSSSRSR

SEQ ID NO: 579 - protein, 5 aa, artificial sequence
  Spacer
CPGPG

SEQ ID NO: 580 - protein, 6 aa, artificial sequence
  Spacer
GSGSGS

SEQ ID NO: 581 - protein, 206 aa, artificial sequence
  RBD Protein
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNL

SEQ ID NO: 582 - protein, 18 aa, Artificial Sequence
  Epitope #1
TESNKKFLPFQQFGRDIA

SEQ ID NO: 583 - protein, 18 aa, Artificial Sequence
  Epitope #2
PSKPSKRSFIEDLLFNKV

SEQ ID NO: 584 - protein, 61 aa, Artificial Sequence
  Epitope #3
VLYENQKLIANQFNSAIGKIQDSLSSTASALGKLQDVVNQNAQALNTLVKQLSSNFGAISS

SEQ ID NO: 585 - protein, 39 aa, Artificial Sequence
  Epitope #4
GAISSVLNDILSRLDKVEAEVQIDRLITGRLQSLQTYVT

(19) United States
(12) Patent Application Publication
(10) Pub. No.: US 2024/0082389 A1
(43) Pub. Date: Mar. 14, 2024

(54) METHODS OF PRODUCING EXTRACELLULAR VESICLES

(71) Applicant: LONZA SALES AG, BASEL (CH)

(72) Inventors: Tim SOOS, Boston, MA (US); Aaron NOYES, Melrose, MA (US); Kevin P. DOOLEY, Boston, MA (US); Eric ZHANG, Cambridge, MA (US)

(73) Assignee: LONZA SALES AG, BASEL (CH)

(21) Appl. No.: 18/246,304

(22) PCT Filed: Sep. 23, 2021
(86) PCT No.: PCT/US2021/051742

§ 371 Date: Mar. 22, 2023

Related U.S. Application Data
(60) Provisional application No. 63/161,331, filed on Mar. 15, 2021.
(60) Provisional application No. 63/082,453, filed on Sep. 23, 2020.

(51) Int. Cl. A61K 39/215; A61K 39/12; A61P 37/02

(52) U.S. Cl. CPC A61K 39/215; A61K 39/12; A61P 37/02; A61K 2039/55555

(57) ABSTRACT

The present disclosure relates to extracellular vesicles (EVs), e.g., exosomes, which can be rapidly modified to comprise an antigen of interest, and the uses of such EVs as vaccines for the treatment of wide range of diseases or disorders. Also provided herein are methods for preparing and manufacturing such EVs for use as vaccines.

### CROSS-REFERENCE TO RELATED APPLICATION

This PCT application claims the priority benefit of U.S. Provisional Application No. 63/082,453, filed on Sep. 23, 2020; and U.S. Provisional Application No. 63/161,331, filed on Mar. 15, 2021; each of which is herein incorporated by reference in its entirety.

### REFERENCE TO SEQUENCE LISTING SUBMITTED ELECTRONICALLY VIA EFS-WEB

The content of the electronically submitted sequence listing (Name: 0132-0297US1_Seqlisting_ST25.txt, Size: 463,375 bytes; and Date of Creation: Nov. 1, 2023) submitted in this application is incorporated herein by reference in its entirety.

### FIELD OF DISCLOSURE

The present disclosure relates to modified extracellular vesicles (EVs) (e.g., exosomes) that are suitable to be used as vaccines. The present disclosure also relates to methods of preparing such EVs, and their uses to treat a range of medical disorders.

### BACKGROUND

While many traditional vaccines (e.g., peptide-based) have been used to treat and/or prevent certain diseases or disorders, they are generally poorly immunogenic and require repeated administrations and/or high doses. See, e.g., Hos, B. J., et al., Front Immunol 9:884 (2018). Additionally, because of manufacturing complexities, compounded by the need for different formulations for different countries and age groups, it often takes multiple years to develop and manufacture a safe and efficacious vaccine. See, e.g., Smith, J., et al., Lancet 378(9789): 428-438 (July 2011). As is apparent with the ongoing COVID-19 pandemic, the inability to rapidly produce a safe and efficacious vaccine could have dire consequences. Accordingly, there is a need for new and more effective vaccines that can be rapidly produced and tailored to any ongoing outbreaks.

### SUMMARY OF DISCLOSURE

Certain aspects of the present disclosure are directed to a method of preparing an extracellular vesicle (EV) for a vaccine, comprising loading an antigen to an EV that has been isolated from a producer cell.

Certain aspects of the present disclosure are directed to a method of manufacturing a vaccine for a disease or disorder, comprising loading an antigen to an extracellular vesicle (EV) that has been isolated from a producer cell.

In some aspects, the vaccine is regionalized or individualized.

In some aspects, the antigen is: (i) linked to the exterior surface of the EV, (ii) linked to the luminal surface of the EV, (iii) in the lumen of the EV, or (iv) any combination thereof.

In some aspects, the EV further comprises an adjuvant. In some aspects, the adjuvant comprises a non-toxic mutant of diphtheria toxin (e.g., CRM-197), peptide adjuvant (e.g., muramyl dipeptide (MDP)), STING agonist, TLR agonist, universal T cell helper peptide (e.g., PADRE), or any combinations thereof. In some aspects, the EV comprises the adjuvant prior to the loading of the antigen to the EV.

In some aspects, the method further comprises loading the adjuvant. In some aspects, the adjuvant is loaded before or after the loading of the antigen. In some aspects, the adjuvant is loaded together with the antigen.

In some aspects, the antigen and/or the adjuvant is linked to the exterior surface and/or the luminal surface of the EV by an anchoring moiety, affinity agent, chemical conjugation, cell penetrating peptide (CPP), split intein, SpyTag/SpyCatcher, ALFA-tag, Streptavidin/Avitag, Sortase, SNAP-tag, ProA/Fc-binding peptide, or any combinations thereof.

In some aspects, the anchoring moiety comprises a sterol (e.g., cholesterol), GM1, lipid, (e.g., fatty acid (e.g., palmitate), ionizable lipid, glycerophospholipid, sphingolipid), vitamin (e.g., tocopherol (e.g., vitamin E), vitamin A, vitamin D, vitamin K), alkyl chain, aromatic ring, small molecule, peptide, including any derivatives thereof, or any combination thereof. In some aspects, the anchoring moiety is attached to the antigen by a linker.

In some aspects, the linker comprises a polypeptide linker, non-polypeptide linker, or both. In some aspects, the linker comprises a hydrophilic linker (e.g., PEG and derivatives). In some aspects, the linker comprises a hydrophobic linker (e.g., alkyl chain, hexamethylene). In some aspects, the linker comprises a cleavable linker (e.g., phosphodiesters, valine citrulline, disulfide bond, acid-labile). In some aspects, the chemical conjugation comprises a maleimide moiety, copper-free, biorthogonal click chemistry (e.g., azide/strained alkyne (DIFO, dibenzocyclooctyne (DBCO), bicyclononyne (BCN)), metal-catalyzed click chemistry (e.g., CuAAC, RuAAC), or any combination thereof.

In some aspects, the antigen comprises a peptide. In some aspects, the peptide comprises a natural peptide, synthetic peptide, or both. In some aspects, the peptide comprises a lysine. In some aspects, the lysine is a N-terminal lysine. In some aspects, the peptide comprises unnatural amino acids with side chains that allow for the binding of an azide, strained alkynes, maleimide, pentafluorophenyl (PFP) esters, or NHS.

In some aspects, the peptide is less than about 150 amino acids in length, less than about 140 amino acids in length, less than about 130 amino acids in length, less than about 120 amino acids in length, less than about 110 amino acids in length, less than about 100 amino acids in length, less than about 90 amino acids in length, less than about 80 amino acids in length, less than about 70 amino acids in length, less than about 60 amino acids in length, less than about 50 amino acids in length, less than about 40 amino acids in length, less than about 30 amino acids in length, less than about 20 amino acids in length, or less than about 10 amino acids in length. In some aspects, the peptide is less than about 100 amino acids in length. In some aspects, the peptide is less than about 80 amino acids in length.

In some aspects, the antigen comprises a single epitope of an antigen. In some aspects, the antigen comprises a concatemer of multiple epitopes of an antigen. In some aspects, one or more of the multiple epitopes are separated by a spacer. In some aspects, the peptide comprises a linear epitope of the protein from which it is derived, a conformational epitope, or both. In some aspects, the spacer comprises the amino acid sequence CPGPG (SEQ ID NO: 579), AAY, GSGSGS (SEQ ID NO: 580), or any combination thereof.

In some aspects, the antigen comprises a CD8+ T cell epitope, CD4+ T cell epitope, B cell epitope, or any combination thereof.

In some aspects, the antigen is derived from and/or comprises a virus, a bacterium, a parasite, a fungus, a protozoa, a tumor, an allergen, a self-antigen, or any combination thereof. In some aspects, the antigen is derived from a virus causing a pandemic. In some aspects, the antigen is derived from a coronavirus, an influenza virus, an Ebola virus, a Chikungunya virus (CHIKV), a Crimean-Congo hemorrhagic fever (CCGF) virus, a Hendra virus, a Lassa virus, a Marburg virus, a monkeypox virus, a Nipah virus, a Hendra virus, a Rift Valley fever (RVF) virus, a Variola virus, a yellow fever virus, a Zika virus, a measles virus, a human immunodeficiency virus (HIV), a hepatitis C virus (HCV), a dengue fever virus (DENY), a parvovirus (e.g., B19 virus), a norovirus, a respiratory syncytial virus (RSV), a lentivirus, an adenovirus, a flavivirus, a filovirus, an alphavirus (e.g., a rhinovirus), a human papillomavirus (HPV), Eastern equine encephalitis (EEE), West Nile virus, Epstein Barr virus (EBV), Cytomegalovirus (CMV), Hepatitis B virus (HBV), John Cunningham virus (JCV), Japanese and tick-borne encephalitis, encephalitic equine viruses, Human Metapneumovirus (hMPV), rabies, or any combination thereof. In some aspects, the antigen is derived from Vibrio cholera, Yersinia pestis, Mycobacterium tuberculosis (MTB), streptococcus (e.g., Streptococcus pyogenes, Streptococcus agalactiae, Streptococcus pneumoniae), staphylococcal (e.g., Staphylococcus aureus), shigella, Escherichia coli, salmonella, chlamydia (e.g., Chlamydia trachomatis), Pseudomonas aeruginosa, Klebsiella pneumoniae, Haemophilus influenza, Clostridia difficile, Plasmodium, Leishmania, Schistosoma, Trypanosoma, Brucella, Cryptosporidium, Entamoeba, Neisseria meningitis, Bacillus subtilis, Haemophilius influenzae, Neisseria gonorrhoeae, Borrelia burgdorferi, Corynebacterium diphteriae, Moraxella catarrhalis, Campylobacter jejuni, Clostridium tetanus, Clostridium perfringens, Treponema pallidum or any combination thereof.

In some aspects, the coronavirus comprises a severe acute respiratory syndrome (SARS) coronavirus, a Middle East respiratory syndrome-related coronavirus (MERS-CoV), or both. In some aspects, the SARS coronavirus comprises SARS-CoV-1, SARS-CoV-2 (COVID-19), or both. In some aspects, the antigen is a universal SARS coronavirus antigen.

In some aspects, the antigen comprises a CD8+ T cell epitope selected from LITGRLQSL (SEQ ID NO: 423), FIAGLIAIV (SEQ ID NO: 425), RLNEVAKNL (SEQ ID NO: 439), VVFLHVTYV (SEQ ID NO: 427), VLNDILSRL (SEQ ID NO: 429), ALNTLVKQL (SEQ ID NO: 445), NLNESLIDL (SEQ ID NO: 431), MEVTPSGTWL (SEQ ID NO: 451), GMSRIGMEV (SEQ ID NO: 452), ILLNKHIDA (SEQ ID NO: 453), ALNTPKDHI (SEQ ID NO: 454), LALLLLDRL (SEQ ID NO: 457), LLLDRLNQL (SEQ ID NO: 458), LQLPQGTTL (SEQ ID NO: 460), RLNQLESKV (SEQ ID NO: 448), or any combination thereof. In some aspects, the antigen comprises a CD8+ T cell epitope selected from FIAGLIAIV (SEQ ID NO: 425), LITGRLQSL (SEQ ID NO: 423), RLNEVAKNL (SEQ ID NO: 439), NLNESLIDL (SEQ ID NO: 431), ALNTLVKQL (SEQ ID NO: 445), or any combination thereof.

In some aspects, the antigen comprises a CD4+ T cell epitope having the amino acid sequence AKFVAAWTLKAAA (SEQ ID NO: 382). In some aspects, the antigen comprises a B cell epitope selected from TESNKKFLPFQQFGRDIA (SEQ ID NO: 582), PSKPSKRSFIEDLLFNKV (SEQ ID NO: 583), or both. In some aspects, the antigen comprises both T cell and B cell epitopes.

In some aspects, the antigen comprises the amino acid sequence VLYENQKLIANQFNSAIGKIQDSLSSTASALGKLQDVVNQNAQALNTLVKQLSSNFGAIS S (SEQ ID NO: 584), GAISSVLNDILSRLDKVEAEVQIDRLITGRLQSLQTYVT (SEQ ID NO: 585), or both.

In some aspects, the loading of the antigen and/or adjuvant to the EV occurs at least about 1 day, at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 2 years, at least about 3 years, at least about 4 years, at least about 5 years, at least 6 years, at least 7 years, at least 8 years, at least 9 years, at least 10 years, at least 15 years, at least 20 years, at least 25 years or more after isolating the EV from the producer cell.

In some aspects, the loading efficiency of the antigen and/or adjuvant to the EVs is increased compared to a reference loading efficiency (e.g., loading efficiency of the antigen and/or adjuvant without the use of an anchoring moiety, affinity agent, chemical conjugation, cell penetrating peptide (CPP), split intein, SpyTag/SpyCatcher, ALFA-tag, Streptavidin/Avitag, Sortase, SNAP-tag, ProA/Fc-binding peptide, or any combinations thereof). In some aspects, the loading efficiency is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, at least about 10,000-fold or more, compared to the reference loading efficiency.

In some aspects, the time required for manufacturing the vaccine (“manufacturing time”) is reduced compared to a reference manufacturing time (e.g., manufacturing time of a method wherein the loading of the antigen occurs by introducing the antigen into the producer cell, or manufacturing time of a method for producing a vaccine that does not comprise an EV, such as a traditional peptide vaccine). In some aspects, the manufacturing time is reduced by at least about 10%, at least about 20%, at least about 30%, at least about 40%, at least about 50%, at least about 60%, at least about 70%, at least about 80%, or at least about 90% or more, compared to the reference manufacturing time. In some aspects, the manufacturing time is less than about 12 months, less than about 11 months, less than about 10 months, less than about 9 months, less than about 8 months, less than about 7 months, less than about 6 months, less than about 5 months, less than about 4 months, less than about 3 months, less than about 2 months, or less than about 1 month. In some aspects, the manufacturing time is less than about 6 months.

In some aspects, the EV further comprises a first scaffold moiety. In some aspects, the EV comprises the first scaffold moiety prior to the loading of the antigen to the EV. In some aspects, the antigen is linked to the first scaffold moiety. In some aspects, the adjuvant is linked to the first scaffold moiety.

In some aspects, the EV further comprises a second scaffold moiety. In some aspects, the EV comprises the second scaffold moiety prior to the loading of the antigen to the EV.

In some aspects, the antigen is linked to the first scaffold moiety, and the adjuvant is linked to the second scaffold moiety.

In some aspects, the first scaffold moiety and the second scaffold moiety are the same. In some aspects, the first scaffold moiety and the second scaffold moiety are different. In some aspects, the first scaffold moiety is a Scaffold X. In some aspects, the first scaffold moiety is a Scaffold Y. In some aspects, the second scaffold moiety is a Scaffold Y. In some aspects, the second scaffold moiety is a Scaffold X.

In some aspects, the Scaffold X comprises a prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), or any combination thereof. In some aspects, the Scaffold X is PTGFRN or a fragment thereof.

In some aspects, the Scaffold Y comprises myristoylated alanine rich Protein Kinase C substrate (the MARCKS protein); myristoylated alanine rich Protein Kinase C substrate like 1 (the MARCKSL1 protein); brain acid soluble protein 1 (the BASP1 protein), and any combination thereof.

In some aspects, the antigen is linked to a first scaffold moiety on the exterior surface of the base EV. In some aspects, the adjuvant is: (i) linked to a second scaffold moiety on the exterior surface of the base EV; (ii) linked to a second scaffold moiety on the luminal surface of the base EV; (iii) in the lumen of the EV; or (iv) any combination of (i), (ii), and (iii).

In some aspects, the EV further comprises a targeting moiety. In some aspects, the EV comprises the targeting moiety prior to the loading of the antigen to the EV. In some aspects, the method further comprises loading the targeting moiety. In some aspects, the targeting moiety are loaded before or after the loading of the antigen. In some aspects, the targeting moiety are loaded together with the antigen. In some aspects, after the loading of the antigen, the EV is capable of inducing a T-cell immune response, a B-cell immune response, or both T-cell and B-cell immune responses.

Certain aspects of the present disclosure are directed to an extracellular vesicle (EV) prepared by any methods disclosed herein.

Certain aspects of the present disclosure are directed to a kit comprising an EV disclosed herein, and instructions for use.

Certain aspects of the present disclosure are directed to a kit comprising a first container and a second container, wherein the first container comprises an extracellular vesicle that has been isolated from a producer cell, and the second container comprises an antigen, wherein combining the first container and the second container results in the loading of the antigen to the EV, such that the antigen is coupled to a surface of the EV.

Certain aspects of the present disclosure are directed to a vaccine comprising an EV disclosed herein, wherein the antigen is capable of eliciting an immune response in a subject that receives an administration of the vaccine. In some aspects, the vaccine is regionalized or individualized.

Certain aspects of the present disclosure are directed to a method of treating a disease or disorder in a subject in need thereof, comprising administering an EV disclosed herein to the subject.

Certain aspects of the present disclosure are directed to a method of treating a disease or disorder in a subject in need thereof, comprising administering a vaccine disclosed herein to the subject. In some aspects, the disease or disorder comprises an infectious disease.

### BRIEF DESCRIPTION OF FIGURES

FIG. 1 shows a schematic illustrating an exemplary process involved in preparing the EV-based vaccines of the present disclosure. The exemplary antigens, attachment moieties, and adjuvants shown can be substituted with any of the other antigens, attachment moieties, and adjuvants provided herein.

FIGS. 2A, 2B, and 2C show exosomes that have been modified to comprise an acceptor domains fused to the N-terminus of a PTGFRN protein. The acceptor domains included: (1) SpyCatcher (diamond), (2) CfaC (square), and (3) ALFANb (triangle). FIG. 2A shows the cell growth of stable HEK293 cells lines engineered to overexpress each acceptor domain fused to PTGFRN during exosome production. FIG. 2B shows the viability of the stable cell pool over the same time period as shown in FIG. 2A. FIG. 2C shows the successful purification of exosomes comprising the acceptor domain-PTGFRN fusion protein using iodixanol density gradient ultracentrifugation. FIG. 2D provides SDS-PAGE analysis showing the transgene expression of each acceptor domain compared to un-engineered exosomes (WT).

FIG. 3 provides a workflow for assessing the loading of NanoLuc fused to a donor domain onto exosomes expressing the cognate acceptor domain fused to PTGFRN. Unbound NanoLuc is removed by ultracentrifugation (UC) and the resulting exosomes are analyzed by a variety of assays.

FIG. 4A provides a workflow for labeling exosomes overexpressing ALFA nanobody (ALFANb) with NanoLuc fused to the ALFAtag. FIG. 4B provides SDS-PAGE analysis post-loading and UC cleanup showing association of NanoLuc-ALFAtag with ALFANb expressing exosomes.

FIG. 5A provides standard curves of NanoLuc fused to ALFA tag or poly-histidine tags. FIG. 5B provides a quantitative analysis of the loading of (i) NanoLuc fused to ALFA tag or (ii) NanoLuc fused to poly-histidine tag on the modified exosomes. The quantitative value was determined using the standard curves from FIG. 5A. The fold-change over the background histidine-tag expression is noted.

FIG. 6A provides a workflow for assessing the stability of the ALFA-NanoLuc interaction over time. FIG. 6B provides a quantitative analysis of the stability of the ALFA-NanoLuc expression compared to NanoLuc fused to a poly-histidine tag.

FIG. 7A provides a workflow for labeling exosomes overexpressing CfaC with NanoLuc fused to the CfaN. FIG. 7B provides SDS-PAGE analysis post-loading and UC cleanup showing association of NanoLuc-CfaN with CfaC expressing exosomes.

FIG. 8A provides standard curves of NanoLuc fused to CfaN or a polyhistidine tag. FIG. 8B provides a quantitative analysis of the loading of (i) NanoLuc fused to CfaN or (ii) NanoLuc fused to a poly-histidine tag on the modified exosomes. The quantitative value was determined using the standard curves from FIG. 8A. The fold-change over the background histidine-tag expression is noted.

FIG. 9A provides the workflow for labeling exosomes overexpressing SpyCatcher with NanoLuc fused to the SpyTag. FIG. 9B provides SDS-PAGE analysis post-loading and UC cleanup showing association of NanoLuc-SpyTag with SpyCatcher expressing exosomes.

FIG. 10A provides standard curves of NanoLuc fused to SpyTag or a poly-histidine tag. FIG. 10B provides a quantitative analysis of the loading of (i) NanoLuc fused to SpyTag or (ii) NanoLuc fused to poly-histidine tag on the modified exosomes. The quantitative value was determined using the standard curves from FIG. 10A. The fold-change over the background histidine-tag expression is noted

FIG. 11 provides a comparison of the loading of NanoLuc fused to one of the following donor domains in the modified exosomes: (i) ALFAtag, (ii) CfaN, and (iii) SpyTag.

FIG. 12 shows a schematic illustrating surface conjugation of an exemplary moiety of interest to a scaffold protein associated with an exosome membrane.

FIG. 13 shows a table listing mechanistic characteristics of exemplary small molecules and oligonucleotides conjugated to exosomes. pAB-pNP: p-aminobenzyl alcohol p-nitrophenyl carbonate; DBCO: dibenzylcyclooctyne.

FIG. 14 shows a table listing lipid-linker-reactive groups as intermediates. The reactive group on the linker and its respective functional group on the moiety of interest is listed.

FIGS. 15A, 15B, 15C, and 15D show examples of MCC-950, a small molecule, conjugation. FIG. 15A shows MCC-950-Val-Cit-Maleimide conjugation. FIG. 15B shows MCC-950-Val-Cit-Methacrylate conjugation. FIG. 15C shows MCC-950-Val-Cit-Pyridinyl Disulfide conjugation. FIG. 15D shows a schematic illustrating the conjugation site of a sulfhydryl group on an exosome bound scaffold protein.

FIGS. 16A-16B show examples of oligonucleotide/protein conjugation. FIG. 16A shows an oligo-maleimide conjugation and a schematic illustrating its conjugation site of a sulfhydryl group on an exosome bound scaffold protein. FIG. 16B shows an oligo-NHS ester conjugation and a schematic illustrating its conjugation site on a primary amine group on an exosome bound scaffold protein.

FIG. 17 shows a table listing the function group on the scaffold protein of exosomes, activation thereof, and functional group on the protein/peptide to be conjugated, and the degradability of the linkage. Due to the same functional groups, options for protein to protein/peptide conjugation is limited to click chemistry. DBCO: dibenzylcyclooctyne.

FIG. 18 shows an example of protein/peptide conjugation (Schiff base enabled).

FIG. 19 shows an example of protein/peptide conjugation (click chemistry).

FIGS. 20A and 20B provide schematic of EVs comprising the ALFA plug and play system described herein. As shown in FIG. 20A, ALFA nanobody (NbALFA) is fused to Scaffold X (e.g., PTGFRN) on the exterior surface of the EV (“NbALFA EV”). The crystal structure shows the stable, non-covalent interaction of the NbALFA with the ALFAtag. FIG. 20B shows the loading of moieties of interest onto the exterior surface of the NbALFA EVs. As further described herein (see, e.g., Example 16), the moieties of interest are fused to an ALFAtag and then, mixed with the NbALFA EVs, resulting in the stable association of the ALFA-tagged moieties of interest to the exterior surface of the NbALFA EVs. The EVs can be loaded with the same moiety of interest (see top drawing) or a mixture of different moiety of interest (see bottom drawing).

FIG. 21 provides SDS-PAGE (top) and Western blot (bottom) results demonstrating that NbALFA EVs can be simultaneously loaded with multiple moieties of interest. Wild-type EVs or NbALFA EVs were mixed with 10 μg of NLuc-ALFAtag or molar equivalent of mouse IL-12 fused to ALFAtag (“mIL12-ALFAtag”).

FIGS. 22A and 22B show anti-RBD antibody responses in mice vaccinated with EVs comprising the RBD region of coronavirus spike protein (fused to the N-terminus of PTGFRN) (“exoRBD”). Control animals were vaccinated with recombinant RBD (“rRBD”) or control EVs (i.e., comprising PTGFRN but not RBD) (“exosomes”). The animals received three total doses of a treatment regimen over a course of four weeks (see arrows). exoRBD was administered to the animals at one of three doses: 8 μg (black circle), 2.6 μg (gray circle), and 1.3 μg (white circle). rRBD was administered to the animals at one of two doses: 8 μg (black square) and 2.6 μg (gray square). FIG. 22A provides a comparison of the total anti-RBD IgG levels in the sera of animals from the different treatment groups. FIG. 22B provides a comparison of the neutralizing activity of the anti-RBD antibodies.

FIGS. 23A and 23B show the effect of STING agonist on anti-RBD antibody responses in mice vaccinated with EVs comprising the RBD region of coronavirus spike protein (fused to the N-terminus of PTGFRN) (“exoRBD”). Animals received two doses (two weeks apart; see arrows) of the following treatment regimen: (i) exoRBD loaded with STING agonist in the lumen of the EV (“exoRBD+STING”; black diamond); or (ii) exoRBD alone (i.e., not loaded with the STING agonist) (“exoRBD”; black square). FIG. 23A provides a comparison of the total anti-RBD IgG levels in the sera of animals from the different treatment groups. FIG. 23B provides a comparison of the neutralizing activity of the anti-RBD antibodies. The dashed line in FIGS. 23A and 23B represent corresponding antibody levels observed in the sera of human subjects who received two doses of a COVID19 mRNA vaccine.

FIGS. 24A and 24B show neutralization and RBD-binding activity, respectively, of anti-RBD antibodies from mice and humans vaccinated against a coronavirus. Mice received two doses (two weeks apart) of one of the following treatment regimen: (i) PBS alone (“1-PBS”); (ii) a mixture of recombinant RBD (2.6 μg), soluble STING agonist (i.e., not loaded in an EV), and control EV (comprising PTGFRN alone; “PrX-exo”) (“rRBD+STING+PrX-exo”); and (iii) exoRBD (2.6 μg) loaded with STING agonist in the lumen of the EV (“exoRBD+STING”). Human subjects received two doses of a COVID19 mRNA vaccine (“Human Vax Donor 2nd”; fourth bar). In FIG. 24A, neutralizing antibody activity is shown as the titer required to elicit 50% neutralization of RBD-hACE2 protein-protein interaction. In FIG. 24B, the ability to bind to recombinant RBD variants of concern (i.e., kappa, delta, alpha, beta, and gamma) are provided for anti-RBD antibodies from vaccinated humans (second bar in each of the groups provided on the x-axis) and mice from the exoRBD+STING group (first bar in each of the groups provided on the x-axis). Binding activity is shown as a fold change relative to binding to wild-type RBD.

FIGS. 25A, 25B, 25C, and 25D provide comparison of antibody isotype switching and germinal center formation in mice that received two doses (two weeks apart) of one of the following: (i) PBS alone (“PBS”), (ii) recombinant RBD alone (“rRBD”), (iii) recombinant RBD in combination with soluble STING agonist (“rRBD+STING”), (iv) EVs comprising the RBD region of coronavirus spike protein (fused to the N-terminus of PTGFRN) (“exoRBD”), and (v) exoRBD loaded with a STING agonist in the lumen of the EV (“exoRBD+STING”). FIG. 25A shows the amount of different anti-RBD IgG isotypes (IgG1, IgG2a, IgG2b, and IgG3) in the sera of mice from groups (ii) (“rRBD”), (iv) (“exoRBD”), and (v) (“exoRBD+STING”). FIG. 25B shows the amount of anti-RBD IgG2a, IgG2b, and IgG3 antibodies relative to the amount of anti-RBD IgG1 antibodies in the sera of mice described in FIG. 25A. FIG. 25C provide a comparison of the amount of germinal center B cells (GL7+CD38−) observed in the draining lymph nodes of mice from groups (i) (“PBS”), (iii) (“rRBD+STING”), and (v) (“exoRBD+STING”). The amount of germinal center B cells is shown as a percentage of total B cells observed in the draining lymph nodes. FIG. 25D provides a comparison of plasma cell formation (shown as a percentage of total non-germinal center B cells) in the draining lymph nodes of mice from groups (i) (“PBS”), (iii) (“rRBD+STING”), and (v) (“exoRBD+STING”).

FIGS. 25E, 25F, and 25G show the effect of STING agonist on T cell responses in mice vaccinated with EVs comprising the RBD region of coronavirus spike protein (fused to the N-terminus of PTGFRN) (“exoRBD”). Animals received two doses (two weeks apart) of the following treatment regimen: (i) exoRBD loaded with STING agonist in the lumen of the EV (“exoRBD+STING”; black triangle); (ii) recombinant RBD in combination with soluble STING agonist (“rRBD+STING”; white square); or (iii) PBS alone (white circle). FIG. 25E provides a comparison of the amount of IFN-γ produced by the splenic T cells after antigen restimulation in vitro. FIG. 25F provides a comparison of the amount of IP-10 produced by the splenic T cells after antigen restimulation in vitro. FIG. 25G provides a comparison of the amount of IL-6 produced by the splenic T cells after antigen restimulation in vitro.

FIGS. 26A and 26B show the use of the plug-and-play system described herein (ALFA-tag) to engineer EVs to comprise the RBD region of a coronavirus spike protein. FIG. 26A provides a schematic illustrating the interaction between ALFA-tagged RBD protein and ALFA nanobody (NbALFA) on the exterior surface of the EV (“exoRBD-ALFA”). FIG. 26B shows the number of ALFA-tagged RBD proteins observed on the exterior surface of the EVs. As further described in Example 8, EVs modified to comprise NbALFA acceptor molecules on the exterior surface of the EVs were loaded with different concentrations (high, medium, or low) of the ALFA-tagged RBD proteins. For comparison purposes, EVs comprising RBD fused to the N-terminus of PTGFRN were used as control.

FIGS. 27A, 27B, 27C, 27D, 27E, and 27F show the in vivo immunogenicity of the exoRBD-ALFA EVs (described in FIG. 26A). As further described in Example 8, mice received three total doses (two weeks apart between doses) of one of the following: (i) PBS alone, (ii) exoRBD-ALFA, (iii) recombinant RBD alone (“rRBD”), (iv) rRBD in combination with soluble STING agonist (“rRBD+STING”), and (v) control EVs (i.e., comprising PTGFRN but not RBD) (“exosomes”). To measure, anti-RBD antibody levels, sera was collected from the animals on days 14, 28, 40, and 61 post initial treatment. FIG. 27A shows total anti-RBD IgG antibody levels in the sera of animals that received the exoRBD-ALFA at three different doses. FIG. 27B shows total anti-RBD IgG antibody levels in the sera of animals that received rRBD (alone or in combination with soluble STING agonist or control exosomes). FIG. 27C shows total anti-RBD IgG antibody levels in the sera of animals that received PBS alone or the control exosomes alone. FIGS. 27D, 27E, and 27F provide individual titration series showing the neutralization activity of the anti-RBD antibodies from a representative animal that were immunized with one of the following: (1) exoRBD-ALFA (10.9 μg) (FIG. 27D), (2) rRBD alone (8 μg) (FIG. 27E), and (3) rRBD (8 μg) in combination with the control exosome (FIG. 27F).

FIG. 28 shows the loading of ALFA-tagged CD40L proteins on EVs modified to comprise ALFA nanobody (NbALFA) on their exterior surface—i.e., “exoCD40L(ALFA).” As described further in Example 9, the ALFA-tagged CD40L protein was loaded on to the modified EVs at three different concentrations: high (left bar; “exoCD40L(ALFA)-high”), medium (middle bar; “exoCD40L(ALFA)-mid”), and low (right bar; “exoCD40L(ALFA)-low”). The successful loading of the ALFA-tagged CD40L protein is shown by the number of ALFA-tagged CD40L proteins observed on the exterior surface of the modified EVs.

FIGS. 29A, 29B, 29C, 29D, 29E, and 29F show the ability of exo-CD40L(ALFA) (described in FIG. 28) to regulate B cells in vitro. FIG. 29A provides a schematic of the experimental design. FIGS. 29B and 29C show the proliferation and survival, respectively, of mouse splenic B cells cultured in vitro for three days in the presence of one of the following: (i) exoCD40L(ALFA)-high, (ii) exoCD40L(ALFA)-mid, (iii) exoCD40L(ALFA)-low, (iv) soluble ALFA-tagged CD40L protein (i.e., not fused to the NbALFA on the exterior surface of the EVs), (v) a multimeric CD40L protein alone, and (vi) EVs modified to comprise NbALFA but not fused to the ALFA-tagged CD40L protein. FIGS. 29D and 29E show the expression of activation markers (IA/IE and CD69, respectively) on mouse splenic B cells cultured as described in FIGS. 29B and 29C. FIG. 29F show the presence of B cell clusters (arrows as an example of a cluster), as observed using IncuCyte Zoom, in the different culture conditions described in FIGS. 29B and 29C.

FIGS. 30A, 30B, 30C, 30D, 30E, 30F, and 30G show the ability of exo-CD40L(ALFA) (described in FIG. 28) to regulate B cell immune response in vivo. As further described in Example 10, wild-type mice were treated with one of the following and immune response assessed three days after treatment: (i) EVs modified to comprise NbALFA but not fused to the ALFA-tagged CD40L protein (“exo-Nb ALFA”); (ii) EVs modified to comprise NbALFA and loaded with ALFA-tagged CD40L protein (“exo-Nb ALFA+mCD40L(ALFA)”); (iii) soluble ALFA-tagged CD40L protein (i.e., not fused to the NbALFA on the exterior surface of the EVs) (“mCD40L(ALFA)”); (iv) agonistic anti-CD40 antibody (100 μg); and (v) PBS alone. FIG. 30A provides the total number of live cells in the spleen of animals from the different treatment groups. FIGS. 30B and 30C show the mean fluorescence intensity (MFI) of the expression of certain activation markers (CD69 and IA/IE, respectively) on mouse splenic B cells. FIGS. 30D and 30E show the mean fluorescence intensity (MFI) of the expression of certain activation markers (CD69 and IA/IE, respectively) on B cells from peritoneal lavage. FIGS. 30F and 30G show the mean fluorescence intensity (MFI) of IA/IE expression on myeloid cells (CD45⁺ CD19⁻ CD11b^{hi}) from the spleen and peritoneal lavage, respectively.

FIGS. 31A and 31B show anti-RBD immune response in mice that received two doses (two weeks apart) of one of the following treatment regimen: (i) exoRBD-ALFA (described in FIG. 26A); (ii) combination of exoRBD-ALFA and exoCD40L-ALFA (described in FIG. 28); (iii) EVs modified to comprise NbALFA acceptor molecules on the exterior surface and loaded with both ALFA-tagged RBD and ALFA-tagged CD40L protein (“exo-ALFA-RBD&CD40L dual”); (iv) exoCD40L-ALFA in combination with an agonistic anti-CD40 antibody (“exo-ALFA-RBD+anti-CD40 Ab”); and (v) PBS alone. FIG. 31A provides a comparison of the total anti-RBD IgG antibody levels in the sera of animals from the different treatment groups. FIG. 31B provides a comparison of the neutralizing activity of the anti-RBD IgG antibodies.

FIG. 32 provide comparison of germinal center formation in the spleen of mice from the different treatment groups described in FIGS. 31A and 31B.

FIGS. 33A, 33B, 33C, 33D, 33E, and 33F show in vitro activity of exoRBD loaded with different adjuvants. FIG. 33A provides a schematic of the experimental design. FIG. 33B shows the structure of the different adjuvants tested: TLR4 agonist (MPLA) and TLR7/8 agonists (3M-052 and R848). FIG. 33C show the in vitro activity of the adjuvants (MPLA, 3M-052, and R848) alone (i.e., not loaded in EVs) as measured using the RAW-Blue reporter assay. FIG. 33D show the in vitro activity of exoRBD loaded with MPLA (“exoRBD-MPLA”), 3M-052 (“exoRBD-3M-052”) or R848 (“exoRBD-R848”) as measured using the RAW-Blue reporter assay. FIG. 33E show the effect of exoRBD-MPLA treatment on cell viability as measured using the CellTiter-Glo assay. Cells treated with control EVs (i.e., comprising PTGFRN but not RBD; “exosomes alone”) were used as control. FIG. 33F show the effect of exoRBD-3M-052 and exoRBD-R848 treatment on cell viability as measured using the CellTiter-Glo assay. Cells treated with PBS alone were used as control.

FIGS. 34A, 34B, 34C, and 34D show in vivo activity of exoRBD loaded with a STING agonist or the adjuvants shown in FIG. 33B (i.e., MPLA, 3M-052, and R848). Wild-type mice received two doses (two weeks apart) of one of the following: (i) exoRBD loaded with a STING agonist (“exoRBD+STING”); (ii) exoRBD loaded with MPLA (“exoRBD+MPLA”); (iii) exoRBD loaded with 3M-052 (“exoRBD+3M-052”); (iv) exoRBD loaded with R848 (“exoRBD+R848”); and (v) exoRBD alone (i.e., not loaded with any adjuvant). FIG. 34A provides a comparison of the total anti-RBD IgG antibody levels in the sera of animals from the different treatment groups. FIG. 34B shows the anti-RBD IgG antibody level in the sera of animals from groups (i), (ii), (iii), and (iv) (i.e., exoRBD loaded with an adjuvant) relative to the corresponding amount in the sera of animals that received exoRBD alone. FIG. 34C provides a comparison of the neutralization activity of the anti-RBD IgG antibodies. The dark dashed lines represent anti-RBD IgG antibody levels or neutralization activity in the sera of human subjects at two weeks after receiving the second dose of a COVID19 mRNA vaccine. FIG. 34D shows the neutralization activity of the anti-RBD IgG antibodies in the sera of animals from groups (i), (ii), (iii), and (iv) (i.e., exoRBD loaded with an adjuvant) relative to the corresponding activity in animals that received exoRBD alone (dashed line).

FIGS. 35A, 35B, 35C, 35D, and 35E show the effect of different dosing regimens (incremental dosing, constant dosing, and bolus dosing) on the total anti-RBD IgG antibody levels in mice treated with either exoRBD or exoRBD loaded with the MPLA adjuvant (“exoRBD-MPLA”) (on the exterior surface of the EVs). Control animals received PBS alone. FIG. 35A provides the experimental design. For “incremental dosing,” the cumulative dose administered was given as 6 doses with each dose being greater (double) than the previous dose. For “constant dosing,” the cumulative dose administered was also given as 6 doses, but each dose was the same as the previous dose. For “bolus dosing,” the cumulative dose was given as 2 doses. FIGS. 35B and 35D provide comparison of the total anti-RBD IgG antibody levels in the sera of animals that received the exoRBD or exoRBD-MPLA, respectively, using the different dosing regimens. The dark dashed line represents anti-RBD IgG antibody levels in the sera of human subjects at two weeks after receiving the second dose of a COVID19 mRNA vaccine. FIGS. 35C and 35E show the total anti-RBD IgG antibody levels in the sera of animals that were treated with exoRBD or exoRBD-MPLA, respectively, using either incremental or constant dosing regimen, relative to the corresponding level in the sera of exoRBD-treated animals using the bolus dosing regimen.

FIGS. 36A, 36B, and 36C show the neutralization activity of anti-RBD IgG antibodies in the sera of animals from the different treatment groups described in FIGS. 35A-35E. FIGS. 36A and 36B provide comparison of the neutralization activity of the anti-RBD IgG antibodies from the sera of animals that received the exoRBD or exoRBD-MPLA, respectively, using the different dosing regimens. The dark dashed lines represent anti-RBD neutralization activity in the sera of human subjects at two weeks after receiving the second dose of a COVID19 mRNA vaccine. FIG. 36C provides a comparison of the fold change in neutralizing antibody activity observed with the incremental and constant dosing, relative to the corresponding activity observed with the bolus dosing.

FIGS. 37A and 37B provide comparison of germinal center formation in the draining lymph nodes (cervical lymph nodes) of animals from the different treatment groups described in FIGS. 35A-35E. FIG. 37A shows the results for animals that received exoRBD. FIG. 37B shows the results for animals that received exoRBD-MPLA.

FIGS. 38A and 38B show the use of the ALFA-tag system described herein to load murine IL-12 protein onto the exterior surface of the EVs. FIG. 38A provides a schematic illustrating the interaction between ALFA-tagged IL-12 protein and ALFA nanobody (NbALFA) on the exterior surface of the EV (“NbALFA-mIL-12 exo”). FIG. 38B shows the number of ALFA-tagged IL-12 proteins observed on the exterior surface of the EVs. As further described in Example 11, EVs modified to comprise NbALFA acceptor molecules on the exterior surface of the EVs were loaded with five different concentrations of the ALFA-tagged IL-12 proteins—first five bars. For comparison purposes, EVs comprising IL-12 fused to the N-terminus of PTGFRN were used as control (last bar; “mIL-12”).

FIGS. 39A and 39B show the in vitro activity of the engineered EVs comprising ALFA-tagged IL-12 protein (described in FIGS. 38A and 38B). FIG. 39A show the potency of NbALFA-mIL-12 exo as measured using a HEK IL-12 cell line reporter assay. As described in Example 11, to produce the NbALFA-mIL-12 exo, the ALFA-tagged IL-12 protein was loaded onto the EVs at five different concentrations: (1) 34 μg (“NbALFA exo+mIL12-ALFA (34 ug/1E11P)”), (2) 11 μg (“NbALFA exo+mIL12-ALFA (11 ug/1E11P)”), (3) 2.3 μg (“NbALFA exo+mIL12-ALFA (2.3 ug/1E11P)”), (4) 0.33 μg (“NbALFA exo+mIL12-ALFA (0.33 ug/1E11P)”), and (5) 0.08 μg (“NbALFA exo+mIL12-ALFA (0.08 ug/1E11P)”). The following EVs were used as controls: (i) EVs comprising IL-12 fused to the N-terminus of PTGFRN (“mIL12”); and (ii) EVs comprising NbALFA conjugated to PTGFRN but not IL-12) (“NbALFA Exo”). The table to the right of FIG. 39A provides both the EC50 and the fold-change relative to the mIL12 control. Values in bold are fold changes in potency greater than mIL12 and in parentheses fold change less than mIL12. FIG. 39B shows the correlation between potency (x-axis) and the IL-12 copy number on the surface of the EVs (y-axis).

FIGS. 40A, 40B, 40C, 40D, and 40E show the in vivo activity of the engineered EVs comprising ALFA-tagged IL-12 protein (described in FIGS. 38A and 38B) in a mouse MC-38 tumor model. FIG. 40A provides the experimental design. The animals received a single intratumoral administration of one of the following: i) PBS only; (ii) recombinant IL-12 protein (at three different concentrations: 160 ng, 445 ng, or 1,554 ng) (“mIL-12”); (iii) NbALFA-mIL-12 exo (at three different concentrations: 160 ng/5×10¹⁰ particles, 445 ng/5×10¹⁰ particles, or 1,554 ng/5×10¹⁰ particles); (iv) control EV #1 (i.e., comprising NbALFA conjugated to PTGFRN but not IL-12) (“NbALFA Exo”); and (v) control EV #2 (i.e., comprising IL-12 genetically fused to the N-terminus of PTGFRN) (“mIL-12 Exo”). FIGS. 40B and 40C provide comparison of the level of IL-12p70 and IFN-γ, respectively, measured in the tumors of animals from the different groups. FIGS. 40D and 40E provide comparison of the level of IL-12p70 and IFN-γ, respectively, measured in the sera of animals from the different groups.

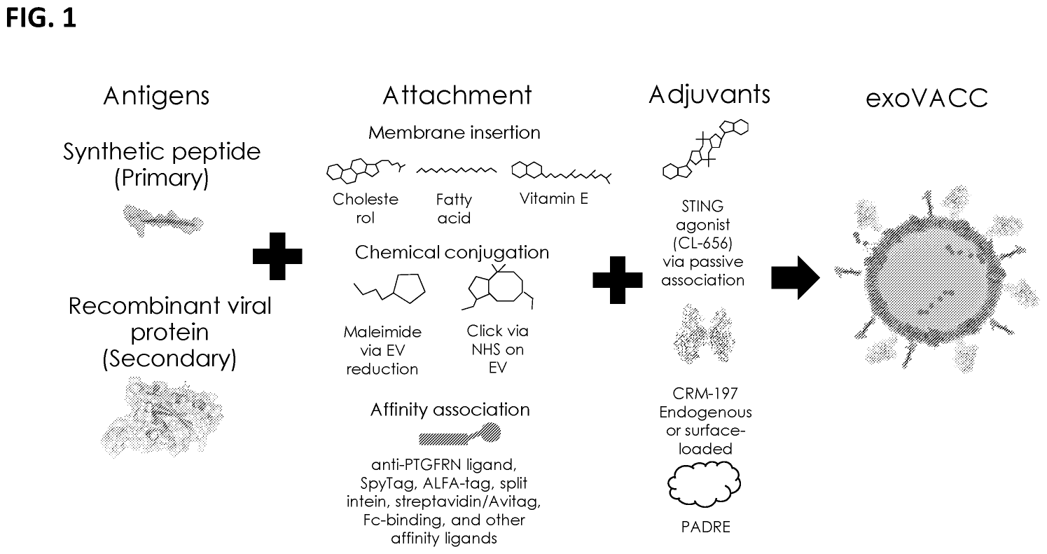

### DETAILED DESCRIPTION OF DISCLOSURE

The present disclosure is directed to extracellular vesicles (EVs) (e.g., exosomes) and methods of rapidly modifying such EVs to deliver one or more therapeutic molecules (e.g., antigen) to a subject in need thereof. In some aspects, the EV-based vaccine platform described herein allows for the use of the same engineered EV (e.g., exosome) to treat a wide range of diseases or disorders, e.g., by simply “plugging” a moiety (e.g., antigen of interest) into the EVs or by rapidly attaching a moiety (e.g., antigen of interest) as a “clip-on” attachment to the EVs. As will be apparent to those skilled in the arts, such EV-based vaccines can be useful in treating diseases or disorders that are regionalized or individualized. Non-limiting examples of the various aspects are shown in the present disclosure.

### Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “extracellular vesicle” or “EV” refers to a cell-derived vesicle comprising a membrane that encloses an internal space. Extracellular vesicles comprise all membrane-bound vesicles (e.g., exosomes, nanovesicles) that have a smaller diameter than the cell from which they are derived. In some aspects, extracellular vesicles range in diameter from 20 nm to 1000 nm, and can comprise various macromolecular payload either within the internal space (i.e., lumen), displayed on the external surface of the extracellular vesicle, and/or spanning the membrane. In some aspects, the payload can comprise nucleic acids, proteins, carbohydrates, lipids, small molecules, and/or combinations thereof. In certain aspects, an extracellular vehicle comprises a scaffold moiety. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, the extracellular vesicles are produced by cells that express one or more transgene products.

As used herein, the term “exosome” refers to an extracellular vesicle with a diameter between 20-300 nm (e.g., between 40-200 nm). Exosomes comprise a membrane that encloses an internal space (i.e., lumen), and, in some aspects, can be generated from a cell (e.g., producer cell) by direct plasma membrane budding or by fusion of the late endosome or multi-vesicular body with the plasma membrane. In certain aspects, an exosome comprises a scaffold moiety. As described infra, exosome can be derived from a producer cell, and isolated from the producer cell based on its size, density, biochemical parameters, or a combination thereof. In some aspects, the EVs, e.g., exosomes, of the present disclosure are produced by cells that express one or more transgene products.

As used herein, the term “nanovesicle” refers to an extracellular vesicle with a diameter between 20-250 nm (e.g., between 30-150 nm) and is generated from a cell (e.g., producer cell) by direct or indirect manipulation such that the nanovesicle would not be produced by the cell without the manipulation. Appropriate manipulations of the cell to produce the nanovesicles include but are not limited to serial extrusion, treatment with alkaline solutions, sonication, or combinations thereof. In some aspects, production of nanovesicles can result in the destruction of the producer cell. In some aspects, population of nanovesicles described herein are substantially free of vesicles that are derived from cells by way of direct budding from the plasma membrane or fusion of the late endosome with the plasma membrane. In certain aspects, a nanovesicle comprises a scaffold moiety. Nanovesicles, once derived from a producer cell, can be isolated from the producer cell based on its size, density, biochemical parameters, or a combination thereof.

As used herein the term “surface-engineered EVs, e.g., exosomes” (e.g., Scaffold X-engineered EVs, e.g., exosomes) refers to an EV, e.g., exosome, with the membrane or the surface of the EV, e.g., exosome, modified in its composition so that the surface of the engineered EV, e.g., exosome, is different from that of the EV, e.g., exosome, prior to the modification or of the naturally occurring EV, e.g., exosome. The engineering can be on the surface of the EV, e.g., exosome, or in the membrane of the EV, e.g., exosome, so that the surface of the EV, e.g., exosome, is changed. For example, the membrane is modified in its composition of a protein, a lipid, a small molecule, a carbohydrate, etc. The composition can be changed by a chemical, a physical, or a biological method or by being produced from a cell previously or concurrently modified by a chemical, a physical, or a biological method. Specifically, the composition can be changed by a genetic engineering or by being produced from a cell previously modified by genetic engineering. In some aspects, a surface-engineered EV, e.g., exosome, comprises an exogenous protein (i.e., a protein that the EV, e.g., exosome, does not naturally express) or a fragment or variant thereof that can be exposed to the surface of the EV, e.g., exosome, or can be an anchoring point (attachment) for a moiety exposed on the surface of the EV, e.g., exosome. In some aspects, a surface-engineered EV, e.g., exosome, comprises a higher expression (e.g., higher number) of a natural exosome protein (e.g., Scaffold X) or a fragment or variant thereof that can be exposed to the surface of the EV, e.g., exosome, or can be an anchoring point (attachment) for a moiety exposed on the surface of the EV, e.g., exosome.

As used herein the term “lumen-engineered exosome” (e.g., Scaffold Y-engineered exosome) refers to an EV, e.g., exosome, with the membrane or the lumen of the EV, e.g., exosome, modified in its composition so that the lumen of the engineered EV, e.g., exosome, is different from that of the EV, e.g., exosome, prior to the modification or of the naturally occurring EV, e.g., exosome. The engineering can be directly in the lumen or in the membrane of the EV, e.g., exosome so that the lumen of the EV, e.g., exosome is changed. For example, the membrane is modified in its composition of a protein, a lipid, a small molecule, a carbohydrate, etc. so that the lumen of the EV, e.g., exosome is modified. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering. In some aspects, a lumen-engineered exosome comprises an exogenous protein (i.e., a protein that the EV, e.g., exosome does not naturally express) or a fragment or variant thereof that can be exposed in the lumen of the EV, e.g., exosome or can be an anchoring point (attachment) for a moiety exposed on the inner layer of the EV, e.g., exosome. In some aspects, a lumen-engineered EV, e.g., exosome, comprises a higher expression of a natural exosome protein (e.g., Scaffold X or Scaffold Y) or a fragment or variant thereof that can be exposed to the lumen of the exosome or can be an anchoring point (attachment) for a moiety exposed in the lumen of the exosome.

The term “modified,” when used in the context of EVs, e.g., exosomes described herein, refers to an alteration or engineering of an EV, e.g., exosome and/or its producer cell, such that the modified EV, e.g., exosome is different from a naturally-occurring EV, e.g., exosome. In some aspects, a modified EV, e.g., exosome described herein comprises a membrane that differs in composition of a protein, a lipid, a small molecular, a carbohydrate, etc. compared to the membrane of a naturally-occurring EV, e.g., exosome (e.g., membrane comprises higher density or number of natural exosome proteins and/or membrane comprises proteins that are not naturally found in exosomes (e.g., antigen, adjuvant, and/or immune modulator). In certain aspects, such modifications to the membrane changes the exterior surface of the EV, e.g., exosome (e.g., surface-engineered EVs, e.g., exosomes described herein). In certain aspects, such modifications to the membrane changes the lumen of the EV, e.g., exosome (e.g., lumen-engineered EVs, e.g., exosomes described herein).

As used herein, the term “scaffold moiety” refers to a molecule that can be used to anchor a payload or any other compound of interest (e.g., antigen, adjuvant, and/or immune modulator) to the EV, e.g., exosome either on the luminal surface or on the exterior surface of the EV, e.g., exosome. In certain aspects, a scaffold moiety comprises a synthetic molecule. In some aspects, a scaffold moiety comprises a non-polypeptide moiety. In some aspects, a scaffold moiety comprises a lipid, carbohydrate, or protein that naturally exists in the EV, e.g., exosome. In some aspects, a scaffold moiety comprises a lipid, carbohydrate, or protein that does not naturally exist in the EV, e.g., exosome. In certain aspects, a scaffold moiety is Scaffold X. In some aspects, a scaffold moiety is Scaffold Y. In further aspects, a scaffold moiety comprises both Scaffold X and Scaffold Y. Non-limiting examples of other scaffold moieties that can be used with the present disclosure include: aminopeptidase N (CD13); Neprilysin, AKA membrane metalloendopeptidase (MME); ectonucleotide pyrophosphatase/phosphodiesterase family member 1 (ENPP1); Neuropilin-1 (NRP1); CD9, CD63, CD81, PDGFR, GPI anchor proteins, lactadherin, LAMP2, and LAMP2B.

As used herein, the term “Scaffold X” refers to exosome proteins that have recently been identified on the surface of exosomes. See, e.g., U.S. Pat. No. 10,195,290, which is incorporated herein by reference in its entirety. Non-limiting examples of Scaffold X proteins include: prostaglandin F2 receptor negative regulator (“the PTGFRN protein”); basigin (“the BSG protein”); immunoglobulin superfamily member 2 (“the IGSF2 protein”); immunoglobulin superfamily member 3 (“the IGSF3 protein”); immunoglobulin superfamily member 8 (“the IGSF8 protein”); integrin beta-1 (“the ITGB1 protein); integrin alpha-4 (“the ITGA4 protein”); 4F2 cell-surface antigen heavy chain (“the SLC3A2 protein”); and a class of ATP transporter proteins (“the ATP1A1 protein,” “the ATP1A2 protein,” “the ATP1A3 protein,” “the ATP1A4 protein,” “the ATP1B3 protein,” “the ATP2B1 protein,” “the ATP2B2 protein,” “the ATP2B3 protein,” “the ATP2B protein”). In some aspects, a Scaffold X protein can be a whole protein or a fragment thereof (e.g., functional fragment, e.g., the smallest fragment that is capable of anchoring another moiety on the exterior surface or on the luminal surface of the EV, e.g., exosome). In some aspects, a Scaffold X can anchor a moiety (e.g., antigen, adjuvant, and/or immune modulator) to the external surface or the luminal surface of the exosome.

As used herein, the term “Scaffold Y” refers to exosome proteins that were newly identified within the lumen of exosomes. See, e.g., International Appl. No. PCT/US2018/061679 (or published US equivalent—US 2020/0347112), which is incorporated herein by reference in its entirety. Non-limiting examples of Scaffold Y proteins include: myristoylated alanine rich Protein Kinase C substrate (“the MARCKS protein”); myristoylated alanine rich Protein Kinase C substrate like 1 (“the MARCKSL1 protein”); and brain acid soluble protein 1 (“the BASP1 protein”). In some aspects, a Scaffold Y protein can be a whole protein or a fragment thereof (e.g., functional fragment, e.g., the smallest fragment that is capable of anchoring a moiety to the luminal surface of the exosome). In some aspects, a Scaffold Y can anchor a moiety (e.g., antigen, adjuvant, and/or immune modulator) to the luminal surface of the EV, e.g., exosome.

As used herein, the term “fragment” of a protein (e.g., therapeutic protein, Scaffold X, or Scaffold Y) refers to an amino acid sequence of a protein that is shorter than the naturally-occurring sequence, N- and/or C-terminally deleted or any part of the protein deleted in comparison to the naturally occurring protein. As used herein, the term “functional fragment” refers to a protein fragment that retains protein function. Accordingly, in some aspects, a functional fragment of a Scaffold X protein retains the ability to anchor a moiety on the luminal surface or on the exterior surface of the EV, e.g., exosome. Similarly, in certain aspects, a functional fragment of a Scaffold Y protein retains the ability to anchor a moiety on the luminal surface of the EV, e.g., exosome. Whether a fragment is a functional fragment can be assessed by any art known methods to determine the protein content of EVs, e.g., exosomes including Western Blots, FACS analysis and fusions of the fragments with autofluorescent proteins like, e.g., GFP. In certain aspects, a functional fragment of a Scaffold X protein retains at least about 50%, at least about 60%, at least about 70%, at least about 80%, at least about 90% or at least about 100% of the ability, e.g., an ability to anchor a moiety, of the naturally occurring Scaffold X protein. In some aspects, a functional fragment of a Scaffold Y protein retains at least about 50%, at least about 60%, at least about 70%, at least about 80%, at least about 90% or at least about 100% of the ability, e.g., an ability to anchor another molecule, of the naturally occurring Scaffold Y protein.

As used herein, the term “variant” of a molecule (e.g., functional molecule, antigen, Scaffold X and/or Scaffold Y) refers to a molecule that shares certain structural and functional identities with another molecule upon comparison by a method known in the art. For example, a variant of a protein can include a substitution, insertion, deletion, frameshift or rearrangement in another protein.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In some aspects, a string of amino acids can be conservatively replaced with a structurally similar string that differs in order and/or composition of side chain family members.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of programs available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at worldwideweb.tcoffee.org, and alternatively available, e.g., from the EBI. It will also be appreciated that the final alignment used to calculate percent sequence identity can be curated either automatically or manually.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, polypeptide variants include, e.g., modified polypeptides. Modifications include, e.g., conservative amino acid substitution, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, covalent attachment of bioorthoganal functionalities (e.g., azide, alkyne, trans-cycloalkyne, tetrazine), cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination. In some aspects, Scaffold X and/or Scaffold Y is modified at any convenient location.

As used herein, when a molecule described herein (e.g., antigen, adjuvant, immune modulator, targeting moiety, affinity ligand, and/or scaffold moiety) is “expressed,” “added,” or “loaded” in an EV (e.g., exosome), it means that the molecule is present in or on the EV. As described herein, an EV (e.g., exosome) can express a molecule of interest on its exterior surface, on its luminal surface, in the lumen, or combinations thereof. In some aspects, a molecule can be exogenously introduced into a producer cell or directly into an EV, such that the EV expresses the molecule of interest. In some aspects, a molecule of interest can be produced separately from an EV and then conjugated or linked to a moiety present in the EV, such that the EV expresses the molecule. For instance, in some aspects, an antigen (e.g., derived from a spike S protein of a coronavirus, e.g., receptor-binding domain of a spike S protein) can be fused to an affinity ligand disclosed herein. Then, the antigen-affinity ligand fusion can be linked or conjugated to a scaffold moiety expressed on the surface of an EV via the affinity ligand. Additional disclosure relating to different methods of expressing a molecule of interest in or on an EV (e.g., exosome) is described elsewhere in the present disclosure.

As used herein, the term “linked to,” “fused,” or “conjugated to” are used interchangeably and refer to a covalent or non-covalent bond formed between a first moiety and a second moiety, e.g., Scaffold X and an antigen (or adjuvant or immune modulator), respectively, e.g., a scaffold moiety expressed in or on the extracellular vesicle and an antigen, e.g., Scaffold X (e.g., a PTGFRN protein), respectively, in the luminal surface of or on the external surface of the extracellular vesicle. In some aspects, a payload disclosed herein (e.g., antigen, adjuvant, and/or immune modulator) and/or a targeting moiety can be directly linked to the exterior surface and/or the luminal surface of an EV (e.g., exosome). As used herein, the term “directly linked,” “directly fused,” or “directly conjugated to” refer to the process of linking (fusing or conjugating) a moiety (e.g., a payload and/or targeting moiety) to the surface of an EV (e.g., exosome) without the use of a scaffold moiety disclosed herein.

As used herein, the term “fusion protein” refers to two or more proteins that are linked or conjugated to each other. For instance, in some aspects, a fusion protein that can be expressed in an EV (e.g., exosome) disclosed herein comprises (i) a payload (e.g., antigen, adjuvant, and/or immune modulator) and (ii) a scaffold moiety (e.g., Scaffold X and/or Scaffold Y). In some aspects, the payload (e.g., antigen, adjuvant, and/or immune modulator) is linked or conjugated to the scaffold moiety via an affinity ligand (e.g., those described herein). In some aspects, a fusion protein that can be expressed in an EV (e.g., exosome) useful for the present disclosure comprises (i) a targeting moiety and (ii) a scaffold moiety (e.g., Scaffold X and/or Scaffold Y). In some aspects, the targeting moiety is linked or conjugated to the scaffold moiety via an affinity ligand (e.g., those described herein). As described herein, in some aspects, EVs (e.g., exosomes) of the present disclosure can express multiple fusion proteins, wherein a first fusion protein comprises (i) a payload (e.g., antigen, adjuvant, and/or immune modulator) and (ii) a scaffold moiety (e.g., Scaffold X and/or Scaffold Y), and wherein a second fusion protein comprises (i) a targeting moiety and (ii) a scaffold moiety (e.g., Scaffold X and/or Scaffold Y).

The term “encapsulated”, or grammatically different forms of the term (e.g., encapsulation, or encapsulating) refers to a status or process of having a first moiety (e.g., antigen, adjuvant, or immune modulator) inside a second moiety (e.g., an EV, e.g., exosome) without chemically or physically linking the two moieties. In some aspects, the term “encapsulated” can be used interchangeably with the terms “in the lumen of.” Non-limiting examples of encapsulating a first moiety (e.g., payload, e.g., antigen, adjuvant, or immune modulator) into a second moiety (e.g., EVs, e.g., exosomes) are disclosed elsewhere herein.

As used herein, the term “producer cell” refers to a cell used for generating an EV, e.g., exosome. A producer cell can be a cell cultured in vitro, or a cell in vivo. A producer cell includes, but not limited to, a cell known to be effective in generating EVs, e.g., exosomes, e.g., HEK293 cells, Chinese hamster ovary (CHO) cells, mesenchymal stem cells (MSCs), BJ human foreskin fibroblast cells, fHDF fibroblast cells, AGE.HN® neuronal precursor cells, CAP® amniocyte cells, adipose mesenchymal stem cells, RPTEC/TERT1 cells. In certain aspects, a producer cell is not an antigen-presenting cell. In some aspects, a producer cell is not a dendritic cell, a B cell, a mast cell, a macrophage, a neutrophil, Kupffer-Browicz cell, cell derived from any of these cells, or any combination thereof. In some aspects, a producer cell is not a naturally-existing antigen-presenting cell (i.e., has been modified). In some aspects, a producer cell is not a naturally-existing dendritic cell, a B cell, a mast cell, a macrophage, a neutrophil, Kupffer-Browicz cell, cell derived from any of these cells, or any combination thereof. Additional disclosures relating to such producer cells are provided elsewhere in the present disclosure. In some aspects, the EVs, e.g., exosomes useful in the present disclosure do not carry an antigen on MHC class I or class II molecule (i.e., antigen is not presented on MHC class I or class II molecule) exposed on the surface of the EV, e.g., exosome, but instead can carry an antigen in the lumen of the EV, e.g., exosome, or on the surface of the EV, e.g., exosome, by attachment to Scaffold X and/or Scaffold Y.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some aspects, isolating or purifying as used herein is the process of removing, partially removing (e.g., a fraction) of the EVs from a sample containing producer cells. In some aspects, an isolated EV composition has no detectable undesired activity or, alternatively, the level or amount of the undesired activity is at or below an acceptable level or amount. In some aspects, an isolated EV composition has an amount and/or concentration of desired EVs at or above an acceptable amount and/or concentration. In some aspects, the isolated EV composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some aspects, isolated EV preparations are substantially free of residual biological products. In some aspects, the isolated EV preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the EV composition contains no detectable producer cells and that only EVs are detectable.

As used herein, the term “loading efficiency” can refer to (i) the amount of time required to load a given amount of moiety (e.g., antigen and/or adjuvant) to an EV; (ii) the amount of moiety (e.g., antigen and/or adjuvant) that can be loaded to an EV in a given amount of time; or (iii) both (i) and (ii).

As used herein, the term “immune modulator” refers to an agent (i.e., payload) that acts on a target (e.g., a target cell) that is contacted with the extracellular vesicle, and regulates the immune system. Non-limiting examples of immune modulator that can be introduced into an EV (e.g., exosome) and/or a producer cell include agents such as, modulators of checkpoint inhibitors, ligands of checkpoint inhibitors, cytokines, derivatives thereof, or any combination thereof. The immune modulator can also include an agonist, an antagonist, an antibody, an antigen-binding fragment, a polynucleotide, such as siRNA, antisense oligonucleotide, a phosphorodiamidate morpholino oligomer (PMO), a peptide-conjugated phosphorodiamidate morpholino oligomer (PPMO), miRNA, lncRNA, mRNA DNA, or a small molecule. Unless indicated otherwise, in some aspects, the terms immune modulator and adjuvants can be used interchangeably. Additional examples of suitable adjuvants/immune modulators are provided elsewhere in the present disclosure.

As used herein, the term a “bio-distribution modifying agent,” which refers to an agent (i.e., payload) that can modify the distribution of extracellular vesicles (e.g., exosomes, nanovesicles) in vivo or in vitro (e.g., in a mixed culture of cells of different varieties). In some aspects, the term “targeting moiety” can be used interchangeably with the term bio-distribution modifying agent. In some aspects, the targeting moiety alters the tropism of the EV (e.g., exosome) (“tropism moiety”). As used herein, the term “tropism moiety” refers to a targeting moiety that when expressed on an EV (e.g., exosome) alters and/or enhances the natural movement of the EV. For example, in some aspects, a tropism moiety can promote the EV to be taken up by a particular cell, tissue, or organ. Non-limiting examples of tropism moieties that can be used with the present disclosure include those that can bind to a marker expressed specifically on a dendritic cell (e.g., Clec9A or DEC205) or T cells (e.g., CD3). In some aspects, a tropism moiety comprises the RBD of a coronavirus spike protein, which can help shift the tropism of EVs comprising the RBD to ACE2 positive cells (e.g., epithelial cells). Unless indicated otherwise, the term “targeting moiety,” as used herein, encompasses tropism moieties. The bio-distribution agent can be a biological molecule, such as a protein, a peptide, a lipid, or a carbohydrate, or a synthetic molecule. For example, the bio-distribution modifying agent can be an affinity ligand (e.g., antibody, VHH domain, phage display peptide, fibronectin domain, camelid, VNAR), a synthetic polymer (e.g., PEG), a natural ligand/molecule (e.g., CD40L, albumin, CD47, CD24, CD55, CD59), a recombinant protein (e.g., XTEN), but not limited thereto.

As used herein, the term “C-type lectin domain family 9 member A” (Clec9a) protein refers to a group V C-type lectin-like receptor (CTLR) that functions as an activation receptor and is expressed on myeloid lineage cells (e.g., DCs). Huysamen et al., J Biol Chem 283(24):16693-701 (2008); U.S. Pat. No. 9,988,431 B2, each of which is herein incorporated by reference in its entirety. Synonyms of Clec9a are known and include CD370, DNGR-1, 5B5, HEEE9341, and C-type lectin domain containing 9A. In some aspects, Clec9a protein is expressed on human cDC1 cells. In some aspects, Clec9a protein is expressed on mouse cDC1 and pDC cells. Unless indicated otherwise, Clec9a, as used herein, can refer to Clec9a from one or more species (e.g., humans, non-human primates, dogs, cats, guinea pigs, rabbits, rats, mice, horses, cattle, and bears).

As used herein, the term “CD3” or “cluster of differentiation 3” refers to the protein complex associated with the T cell receptor (TCR). The CD3 molecule is made up of four distinct chains (CD3γ, CD36, and two CD3c chains). These chains associate with the T-cell receptor (TCR) and the ζ-chain to generate an activation signal in T lymphocytes. The TCR, ζ-chain, and CD3 molecules together constitute the TCR complex. CD3 molecules are expressed on all T cells, including both CD4+ T cells and CD8+ T cells. Unless indicated otherwise, CD3, as used herein, can refer to CD3 from one or more species (e.g., humans, non-human primates, dogs, cats, guinea pigs, rabbits, rats, mice, horses, cattle, and bears).

As used herein, the term “payload” refers to an agent that acts on a target (e.g., a target cell) that is contacted with the EV (e.g., exosome). In some aspects, unless indicated otherwise, the term payload can be used interchangeably with the term “biologically active molecules.” Non-limiting examples of payload that can be included on the EV, e.g., exosome, are an antigen, an adjuvant, and/or an immune modulator. Payloads that can be introduced into an EV, e.g., exosome, and/or a producer cell include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, siRNA, antisense oligonucleotide, a phosphorodiamidate morpholino oligomer (PMO), a peptide-conjugated phosphorodiamidate morpholino oligomer (PPMO), or combinations thereof), amino acids (e.g., amino acids comprising a detectable moiety or a toxin or that disrupt translation), polypeptides (e.g., enzymes), lipids, carbohydrates, peptides (e.g., cell penetrating peptides), and small molecules (e.g., small molecule drugs and toxins). In certain aspects, a payload comprises an antigen (e.g., RBD of a coronavirus spike protein). As used herein, the term “antigen” refers to any agent that when introduced into a subject elicits an immune response (cellular or humoral) to itself.

As used herein, the term “affinity ligand” refers to a molecule that can selectively and preferentially bind to a specific marker, e.g., expressed on a target cell or on EVs, e.g, a scaffold moiety, e.g., PTGFRN on EVs. As described herein, in some aspects, an affinity ligand comprises a peptide (e.g., linear peptide) or protein that can increase the binding of a molecule of interest (e.g., antigen, adjuvant, immune modulator, and/or targeting moiety) to a moiety on the surface of EVs, e.g., a scaffold moiety disclosed herein. Non-limiting examples of affinity ligands that can be used with the present disclosure include an antibody, phage display peptide, fibronectin domain, camelid, VNAR, VHH domain, and combinations thereof. As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(abl)₂, Fv, dAb, and Fd fragments, diabodies, and antibody-related polypeptides. Antibody includes bispecific antibodies and multispecific antibodies so long as they exhibit the desired biological activity or function. Further description of affinity ligands that can be used with an EV (e.g., exosome) are provided elsewhere in the present disclosure (see, e.g., section II.G).

As used herein, the term “acceptor domain” refers to a protein sequence that forms a stable interaction (either covalent or non-covalent) with a cognate protein “donor domain.” As demonstrated herein, in some aspects, acceptor domains can be displayed on the surface of EVs (e.g., exosomes) via fusion to a scaffold moiety (e.g., PTGFRN). In some aspects, by mixing the acceptor EVs (i.e., EVs that comprise the acceptor domain) with a soluble donor (comprising the donor domain and a target molecule of interest), it can be possible to display the target molecule of interest on the surface of EVs (e.g., exosomes).

The terms “individual,” “subject,” “host,” and “patient,” are used interchangeably herein and refer to any mammalian subject for whom diagnosis, treatment, or therapy is desired, particularly humans. The compositions and methods described herein are applicable to both human therapy and veterinary applications. In some aspects, the subject is a mammal, and in some aspects, the subject is a human. As used herein, a “mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

As used herein, the term “substantially free” means that the sample comprising EVs, e.g., exosomes, comprise less than about 10% of macromolecules by mass/volume (m/v) percentage concentration. Some fractions can contain less than about 0.001%, less than about 0.01%, less than about 0.05%, less than about 0.1%, less than about 0.2%, less than about 0.3%, less than about 0.4%, less than about 0.5%, less than about 0.6%, less than about 0.7%, less than about 0.8%, less than about 0.9%, less than about 1%, less than about 2%, less than about 3%, less than about 4%, less than about 5%, less than about 6%, less than about 7%, less than about 8%, less than about 9%, or less than about 10% (m/v) of macromolecules.

As used herein, the term “macromolecule” means nucleic acids, contaminant proteins, lipids, carbohydrates, metabolites, or a combination thereof.

As used herein, the term “conventional exosome protein” means a protein previously known to be enriched in exosomes, including but is not limited to CD9, CD63, CD81, PDGFR, GPI anchor proteins, lactadherin LAMP2, and LAMP2B, a fragment thereof, or a peptide that binds thereto.

“Administering,” as used herein, means to give a composition comprising an EV, e.g., exosome, disclosed herein to a subject via a pharmaceutically acceptable route. Routes of administration can be intravenous, e.g., intravenous injection and intravenous infusion. Additional routes of administration include, e.g., subcutaneous, intramuscular, oral, nasal, intrathecal, and pulmonary administration. EVs, e.g., exosomes can be administered as part of a pharmaceutical composition comprising at least one excipient. Examples of additional suitable routes of administration are provided elsewhere in the present disclosure.

An “immune response,” as used herein, refers to a biological response within a vertebrate against foreign agents or abnormal, e.g., coronavirus, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, other abnormal cells, or, in cases of autoimmunity or pathological inflammation, normal human cells or tissues. As used herein, immune response comprises a cellular immune response, a humoral immune response, an innate cell immune response, or a combination thereof. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell. Accordingly an immune response can comprise a humoral immune response (e.g., mediated by B-cells), cellular immune response (e.g., mediated by T cells), or both humoral and cellular immune responses. In some aspects, an immune response is an “inhibitory” immune response. An “inhibitory” or “tolerogenic” immune response is an immune response that blocks or diminishes the effects of a stimulus (e.g., antigen). In certain aspects, the inhibitory immune response comprises the production of inhibitory antibodies against the stimulus. In some aspects, the inhibitory immune response comprises the induction of tolerogenic cells, such as regulatory T cells (e.g., FoxP3+ regulatory CD4+ T cells). In some aspects, the inhibitory immune response comprises the production of tolerogenic cytokines/chemokines (e.g., IL-10 or TGF-β). In some aspects, an immune response is a “stimulatory” immune response. A “stimulatory” immune response comprises an immune response that results in the generation of effectors cells (e.g., cytotoxic T lymphocytes) that can destroy and clear a target antigen of coronaviruses. In some aspects, a stimulatory immune response comprises the production of antibodies that can specifically bind and neutralize an antigen.

As used herein, the term “cellular immune response” can be used interchangeably with the term “cell-mediated immune response” and refers to an immune response that does not predominantly involve antibodies. Instead, a cellular immune response involves the activation of different immune cells (e.g., phagocytes and antigen-specific cytotoxic T-lymphocytes) that produce various effector molecules (e.g., cytokines, perforin, granzymes) upon activation (e.g., via antigen stimulation). As used herein, the term “humoral immune response” refers to an immune response predominantly mediated by macromolecules found in extracellular fluids, such as secreted antibodies, complement proteins, and certain antimicrobial peptides. The term “antibody-mediated immune response” refers to an aspect of a humoral immune response that is mediated by antibodies.

As used herein, the term “immune cells” refers to any cells of the immune system that are involved in mediating an immune response. Non-limiting examples of immune cells include a T lymphocyte, B lymphocyte, natural killer (NK) cell, macrophage, eosinophil, mast cell, dendritic cell, neutrophil, or combination thereof. In some aspects, an immune cell expresses CD3. In certain aspects, the CD3-expressing immune cells are T cells (e.g., CD4+ T cells or CD8+ T cells). In some aspects, an immune cell that can be targeted with a targeting moiety disclosed herein (e.g., anti-CD3) comprises a naïve CD4+ T cell. In some aspects, an immune cell comprises a memory CD4+ T cell. In some aspects, an immune cell comprises an effector CD4+ T cell. In some aspects, an immune cell comprises a naïve CD8+ T cell. In some aspects, an immune cell comprises a memory CD8+ T cell. In some aspects, an immune cell comprises an effector CD8+ T cell. In some aspects, an immune cell is a dendritic cell. In certain aspects, a dendritic cell comprises a plasmacytoid dendritic cell (pDC), a conventional dendritic cell 1 (cDC1), a conventional dendritic cell 2 (cDC2), inflammatory monocyte derived dendritic cells, Langerhans cells, dermal dendritic cells, lysozyme-expressing dendritic cells (LysoDCs), Kupffer cells, or any combination thereof. Accordingly, in certain aspects, an immune cell that an EV disclosed herein (e.g., exosomes) can specifically target includes a conventional dendritic cell 1 (cDC1) and/or plasmacytoid dendritic cells (pDC).

As used herein, the term “T cell” or “T-cell” refers to a type of lymphocyte that matures in the thymus. T cells play an important role in cell-mediated immunity and are distinguished from other lymphocytes, such as B cells, by the presence of a T-cell receptor on the cell surface. T-cells include all types of immune cells expressing CD3, including but not limited to T-helper cells (CD4+ cells), cytotoxic T-cells (CD8+ cells), natural killer T-cells, T-regulatory cells (Treg), T follicular helper (Tfh) cells, peripheral Tfh cells, mucosal-associated invariant T (MATT) cells, and gamma-delta T cells. As used herein, the term “B cell” or “B-cell” refers to a type of lymphocytes that originate from the bone marrow, and play a role in humoral immunity by producing antibodies. In some aspects, B cells can also have regulatory roles, such as B-regulatory cells (Breg).

A “naïve” T cell refers to a mature T cell that remains immunologically undifferentiated (i.e., not activated). Following positive and negative selection in the thymus, T cells emerge as either CD4+ or CD8+ naïve T cells. In their naïve state, T cells express L-selectin (CD62L+), IL-7 receptor-α (IL-7R-α), and CD132, but they do not express CD25, CD44, CD69, or CD45RO. As used herein, “immature” can also refers to a T cell which exhibits a phenotype characteristic of either a naïve T cell or an immature T cell, such as a TSCM cell or a TCM cell. For example, an immature T cell can express one or more of L-selectin (CD62L+), IL-7Rα, CD132, CCR7, CD45RA, CD45RO, CD27, CD28, CD95, CXCR3, and LFA-1. Naïve or immature T cells can be contrasted with terminal differentiated effector T cells, such as T_{EM} cells and T_{EFF} cells.

As used herein, the term “effector” T cells or “T_{EFF}” cells refers to a T cell that can mediate the removal of a pathogen or cell without requiring further differentiation. Thus, effector T cells are distinguished from naive T cells and memory T cells, and these cells often have to differentiate and proliferate before becoming effector cells.

As used herein, the term “memory” T cells refer to a subset of T cells that have previously encountered and responded to their cognate antigen. In some aspects, the term is synonymous with “antigen-experienced” T cells. In some aspects, memory T cells can be effector memory T cells or central memory T cells. In some aspects, the memory T cells are tissue-resident memory T cells. As used herein, the term “tissue-resident memory T cells” or “TRM cells” refers to a lineage of T cells that occupies tissues (e.g., skin, lung, gastrointestinal tract) without recirculating. TRM cells are transcriptionally, phenotypically and functionally distinct from central memory and effector memory T cells which recirculate between blood, the T cell zones of secondary lymphoid organs, lymph and nonlymphoid tissues. One of the roles of TRM cells is to provide immune protection against infection in extralymphoid tissues.

As used herein, the term “dendritic cells” or “DCs” refers to a class of bone-marrow-derived immune cells that are capable of processing extracellular and intracellular proteins and to present antigens in the context of MHC molecules to prime naïve T cells. In some aspects, dendritic cells can be divided into further subtypes, such as conventional dendritic cell 1 (cDC1), conventional dendritic cell 2 (cDC2), plasmacytoid dendritic cell (pDC), inflammatory monocyte derived dendritic cells, Langerhans cells, dermal dendritic cells, lysozyme-expressing dendritic cells (LysoDCs), Kupffer cells, and combinations thereof. In certain aspects, the different DC subsets can be distinguished based on their phenotypic expression. For example, in some aspects, human cDC1 cells are CD1c⁻ and CD141⁺. In some aspects, human cDC2 cells are CD1c⁺ and CD141⁻. In some aspects, human pDC cells are CD123⁺. In some aspects, mouse cDC1 cells are XCR1⁺, Clec9a⁺, and Sirpa⁻. In some aspects, mouse cDC2 cells are CD8⁺, CD11b⁺, Sirpa⁺, XCR1⁻, and CD1c,b⁺. In some aspects, mouse pDC cells are CD137⁺, XCR1⁻, and Sirpa⁻. Other phenotypic markers for distinguishing the different DC subsets are known in the art. See, e.g., Collin et al., Immunology 154(1): 3-20 (2018). In some aspects, the different DC subsets can be distinguished based on their functional properties. For example, in certain aspects, pDCs produce large amounts of IFN-α, while cDC1s and cDC2s produce inflammatory cytokines, such as IL-12, IL-6, and TNF-α. Other methods of distinguishing the different DC subsets are known in the art. See, e.g., U.S. Pat. Nos. 8,426,565 B2 and 9,988,431, each of which is herein incorporated by reference in its entirety.

The term “immunoconjugate,” as used herein, refers to a compound comprising a binding molecule (e.g., an antibody) and one or more moieties, e.g., therapeutic or diagnostic moieties, chemically conjugated to the binding molecule. In general, an immunoconjugate is defined by a generic formula: A−(L−M)n, wherein A is a binding molecule (e.g., an antibody), L is an optional linker, and M is a heterologous moiety which can be for example a therapeutic agent, a detectable label, etc., and n is an integer. In some aspects, multiple heterologous moieties can be chemically conjugated to the different attachment points in the same binding molecule (e.g., an antibody). In some aspects, multiple heterologous moieties can be concatenated and attached to an attachment point in the binding molecule (e.g., an antibody). In some aspects, multiple heterologous moieties (being the same or different) can be conjugated to the binding molecule (e.g., an antibody).

Immunoconjugates can also be defined by the generic formula in reverse order. In some aspects, the immunoconjugate is an “antibody-Drug Conjugate” (“ADC”). In the context of the present disclosure, the term “immunoconjugate” is not limited to chemically or enzymatically conjugates molecules. The term “immunoconjugate” as used in the present disclosure also includes genetic fusions. In some aspects of the present disclosure, the biologically active molecule is an immunoconjugate. The terms “antibody-drug conjugate” and “ADC” are used interchangeably and refer to an antibody linked, e.g., covalently, to a therapeutic agent (sometimes referred to herein as agent, drug, or active pharmaceutical ingredient) or agents. In some aspects of the present disclosure, the biologically active molecule (i.e., a payload) is an antibody-drug conjugate.

“Treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means inducing an immune response in a subject against an antigen.

“Prevent” or “preventing,” as used herein, refers to decreasing or reducing the occurrence or severity of a particular outcome. In some aspects, preventing an outcome is achieved through prophylactic treatment.

### Methods of the Present Disclosure

### Methods of Preparing/Manufacturing EV-Based Vaccines

Provided herein is a method of making a vaccine in efficient, speedy, and convenient ways, optionally with multiple antigens. In some aspects, provided herein is a method of preparing an extracellular vesicle (EV) (e.g., exosome) for a vaccine, comprising adding an antigen to an EV that has been isolated from a producer cell. In some aspects, provided herein is a method of manufacturing a vaccine for a disease or disorder, comprising adding an antigen to an EV (e.g., exosome) that has been isolated from a producer cell. Non-limiting examples of antigens that can be used with the present methods are provided elsewhere in the present disclosure.

As used herein, EVs (e.g., exosomes) that have been “isolated from a producer cell” refer to EVs (e.g., exosomes) that exist independent of the cells from which they are produced. In some aspects, once produced, the EVs (e.g., exosomes) that are useful for the present disclosure are purified or extracted from a culture containing the producer cells, and stored in a separate container until they are ready for further use (e.g., to add one or more antigens disclosed herein). Such EVs (e.g., exosomes) are also referred to herein as “base EVs” or “base exosomes.”

Not to be bound by any one theory, the use of such base EVs (e.g., exosomes) can greatly improve one or more aspects of producing vaccines, particularly at a large manufacturing scale. While many traditional vaccines (e.g., peptide-based) have been used to treat and/or prevent certain diseases or disorders, they are generally poorly immunogenic and require repeated administrations and/or high doses. See, e.g., Hos, B. J., et al., Front Immunol 9:884 (2018), which is incorporated herein by reference in its entirety. Additionally, because of manufacturing complexities, compounded by the need for different formulations for different countries and age groups, it often takes multiple years to develop and manufacture a safe and efficacious vaccine. See, e.g., Smith, J., et al., Lancet 378(9789): 428-438 (July 2011), which is incorporated herein by reference in its entirety. As is apparent with the ongoing COVID-19 pandemic, the inability to rapidly produce a safe and efficacious vaccine could have dire consequences.

In some aspects, the EV-based vaccines prepared using the methods disclosed herein differ from traditional vaccines in that the EVs (e.g., exosomes) can be rapidly engineered to comprise a moiety of interest (e.g., an antigen).

Accordingly, in some aspects, with the methods disclosed herein, the time required for manufacturing or producing a vaccine (“manufacturing time”) is reduced compared to a reference manufacturing time. In certain aspects, the reference manufacturing time refers to the time required to manufacture or produce a non-EV-based vaccine. In some aspects, the reference manufacturing time refers to the time required to manufacture or produce an EV-based vaccine wherein the antigen is not added to EVs (e.g., exosomes) that have been isolated from the producer cell (e.g., by introducing the antigen into the producer cell, such that when the EVs are produced, they comprise the antigen). In certain aspects, the manufacturing time is reduced by at least about 10%, at least about 20%, at least about 30%, at least about 40%, at least about 50%, at least about 60%, at least about 70%, at least about 80%, or at least about 90% or more, compared to the reference manufacturing time. In some aspects, the manufacturing time is less than about 12 months, less than about 11 months, less than about 10 months, less than about 9 months, less than about 8 months, less than about 7 months, less than about 6 months, less than about 5 months, less than about 4 months, less than about 3 months, less than about 2 months, or less than about 1 month. In some aspects, the manufacturing time is less than about 6 months.

As will be apparent to those skilled in the arts, the ability to simply “plug” or “clip on” an antigen of interest to a base EV (i.e., EVs that have been isolated from a producer cell) can be greatly advantageous when seeking to treat a disease or disorder that is more regional and/or more prevalent in certain individual or subsets of individuals (e.g., age group). As described herein, a base EV can differ from a naturally existing EV. For example, the base EVs can be genetically modified (e.g., by introducing a moiety of interest into the producer cells during production) or they can be modified after the EVs are produced and isolated from the producer cells.

Accordingly, in some aspects, EV-based vaccines that can be produced or manufactured using the methods described herein are regionalized vaccines. As used herein, the term “regionalized vaccines” or “regional vaccines” refer to vaccines that are tailored to certain regions of the world. For instance, geographic isolation of certain genetic subtypes/serotypes of an infectious pathogen (e.g. virus) could require a more customized vaccine as opposed to a vaccine designed to address the extensive diversity of the pathogen worldwide. In some aspects, the methods disclosed herein can be used to produce or manufacture such regionalized vaccines by adding an antigen to an EV (e.g., exosome) that has been isolated from a producer cell, wherein the antigen has been determined to be associated with a particular pathogen (or genetic subtype/serotype of a pathogen) prevalent within a certain region of the world. Non-limiting examples of such pathogens are provided elsewhere in the present disclosure.

In some aspects, EV-based vaccines that can be produced or manufactured using the methods described herein are individualized vaccines. As used herein, the term “individualized vaccines” and “personalized vaccines” can be used interchangeably and refer to vaccines that are tailored to a specific individual or subsets of individuals. Such a personalized vaccine could be of particular interest, e.g., for a cancer vaccine using neoantigens, since many neoantigens are specific for the particular cancer cells of an individual or subsets of individuals (e.g., those who share certain genetic background). In some aspects, the methods disclosed herein can be used to produce or manufacture such regionalized vaccines by adding an antigen to an EV (e.g., exosome) that has been isolated from a producer cell, wherein the antigen has been determined to have (or likely to have) a therapeutic effect (e.g., induces an immune response) in the particular individual or subset of individuals.

As will be apparent to those skilled in the arts, methods of identifying regional- and/or individual-specific antigens are known in the art. See, e.g., US 2009/0169576; US 2014/0178438; Sakkhachornphop, S., et al., J Virol Methods 217: 70-8 (June 2015); and Xu, K., et al., Sci Rep 8(1): 1067 (January 2018), each of which is incorporated herein by reference in its entirety.

In some aspects, an EV-based vaccine that can be prepared or manufactured using the methods described herein can comprise one or more additional moieties, such as those that are capable of enhancing the therapeutic efficacy of the vaccine.

In some aspects, the additional moiety comprises an adjuvant. Accordingly, in some aspects, a base EV (e.g., exosome) that can be used with the methods disclosed herein comprises an adjuvant, such that the adjuvant is present in the EV prior to the addition of the antigen. In certain aspects, the method of preparing or manufacturing an EV-based vaccine provided herein further comprises adding an adjuvant to an EV (e.g., exosome) that has been isolated from a producer cell (i.e., base EV). In some aspects, the adjuvant is added to the EV before adding the antigen. In some aspects, the adjuvant is added to the EV after adding the antigen. In some aspects, the adjuvant is added to the EV together with the antigen. Non-limiting examples of adjuvants that can be used with the present methods are provided elsewhere in the present disclosure.

In some aspects, the additional moiety comprises a targeting moiety. Accordingly, in some aspects, a base EV (e.g., exosome) that can be used with the methods disclosed herein further comprises a targeting moiety, such that one or more of the additional moieties are present in the EV prior to the addition of the antigen. In certain aspects, the method of preparing or manufacturing an EV-based vaccine provided herein further comprises adding a targeting moiety to an EV (e.g., exosome) that has been isolated from a producer cell (i.e., base EV). In some aspects, the targeting moiety is added to the EV before adding the antigen. In some aspects, the targeting moiety is added to the EV after adding the antigen. In some aspects, the targeting moiety is added to the EV together with the antigen. Non-limiting examples of targeting moiety that can be used with the present methods are provided elsewhere in the present disclosure.

As described herein, in producing or manufacturing an EV-based vaccine with the methods disclosed herein, an antigen or any other molecules of interest (e.g., adjuvant and/or targeting moiety) can be added to the base EV, such that the antigen (or any other molecule of interest) is associated with a surface of the EV or in the lumen of the EV. For instance, in some aspects, an antigen is: (i) linked directly to the exterior surface of the EV, (ii) linked directly to the luminal surface of the EV, (iii) in the lumen of the EV, or (iv) any combination thereof. In some aspects, an adjuvant is: (i) linked directly to the exterior surface of the EV, (ii) linked directly to the luminal surface of the EV, (iii) in the lumen of the EV, or (iv) any combination thereof. In some aspects, a targeting moiety is: (i) linked directly to the exterior surface of the EV, (ii) linked directly to the luminal surface of the EV, (iii) in the lumen of the EV, or (iv) any combination thereof.

In some aspects, any suitable method can be used to link an antigen or any other molecules of interest (e.g., adjuvant and/or targeting moiety) to an exterior surface and/or luminal surface of the EV (e.g., exosome). In certain aspects, the antigen or any other molecules of interest (e.g., adjuvant and/or targeting moiety) is linked to the exterior surface and/or the luminal surface of the EV by any suitable coupling strategies known in the art. In some aspects, the coupling strategy comprises: an anchoring moiety, affinity agent, chemical conjugation, cell penetrating peptide (CPP), split intein, SpyTag/SpyCatcher, ALFA-tag, Streptavidin/Avitag, Sortase, SNAP-tag, ProA/Fc-binding peptide, or any combinations thereof. In some aspects, the anchoring moiety comprises a sterol (e.g., cholesterol), GM1, lipid (e.g., fatty acid (e.g., palmitate), ionizable lipid, glycerophospholipid, sphingolipid), vitamin (e.g., tocopherol (e.g., vitamin E), vitamin A, vitamin D, vitamin K), alkyl chain, aromatic ring, small molecule, peptide, including any derivatives thereof, or any combination thereof. In some aspects, the chemical conjugation comprises a maleimide moiety, copper-free, biorthogonal click chemistry (e.g., azide/strained alkyne (e.g., DIFO, dibenzocyclooctyne (DBCO), bicyclononyne (BCN)), metal-catalyzed click chemistry (e.g., CuAAC, RuAAC), or any combination thereof. Additional description relating to the different approaches of linking an antigen or any other molecules of interest (e.g., adjuvant and/or targeting moiety) are provided elsewhere in the present disclosure.

In some aspects, an EV (e.g., exosome) that can be prepared or manufactured using the methods described herein can further comprise a scaffold moiety. In certain aspects, the EV comprises the scaffold moiety prior to the addition of the antigen to the EV. In some aspects, the methods of preparing or manufacturing EV-based vaccines described herein further comprise adding a scaffold moiety to an EV (e.g., exosome) that has been isolated from a producer cell (i.e., base EV). In some aspects, the scaffold moiety is added to the EV before adding the antigen. In some aspects, the scaffold moiety is added to the EV after adding the antigen. In some aspects, the scaffold moiety is added to the EV together with the antigen.

Accordingly, in some aspects, an antigen is linked to a scaffold moiety on the exterior surface and/or luminal surface of the EV (e.g., exosome). In some aspects, an adjuvant is linked to a scaffold moiety on the exterior surface and/or luminal surface of the EV (e.g., exosome). In some aspects, a targeting moiety is linked to a scaffold moiety on the exterior surface and/or luminal surface of the EV (e.g., exosome). In some aspects, any combination of an antigen, adjuvant, and/or targeting moiety are linked to a scaffold moiety on the exterior surface and/or luminal surface of the EV.

In some aspects, the scaffold moiety comprises Scaffold X (e.g., PTGFRN or a fragment thereof). In some aspects, the scaffold moiety comprises Scaffold Y (e.g., BASP-1 or a fragment thereof). In some aspects, the scaffold moiety comprises both Scaffold X (e.g., PTGFRN or a fragment thereof) and Scaffold Y (e.g., BASP-1 or a fragment thereof).

Non-limiting examples of scaffold moieties that can be used with the present methods are provided elsewhere in the present disclosure.

### Methods of Producing EVs (e.g., Exosomes)

As will be apparent from the present disclosure, one of the features of the EV-based vaccine platform disclosed herein is that the base EVs (e.g., exosomes) can be produced and stored indefinitely until they are to be used with the methods disclosed herein. For instance, as described herein, in producing the base EVs (e.g., exosomes), they can be initially produced to comprise one or more moieties of interest, such as those that could be beneficial in a wide range of diseases or disorders (e.g., adjuvant and/or targeting moiety). Then, when needed (e.g., in the presence of a pandemic), the base EVs can be rapidly modified to comprise an antigen of interest, and thereby, produce or manufacture a vaccine that meets the need. Such antigens can be added to the base EVs at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 2 years, at least about 3 years, at least about 4 years, or at least about 5 year or more after isolating the base EV from the producer cell.

Accordingly, in some aspects, the present disclosure is also directed to methods of producing the base EVs (e.g., exosomes) described herein. In some aspects, the method comprises: obtaining the base EV (e.g., exosome) from a producer cell, wherein the producer cell contains one or more molecules of interest described herein (e.g., adjuvant, targeting moiety and/or scaffold moiety); and optionally isolating the obtained EV (e.g., exosome). In certain aspects, the producer cell contains an adjuvant. In some aspects, the producer cell contains a targeting moiety. In certain aspects, the producer contains any combination of an adjuvant and/or targeting moiety. In some aspects, the method comprises: modifying a producer cell by introducing one or more molecules of interest (e.g., adjuvant, targeting moiety, and/or scaffold moiety); obtaining the EV (e.g., exosome) from the modified producer cell; and optionally isolating the obtained EV (e.g., exosome).

In some aspects, the producer cell can be a mammalian cell line, a plant cell line, an insect cell line, a fungi cell line, or a prokaryotic cell line. In certain aspects, the producer cell is a mammalian cell line. Non-limiting examples of mammalian cell lines include: a human embryonic kidney (HEK) cell line, a Chinese hamster ovary (CHO) cell line, an HT-1080 cell line, a HeLa cell line, a PERC-6 cell line, a CEVEC cell line, a fibroblast cell line, an amniocyte cell line, an epithelial cell line, a mesenchymal stem cell (MSC) cell line, and combinations thereof. In certain aspects, the mammalian cell line comprises HEK-293 cells, BJ human foreskin fibroblast cells, fHDF fibroblast cells, AGE.HN® neuronal precursor cells, CAP® amniocyte cells, adipose mesenchymal stem cells, RPTEC/TERT1 cells, or combinations thereof. In some aspects, the producer cell is a primary cell. In certain aspects, the primary cell can be a primary mammalian cell, a primary plant cell, a primary insect cell, a primary fungi cell, or a primary prokaryotic cell.

In some aspects, the producer cell is not an immune cell, such an antigen presenting cell, a T cell, a B cell, a natural killer cell (NK cell), a macrophage, a T helper cell, or a regulatory T cell (Treg cell). In some aspects, the producer cell is not an antigen presenting cell (e.g., dendritic cells, macrophages, B cells, mast cells, neutrophils, Kupffer-Browicz cell, or a cell derived from any such cells).

In some aspects, the one or more moieties, e.g., targeting moiety and/or scaffold moiety, can be a transgene or mRNA, and introduced into the producer cell by transfection, viral transduction, electroporation, extrusion, sonication, cell fusion, or other methods that are known to the skilled in the art.

In some aspects, the one or more moieties, e.g., targeting moiety and/or scaffold moiety, is introduced to the producer cell by transfection. In some aspects, the one or more moieties can be introduced into suitable producer cells using synthetic macromolecules, such as cationic lipids and polymers (Papapetrou et al., Gene Therapy 12: S118-S130 (2005)). In some aspects, the cationic lipids form complexes with the one or more moieties through charge interactions. In some of these aspects, the positively charged complexes bind to the negatively charged cell surface and are taken up by the cell by endocytosis. In some aspects, a cationic polymer can be used to transfect producer cells. In some of these aspects, the cationic polymer is polyethylenimine (PEI). In certain aspects, chemicals such as calcium phosphate, cyclodextrin, or polybrene, can be used to introduce the one or more moieties to the producer cells. The one or more moieties can also be introduced into a producer cell using a physical method such as particle-mediated transfection, “gene gun”, biolistics, or particle bombardment technology (Papapetrou et al., Gene Therapy 12: S118-S130 (2005)). A reporter gene such as, for example, beta-galactosidase, chloramphenicol acetyltransferase, luciferase, or green fluorescent protein can be used to assess the transfection efficiency of the producer cell.

In certain aspects, the one or more moieties are introduced to the producer cell by viral transduction. A number of viruses can be used as gene transfer vehicles, including moloney murine leukemia virus (MMLV), adenovirus, adeno-associated virus (AAV), herpes simplex virus (HSV), lentiviruses, and spumaviruses. The viral mediated gene transfer vehicles comprise vectors based on DNA viruses, such as adenovirus, adeno-associated virus and herpes virus, as well as retroviral based vectors.

In certain aspects, the one or more moieties are introduced to the producer cell by electroporation. Electroporation creates transient pores in the cell membrane, allowing for the introduction of various molecules into the cell. In some aspects, DNA and RNA as well as polypeptides and non-polypeptide therapeutic agents can be introduced into the producer cell by electroporation.

In certain aspects, the one or more moieties introduced to the producer cell by microinjection. In some aspects, a glass micropipette can be used to inject the one or more moieties into the producer cell at the microscopic level.

In certain aspects, the one or more moieties are introduced to the producer cell by extrusion.

In certain aspects, the one or more moieties are introduced to the producer cell by sonication. In some aspects, the producer cell is exposed to high intensity sound waves, causing transient disruption of the cell membrane allowing loading of the one or more moieties.

In certain aspects, the one or more moieties are introduced to the producer cell by cell fusion. In some aspects, the one or more moieties are introduced by electrical cell fusion. In some aspects, polyethylene glycol (PEG) is used to fuse the producer cells. In further aspects, sendai virus is used to fuse the producer cells.

In some aspects, the one or more moieties are introduced to the producer cell by hypotonic lysis. In such aspects, the producer cell can be exposed to low ionic strength buffer causing them to burst allowing loading of the one or more moieties. In some aspects, controlled dialysis against a hypotonic solution can be used to swell the producer cell and to create pores in the producer cell membrane. The producer cell is subsequently exposed to conditions that allow resealing of the membrane.

In some aspects, the one or more moieties are introduced to the producer cell by detergent treatment. In certain aspects, producer cell is treated with a mild detergent which transiently compromises the producer cell membrane by creating pores allowing loading of the one or more moieties. After producer cells are loaded, the detergent is washed away thereby resealing the membrane.

In some aspects, the one or more moieties introduced to the producer cell by receptor mediated endocytosis. In certain aspects, producer cells have a surface receptor which upon binding of the one or more moieties induces internalization of the receptor and the associated moieties.

In some aspects, the one or more moieties are introduced to the producer cell by filtration. In certain aspects, the producer cells and the one or more moieties can be forced through a filter of pore size smaller than the producer cell causing transient disruption of the producer cell membrane and allowing the one or more moieties to enter the producer cell.

In some aspects, the producer cell is subjected to several freeze thaw cycles, resulting in cell membrane disruption allowing loading of the one or more moieties.

In some aspects, therefore, the EVs produced prior to the loading of an antigen can be equipped with an adjuvant, targeting moiety, and/or scaffold moiety and are ready to be loaded with one or more antigens.

### Methods of Isolating an EV, e.g., Exosome

As described herein, in producing the EV-based vaccines of the present disclosure, the base EVs (e.g., exosomes), once produced (e.g., as described above), are isolated from the producer cells. Accordingly, in some aspects, methods of producing or manufacturing the EV-based vaccines comprise isolating the base EV (e.g., exosome) from the producer cells. In certain aspects, the base EVs (e.g., exosomes) are released by the producer cell into the cell culture medium. It is contemplated that all known manners of isolation of EVs (e.g., exosomes) are deemed suitable for use herein. For example, physical properties of EVs (e.g., exosomes) can be employed to separate them from a medium or other source material, including separation on the basis of electrical charge (e.g., electrophoretic separation), size (e.g., filtration, molecular sieving, etc.), density (e.g., regular or gradient centrifugation), Svedberg constant (e.g., sedimentation with or without external force, etc.). Alternatively, or additionally, isolation can be based on one or more biological properties, and include methods that can employ surface markers (e.g., for precipitation, reversible binding to solid phase, FACS separation, specific ligand binding, non-specific ligand binding, affinity purification etc.).

Isolation and enrichment can be done in a general and non-selective manner, typically including serial centrifugation. Alternatively, isolation and enrichment can be done in a more specific and selective manner, such as using EV (e.g., exosome) or producer cell-specific surface markers. For example, specific surface markers can be used in immunoprecipitation, FACS sorting, affinity purification, and magnetic separation with bead-bound ligands.

In some aspects, one or more chromatography can be utilized to isolate the EVs (e.g., exosomes). In some aspects, the EVs can be isolated with size exclusion chromatography techniques are known in the art. Exemplary, non-limiting techniques are provided herein. In some aspects, a void volume fraction is isolated and comprises the EVs (e.g., exosomes) of interest. Further, in some aspects, the EVs (e.g., exosomes) can be further isolated after chromatographic separation by centrifugation techniques (of one or more chromatography fractions), as is generally known in the art. In some aspects, for example, density gradient centrifugation can be utilized to further isolate the extracellular vesicles. In certain aspects, it can be desirable to further separate the producer cell-derived EVs (e.g., exosomes) from EVs (e.g., exosomes) of other origin. For example, the producer cell-derived EVs (e.g., exosomes) can be separated from non-producer cell-derived EVs (e.g., exosomes) by immunosorbent capture using an antigen antibody specific for the producer cell.

In some aspects, the isolation of EVs (e.g., exosomes) can involve combinations of methods that include, but are not limited to, differential centrifugation, size-based membrane filtration, immunoprecipitation, FACS sorting, and magnetic separation.

Exemplary methods of isolating one or more EVs are disclosed in PCT Application No. PCT/US2020/024038, filed Mar. 20, 2020, and published as WO 2020/191369 on Sep. 24, 2020, which is incorporated herein by reference in its entirety.

### Methods of Modifying EVs (e.g., Exosomes)

As described herein, to produce the EV-based vaccines of the present disclosure, the methods provided herein comprise modifying the base EV (e.g., exosome) to comprise an antigen, e.g., in combination with one or more molecules of interest described herein (e.g., adjuvant, targeting moiety, and/or scaffold moiety). Accordingly, in certain aspects, a method of producing or manufacturing an EV-based vaccine described herein comprises modifying the EV (e.g., exosome) that has been isolated from the producer cell (i.e., base EV) by directly introducing the antigen and/or other molecules of interest described herein (e.g., adjuvant, targeting moiety, and/or scaffold moiety).

In some aspects, the antigen and/or other molecules of interest described herein (e.g., adjuvant, targeting moiety, and/or scaffold moiety) are introduced to the EV by transfection. In some aspects, the antigen and/or other molecules of interest described herein (e.g., adjuvant, targeting moiety, and/or scaffold moiety) can be introduced into the EV (e.g., exosome) using synthetic macromolecules such as cationic lipids and polymers, such as those described in Papapetrou et al., Gene Therapy 12: S118-S130 (2005), which is incorporated herein by reference in its entirety. In certain aspects, chemicals such as calcium phosphate, cyclodextrin, or polybrene, can be used to introduce the antigen and/or other molecules of interest described herein (e.g., adjuvant, targeting moiety, and/or scaffold moiety) to the EV (e.g., exosome).

In certain aspects, the antigen and/or other molecules of interest described herein (e.g., adjuvant, targeting moiety, and/or scaffold moiety) are introduced to the EV (e.g., exosome) by electroporation. In some aspects, the EVs (e.g., exosomes) are exposed to an electrical field which causes transient holes in the EV (e.g., exosome) membrane, allowing loading of the antigen and/or other molecules of interest described herein (e.g., adjuvant, targeting moiety, and/or scaffold moiety).

In certain aspects, the antigen and/or other molecules of interest described herein (e.g., adjuvant, targeting moiety, and/or scaffold moiety) are introduced to the EV (e.g., exosome) by microinjection. In some aspects, a glass micropipette can be used to inject the antigen and/or other molecules of interest described herein (e.g., adjuvant, targeting moiety, and/or scaffold moiety) directly into the EV (e.g., exosome) at the microscopic level.

In certain aspects, the antigen and/or other molecules of interest described herein (e.g., adjuvant, targeting moiety, and/or scaffold moiety) are introduced to the EV (e.g., exosome) by extrusion.

In certain aspects, the antigen and/or other molecules of interest described herein (e.g., adjuvant, targeting moiety, and/or scaffold moiety) are introduced to the EV (e.g., exosome) by sonication. In some aspects, EVs (e.g., exosomes) are exposed to high intensity sound waves, causing transient disruption of the EV (e.g., exosome) membrane allowing loading of the antigen and/or other molecules of interest described herein (e.g., adjuvant, targeting moiety, and/or scaffold moiety).

As described herein, in some aspects, antigen and/or other molecules of interest described herein (e.g., adjuvant, targeting moiety, and/or scaffold moiety) can be conjugated to the surface of the EV (e.g., exosome). Conjugation can be achieved chemically or enzymatically, by methods known in the art. In some aspects, conjugation can occur via non-covalent lipid association.

In some aspects, the EV (e.g., exosome) comprises antigen and/or other molecules of interest described herein (e.g., adjuvant, targeting moiety, and/or scaffold moiety) that are chemically conjugated. Chemical conjugation can be accomplished by covalent bonding of one or more moieties to another molecule, with or without use of a linker. The formation of such conjugates is within the skill of artisans and various techniques are known for accomplishing the conjugation, with the choice of the particular technique being guided by the materials to be conjugated. In certain aspects, polypeptides are conjugated to the EV (e.g., exosome). In some aspects, non-polypeptides, such as lipids, carbohydrates, nucleic acids, and small molecules, are conjugated to the EV (e.g., exosome).

In some aspects, the antigen and/or other molecules of interest described herein (e.g., adjuvant, targeting moiety, and/or scaffold moiety) are introduced to the EV (e.g., exosome) by hypotonic lysis. In such aspects, the EVs (e.g., exosomes) can be exposed to low ionic strength buffer causing them to burst allowing loading of the antigen and/or other molecules of interest described herein (e.g., adjuvant, targeting moiety, and/or scaffold moiety). In some aspects, controlled dialysis against a hypotonic solution can be used to swell the EV (e.g., exosome) and to create pores in the EV (e.g., exosome) membrane. The EV (e.g., exosome) is subsequently exposed to conditions that allow resealing of the membrane.

In some aspects, the antigen and/or other molecules of interest described herein (e.g., adjuvant, targeting moiety, and/or scaffold moiety) are introduced to the EV (e.g., exosome) by detergent treatment. In certain aspects, extracellular vesicles are treated with a mild detergent which transiently compromises the EV (e.g., exosome) membrane by creating pores allowing loading of the antigen and/or other molecules of interest described herein (e.g., adjuvant, targeting moiety, and/or scaffold moiety). After EVs (e.g., exosomes) are loaded, the detergent is washed away thereby resealing the membrane.

In some aspects, the antigen and/or other molecules of interest described herein (e.g., adjuvant, targeting moiety, and/or scaffold moiety) are introduced to the EV (e.g., exosome) by receptor mediated endocytosis. In certain aspects, EVs (e.g., exosomes) have a surface receptor which upon binding of the antigen and/or other molecules of interest described herein (e.g., adjuvant, targeting moiety, and/or scaffold moiety) induces internalization of the receptor and the associated moieties.

In some aspects, the antigen and/or other molecules of interest described herein (e.g., adjuvant, targeting moiety, and/or scaffold moiety) are introduced to the EV (e.g., exosome) by mechanical firing. In certain aspects, extracellular vesicles can be bombarded with antigen and/or other molecules of interest described herein (e.g., adjuvant, targeting moiety, and/or scaffold moiety) attached to a heavy or charged particle such as gold microcarriers. In some of these aspects, the particle can be mechanically or electrically accelerated such that it traverses the EV (e.g., exosome) membrane.

In some aspects, extracellular vesicles (EVs) are subjected to several freeze thaw cycles, resulting in EV (e.g., exosome) membrane disruption allowing loading of the antigen and/or other molecules of interest described herein (e.g., adjuvant, targeting moiety, and/or scaffold moiety).

Non-limiting examples of such molecules, e.g., antigens, adjuvants, targeting moieties, and/or scaffold moieties are disclosed in PCT Application No. PCT/US2020/024023, which published as WO 2020/191361 on Sep. 24, 2020, each of which is incorporated herein by reference in its entirety.

### Methods of Treatment

In some aspects, the present disclosure also relates to methods of preventing and/or treating a disease or disorder in a subject in need thereof. As described herein, the EV-based vaccines of the present disclosure differ from traditional vaccines in that the EVs (e.g., exosomes) can be rapidly modified to any antigen of interest, such as those associated with an ongoing pandemic. Because they can be produced and manufactured much more quickly than traditional vaccines, the EV-based vaccines disclosed herein can be more effective in controlling a disease outbreak and prevent a potential pandemic, compared to traditional vaccines. Additionally, as described further elsewhere in the present disclosure, the EV-based vaccines described herein can be particularly useful as regionalized vaccines and/or individualized vaccines.

EVs (e.g., exosomes) of the present disclosure can be administered to a subject by any useful method and/or route known in the art. In some aspects, the EVs (e.g., exosomes) are administered intravenously to the circulatory system of the subject. In some aspects, the EVs (e.g., exosomes) are infused in suitable liquid and administered into a vein of the subject.

In some aspects, the EVs (e.g., exosomes) are administered intra-arterially to the circulatory system of the subject. In some aspects, the EVs (e.g., exosomes) are infused in suitable liquid and administered into an artery of the subject.

In some aspects, the EVs (e.g., exosomes) are administered to the subject by intranasal administration. In some aspects, the EVs (e.g., exosomes) can be insufflated through the nose in a form of either topical administration or systemic administration. In certain aspects, the EVs (e.g., exosomes) are administered as nasal spray. In some aspects, intranasal administration can allow for the effective delivery of an EV (e.g., exosome) disclosed herein to the gastrointestinal tissues. Such EVs (e.g., exosomes) delivered to the gastrointestinal tissues could be useful in providing protection against various gut-associated pathogens.

In some aspects, the EVs (e.g., exosomes) are administered to the subject by intraperitoneal administration. In some aspects, the EVs (e.g., exosomes) are infused in suitable liquid and injected into the peritoneum of the subject. In some aspects, the intraperitoneal administration results in distribution of the EVs (e.g., exosomes) to the lymphatics. In some aspects, the intraperitoneal administration results in distribution of the EVs (e.g., exosomes) to the thymus, spleen, and/or bone marrow. In some aspects, the intraperitoneal administration results in distribution of the EVs (e.g., exosomes) to one or more lymph nodes. In some aspects, the intraperitoneal administration results in distribution of the EVs (e.g., exosomes) to one or more of the cervical lymph node, the inguinal lymph node, the mediastinal lymph node, or the sternal lymph node. In some aspects, the intraperitoneal administration results in distribution of the EVs (e.g., exosomes) to the pancreas.

Non-limiting examples of other routes of administration that can be used to administer the EVs (e.g., exosomes) disclosed herein include parenteral, topical, oral, subcutaneous, intradermal, transdermal, rectal, intraperitoneal, intramuscular, sublingual, intrathecal, or combinations thereof.

As disclosed herein, in some aspects, EVs (e.g., exosomes) disclosed herein can be administered to a subject in combination with one or more additional agents, e.g., adjuvants. In certain aspects, the one or more additional agents and the EVs (e.g., exosomes) are administered concurrently. In some aspects, the one or more additional agents and the EVs (e.g., exosomes) are administered sequentially. In some aspects, the EVs (e.g., exosomes) are administered to the subject prior to administering the one or more additional agents. In certain aspects, the EVs (e.g., exosome) are administered to the subject after administering the one or more additional agents. As used herein, the term “agents” refers to any agents that can be used in combination with the present EVs, e.g., in treating an infectious disease or disorder disclosed herein). In some aspects, the one or more additional agents that can be used in combination with the EVs (e.g., exosomes) of the present disclosure include a payload (e.g., antigen, adjuvant, and/or immune modulator) which is not expressed in an EV (e.g., exosome). For instance, a treatment method disclosed herein can comprise administering to a subject in need thereof (i) an antigen-expressing EV (e.g., exosome) and (ii) an antigen that is the same or different from that expressed in the EV (e.g., soluble antigen).

EVs described herein can be administered to a subject using any suitable devices known in the art. Non-limiting examples of such devices include: pumps, patches, microneedles, dissolvable strips, and a combination thereof.

In some aspects, a subject that can be treated with the present disclosure is a human. In some aspects, a subject is a non-human mammal (e.g., non-human primates, dogs, cats, guinea pigs, rabbits, rats, mice, horses, cattle, chickens, birds, and bears). Accordingly, in some aspects, the EVs (e.g., exosomes) disclosed herein can be used to improve the health of an animal (i.e., non-human mammal).

### Extracellular Vesicles, e.g., Exosomes

Disclosed herein are EVs (e.g., exosomes) that are suitable as vaccines and prepared or manufactured using the methods described herein. As described herein, EVs (e.g., exosomes) described herein differ from other vaccine platforms for treating diseases and disorders in that the EVs comprise one or more of the following properties: (i) flexibility of moiety (e.g., antigen) display, (ii) diverse adjuvant and immunomodulatory combinations, (iii) enhanced cell-specific tropism, (iv) enhanced clearance inhibition, or (v) any combination thereof.

In some aspects, EVs (e.g., exosomes) of the present disclosure provide flexibility of moiety display. For instance, the moieties of interest (e.g., antigen) (i) can be directly linked to a surface of the EV (e.g., exterior surface and/or luminal surface), (ii) can be linked to a scaffold moiety (e.g., Scaffold X and/or Scaffold Y) and then expressed on a surface of the EV (e.g., exterior surface and/or luminal surface), (iii) can be expressed in the lumen of the EV, or (iv) combinations thereof. Such ability to rapidly engineer EVs (e.g., exosomes) is particularly useful in developing EV (e.g., exosome)-based vaccines for treating the diseases and disorders described herein. For instance, a single EV (e.g., exosome) engineered to express certain payloads and/or targeting moieties can be used in treating a wide range of diseases or disorders by simply “plugging” a moiety (e.g., antigen of interest) into the EVs (or rapidly attaching a moiety (e.g., antigen of interest) as a “clip-on” attachment to the EVs). Methods of producing such modular or “plug and play” EVs are provided elsewhere in the present disclosure.

In some aspects, EVs (e.g., exosomes) of the present disclosure allow for the diverse combinations of different moieties of interest (e.g., antigens, adjuvants, immunomodulators, and/or targeting moieties). In certain aspects, the EVs (e.g., exosomes) allow for the combination of a wide range of adjuvants and immunomodulators. Non-limiting examples of adjuvants and immunomodulators that can be combined in a single EV (e.g., exosome) include small molecule agonists (e.g., STING), small molecule antagonists, co-stimulatory proteins, anti-sense and bacterial adjuvant oligonucleotides. Additional disclosure relating to the different moieties that can be combined together are provided elsewhere in the present disclosure.

In some aspects, EVs (e.g., exosomes) described herein can be engineered to exhibit enhanced cell-specific tropism. For instance, the EVs can be engineered to express on their exterior surface a targeting moiety (e.g., antibodies and/or proteins) that can specifically bind to a marker on a specific cell. In some aspects, EVs (e.g., exosomes) described herein can be engineered to induce certain types of immune responses (e.g., T cell, B cell, and/or Treg/tolerogenic immune responses). Additional disclosure relating to such properties are provided elsewhere in the present disclosure.

The EVs, e.g., exosomes, useful in the present disclosure have been engineered to produce multiple (e.g., at least two) exogenous biologically active molecules (e.g., an antigen, an antibody or an antigen-binding fragment thereof, an adjuvant, and/or an immune modulator), and/or other moieties (e.g., a targeting moiety) together in a single EV, e.g., exosome. In some aspects, an EV (e.g., exosome) comprises three exogenous biologically active molecules. In some aspects, an EV (e.g., exosome) comprises four exogenous biologically active molecules. In further aspects, an EV (e.g., exosome) comprises five or more exogenous biologically active molecules. In some aspects, an EV (e.g., exosome) comprises 6, 7, 8, 9, 10, 11, 12, 13, 14, 15, 16, 17, 18, 19, 20 or more exogenous biologically active molecules.

In some aspects, an EV (e.g., exosome) comprises two or more exogenous biologically active molecules, e.g., (i) one or more antigens and (ii) one or more adjuvants. In some aspects, an EV (e.g., exosome) comprises two or more exogenous biologically active molecules, e.g., (i) one or more antigens and (ii) one or more immune modulators. In further aspects, an EV (e.g., exosome) comprises two or more exogenous biologically active molecules, e.g., (i) one or more antigens, (ii) one or more immune modulators, and (iii) one or more adjuvants. In certain aspects, an EV (e.g., exosome) can further comprise one or more additional moieties, e.g., targeting moieties. In some aspects, an antigen is not expressed (or presented) on major histocompatibility complex I and/or II molecules. In some aspects, while an antigen in the EV, e.g., exosome, is not expressed or presented as part of the MHC class I or II complex, the EV, e.g., exosome, can still contain MHC class I/II molecules on the surface of the EV, e.g., exosome. Accordingly, in certain aspects, EVs, e.g., exosomes, disclosed herein do not directly interact with T-cell receptors (TCRs) of T cells to induce an immune response against the antigen. Similarly, in certain aspects, EVs, e.g., exosomes, of the present disclosure do not transfer the antigen directly to the surface of the target cell (e.g., dendritic cell) through cross-dressing. Cross-dressing is a mechanism commonly used by EVs, e.g., exosomes, derived from dendritic cells (DEX) to induce T cell activation. See Pitt, J. M., et al., J Clin Invest 126(4): 1224-32 (2016). In some aspects, the EVs, e.g., exosomes, of the present disclosure are engulfed by antigen presenting cells and can be expressed on the surface of the antigen presenting cells as MHC class I and/or MHC class II complex.

As described supra, EVs, e.g., exosomes, described herein are extracellular vesicles with a diameter between about 20-300 nm. In certain aspects, an EV, e.g., exosome, of the present disclosure has a diameter between about 20-80 nm, between about 20-100 nm, between about 20-200 nm, between about 80-300 nm, between about 80-290 nm, between about 80-280 nm, between about 80-270 nm, between about 80-260 nm, between about 80-250 nm, between about 80-240 nm, between about 80-230 nm, between about 80-220 nm, between about 80-210 nm, between about 80-200 nm, between about 80-190 nm, between about 80-180 nm, between about 80-170 nm, between about 80-160 nm, between about 80-150 nm, between about 80-140 nm, between about 80-130 nm, between about 80-120 nm, between about 80-110 nm, between about 80-100 nm, between about 80-90 nm, between about 90-300 nm, between about 90-290 nm, between about 90-280 nm, between about 90-270 nm, between about 90-260 nm, between about 90-250 nm, between about 90-240 nm, between about 90-230 nm, between about 90-220 nm, between about 90-210 nm, between about 90-200 nm, between about 90-190 nm, between about 90-180 nm, between about 90-170 nm, between about 90-160 nm, between about 90-150 nm, between about 90-140 nm, between about 90-130 nm, between about 90-120 nm, between about 90-110 nm, between about 90-100 nm, between about 100-300 nm, between about 110-290 nm, between about 120-280 nm, between about 130-270 nm, between about 140-260 nm, between about 150-250 nm, between about 160-240 nm, between about 170-230 nm, between about 180-220 nm, or between about 190-210 nm. The size of the EV, e.g., exosome, described herein can be measured according to methods described, infra.

In some aspects, an EV, e.g., exosome, of the present disclosure comprises a bi-lipid membrane (“EV, e.g., exosome, membrane”), comprising an interior surface and an exterior surface. In certain aspects, the interior surface faces the inner core (i.e., lumen) of the EV, e.g., exosome. In certain aspects, the exterior surface can be in contact with the endosome, the multivesicular bodies, or the membrane/cytoplasm of a producer cell or a target cell.

In some aspects, the EV, e.g., exosome, membrane comprises lipids and fatty acids. In some aspects, the EV, e.g., exosome, membrane comprises phospholipids, glycolipids, fatty acids, sphingolipids, phosphoglycerides, sterols, cholesterols, and phosphatidylserines.

In some aspects, the EV, e.g., exosome, membrane comprises an inner leaflet and an outer leaflet. The composition of the inner and outer leaflet can be determined by transbilayer distribution assays known in the art, see, e.g., Kuypers et al., Biohim Biophys Acta 1985 819:170. In some aspects, the composition of the outer leaflet is between approximately 70-90% choline phospholipids, between approximately 0-15% acidic phospholipids, and between approximately 5-30% phosphatidylethanolamine. In some aspects, the composition of the inner leaflet is between approximately 15-40% choline phospholipids, between approximately 10-50% acidic phospholipids, and between approximately 30-60% phosphatidylethanolamine.

In some aspects, the EV, e.g., exosome, membrane comprises one or more polysaccharide, such as glycan.

In some aspects, the EV, e.g., exosome, membrane further comprises one or more scaffold moieties, which are capable of anchoring, e.g., an antigen and/or an adjuvant and/or an immune modulator, to the EV, e.g., exosome, (e.g., either on the luminal surface or on the exterior surface). In certain aspects, scaffold moieties are polypeptides (“exosome proteins”). In some aspects, scaffold moieties are non-polypeptide moieties. In some aspects, exosome proteins include various membrane proteins, such as transmembrane proteins, integral proteins and peripheral proteins, enriched on the exosome membranes. They can include various CD proteins, transporters, integrins, lectins, and cadherins. In certain aspects, a scaffold moiety (e.g., exosome protein) comprises Scaffold X. In some aspects, a scaffold moiety (e.g., exosome protein) comprises Scaffold Y. In further aspects, a scaffold moiety (e.g., exosome protein) comprises both a Scaffold X and a Scaffold Y.

In some aspects, an EV, e.g., exosome, disclosed herein is capable of delivering a payload (e.g., an antigen, an adjuvant, and/or an immune modulator) to a target. The payload is an agent that acts on a target (e.g., a target cell) that is contacted with the EV. Contacting can occur in vitro or in a subject. Non-limiting examples of payloads that can be introduced into an EV include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, siRNA, antisense oligonucleotide, a phosphorodiamidate morpholino oligomer (PMO), or a peptide-conjugated phosphorodiamidate morpholino oligomer (PPMO)), amino acids (e.g., amino acids comprising a detectable moiety or a toxin that disrupt translation), polypeptides (e.g., enzymes), lipids, carbohydrates, and small molecules (e.g., small molecule drugs and toxins).

As demonstrated herein, in some aspects, EVs (e.g., exosomes) of the present disclosure are capable of inducing effector and memory T cells. In certain aspects, the memory T cells are tissue-resident memory T cells. Such EVs (e.g., exosomes) could be particularly useful as vaccines for certain infectious diseases, coronavirus, e.g., SARS-CoV-1 and/or SARS-CoV-2 (COVID-19) virus.

In some aspects, EVs (e.g., exosomes) disclosed herein are inherently capable of inducing the activation of a signaling pathway involved in an immune response. In certain aspects, the signaling pathway involved in an immune response comprises toll-like receptors (TLRs), retinoid acid-inducible gene I (RIG-I)-like receptors (RLRs), stimulator of interferon genes (STING) pathway, or combinations thereof. In some aspects, the activation of such signaling pathway can result in the production of a type I interferon. For example, in certain aspects, the bi-lipid membrane of an EV (e.g., exosome) disclosed herein comprises one or more lipids that share one of the following features: (i) unsaturated lipid tail, (ii) dihydroimidazole linker, (iii) cyclic amine head groups, and (iv) combinations thereof. Lipids with such features have been shown to activate the TLR/RLR-independent STING pathway. See Miao et al., Nature Biotechnology 37:1174-1185 (October 2019), which is herein incorporated by reference in its entirety.

### Antigen

In some aspects, an antigen that can be added to a base EV (e.g., exosome) to produce the EV-based vaccines disclosed herein comprises any antigen known in the art, which is capable of eliciting a beneficial immune response in a subject. As used herein, a “beneficial immune response” is an immune response that is capable of treating (e.g., reducing and/or alleviating one or more symptoms) and/or preventing a disease or disorder, such as those described herein.

In some aspects, an antigen comprises a peptide. In certain aspects, the peptide comprises a natural peptide (e.g., such as that derived from a naturally-existing organism, e.g., virus). In some aspects, the peptide comprises a synthetic peptide. In some aspects, the peptide comprises both natural and synthetic peptides.

In some aspects, the peptide is less than about 150 amino acids in length, less than about 140 amino acids in length, less than about 130 amino acids in length, less than about 120 amino acids in length, less than about 110 amino acids in length, less than about 100 amino acids in length, less than about 90 amino acids in length, less than about 80 amino acids in length, less than about 70 amino acids in length, less than about 60 amino acids in length, less than about 50 amino acids in length, less than about 40 amino acids in length, less than about 30 amino acids in length, less than about 20 amino acids in length, or less than about 10 amino acids in length. In certain aspects, the peptide is less than about 100 amino acids in length. In some aspects, the peptide is less than about 80 amino acids in length.

In some aspects, an antigen useful for the present disclosure is a polynucleotide, e.g., an mRNA. In some aspects, an antigen useful for the disclosure is a synthetic mRNA encoding an epitope.

As described herein, in some aspects, an antigen can be linked to the exterior surface and/or luminal surface of EVs (e.g., exosomes) by various methods, including, but not limited to, anchoring moieties, affinity agents, chemical conjugation, or combinations thereof. To improve the attachment of the antigens to a surface of the EVs (e.g., exosomes) using such methods, the antigens described herein can be further modified. In certain aspects, an antigen comprises a peptide, which has been modified to contain a N-terminal lysine.

In some aspects, such a modification allows for the attachment of the antigen to a surface of the EV (e.g., exosome) with chemical conjugation. For example, to enable click chemistry conjugation, an azide or strained alkyne (e.g., difluorinated cyclooctyne (DIFO), dibenzocyclooctyne (DBCO), bicyclononyne (BCN)) would have to be attached (or linked) to the EV (e.g., exosome) and/or to the antigen. In certain aspects, DIFO conjugates to the primary amine side chain on the N-terminal (of any amino acid) of the antigen, which in turn can interact with the azide that can be attached to a surface of the EV. In some aspects, the azide can be attached to the antigen (via the primary amine side chain on the N-terminal, e.g., lysine or any other suitable amino acid), and the strained alkyne can be attached to a surface of the EV (e.g., exosome).

In some aspects, modifying the antigen to comprise a N-terminal lysine can also be useful in linking the antigens to a surface of the EVs (e.g., exosomes) using anchoring moieties. In such aspects, the anchoring moiety (i.e., cholesterol, fatty acid, and/or vitamin E) can attach to the N-terminal lysine via the primary amine side chain. Once attached, the antigens can be readily inserted into the membrane of the EVs (e.g., exosomes) via the anchoring moieties.

As will be apparent to those skilled in the arts, the above described approaches to linking an antigen to the exterior surface and/or luminal surface of the EVs (e.g., exosome) can also be performed by modifying one or more proteins on the EVs to contain unnatural amino acids with side chains to allow for the binding of molecules such as the azide, strained alkyne (e.g., difluorinated cyclooctyne (DIFO), dibenzocyclooctyne (DBCO), bicyclononyne (BCN)), or combinations thereof. Additional disclosure regarding such approaches to linking an antigen to a surface of the EVs (e.g., exosomes) are provided elsewhere in the present disclosure. While the above disclosures are provided in the context of antigens, it will be readily apparent to those skilled in the arts that similar approaches can be used to link other moieties of interest (e.g., adjuvant, targeting moiety, and/or scaffold moiety) to a surface of the EVs (e.g., exosome).

As is apparent from the present disclosure, an antigen useful for the present disclosure can comprise various structure and/or length. In some aspects, the differences in the structure and/or length can affect the potency of the EV-based vaccines described herein. In some aspects, the antigen comprises a linear epitope of a protein from which it is derived (e.g., T and/or B cell antigen of a coronavirus), a conformational epitope, or both.

Non-limiting examples of possible antigen structure are provided below. As described herein, any single aspect of the exemplary structures can be modified. For instance, the length of any of the components noted below can be modified (lengthened or shortened). Additionally, any of the components can be removed (e.g., remove a spacer) or additional components can be added (e.g., add more spacers).

In some aspects, the antigen comprises a single antigen. In some aspects, the antigen comprises a T cell antigen, which comprises a CD8+ T cell epitope, CD4+ T cell epitope, or both. For example, in certain aspects, an antigen has the following structure: (first flanking region)-(T cell antigen)-(second flanking region). In some aspects, the T cell antigen comprises a CD8+ T cell epitope and is 9 amino acids in length (e.g., T cell antigen of a coronavirus), and each of the first and second flanking regions is 5 amino acids in length, such that the entire antigen is 19 amino acids in length. In some aspects, the CD8+ T cell epitope comprises between about 8 to about 12 amino acids in length. In some aspects, the CD8+ T cell epitope is about 8 amino acids in length. In some aspects, the CD8+ T cell epitope is about 9 amino acids in length. In some aspects, the CD8+ T cell epitope is about 10 amino acids in length. In some aspects, the CD8+ T cell epitope is about 11 amino acids in length. In some aspects, the CD8+ T cell epitope is about 12 amino acids in length. In some aspects, the T cell antigen comprises a CD4+ T cell epitope and is 25 amino acids in length (e.g., T cell antigen of a coronavirus), and each of the first and second flanking regions is 5 amino acids in length, such that the entire antigen is 35 amino acids in length. As described herein, in some aspects, the antigen comprises a lysine. In some aspects, lysine comprises a N-terminal lysine.

In some aspects, the antigen comprises a B cell antigen. In certain aspects, such antigen has the following structure: (B cell antigen)-(spacer)-(T helper peptide). In certain aspects, the B cell antigen (e.g., cognate B cell epitope of S2 antigen of a coronavirus) is 31 amino acids in length, the spacer is 3 amino acids in length, and the T helper peptide (e.g., PADRE) is 13 amino acids in length, such that the entire antigen is 47 amino acids in length. Non-limiting examples of spacers that can be used comprise one the following amino acid sequences: CPGPG (SEQ ID NO: 579), AAY, GSGSGS (SEQ ID NO: 580), or combinations thereof. As described herein, in some aspects, the antigen comprises a lysine. In some aspects, the lysine comprises N-terminal lysine.

In some aspects, the antigen comprises a concatemer of multiple epitopes of an antigen. For instance, in certain aspects, an antigen has the following structure: (first flanking region)-(first T cell antigen)-(first spacer)-(second T cell antigen)-(second spacer)-(third T cell antigen)-(second flanking region). In some aspects, the first and second flanking regions are 5 amino acids in length; the first, second, and third T cell antigens are 9 amino acids in length; and the first and second spacers are 3 amino acids in length, such that the entire antigen is 43 amino acids in length. As described herein, in some aspects, the antigen comprises a N-terminal lysine.

In some aspects, the potency of the EV-based vaccines of the present disclosure can be regulated by modifying the structure and/or overall length (e.g., the lengths of the flanking region, spacers, and/or T and B cell antigens).

As described elsewhere in the present disclosure, the EV-based vaccines described herein can be used to treat a wide range of diseases and disorders, e.g., by simply adding an antigen of interest to the base EV (e.g., exosome). In some aspects, the antigen is derived from and/or comprises a virus, a bacterium, a parasite, a fungus, a protozoa, a tumor, an allergen, a self-antigen, or any combination thereof.

In some aspects, the antigen is derived from a virus. In some aspects, the antigen is derived from a virus causing a pandemic. As used herein, the term “pandemic” refers to the rapid spread of a certain disease, involving a wide area, and a large proportion of the population, which can form a worldwide epidemic across provincial, national, or even continental borders in a short period of time. In certain aspects, an antigen that can be added to an EV (e.g., exosome) to produce an EV-based vaccine described herein is derived from a virus selected from a coronavirus, an influenza virus, an Ebola virus, a Chikungunya virus (CHIKV), a Crimean-Congo hemorrhagic fever (CCGF) virus, a Hendra virus, a Lassa virus, a Marburg virus, a monkeypox virus, a Nipah virus, a Hendra virus, a Rift Valley fever (RVF) virus, a Variola virus, a yellow fever virus, a Zika virus, a measles virus, a human immunodeficiency virus (HIV), a hepatitis C virus (HCV), a dengue fever virus (DENY), a parvovirus (e.g., B19 virus), a norovirus, a respiratory syncytial virus (RSV), a lentivirus, an adenovirus, a flavivirus, a filovirus, an alphavirus (e.g., a rhinovirus), a human papillomavirus (HPV), Eastern equine encephalitis (EEE), West Nile Virus, Epstein Barr virus (EBV), Cytomegalovirus (CMV), Hepatitis B virus (HBV), John Cunningham virus (JCV), Japanese and tick-borne encephalitis, encephalitic equine viruses, Human Metapneumovirus (hMPV), rabies, or any combination thereof.

In some aspects, the antigen is derived from a non-viral pathogen (e.g., a bacterium, parasite, fungus, protozoa, or combinations thereof). Non-limiting examples of such pathogens include: Vibrio cholera, Yersinia pestis, Mycobacterium tuberculosis (MTB), streptococcus (e.g., Streptococcus pyogenes, Streptococcus agalactiae, Streptococcus pneumoniae), staphylococcal (e.g., Staphylococcus aureus), shigella, Escherichia coli, salmonella, chlamydia (e.g., Chlamydia trachomatis), Pseudomonas aeruginosa, Klebsiella pneumoniae, Haemophilus influenza, Clostridia difficile, Plasmodium, Leishmania, Schistosoma, Trypanosoma, Brucella, Cryptosporidium, Entamoeba, Neisseria meningitis, Bacillus subtilis, Haemophilius influenzae, Neisseria gonorrhoeae, Borrelia burgdorferi, Corynebacterium diphteriae, Moraxella catarrhalis, Campylobacter jejuni, Clostridium tetanus, Clostridium perfringens, Treponema pallidum, or any combination thereof.

In some aspects, an antigen useful for producing the EV-based vaccines of the present disclosure is derived from a coronavirus. In some aspects, the coronavirus comprises SARS-CoV-1 and/or SARS-CoV-2 (COVID-19). In some aspects, the coronavirus comprises Middle East respiratory syndrome-related coronavirus (MERS-CoV; also known as EMC/2012). Unless indicated otherwise, an antigen that can be expressed in an EV (e.g., exosome) disclosed herein can be derived from any species of coronavirus.

In some aspects, an EV (e.g., exosome) disclosed herein comprises a single antigen. In some aspects, an EV (e.g., exosome) disclosed herein comprises multiple antigens. In certain aspects, each of the multiple antigens is different. For instance, in some aspects, the multiple antigens can be derived from the same species of coronavirus (e.g., multiple different proteins of COVID-19). In some aspects, the multiple antigens can be derived from different species of coronavirus (e.g., one or more proteins from SARS-CoV-1, SARS-CoV-2, and MERS-CoV). In some aspects, an EV (e.g., exosome) disclosed herein comprises at least 2, 3, 4, 5, 6, 7, 8, 9, 10 or more different antigens. As disclosed herein, an antigen can be linked to a surface of an EV (e.g., exosome) using a scaffold moiety (e.g., Scaffold X and/or Scaffold Y). In certain aspects, an antigen can be directly linked (i.e., without the use of a scaffold moiety) to a surface of an EV (e.g., exosome). In some aspects, an antigen can be in the lumen of the EV (e.g., exosome).

In some aspects, the antigen useful for the present disclosure is a universal antigen that is capable of inducing an immune response against any SARS coronavirus, e.g., SARS-CoV-1 and SARS-CoV-2 (COVID-19) virus. Therefore, in some aspects, the antigen useful for the present disclosure comprises an amino acid sequence that has at least about 90%, at least about 95%, at least about 96%, at least about 97%, at least about 98%, at least about 99%, or about 100% identical to at least five consecutive amino acids from a protein of a SARS coronavirus.

In some aspects, the antigen useful for the present disclosure comprises a receptor binding motif (also known as the “receptor-binding domain” (RBD)) of a spike protein derived from a SARS-CoV-1 and/or SARS-CoV-2 (COVID-19) virus. As used herein, the term “receptor-binding domain” or “RBD” includes all known RBDs of a coronavirus, such as that set forth in SEQ ID NO: 581, and any variants thereof. In some aspects, the antigen comprises at least five amino acids of the extracellular domain of an S protein from a SARS-CoV-1 and/or SARS-CoV-2 (COVID-19) virus. A spike protein of a coronavirus disclosed herein comprises a trimeric class I fusion protein, which can be in the down (closed) or up (open) conformation. Additional disclosures relating to the spike protein of a coronavirus disclosed herein, as well as the different subunits (e.g., RBD), are provided in Du, L., et al., Nature Reviews Microbiology 7:226-236 (2009), which is herein incorporated by reference in its entirety.

In some aspects, the antigen useful for the present disclosure comprises at least five amino acids from spike protein 51, S2, and/or S2′ from a SARS-CoV-1 and/or SARS-CoV-2 (COVID-19) virus. In some aspects, the antigen useful for the present disclosure comprises at least five amino acids from spike protein 51 from a SARS-CoV-1 and/or SARS-CoV-2 (COVID-19) virus. In some aspects, the antigen useful for the present disclosure comprises at least five amino acids from spike protein S2 from a SARS-CoV-1 and/or SARS-CoV-2 (COVID-19) virus. In some aspects, the antigen comprises a highly conserved region of a coronavirus spike S2 protein that lacks glycosylation (“glycan hole”). See, e.g., Yuan et al., Nat Commun 8:15092 (April 2017), which is incorporated herein by reference in its entirety. In some aspects, the antigen useful for the present disclosure comprises at least five amino acids from spike protein S2′ from a SARS-CoV-1 and/or SARS-CoV-2 (COVID-19) virus. In certain aspects, the antigen comprises a linear epitope derived from the S2 protein. In some aspects, the antigen comprises a conformation epitope of the S2 protein, wherein the conformational epitope is capable of eliciting an immune response and/or is capable of folding into a fold naturally found in the S2 protein.

In some aspects, the antigen comprises an amino acid epitope derived from a coronavirus, e.g., a coronavirus, e.g., SARS-CoV-1 and/or SARS-CoV-2 (COVID-19) virus. In some aspects, the EV comprises at least 2, 3, 4, 5, 6, 7, 8, 9, 10 or more antigens, wherein the first antigen is derived from a coronavirus, e.g., SARS-CoV-1 and/or SARS-CoV-2 (COVID-19) virus. In some aspects, the second antigen is also derived from a coronavirus, e.g., a SARS-CoV-1 and/or SARS-CoV-2 (COVID-19) virus. In some aspects, the second antigen is not derived from a coronavirus, e.g., a SARS-CoV-1 and/or SARS-CoV-2 (COVID-19) virus. In some aspects, the first and second antigens are derived from a coronavirus, e.g., a SARS-CoV-1 and/or SARS-CoV-2 (COVID-19) virus. In some aspects, the first and second antigens are the same. In some aspects, the first and second antigens are the same. In some aspects, the first antigen is derived from a coronavirus, e.g., a SARS-CoV-1 and/or SARS-CoV-2 (COVID-19) virus, and the second antigen is not derived from a coronavirus, e.g., a SARS-CoV-1 and/or SARS-CoV-2 (COVID-19) virus.

In some aspects, the antigen derived from a coronavirus, e.g., a SARS-CoV-1 and/or SARS-CoV-2 (COVID-19) virus is derived from a spike (S) protein. In some aspects, the antigen comprises at least 5, 6, 7, 8, 9, 10, 11, 12, 13, 14, 15, 16, 17, 18, 19, or 20 amino acids of the S protein.

In some aspects, the antigen derived from a coronavirus, e.g., a SARS-CoV-1 and/or SARS-CoV-2 (COVID-19) virus is derived from an envelope (E) protein. In some aspects, the antigen comprises at least 5, 6, 7, 8, 9, 10, 11, 12, 13, 14, 15, 16, 17, 18, 19, or 20 amino acids of the E protein.

In some aspects, the antigen derived from a coronavirus, e.g., a SARS-CoV-1 and/or SARS-CoV-2 (COVID-19) virus is derived from a membrane (M) protein. In some aspects, the antigen comprises at least 5, 6, 7, 8, 9, 10, 11, 12, 13, 14, 15, 16, 17, 18, 19, or 20 amino acids of the M protein.

In some aspects, a first antigen is derived from an S protein of a coronavirus, e.g., a SARS-CoV-1 and/or SARS-CoV-2 (COVID-19) virus, e.g., a COVID-19 virus, and the second antigen is derived from an antigen from a coronavirus, e.g., an S protein of a coronavirus, e.g., a SARS-CoV-1 and/or SARS-CoV-2 (COVID-19) virus. In some aspects, a first antigen is derived from an S protein of a coronavirus, e.g., a SARS-CoV-1 and/or SARS-CoV-2 (COVID-19) virus, e.g., a COVID-19 virus, and the second antigen is derived from an E protein of a coronavirus, e.g., a SARS-CoV-1 and/or SARS-CoV-2 (COVID-19) virus. In some aspects, a first antigen is derived from an S protein of a coronavirus, e.g., a SARS-CoV-1 and/or SARS-CoV-2 (COVID-19) virus, and the second antigen is derived from an M protein of a coronavirus, e.g., a SARS-CoV-1 and/or SARS-CoV-2 (COVID-19) virus.

In some aspects, a first antigen is derived from an E protein of a coronavirus, e.g., a SARS-CoV-1 and/or SARS-CoV-2 (COVID-19) virus, and the second antigen is derived from an S protein of a coronavirus, e.g., a SARS-CoV-1 and/or SARS-CoV-2 (COVID-19) virus. In some aspects, a first antigen is derived from an E protein of a coronavirus, e.g., a SARS-CoV-1 and/or SARS-CoV-2 (COVID-19) virus, and the second antigen is derived from an E protein of a coronavirus, e.g., a SARS-CoV-1 and/or SARS-CoV-2 (COVID-19) virus. In some aspects, a first antigen is derived from an E protein of a coronavirus, e.g., a SARS-CoV-1 and/or SARS-CoV-2 (COVID-19) virus, and the second antigen is derived from an M protein of a coronavirus, e.g., a SARS-CoV-1 and/or SARS-CoV-2 (COVID-19) virus.

In some aspects, a first antigen is derived from an M protein of a coronavirus, e.g., a SARS-CoV-1 and/or SARS-CoV-2 (COVID-19) virus, and the second antigen is derived from an S protein of a coronavirus, e.g., a SARS-CoV-1 and/or SARS-CoV-2 (COVID-19) virus. In some aspects, a first antigen is derived from an M protein of a coronavirus, e.g., a SARS-CoV-1 and/or SARS-CoV-2 (COVID-19) virus, and the second antigen is derived from an E protein of a coronavirus, e.g., a SARS-CoV-1 and/or SARS-CoV-2 (COVID-19) virus. In some aspects, a first antigen is derived from an M protein of a coronavirus, e.g., a SARS-CoV-1 and/or SARS-CoV-2 (COVID-19) virus, and the second antigen is derived from an M protein of a coronavirus, e.g., a SARS-CoV-1 and/or SARS-CoV-2 (COVID-19) virus. Exemplary coronavirus sequences are disclosed below:

TABLE 1
SARS-CoV-1 Spike Protein (UniProtKB-P59594 (SPIKE_CVHSA))
MFIFLLFLTL TSGSDLDRCT TEDDVQAPNY TQHTSSMRGV YYPDEIFRSD
TLYLTQDLFL PFYSNVTGFH TINHTFGNPV IPFKDGIYFA ATEKSNVVRG
WVFGSTMNNK SQSVIIINNS TNVVIRACNF ELCDNPFFAV SKPMGTQTHT
MIFDNAFNCT FEYISDAFSL DVSEKSGNFK HLREFVFKNK DGFLYVYKGY
QPIDVVRDLP SGFNTLKPIF KLPLGINITN FRAILTAFSP AQDIWGTSAA
AYFVGYLKPT TFMLKYDENG TITDAVDCSQ NPLAELKCSV KSFEIDKGIY
QTSNFRVVPS GDVVRFPNIT NLCPFGEVEN ATKFPSVYAW ERKKISNCVA
DYSVLYNSTF FSTFKCYGVS ATKLNDLCFS NVYADSFVVK GDDVRQIAPG
QTGVIADYNY KLPDDFMGCV LAWNTRNIDA TSTGNYNYKY RYLRHGKLRP
FERDISNVPF SPDGKPCTPP ALNCYWPLND YGFYTTTGIG YQPYRVVVLS
FELLNAPATV CGPKLSTDLI KNQCVNENEN GLTGTGVLTP SSKRFQPFQQ
FGRDVSDFTD SVRDPKTSEI LDISPCSFGG VSVITPGTNA SSEVAVLYQD
VNCTDVSTAI HADQLTPAWR IYSTGNNVFQ TQAGCLIGAE HVDTSYECDI
PIGAGICASY HTVSLLRSTS QKSIVAYTMS LGADSSIAYS NNTIAIPTNF
SISITTEVMP VSMAKTSVDC NMYICGDSTE CANLLLQYGS FCTQLNRALS
GIAAEQDRNT REVFAQVKQM YKTPTLKYFG GENFSQILPD PLKPTKRSFI
EDLLFNKVTL ADAGFMKQYG ECLGDINARD LICAQKENGL TVLPPLLTDD
MIAAYTAALV SGTATAGWTF GAGAALQIPF AMQMAYRENG IGVTQNVLYE
NQKQIANQFN KAISQIQESL TTTSTALGKL QDVVNQNAQA LNTLVKQLSS
NFGAISSVLN DILSRLDKVE AEVQIDRLIT GRLQSLQTYV TQQLIRAAEI
RASANLAATK MSECVLGQSK RVDFCGKGYH LMSFPQAAPH GVVFLHVTYV
PSQERNFTTA PAICHEGKAY FPREGVFVEN GTSWFITQRN FFSPQIITTD
NTFVSGNCDV VIGIINNTVY DPLQPELDSF KEELDKYFKN HTSPDVDLGD
ISGINASVVN IQKEIDRLNE VAKNLNESLI DLQELGKYEQ YIKWPWYVWL
GFIAGLIAIV MVTILLCCMT SCCSCLKGAC SCGSCCKEDE DDSEPVLKGV
KLHYT (SEQ ID NO: 417)
Extracellular domain: 14-1195
Transmembrane domain: 1196-1216
Cytoplasmic domain: 1217-1255

| | | |
|---|---|---|
| Signal peptide: | 1-13 | |
| Chain | 14-1255 | Spike glycoprotein |
| Chain | 14-667 | Spike protein S1 |
| Chain | 668-1255 | Spike protein S2 |
| Chain | 798-1255 | Spike protein S2′ |

SARS-CoV-1 Envelop Protein (UniProtKB-P59637 (VEMP_CVHSA))
MYSFVSEETG TLIVNSVLLF LAFVVFLLVT LAILTALRLC AYCCNIVNVS
LVKPTVYVYS RVKNLNSSEG VPDLLV (SEQ ID NO: 418)
SARS-CoV-1 Membrane Protein (UniProtKB-P59596 (VME1_CVHSA))
MADNGTITVE ELKQLLEQWN LVIGFLFLAW IMLLQFAYSN RNRFLYIIKL
VFLWLLWPVT LACFVLAAVY RINWVTGGIA IAMACIVGLM WLSYFVASER
LFARTRSMWS FNPETNILLN VPLRGTIVTR PLMESELVIG AVIIRGHLRM
AGHSLGRCDI KDLPKEITVA TSRTLSYYKL GASQRVGTDS GFAAYNRYRI
GNYKLNTDHA GSNDNIALLV Q (SEQ ID NO: 419)

In some aspects, an antigen (e.g., for inducing a humoral immune response) that is useful for the present disclosure is derived from a region of a coronavirus other than the receptor binding domain of a spike protein (S1 protein) and/or other conserved regions (e.g., within the M and/or N protein). In certain aspects, an EV (e.g., exosome) comprising such an antigen can reduce the risk of antibody dependent enhancement of viral infection.

As described herein, in some aspects, an antigen derived from a coronavirus is expressed in an EV (e.g., exosome) linked to a scaffold moiety (e.g., Scaffold X and/or Scaffold Y). In some aspects, a coronavirus antigen disclosed herein can be linked directly to the surface (e.g., exterior surface) of an EV (e.g., exosome). As further described elsewhere in the present disclosure, in some aspects, a coronavirus antigen that can be expressed in an EV (e.g., exosome) of the present disclosure comprises a spike protein derived from a coronavirus. In certain aspects, the coronavirus comprises SARS-CoV-1, SARS-CoV-2 (COVID-19), MERS-CoV, or combinations thereof.

In some aspects, a coronavirus spike protein (i.e., antigen) that can be expressed in an EV (e.g., exosome) comprises the entire (i.e., full-length) trimeric protein (i.e., “spike trimer”). Accordingly, in certain aspects, the spike trimer is linked directly to the surface (e.g., exterior surface) of an EV (e.g., exosome).

In some aspects, a coronavirus spike protein (i.e., antigen) that can be expressed in an EV (e.g., exosome) comprises a monomeric subunit of the trimeric spike protein (i.e., “spike monomer”). In some aspects, the spike monomer is linked directly to the surface (e.g., exterior surface) of an EV (e.g., exosome). In some aspects, the spike monomer is expressed on the surface (e.g., exterior surface) of an EV (e.g., exosome) linked to a scaffold moiety disclosed herein (e.g., Scaffold X and/or Scaffold Y). In certain aspects, the spike monomer is expressed on the exterior surface of an EV (e.g., exosome) linked to a Scaffold X.

In some aspects, a coronavirus spike protein (i.e., antigen) that can be expressed in an EV (e.g., exosome) comprises one or more subunits of a full-length spike protein (i.e., “exo-split-Spike”). The structure of the coronavirus spike protein, along with its different subunits, is known in the art. See, e.g., Fang Li, Annu Rev Virol 3(1): 237-261 (September 2016). In some aspects, any of the subunits of a coronavirus spike protein can be expressed in an EV (e.g., exosome) of the present disclosure. As described herein, in some aspects, the one or more subunits of a spike protein comprises a receptor-binding domain (RBD) of the spike protein. In some aspects, the RBD is linked directly to the surface (e.g., exterior surface) of an EV (e.g., exosome). In some aspects, the RBD is expressed on the surface (e.g., exterior surface) of an EV (e.g., exosome) linked to a scaffold moiety disclosed herein (e.g., Scaffold X and/or Scaffold Y). In certain aspects, the RBD of a coronavirus spike protein is expressed on the exterior surface of an EV (e.g., exosome) linked to a Scaffold X. As will be apparent to those skilled in the art,

As described herein, in some aspects, an EV (e.g., exosome) described herein can express multiple (e.g., two or more) coronavirus antigens (e.g., disclosed herein). In certain aspects, an EV (e.g., exosome) disclosed herein can express a spike protein (e.g., full-length protein or subunit thereof) and a coronavirus antigen comprising a T cell epitope (“T-antigen”). In some of these aspects, the spike protein antigen (e.g., receptor-binding domain) can be expressed on exterior surface of the EV (e.g., exosome) while the T-antigen is expressed on the luminal surface of the EV (e.g., exosome).

Accordingly, in some aspects, an EV (e.g., exosome) disclosed herein comprises: (i) a spike protein antigen (e.g., receptor-binding domain) and (ii) a T-antigen, wherein the spike protein antigen is linked to a Scaffold X (e.g., at the N-terminus) on the exterior surface of the EV (e.g., exosome), and the T-antigen is linked to a Scaffold X (e.g., at the C-terminus) on the luminal surface of the EV (e.g., exosome). In some aspects, an EV (e.g., exosome) comprises: (i) a spike protein antigen (e.g., receptor-binding domain) and (ii) a T-antigen, wherein the spike protein antigen is linked to a Scaffold X (e.g., at the N-terminus) on the exterior surface of the EV (e.g., exosome), and the T-antigen is linked to a Scaffold Y on the luminal surface of the EV (e.g., exosome). In some aspects, an EV (e.g., exosome) comprises: (i) a spike protein antigen (e.g., receptor-binding domain) and (ii) a T-antigen, wherein the spike protein antigen is linked to a Scaffold X (e.g., at the N-terminus) on the exterior surface of the EV, and the T-antigen is linked directly to the luminal surface of the EV (e.g., exosome). In some aspects, an EV (e.g., exosome) comprises: (i) a spike protein antigen (e.g., receptor-binding domain) and (ii) a T-antigen, wherein the spike protein antigen is linked directly to the exterior surface of the EV (e.g., exosome), and the T-antigen is linked to a Scaffold X (e.g., at the C-terminus) on the luminal surface of the EV (e.g., exosome). In some aspects, an EV (e.g., exosome) comprises (i) a spike protein antigen (e.g., receptor-binding domain) and (ii) a T-antigen, wherein the spike protein antigen is linked directly to the exterior surface of the EV (e.g., exosome), and the T-antigen is linked to a Scaffold Y on the luminal surface of the EV. In some aspects, an EV (e.g., exosome) comprises (i) a spike protein antigen (e.g., receptor-binding domain) and (ii) a T-antigen, wherein the spike protein antigen is linked directly to the exterior surface of the EV (e.g., exosome), and the T-antigen is linked directly to the luminal surface of the EV.

As described herein, in some aspects, an EV (e.g., exosome) comprising a spike protein antigen and a T-antigen described above can further express one or more additional moieties disclosed herein (e.g., adjuvant, immune modulator, and/or targeting moiety).

In some aspects, an antigen that can be added to an EV (e.g., exosome) to produce the EV-based vaccines described herein a CD8+ T cell epitope of a coronavirus T cell antigen, such as those described in Wang et al., J Virol 78(11): 5612-8 (June 2004); Wang et al., Blood 104(1): 200-6 (July 2004); Tsao et al., Biochem Biophys Res Commun 344(1):63-71 (May 2006); Lv et al., BMC Immunol 10:61 (December 2009); and Ahmed et al., Viruses 12(3): 254 (March 2020); Grifoni et al., Cell Host & Microbe 27: 671-680 (April 2020); each of which is incorporated herein by reference in its entirety. See also Table 2 (below).

TABLE 2
Exemplary Antigens Comprising T Cell Epitope of Coronavirus
Antigen Sequence

LLLNCLWSV (HCoV-229e, 77-85) (SEQ ID NO: 420)
FKDGIYFAA (SARS-CoV, 83-91) (SEQ ID NO: 421)
LITGRLAAL (HCoV-229e, 881-889) (SEQ ID NO: 422)
LITGRLQSL (SARS-CoV, 978-986) (SEQ ID NO: 423)
ISVVLIFVV (HCoV-229e, 1121-1129) (SEQ ID NO: 424)
FIAGLIAIV (SARS-CoV, 1203-1211) (SEQ ID NO: 425)
GILGFVFTL (influenza virus, 58-66) (SEQ ID NO: 426)
VVFLHVTYV (SEQ ID NO: 427)
RLQSLQTYV (SEQ ID NO: 428)
VLNDILSRL (SEQ ID NO: 429)
VLYENQKQI (SEQ ID NO: 430)
NLNESLIDL (SEQ ID NO: 431)
LLLQYGSFC (SEQ ID NO: 432)
KLPDDFMGC (SEQ ID NO: 433)
ILPDPLKPT (SEQ ID NO: 434)
MIFDNAFNC (SEQ ID NO: 435)
FIFLLFLTL (SEQ ID NO: 436)
VLYQDVNCT (SEQ ID NO: 437)
IITTDNTFV (SEQ ID NO: 438)
RLNEVAKNL (SEQ ID NO: 439)
LLTDDMIAA (SEQ ID NO: 440)
LLQYGSFCT (SEQ ID NO: 441)
ILLCCMTSC (SEQ ID NO: 442)
LLFNKVTLA (SEQ ID NO: 443)
TMNNKSQSV (SEQ ID NO: 444)
ALNTLVKQL (SEQ ID NO: 445)
ELCDNPFFA (SEQ ID NO: 446)
RLNQLESKV (SEQ ID NO: 448)
ILLNKHID (SEQ ID NO: 449)
AFFGMSRIGMEVTPSGTW (SEQ ID NO: 450)
MEVTPSGTWL (SEQ ID NO: 451)
GMSRIGMEV (SEQ ID NO: 452)
ILLNKHIDA (SEQ ID NO: 453)
ALNTPKDHI (SEQ ID NO: 454)
IRQGTDYKHWPQIAQFA (SEQ ID NO: 455)
KHWPQIAQFAPSASAFF (SEQ ID NO: 456)
LALLLLDRL (SEQ ID NO: 457)
LLLDRLNQL (SEQ ID NO: 458)
LLNKHIDAYKTFPPTEPK (SEQ ID NO: 459)
LQLPQGTTL (SEQ ID NO: 460)
AQFAPSASAFFGMSR (SEQ ID NO: 461)
AQFAPSASAFFGMSRIGM (SEQ ID NO: 462)
RRPQGLPNNTASWFT (SEQ ID NO: 463)
YKTFPPTEPKKDKKKK (SEQ ID NO: 464)
GAALQIPFAMQMAYRF (SEQ ID NO: 465)
MAYRFNGIGVTQNVLY (SEQ ID NO: 466)
QLIRAAEIRASANLAATK (SEQ ID NO: 467)
QALNTLVKQLSSNFGAI (SEQ ID NO: 470)
VLNDILSRL (SEQ ID NO: 472)
GLIAIVMVTI (SEQ ID NO: 474), LQYGSFCT (SEQ ID NO: 475), RLDKVEAEV
(SEQ ID NO: 476), FPRGQGVPI (SEQ ID NO: 447), LLLLDRLNQ (SEQ ID NO:
468),
LLLLDRLNQL (SEQ ID NO: 469), AQFAPSASA (SEQ ID NO: 471),
TTLPKGFYA (SEQ ID NO: 473), VLQLPQGTTL (SEQ ID NO: 477)
GYQPYRVVVL (SEQ ID NO: 478), PYRVVVLSF (SEQ ID NO: 479), LSPRWYFYY
(SEQ ID NO: 480)
DSFKEELDKY (SEQ ID NO: 481), LIDLQELGKY (SEQ ID NO: 482), GTTLPKGFY
(SEQ ID NO: 483), VTPSGTWLTY (SEQ ID NO: 484)
GSFCTQLNR (SEQ ID NO: 485), GVVFLHVTY (SEQ ID NO: 486), AQALNTLVK
(SEQ ID NO: 487), MTSCCSCLK (SEQ ID NO: 488), ASANLAATK (SEQ ID NO:
489), SLIDLQELGK (SEQ ID NO: 490), SVLNDILSR (SEQ ID NO: 491),
TQNVLYENQK (SEQ ID NO: 492), CMTSCCSCLK (SEQ ID NO: 493),
VQIDRLITGR (SEQ ID NO: 494), KTFPPTEPK (SEQ ID NO: 495), KTFPPTEPKK
(SEQ ID NO: 496), ASAFFGMSR (SEQ ID NO: 497), ATEGALNTPK (SEQ ID NO:
498), QLPQGTTLPK (SEQ ID NO: 499), QQQGQTVTK (SEQ ID NO: 500),
QQQQGQTVTK (SEQ ID NO: 501), SASAFFGMSR (SEQ ID NO: 502),
SQASSRSSSR (SEQ ID NO: 503), TPSGTWLTY (SEQ ID NO: 504)
FPNITNLCPF (SEQ ID NO: 505), APHGVVFLHV (SEQ ID NO: 506),
APSASAFFGM (SEQ ID NO: 507)
GAALQIPFAMQMAYR (SEQ ID NO: 508), GWTFGAGAALQIPFA (SEQ ID NO:
509), IDRLITGRLQSLQTY (SEQ ID NO: 510), ISGINASVVNIQKEI (SEQ ID NO:
511), LDKYFKNHTSPDVDL (SEQ ID NO: 512), LGDISGINASVVNIQ (SEQ ID NO:
513), LGFIAGLIAIVMVTI (SEQ ID NO: 514), LNTLVKQLSSNFGAI (SEQ ID NO:
515), LQDVVNQNAQALNTL (SEQ ID NO: 516), LQSLQTYVTQQLIRA (SEQ ID
NO: 517), LQTYVTQQLIRAAEI (SEQ ID NO: 518), AQKFNGLTVLPPLLT (SEQ ID
NO: 519), PCSFGGVSVITPGTN (SEQ ID NO: 520), QIPFAMQMAYRENGI (SEQ
ID NO: 521), QQLIRAAEIRASANL (SEQ ID NO: 522), QTYVTQQLIRAAEIR (SEQ
ID NO: 523), AYRFNGIGVTQNVLY (SEQ ID NO: 524), SSNFGAISSVLNDIL (SEQ
ID NO: 525), TGRLQSLQTYVTQQL (SEQ ID NO: 526), WLGFIAGLIAIVMVT
(SEQ ID NO: 527), CVNFNFNGLTGTGVL (SEQ ID NO: 528),
DKYFKNHTSPDVDLG (SEQ ID NO: 529), IDAYKTFPPTEPKKD (SEQ ID NO: 530),
MSRIGMEVTPSGTWL (SEQ ID NO: 531), NKHIDAYKTFPPTEP (SEQ ID NO:
532), VLQLPQGTTLPKGFY (SEQ ID NO: 533)
LQIPFAMQM (SEQ ID NO: 534), RVDFCGKGY (SEQ ID NO: 535)
YEQYIKWPWY (SEQ ID NO: 536)
GRLQSLQTY (SEQ ID NO: 537),
VRFPNITNL (SEQ ID NO: 538)
DVVNQNAQALNTLVKQL (SEQ ID NO: 539)
EAEVQIDRLITGRLQSL (SEQ ID NO: 540)
EIDRLNEVAKNLNESLIDLQELGKYEQY (SEQ ID NO: 541)
EVAKNLNESLIDLQELG (SEQ ID NO: 542)
GAALQIPFAMQMAYRFN (SEQ ID NO: 543)
GAGICASY (SEQ ID NO: 544)
AISSVLNDILSRLDKVE (SEQ ID NO: 545)
GSFCTQLN (SEQ ID NO: 546)
ILSRLDKVEAEVQIDRL (SEQ ID NO: 547)
KGIYQTSN (SEQ ID NO: 548)
AMQMAYRF (SEQ ID NO: 549)
KNHTSPDVDLGDISGIN (SEQ ID NO: 550)
MAYRENGIGVTQNVLYE (SEQ ID NO: 551)
AATKMSECVLGQSKRVD (SEQ ID NO: 552)
PFAMQMAYRENGIGVTQ (SEQ ID NO: 553)
QLIRAAEIRASANLAAT (SEQ ID NO: 554)
QQFGRD (SEQ ID NO: 555)
RASANLAATKMSECVLG (SEQ ID NO: 556)
RLITGRLQSLQTYVTQQ (SEQ ID NO: 557)
SLQTYVTQQLIRAAEIR (SEQ ID NO: 558)
DLGDISGINASVVNIQK (SEQ ID NO: 559)
FFGMSRIGMEVTPSGTW (SEQ ID NO: 560)
GLPNNTASWFTALTQHGK (SEQ ID NO: 561)
GTTLPK (SEQ ID NO: 562)
KHIDAYKTFPPTEPKKDKKK (SEQ ID NO: 563)
YNVTQAFGRRGPEQTQGNF (SEQ ID NO: 564)
KTFPPTEPKKDKKKK (SEQ ID NO: 565)
LLPAAD (SEQ ID NO: 566)
LNKHIDAYKTFPPTEPK (SEQ ID NO: 567)
LPQGTTLPKG (SEQ ID NO: 568)
LPQRQKKQ (SEQ ID NO: 569)
PKGFYAEGSRGGSQASSR (SEQ ID NO: 570)
QFAPSASAFFGMSRIGM (SEQ ID NO: 571)
QGTDYKHW (SEQ ID NO: 572)
QLPQGTTLPKGFYAE (SEQ ID NO: 573)
QLPQGTTLPKGFYAEGSR (SEQ ID NO: 574)
QLPQGTTLPKGFYAEGSRGGSQ (SEQ ID NO: 575)
TFPPTEPK (SEQ ID NO: 576)
SQASSRSS (SEQ ID NO: 577)
SRGGSQASSRSSSRSR (SEQ ID NO: 578)

In some aspects, the antigen comprises a neuronal protein. In certain aspects, the neuronal protein can be misfolded, wherein the misfolded neuronal protein can result in a neurological disorder. In some aspects, the neuronal protein comprises amyloid beta (Aβ), tau, alpha-synuclein (αSyn), dipeptide repeat (DPR) proteins (e.g., poly-Gly-Ala (poly-GA)), mutant Huntingtin (HTT) protein, TDP-43, or combinations thereof. Non-limiting examples of neurological disorders that are associated with such misfolded neuronal proteins can include a brain tumor, neoplastic meningitis, leptomeningeal cancer disease (LMD), amyotrophic lateral sclerosis (ALS), frontotemporal dementia (FTD), Parkinson's disease (PD), Huntington's disease (HD), Alzheimer's disease (AD), Lewy body dementia (LTD), spinocerebellar ataxia (e.g., type 8 (SCAB), SCA10, SCA12, SCA31, SCA36), Huntington's disease like-2, ataxia (e.g., Friedreich ataxia), muscular dystrophy (e.g., oculopharyngeal muscular dystrophy), or combinations thereof.

Accordingly, in some aspects, an EV disclosed herein comprises a neuronal protein as an antigen, wherein the antigen comprises dipeptide repeat (DPR) proteins. In certain aspects, the dipeptide DPR proteins are derived from repeat associated non-ATG (RAN) translation (referred to herein as RAN protein) (e.g., C9 RAN proteins). In some aspects, the dipeptide DPR proteins comprise poly-GA (i.e., repeats of glycine-alanine residues). In some aspects, the poly-GA comprises at least about 2, at least about 3, at least about 4, at least about 5, at least about 6, at least about 7, at least about 8, at least about 9, at least about 10, at least about 15, at least about 20, at least about 25, at least about 30 or more repeats of glycine-alanine residues). In certain aspects, the poly-GA comprises 10 repeats of glycine-alanine residues. In certain aspects, the poly-GA are specific for B cells (referred to herein as “poly-GA B cell antigen”). In certain aspects, the dipeptide DPR protein comprises poly-GR (i.e., repeats of glycine-arginine residues). In some aspects, the dipeptide DPR protein comprises poly-GP (i.e., repeats of glycine-proline residues). In some aspects, the dipeptide DPR protein comprises poly-PA (i.e., repeats of proline-alanine residues). In some aspects, the dipeptide DPR protein comprises poly-PR (i.e., repeats of proline-arginine residues). In some aspects, the dipeptide DPR protein comprises poly-G (i.e., repeats of glutamine residues). It will be apparent to those skilled in the art that, unless indicated otherwise, disclosures provided herein relating to poly-GA can equally apply to the other dipeptide DPR proteins provided herein. As described herein, such EVs can be used to treat neurological disorders associated with a hexanucleotide GGGGCC repeat expansion in the C9orf72 gene (e.g., C9FTD/ALS). A pathological hallmark that can be observed in C9orf72 repeat expansion carriers include formation of RNA foci and deposition of dipeptide repeat (DPR) proteins derived from repeat associated non-ATG (RAN) translation. See Nguyen et al., Annu Rev Neurosci 42:227-247 (July 2019), which is incorporated herein by reference in its entirety.

In some aspects, an EV comprising a neuronal protein described above (e.g., C9 RAN proteins, e.g., poly GA B cell antigen) are capable of inducing a B cell-specific immune response (e.g., does not induce a T cell immune response that is harmful to the subject that receives the EV). Accordingly, in some aspects, an EV disclosed herein (e.g., comprising a C9 RAN protein, e.g., poly GA B cell antigen) is capable of stimulating antibody producing B cells without activating harmful T cells. In some aspects, harmful T cells comprise T cells that exhibit excessive cytotoxicity activity (e.g., produces excessive inflammatory mediators resulting in damage to healthy cells/tissues), that induces an autoimmune response, that are not involved in treatment of the disease or disorder (e.g., does not play a role in the activation of the antibody producing B cells), or combinations thereof. In certain aspects, an EV comprising a neuronal protein described herein (e.g., C9 RAN proteins, e.g., poly GA B cell antigen) can exhibit one or more of the following when administered to a subject: (i) decrease GA protein aggregate; (ii) improve proteasome function, (iii) increase autophagy, (iv) decrease neuroinflammation, (v) decrease motor neuron loss, (vi) increase anti-polyGA antibody production, (vii) improve behavioral function, and (viii) combinations thereof.

In some aspects, the antigen comprises a fragment of the amyloid-0 protein. In certain aspects, the fragment of the amyloid-β protein comprises the amino acid sequence DAEFRHDSGYEVHHQKLVFFAEDVGSNKGAIIGLMVGGVVIA (SEQ ID NO: 379) (i.e., amino acid residues 1-42 of amyloid-β protein). Accordingly, in some aspects, an EV (e.g., exosome) disclosed herein comprises an antigen, wherein the antigen comprises one or more epitopes of an amyloid-β protein fragment set forth in SEQ ID NO: 379. In some aspects, the one or more epitopes comprise amino acid residues 1-42 of SEQ ID NO: 379. In some aspects, the one or more epitopes comprise amino acid residues 1-6 of SEQ ID NO: 379. In some aspects, the one or more epitopes comprise amino acid residues 1-7 of SEQ ID NO: 379. In some aspects, the one or more epitopes comprise amino acid residues 1-12 of SEQ ID NO: 379. In some aspects, the one or more epitopes comprise amino acid residues 1-14 of SEQ ID NO: 379. In certain aspects, the one or more epitopes comprise amino acid residues 1-15 of SEQ ID NO: 379. In some aspects, the one or more epitopes comprise amino acid residues 1-11 and 18-27 of SEQ ID NO: 379. In some aspects, the one or more epitopes comprise amino acid residues 13-28 of SEQ ID NO: 379. In some aspects, the one or more epitopes comprise amino acid residues 12-23 of SEQ ID NO: 379. In some aspects, the one or more epitopes comprise amino acid residues 3-6 of SEQ ID NO: 379. In some aspects, the one or more epitopes comprise amino acid residues 1-5 of SEQ ID NO: 379. In some aspects, the one or more epitopes comprise amino acid residues 35-40 of SEQ ID NO: 379. In some aspects, an EV of the present disclosure comprises an antigen, wherein the antigen comprises a combination (e.g., 2, 3, 4, 5, 6, 7, 8, 9, 10, 11, 12, or 13) of the epitopes described above.

As described herein, in some aspects, an EV that can be used in treating a neurological disorder comprises an antigen (e.g., expressed on the outer surface of the EV), wherein the antigen comprises a B cell epitope, CD4+ T cell epitope, or both. In certain aspects, the B cell epitope comprises the amino acid sequence AEFRHD (SEQ ID NO: 380) (i.e., amino acid residues 2-7 of amyloid-β protein fragment set forth in SEQ ID NO: 379). In some aspects, the B cell epitope comprises the amino acid sequence (GA)₁₀₋₂₀ (polyGA) (SEQ ID NO: 381). In some aspects, the CD4+ T cell epitope comprises the amino acid sequence AKFVAAWTLKAAA (SEQ ID NO: 382) (PADRE). In some aspects, the CD4+ T cell epitope comprises the amino acid sequence QYIKANSKFIGITE (SEQ ID NO: 383) (amino acid residues 830-843 of tetanus). In some aspects, the CD4+ T cell epitope comprises the amino acid sequence QSIALSSLMVAQAIP (SEQ ID NO: 384) (amino acid residues 356-370 of diphtheria toxin).

In some aspects, an antigen comprises a self-antigen. Non-limiting examples of self-antigens are provided elsewhere in the present disclosure.

As described herein, in some aspects, an EV described herein can comprise an antagonist that specifically targets any of the antigens described above. For example, in certain aspects, the antagonist can target any of the neuronal proteins described herein (e.g., C9 RAN proteins, e.g., poly GA B cell antigen). Non-limiting examples of antagonists include antibodies, mRNA, miRNA, siRNA, antisense oligonucleotide (ASO), phosphorodiamidate morpholino oligomer (PMO), peptide-conjugated phosphorodiamidate morpholino oligomer (PPMO), shRNA, lncRNA, dsDNA, or combinations thereof.

As demonstrated herein, by modifying the particular location at which an antigen is associated with the EV (e.g., on the exterior surface, on the luminal surface, and/or in the lumen), an EV-based vaccine of the present disclosure is capable of eliciting a strong immune response without the need for additional moieties described herein (e.g., adjuvant, immune modulator, and/or targeting moieties). Accordingly, in some aspects, an EV described herein comprises an antigen, wherein the antigen is associated with the exterior surface of the EV. As further described elsewhere in the present disclosure, in some aspects, the antigen is fused to the N-terminus of a Scaffold X moiety (e.g., PTGFRN) expressed on the exterior surface of the EV. In some aspects, the antigen is associated with the exterior surface of the EV using any of the coupling strategies described herein (e.g., ALFA-tag). In certain aspects, the EV comprising the antigen does not comprise an adjuvant, immune modulator, and/or targeting moiety.

### Adjuvants

As described herein, in addition to the antigens described above, an EV-based vaccine described herein further comprises an adjuvant. In some aspects, a base EV (e.g., exosome) that can be used with the methods disclosed herein comprises an adjuvant, such that the adjuvant is present in the EV prior to the addition of the antigen. For instance, in some aspects, the adjuvant can be introduced into a producer cell when producing the base EVs (e.g., exosomes). In some aspects, the adjuvant can be added to the EVs (e.g., exosomes) after being isolated from the producer cells. In such aspects, the adjuvant can be added to the isolated EVs (e.g., exosomes) before adding the antigen. In some aspects, the adjuvant is added to the EV after adding the antigen. In some aspects, the adjuvant is added to the EV together with the antigen.

As used herein, the term “adjuvant” refers to any substance that enhances the therapeutic effect of the EV-based vaccines (e.g., increasing an immune response to the antigen). Accordingly, EVs, e.g., exosomes, described herein comprising an adjuvant are capable of increasing an immune response, e.g., to an antigen, by at least about 5%, at least about 10%, at least about 20%, at least about 30%, at least about 40%, at least about 50%, at least about 60%, at least about 70%, at least about 80%, at least about 90%, at least about 100%, at least about 250%, at least about 500%, at least about 750%, at least about 1,000% or more or more, compared to a reference (e.g., corresponding EV without the adjuvant or a non-EV delivery vehicle comprising an antigen alone or in combination with the adjuvant). In some aspects, incorporating an adjuvant disclosed herein to an EV (e.g., exosome) can increase an immune response, e.g., to an antigen, by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, at least about 10,000-fold or more, compared to a reference (e.g., corresponding EV comprising the antigen alone or a non-EV delivery vehicle comprising an antigen alone or in combination with the adjuvant).

In some aspects, EVs (e.g., exosomes) described herein (e.g., those that can be used as vaccines) comprises a single adjuvant. In some aspects, an EV (e.g., exosome) disclosed herein comprises multiple adjuvants. In such aspects, some or all of the multiple adjuvants: (i) can be introduced into the producer cells, such that the base EVs (e.g., exosome) are produced comprising the adjuvants; (ii) can be added to the EVs (e.g., exosomes) are they are isolated from the producer cells but prior to the addition of the antigen; (iii) can be added to the EVs (e.g., exosomes) after the addition of the antigen; (iv) can be added to the EVs (e.g., exosomes) together with the antigen; or (v) any combination thereof.

In some aspects, each of the multiple adjuvants is different. In some aspects, an EV (e.g., exosome) disclosed herein comprises at least 2, 3, 4, 5, 6, 7, 8, 9, 10 or more different adjuvants. As disclosed herein, an adjuvant can be linked to a surface of an EV (e.g., exosome) using a scaffold moiety (e.g., Scaffold X and/or Scaffold Y). In certain aspects, an adjuvant can be directly linked (i.e., without the use of a scaffold moiety) to a surface of an EV (e.g., exosome). In some aspects, an adjuvant can be in the lumen of the EV (e.g., exosome).

Non-limiting examples of adjuvants that can be used with the present disclosure include: Stimulator of Interferon Genes (STING) agonist, a toll-like receptor (TLR) agonist, an inflammatory mediator, RIG-I agonists, alpha-gal-cer (NKT agonist), heat shock proteins (e.g., HSP65 and HSP70), C-type lectin agonists (e.g., beta glucan (Dectin 1), chitin, and curdlan), and combinations thereof.

In some aspects, incorporating an adjuvant (e.g., such as those disclosed herein) to an EV (e.g., exosome) can broaden an immune response induced by the EV. As used herein, to “broaden an immune response” refers to enhancing the diversity of an immune response. In some aspects, the diversity of an immune response can be enhanced through epitope spreading (i.e., inducing and/or increasing an immune response (cellular and/or humoral immune response) against a greater number/variety of epitopes on an antigen). In some aspects, the diversity of an immune response can be enhanced through the production of different and/or multiple antibody isotypes (e.g., IgG, IgA, IgD, IgM, and/or IgE).

In some aspects, an adjuvant (e.g., such as those disclosed herein) can also help regulate the type of immune response induced by the EV (e.g., exosome). For example, in some aspects, incorporating an adjuvant to an EV (e.g., exosome) can help drive an immune response towards a more Th1 phenotype. As used herein, a “Th1” immune response is generally characterized by the production of IFN-γ, which can activate the bactericidal activities of innate cells (e.g., macrophages), help induce B cells to make opsonizing (marking for phagocytosis) and complement-fixing antibodies, and/or lead to cell-mediated immunity (i.e., not mediated by antibodies). In general, Th1 responses are more effective against intracellular pathogens (viruses and bacteria that are inside host cells).

In some aspects, incorporating an adjuvant to an EV (e.g., exosome) can help drive an immune response towards a more Th2 phenotype. As used herein, a “Th2” immune response can be characterized by the release of certain cytokines, such as IL-5 (induces eosinophils in the clearance of parasites) and IL-4 (facilitates B cell isotype switching). In general, Th2 responses are more effective against extracellular bacteria, parasites including helminths and toxins.

In some aspects, incorporating an adjuvant to an EV (e.g., exosome) can help drive an immune response towards a more Th17 phenotype. As used herein, a “Th17” immune response is mediated by Th17 cells. As used herein, “Th17 cells” refer to a subset of CD4+ T cells characterized by the production of pro-inflammatory cytokines, such as IL-17A, IL-17F, IL-21, IL-22, and granulocyte-macrophage colony-stimulating factor (GM-CSF). Th17 cells are generally thought to play an important role in host defense against infection, by recruiting neutrophils and macrophages to infected tissues.

In some aspects, incorporating an adjuvant to an EV (e.g., exosome) can help drive an immune response towards a more cellular immune response (e.g., T-cell mediated). In some aspects, incorporating an adjuvant to an EV (e.g., exosome) can help drive an immune response towards a more humoral immune response (e.g., antibody-mediated).

In some aspects, an adjuvant induces the activation of a cytosolic pattern recognition receptor. Non-limiting examples of cytosolic pattern recognition receptor includes: stimulator of interferon genes (STING), retinoic acid-inducible gene I (RIG-1), Melanoma Differentiation-Associated protein 5 (MDA5), Nucleotide-binding oligomerization domain, Leucine rich Repeat and Pyrin domain containing (NLRP), inflammasomes, or combinations thereof. In certain aspects, an adjuvant is a STING agonist. Stimulator of Interferon Genes (STING) is a cytosolic sensor of cyclic dinucleotides that is typically produced by bacteria. Upon activation, it leads to the production of type I interferons (e.g., IFN-α (alpha), IFN-β (beta), IFN-κ (kappa), IFN-δ (delta), IFN-ε (epsilon), IFN-τ (tau), IFN-ω (omega), and IFN-ζ (zeta, also known as limitin)) and initiates an immune response. In certain aspects, the STING agonist comprises a cyclic dinucleotide STING agonist or a non-cyclic dinucleotide STING agonist. As described herein, in some aspects, the STING agonist is loaded in the lumen of the EV (e.g., exosome). In some aspects, such EVs (e.g., exosomes) are referred to herein as “exoSTING.” Non-limiting examples of exoSTING are provided in International Publication No. WO 2019183578A1, which is herein incorporated by reference in its entirety. Further disclosures of useful STING agonists are also provided throughout the present disclosure.

Cyclic purine dinucleotides such as, but not limited to, cGMP, cyclic di-GMP (c-di-GMP), cAMP, cyclic di-AMP (c-di-AMP), cyclic-GMP-AMP (cGAMP), cyclic di-IMP (c-di-IMP), cyclic AMP-IMP (cAIMP), and any analogue thereof, are known to stimulate or enhance an immune or inflammation response in a patient. The CDNs can have 2′2′, 2′3′, 2′5′, 3′3′, or 3′5′ bonds linking the cyclic dinucleotides, or any combination thereof.

Cyclic purine dinucleotides can be modified via standard organic chemistry techniques to produce analogues of purine dinucleotides. Suitable purine dinucleotides include, but are not limited to, adenine, guanine, inosine, hypoxanthine, xanthine, isoguanine, or any other appropriate purine dinucleotide known in the art. The cyclic dinucleotides can be modified analogues. Any suitable modification known in the art can be used, including, but not limited to, phosphorothioate, biphosphorothioate, fluorinate, and difluorinate modifications.

Non cyclic dinucleotide agonists can also be used, such as 5,6-Dimethylxanthenone-4-acetic acid (DMXAA), or any other non-cyclic dinucleotide agonist known in the art.

Non-limiting examples of STING agonists that can be used with the present disclosure include: DMXAA, STING agonist-1, ML RR-52 CDA, ML RR-S2c-di-GMP, ML-RR-S2 cGAMP, 2′3′-c-di-AM(P5)2, 2′3′-cGAMP, 2′3′-cGAMPdFHS, 3′3′-cGAMP, 3′3′-cGAMPdFSH, cAIMP, cAIM(PS)2, 3′3′-cAIMP, 3′3′-cAIMPdFSH, 2′2′-cGAMP, 2′3′-cGAM(P5)2, 3′3′-cGAMP, and combinations thereof. Non-limiting examples of the STING agonists can be found at U.S. Pat. No. 9,695,212, WO 2014/189805 A1, WO 2014/179335 A1, WO 2018/100558 A1, U.S. Pat. No. 10,011,630 B2, WO 2017/027646 A1, WO 2017/161349 A1, and WO 2016/096174 A1, each of which is incorporated by reference in its entirety.

In some aspects, the STING agonist useful for the present disclosure comprises the compound or a pharmaceutically acceptable salt thereof. See WO 2016/096174 A1, which is incorporated herein by reference in its entirety.

In some aspects, the STING agonist useful for the present disclosure comprises a compound described in WO 2014/093936, WO 2014/189805, WO 2015/077354, Cell reports 11, 1018-1030 (2015), WO 2013/185052, Sci. Transl. Med. 283,283ra52 (2015), WO 2014/189806, WO 2015/185565, WO 2014/179760, WO 2014/179335, WO 2015/017652, WO 2016/096577, WO 2016/120305, WO 2016/145102, WO 2017/027646, WO 2017/075477, WO 2017/027645, WO 2018/100558, WO 2017/175147, and/or WO 2017/175156, the contents of which are incorporated herein by reference in their entireties.

In some aspects, the STING agonist useful for the present disclosure is CL606, CL611, CL602, CL655, CL604, CL609, CL614, CL656, CL647, CL626, CL629, CL603, CL632, CL633, CL659, or a pharmaceutically acceptable salt thereof. In some aspects, the STING agonist useful for the present disclosure is CL606 or a pharmaceutically acceptable salt thereof. In some aspects, the STING agonist useful for the present disclosure is CL611 or a pharmaceutically acceptable salt thereof. In some aspects, the STING agonist useful for the present disclosure is CL602 or a pharmaceutically acceptable salt thereof. In some aspects, the STING agonist useful for the present disclosure is CL655 or a pharmaceutically acceptable salt thereof. In some aspects, the STING agonist useful for the present disclosure is CL604 or a pharmaceutically acceptable salt thereof. In some aspects, the STING agonist useful for the present disclosure is CL609 or a pharmaceutically acceptable salt thereof. In some aspects, the STING agonist useful for the present disclosure is CL614 or a pharmaceutically acceptable salt thereof. In some aspects, the STING agonist useful for the present disclosure is CL656 or a pharmaceutically acceptable salt thereof. In some aspects, the STING agonist useful for the present disclosure is CL647 or a pharmaceutically acceptable salt thereof. In some aspects, the STING agonist useful for the present disclosure is CL626 or a pharmaceutically acceptable salt thereof. In some aspects, the STING agonist useful for the present disclosure is CL629 or a pharmaceutically acceptable salt thereof. In some aspects, the STING agonist useful for the present disclosure is CL603 or a pharmaceutically acceptable salt thereof. In some aspects, the STING agonist useful for the present disclosure is CL632 or a pharmaceutically acceptable salt thereof. In some aspects, the STING agonist useful for the present disclosure is CL633 or a pharmaceutically acceptable salt thereof. In some aspects, the STING agonist useful for the present disclosure is CL659 or a pharmaceutically acceptable salt thereof.

In some aspects, STING agonists can be modified to allow for better expression of the agonists on the surface of the EV (e.g., exterior and/or luminal surface of the EV, e.g., exosome, (e.g., linked to a scaffold moiety disclosed herein, e.g., Scaffold X and/or Scaffold Y)). Any of the modifications described above can be used.

In some aspects, an adjuvant is a TLR agonist. Non-limiting examples of TLR agonists include: TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, (3-defensin 2, fibronectin EDA, HMGB1, snapin, tenascin C, monophosphoryl lipid A (MPLA)), a TLR5 agonist (e.g., flagellin), a TLR6 agonist, a TLR7/8 agonist (e.g., single-stranded RNA, CpG-A, Poly G10, Poly G3, Resiquimod (R848), 3M-052), a TLR9 agonist (e.g., unmethylated CpG DNA), and combinations thereof. Non-limiting examples of TLR agonists can be found at WO2008115319A2, US20130202707A1, US20120219615A1, US20100029585A1, WO2009030996A1, WO2009088401A2, and WO2011044246A1, each of which is incorporated by reference in its entirety. In some aspects, the adjuvant is a TLR4 agonist. In some aspects, the TLR4 agonist comprises MPLA. In some aspects, the adjuvant is a TLR7/8 agonist. In some aspects, the TLR7/8 agonist comprises Resiquimod (R848). In some aspects, the TLR7/8 agonist comprises 3M-052.

In some aspects, an adjuvant comprises a TNF superfamily member (e.g., TNFα, TNF-C, OX40L, CD40L, FasL, LIGHT, TL1A, CD27L, Siva, CD153, 4-1BB ligand, TRAIL, RANKL, TWEAK, APRIL, BAFF, CAMLG, NGF, BDNF, NT-3, NT-4, GITR ligand, and EDA-2). In some aspects, the immune modulator is an activator of CD40. In some aspects, the activator of CD40 is an agonistic anti-CD40 antibody. In some aspects, the activator of CD40 is a CD40 ligand (CD40L) protein. In some aspects, the CD40L is a monomeric CD40L. In some aspects, the CD40L is a trimeric CD40L.

In some aspects, an adjuvant comprises a cytokine or a binding partner of a cytokine. In some aspects, the cytokine is selected from (i) common gamma chain family of cytokines; (ii) IL-1 family of cytokines; (iii) hematopoietic cytokines; (iv) interferons (e.g., type I, type II, or type III); (v) TNF family of cytokines; (vi) IL-17 family of cytokines; (vii) damage-associated molecular patterns (DAMPs); (viii) tolerogenic cytokines; or (ix) combinations thereof. In certain aspects, the cytokine comprises IL-2, IL-4, IL-7, IL-10, IL-12, IL-15, IFN-γ, IL-1α, IL-1β, IL-1ra, IL-18, IL-33, IL-36α, IL-36β, IL-36γ, IL-36ra, IL-37, IL-38, IL-3, IL-5, IL-6, IL-11, IL-13, IL-23, granulocyte-macrophage colony stimulating factor (GM-CSF), granulocyte-colony stimulating factor (G-CSF), leukemia inhibitory factor (LIF), stem cell factor (SCF), thrombopoietin (TPO), macrophage-colony stimulating factor (M-CSF), erythropoieticn (EPO), Flt-3, IFN-α, IFN-γ, IL-19, IL-20, IL-22, TNF-α, TNF-β, BAFF, APRIL, lymphotoxin beta (TNF-γ), IL-17A, IL-17B, IL-17C, IL-17D, IL-17E, IL-17F, TSLP, IL-35, IL-27, TGF-β, or combinations thereof. In some aspects, the adjuvant comprises IL-12.

In some aspects, an adjuvant is an inflammatory mediator.

In some aspects, one or more antigens are expressed on the exterior surface or in the lumen (e.g., on the luminal surface) of the EV, e.g., exosome. In some aspects, an adjuvant is expressed on the exterior surface or in the luminal surface of the EVs, e.g., exosomes, directly connected to the lipid bilayer. In such aspects, the antigen, e.g., a first antigen and/or a second antigen, and/or the adjuvant can be linked to a scaffold moiety (e.g., Scaffold X and/or Scaffold Y).

### Additional Moieties of Interest

In some aspects, an EV-based vaccine described herein can comprise additional moieties of interest (e.g., in combination with the antigen and/or adjuvants described above), which can further enhance the therapeutic effects of the EVs (e.g., exosomes), e.g., when administered to a subject. For instance, the additional moiety of interest can help direct EV uptake (e.g., targeting moiety), activate, or block cellular pathways to enhance the combinatorial effects associated with the EVs (e.g., exosomes), e.g., when administered to a subject as a vaccine.

In some aspects, the additional moiety of interest comprises a targeting moiety that can modify the distribution of the EVs in vivo or in vitro. In some aspects, the targeting moiety can be a biological molecule, such as a protein, a peptide, a lipid, or a synthetic molecule.

In some aspects, a targeting moiety (e.g., tropism moiety) of the present disclosure specifically binds to a marker for a particular type of cell. In some aspects, the cell is an immune cell, e.g., dendritic cell. In certain aspects, the marker is expressed only on dendritic cells. In some aspects, dendritic cells comprise a progenitor (Pre) dendritic cells, inflammatory mono dendritic cells, plasmacytoid dendritic cell (pDC), a myeloid/conventional dendritic cell 1 (cDC1), a myeloid/conventional dendritic cell 2 (cDC2), inflammatory monocyte derived dendritic cells, Langerhans cells, dermal dendritic cells, lysozyme-expressing dendritic cells (LysoDCs), Kupffer cells, nonclassical monocytes, or any combination thereof. Markers that are expressed on these dendritic cells are known in the art. See, e.g., Collin et al., Immunology 154(1):3-20 (2018). In some aspects, the targeting moiety (e.g., tropism moiety) is a protein, wherein the protein is an antibody or a fragment thereof that can specifically bind to a marker selected from lymphocyte antigen 75 (DEC205), C-type lectin domain family 9 member A (CLEC9A), C-type lectin domain family 6 (CLEC6), C-type lectin domain family 4 member A (also known as DCIR or CLEC4A), Dendritic Cell-Specific Intercellular adhesion molecule-3-Grabbing Non-integrin (also known as DC-SIGN or CD209), lectin-type oxidized LDL receptor 1 (LOX-1), macrophage receptor with collagenous structure (MARCO), C-type lectin domain family 12 member A (CLEC12A), C-type lectin domain family 10 member A (CLEC10A), DC-asialoglycoprotein receptor (DC-ASGPR), DC immunoreceptor 2 (DCIR2), Dectin-1, macrophage mannose receptor (MMR), BDCA-2 (CD303, CLEC4C), Dectin-2, Bst-2 (CD317), Langerin, CD206, CD11b, CD11c, CD123, CD304, XCR1, AXL, Siglec 6, CD209, SIRPA, CX3CR1, GPR182, CD14, CD16, CD32, CD34, CD38, CD10, or any combination thereof. In some aspects, a marker useful for the present disclosure comprises a C-type lectin like domain. In certain aspects, a marker is Clec9a and the dendritic cell is cDC1

In some aspects, a targeting moiety disclosed herein can bind to both human and mouse Clec9a, including any variants thereof. In some aspects, a targeting moiety of the present disclosure can bind to Clec9a from other species, including but not limited to chimpanzee, rhesus monkey, dog, cow, horse, or rat. Sequences for such Clec9a protein are known in the art. See, e.g., U.S. Pat. No. 8,426,565 B2, which is herein incorporated by reference in its entirety.

In some aspects, a targeting moiety (e.g., tropism moiety) of the present disclosure specifically binds to a marker for a T cell. In certain aspects, the T cell is a CD4+ T cell. In some aspects, the T cell is a CD8+ T cell.

In some aspects, a targeting moiety (e.g., tropism moiety) disclosed herein binds to human CD3 protein or a fragment thereof. Sequences for human CD3 protein are known in the art.

In some aspects, a targeting moiety disclosed herein can bind to both human and mouse CD3, including any variants thereof. In some aspects, a targeting moiety of the present disclosure can bind to CD3 from other species, including but not limited to chimpanzee, rhesus monkey, dog, cow, horse, or rat. Sequences for such CD3 protein are also known in the art.

In some aspects, a targeting moiety useful for the present disclosure binds to markers expressed on epithelial cells. For instance, in some aspects, a targeting moiety comprises the RBD of a coronavirus spike protein, which can specifically bind to ACE2-positive cells, such as epithelial cells.

As described supra, a targeting moiety disclosed herein can comprise a peptide, an antibody or an antigen binding fragment thereof, a chemical compound, or any combination thereof.

In some aspects, the targeting moiety is an antibody or an antigen binding fragment thereof. In certain aspects, a targeting moiety is a single-chain Fv antibody fragment. In certain aspects, a targeting moiety is a single-chain F(ab) antibody fragment. In certain aspects, a targeting moiety is a nanobody. In certain aspects, a targeting moiety is a monobody.

In some aspects, an EV, e.g., exosome, disclosed herein can be surface engineered to adjust its properties, e.g., biodistribution, e.g., via incorporation of immuno-affinity ligands or cognate receptor ligands. For example, EV, e.g., exosomes, disclosed herein can be surface engineered to direct them to a specific cellular type, e.g., Schwann cells, sensory neurons, motor neurons, or meningeal macrophages, or can be surface engineered to enhance their migration to a specific compartment, e.g., to the CNS in order to improve intrathecal compartment retention.

In some aspects, an EV, e.g., exosome, for delivery to the CNS disclosed herein comprises a bio-distribution modifying agent or targeting moiety.

EVs, e.g., exosomes, exhibit preferential uptake in discrete cell types and tissues, and their tropism can be directed by adding proteins to their surface that interact with receptors on the surface of target cells.

In some aspects, a tropism moiety can increase uptake of the EV, e.g., an exosome, by a cell. In some aspects, the tropism moiety that can increase uptake of the EV, e.g., an exosome, by a cell comprises a lymphocyte antigen 75 (also known as DEC205 or CD205), C-type lectin domain family 9 member A (CLEC9A), C-type lectin domain family 6 (CLEC6), C-type lectin domain family 4 member A (also known as DCIR or CLEC4A), Dendritic Cell-Specific Intercellular adhesion molecule-3-Grabbing Non-integrin (also known as DC-SIGN or CD209), lectin-type oxidized LDL receptor 1 (LOX-1), macrophage receptor with collagenous structure (MARCO), C-type lectin domain family 12 member A (CLEC12A), C-type lectin domain family 10 member A (CLEC10A), DC-asialoglycoprotein receptor (DC-ASGPR), DC immunoreceptor 2 (DCIR2), Dectin-1, macrophage mannose receptor (MMR), BDCA-2 (CD303, CLEC4C), Dectin-2, BST-2 (CD317), Langerin, CD206, CD11b, CD11c, CD123, CD304, XCR1, AXL, SIGLEC 6, CD209, SIRPA, CX3CR1, GPR182, CD14, CD16, CD32, CD34, CD38, CD10, anti-CD3 antibody, or any combination thereof.

In some aspects, when tropism to the central nervous system is desired, an EV, e.g., exosome, of the present disclosure can comprise a tissue or cell-specific target ligand, which increases EV, e.g., exosome, tropism to a specific central nervous system tissue or cell. In some aspects, the cell is a glial cell. In some aspects, the glial cell is an oligodendrocyte, an astrocyte, an ependymal cell, a microglia cell, a Schwann cell, a satellite glial cell, an olfactory ensheathing cell, or a combination thereof. In some aspects, the cell is a neural stem cell. In some aspects, the cell-specific target ligand, which increases EV, e.g., exosome, tropism to a Schwann cells, binds to a Schwann cell surface marker, such as Myelin Basic Protein (MBP), Myelin Protein Zero (P0), P75NTR, NCAM, PMP22, or any combination thereof. In some aspects, the cell-specific tropism moiety comprises an antibody or an antigen-binding portion thereof, an aptamer, or an agonist or antagonist of a receptor expressed on the surface of the Schwann cell.

In certain aspects, the tropism moiety is linked, e.g., chemically linked via a maleimide moiety, to a scaffold moiety, e.g., a Scaffold X protein or a fragment thereof, on the exterior surface of the EV, e.g., exosome. Tropism can be further improved by the attachment of a half-life extension moiety (e.g., albumin or PEG), or any combination thereof to the external surface of an EV, e.g., exosome of the present disclosure.

Pharmacokinetics, biodistribution, and in particular, tropism and retention in the desired tissue or anatomical location can also be accomplish by selecting the appropriate administration route (e.g., intrathecal administration or intraocular administration to improve tropism to the central nervous system).

Surface antigens useful in the present disclosure include, but are not limited to, antigens that label a cell as a “self” cell. In some aspects, the surface antigen is selected from CD47, CD24, a fragment thereof, and any combination thereof. In certain aspects, the surface antigen comprises CD24, e.g., human CD24. In some aspects, the surface antigen comprises a fragment of CD24, e.g., human CD24. In certain aspects, the EV, e.g., exosome, is modified to express CD47 or a fragment thereof on the exterior surface of the EV, e.g., exosome.

In some aspects, the additional moiety of interest comprises an immune modulator (e.g., along with an antigen, adjuvant, and/or additional moiety of interest described herein). Accordingly, in certain aspects, a base EV (e.g., exosome) that can be used to produce or manufacture an EV-based vaccine described herein comprises the immune modulator (e.g., alone or in combination with the adjuvant and/or additional moiety of interest described herein), such that the immune modulator is present in the EV prior to the addition of the antigen. For instance, in some aspects, the immune modulator can be introduced into a producer cell when producing the base EVs (e.g., exosomes). In some aspects, the immune modulator can be added to the EVs (e.g., exosomes) after being isolated from the producer cells. In such aspects, the immune modulator can be added to the isolated EVs (e.g., exosomes) before adding the antigen. In some aspects, the immune modulator is added to the EV after adding the antigen. In some aspects, the immune modulator is added to the EV together with the antigen.

In some aspects, an immune modulator comprises an inhibitor for a negative checkpoint regulator or an inhibitor for a binding partner of a negative checkpoint regulator. In certain aspects, the negative checkpoint regulator comprises cytotoxic T-lymphocyte-associated protein 4 (CTLA-4), programmed cell death protein 1 (PD-1), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, CD73, or any combination thereof.

In some aspects, the immunomodulating component is a protein, a peptide, a glycolipid, or a glycoprotein.

### Anchoring Moieties (AM)

As described herein, in some aspects, an antigen, adjuvant, and/or one or more additional moieties of interest (e.g., targeting moiety and/or immune modulator) can be linked to an exterior surface and/or luminal surface of the EVs using an anchoring moiety. In some aspects, the antigen, adjuvant, and/or one or more additional moieties of interest (e.g., targeting moiety and/or immune modulator) is linked directly to the anchoring moiety or via a linker. In some aspects, the antigen, adjuvant, and/or one or more additional moieties of interest (e.g., targeting moiety and/or immune modulator) can be attached to an anchoring moiety or linker combination via reaction between a “reactive group” (RG; e.g., amine, thiol, hydroxy, carboxylic acid, or azide) with a “reactive moiety” (RM; e.g., maleimide, succinate, NHS).

In some aspect, an anchoring moiety can be chemically conjugated to the antigen, adjuvant, and/or one or more additional moieties of interest (e.g., targeting moiety and/or immune modulator) to enhance its hydrophobic character. Non-limiting examples of such anchoring moieties are provided further below.

As described elsewhere in the present disclosure, in some aspects, the antigen, adjuvant, and/or one or more additional moieties of interest (e.g., targeting moiety and/or immune modulator) are modified, such that they include a free amine group at the N-terminus (e.g., comprising a N-terminal lysine). In some aspects, the anchoring moiety is conjugated to the free amine group at the N-terminus either directly or via one or more linkers.

### Exemplary Anchoring Moieties

In some aspects, anchoring moieties that can be used to link an antigen, adjuvant, and/or one or more additional moieties of interest (e.g., targeting moiety and/or immune modulator) to the exterior surface and/or luminal surface of the EV (e.g., exosome) comprises: a sterol (e.g., cholesterol), GM1, a lipid (e.g., fatty acid (e.g., palmitate), ionizable lipid, glycerophospholipid, sphingolipid), a vitamin (e.g., tocopherol (e.g., vitamin E)), vitamin A, vitamin D, vitamin K), a small molecule, a peptide (e.g., cell penetrating peptide), including any derivatives thereof, or a combination thereof.

In some aspects, the anchoring moiety is a lipid. A lipid anchoring moiety can be any lipid known in the art, e.g., palmitic acid or glycosylphosphatidylinositols. Non-limiting examples of other suitable lipids are described in Fahy et al., Biochim Biophys Acta 1811(11): 637-647 (November 2011), which is incorporated herein by reference in its entirety. In some aspects, the lipid is a fatty acid, phosphatide, phospholipid (e.g., phosphatidyl choline, phosphatidyl serine, or phosphatidyl ethanolamine), or analogue thereof (e.g. phophatidylcholine, lecithin, phosphatidylethanolamine, cephalin, or phosphatidylserine or analogue or portion thereof, such as a partially hydrolyzed portion thereof). In some aspects, the lipid comprises an ionizable lipid. In some aspects, the lipid comprises a glycerophospholipid. In some aspects, the lipid comprises a sphingolipid.

Generally, anchoring moieties are chemically attached. However, an anchoring moiety can be attached to an antigen, adjuvant, and/or one or more additional moieties of interest (e.g., targeting moiety and/or immune modulator) enzymatically.

Some types of membrane anchors that can be used to practice the methods of the present disclosure are presented in the following table:

| Modification | Modifying Group |
|---|---|
| S-Palmitoylation | 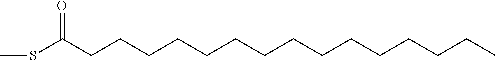 [SMILES: CCCCCCCCCCCCCCCC(=O)SC] |
| N-Palmitoylation | 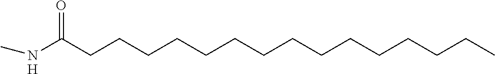 |
| N-Myristoylation | 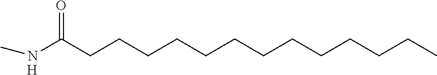 |
| O-Acylation | 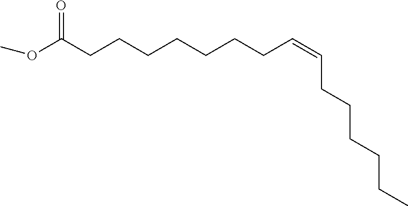 [SMILES: CCCCCC/C=C\CCCCCCCC(=O)OC] |
| Farnesylation | 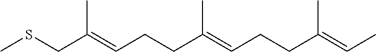 [SMILES: C/C=C(\C)CC/C=C(\C)CC/C=C(\C)CSC] |
| Geranylgeranylation | 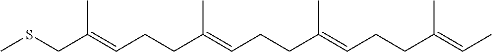 [SMILES: C/C=C(\C)CC/C=C(\C)CC/C=C(\C)CC/C=C(\C)CSC] |
| Cholesterol | 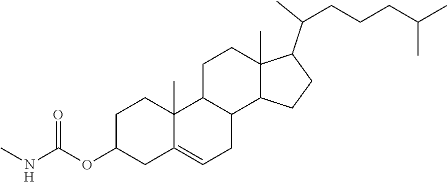 |

In some aspects, an anchoring moiety of the present disclosure can comprise two or more types of anchoring moieties disclosed herein. For example, in some aspects, an anchoring moiety can comprise two lipids, e.g., a phospholipids and a fatty acid, or two phospholipids, or two fatty acids, or a lipid and a vitamin, or cholesterol and a vitamin.

In some aspects, the anchoring moiety useful for the present disclosure comprises a sterol, steroid, hopanoid, hydroxysteroid, secosteroid, or analog thereof with lipophilic properties. In some aspects, the anchoring moiety comprises a sterol, such as a phytosterol, mycosterol, or zoosterol. Exemplary zoosterols include cholesterol and 24S-hydroxycholesterol; exemplary phytosterols include ergosterol (mycosterol), campesterol, sitosterol, and stigmasterol. In some aspects, the sterol is selected from ergosterol, 7-dehydrocholesterol, cholesterol, 24S-hydroxycholesterol, lanosterol, cycloartenol, fucosterol, saringosterol, campesterol, f3-sitosterol, sitostanol, coprostanol, avenasterol, or stigmasterol. Sterols can be found either as free sterols, acylated (sterol esters), alkylated (steryl alkyl ethers), sulfated (sterol sulfate), or linked to a glycoside moiety (steryl glycosides), which can be itself acylated (acylated sterol glycosides). In some aspects, the anchoring moiety is a cholesterol.

In some aspects, the anchoring moiety comprises a steroid. In some aspects, the steroid is selected from dihydrotestosterone, uvaol, hecigenin, diosgenin, progesterone, or cortisol.

In some aspects, the anchoring moiety is a fatty acid. In some aspects, the fatty acid is a short-chain, medium-chain, or long-chain fatty acid. In some aspects, the fatty acid is a saturated fatty acid. In some aspects, the fatty acid is an unsaturated fatty acid. In some aspects, the fatty acid is a monounsaturated fatty acid. In some aspects, the fatty acid is a polyunsaturated fatty acid, such as an omega-3 or omega-6 fatty acid.

In some aspects, the anchoring moiety comprises a phospholipid. Phospholipids are a class of lipids that are a major component of all cell membranes. They can form lipid bilayers because of their amphiphilic characteristic. The structure of the phospholipid molecule generally consists of two hydrophobic fatty acid “tails” and a hydrophilic “head” consisting of a phosphate group. For example, a phospholipid can be a lipid according to the following formula:

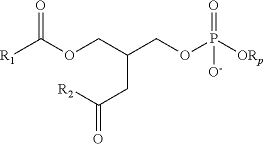

in which Rₚ represents a phospholipid moiety and R₁ and R₂ represent fatty acid moieties with or without unsaturation that can be the same or different.

In some aspects, an antigen, adjuvant, and/or one or more additional moieties of interest (e.g., targeting moiety and/or immune modulator) is linked to an anchoring moiety disclosed herein via a linker combination, which can comprise any combination of cleavable and/or non-cleavable linkers. Accordingly, in some aspects, an EV described herein can comprise multiple coupling strategies described herein (e.g., a cell penetrating peptide and a linker). Not to be bound by any one theory, one of the functions of a linker combination is to provide the optimal spacing between the anchoring moiety and the antigen, adjuvant, and/or one or more additional moieties of interest (e.g., targeting moiety and/or immune modulator).

Linkers can be susceptible to cleavage (“cleavable linker”) thereby facilitating release of the biologically active molecule. Thus, in some aspects, a linker combination disclosed herein can comprise a cleavable linker. Such cleavable linkers can be susceptible, for example, to acid-induced cleavage, photo-induced cleavage, peptidase-induced cleavage, esterase-induced cleavage, and disulfide bond cleavage, at conditions under which the biologically active molecule remains active. Alternatively, linkers can be substantially resistant to cleavage (“non-cleavable linker”). In some aspects, the cleavable linker comprises a spacer. In some aspects the spacer is PEG.

In some aspects, a linker combination comprises at least 2, at least 3, at least 4, at least 5, or at least 6 or more different linkers disclosed herein. In some aspects, linkers in a linker combination can be linked by an ester linkage (e.g., phosphodiester or phosphorothioate ester).

In some aspects, the linker is direct bond between an anchoring moiety and an antigen, adjuvant, and/or one or more additional moieties of interest (e.g., targeting moiety and/or immune modulator).

In some aspects, the linker combination comprises a “non-cleavable liken” Non-cleavable linkers are any chemical moiety capable of linking two or more components of an EV (e.g., exosome) disclosed herein, such as an antigen, adjuvant, and/or one or more additional moieties of interest (e.g., targeting moiety and/or immune modulator), in a stable, covalent manner and does not fall off under the categories listed above for cleavable linkers. Thus, non-cleavable linkers are substantially resistant to acid-induced cleavage, photo-induced cleavage, peptidase-induced cleavage, esterase-induced cleavage and disulfide bond cleavage.

Furthermore, non-cleavable refers to the ability of the chemical bond in the linker or adjoining to the linker to withstand cleavage induced by an acid, photolabile-cleaving agent, a peptidase, an esterase, or a chemical or physiological compound that cleaves a disulfide bond, at conditions under which a cyclic dinucleotide and/or the antibody does not lose its activity. In some aspects, the biologically active molecule is attached to the linker via another linker, e.g., a self-immolative linker.

In some aspects, the linker combination comprises a non-cleavable linker comprising, e.g., tetraethylene glycol (TEG), hexaethylene glycol (HEG), polyethylene glycol (PEG), thiosuccinimide, non-peptidyl amide, or any combination thereof. In some aspects, the non-cleavable linker comprises a spacer unit to link the biologically active molecule to the non-cleavable linker.

In some aspects, one or more non-cleavable linkers comprise smaller units (e.g., HEG, TEG, glycerol, C2 to C12 alkyl, and the like) linked together. In one aspect, the linkage is an ester linkage (e.g., phosphodiester or phosphorothioate ester) or other linkage.

In some aspects, the linker combination comprises a non-cleavable linker, wherein the non-cleavable linker comprises a polyethylene glycol (PEG) characterized by a formula R³—(O—CH₂—CH₂)ₙ— or R³—(0-CH₂—CH₂)ₙ—O— with R³ being hydrogen, methyl or ethyl and n having a value from 2 to 200. In some aspects, the linker comprises a spacer, wherein the spacer is PEG.

In some aspects, the PEG linker is an oligo-ethylene glycol, e.g., diethylene glycol, triethylene glycol, tetra ethylene glycol (TEG), pentaethylene glycol, or a hexaethylene glycol (HEG) linker.

In some aspects, n has a value of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 189, 181, 182, 183, 184, 185, 186, 187, 188, 189, 190, 191, 192, 193, 194, 195, 196, 197, 198, 199, or 200.

In some aspects, n is between 2 and 10, between 10 and 20, between 20 and 30, between 30 and 40, between 40 and 50, between 50 and 60, between 60 and 70, between 70 and 80, between 80 and 90, between 90 and 100, between 100 and 110, between 110 and 120, between 120 and 130, between 130 and 140, between 140 and 150, between 150 and 160, between 160 and 170, between 170 and 180, between 180 and 190, or between 190 and 200.

In some specific aspects, n has a value from 3 to 200, from 3 to 20, from 10 to 30, or from 9 to 45.

In some aspects, the PEG is a branched PEG. Branched PEGs have three to ten PEG chains emanating from a central core group.

In some aspects, the PEG moiety is a monodisperse polyethylene glycol. In the context of the present disclosure, a monodisperse polyethylene glycol (mdPEG) is a PEG that has a single, defined chain length and molecular weight. mdPEGs are typically generated by separation from the polymerization mixture by chromatography. In certain formulae, a monodisperse PEG moiety is assigned the abbreviation mdPEG.

In some aspects, the PEG is a Star PEG. Star PEGs have 10 to 100 PEG chains emanating from a central core group.

In some aspects, the PEG is a Comb PEGs. Comb PEGs have multiple PEG chains normally grafted onto a polymer backbone.

In certain aspects, the PEG has a molar mass between 100 g/mol and 3000 g/mol, particularly between 100 g/mol and 2500 g/mol, more particularly of approx. 100 g/mol to 2000 g/mol. In certain aspects, the PEG has a molar mass between 200 g/mol and 3000 g/mol, particularly between 300 g/mol and 2500 g/mol, more particularly of approx. 400 g/mol to 2000 g/mol.

In some aspects, the PEG is PEG₁₀₀, PEG₂₀₀, PEG₃₀₀, PEG₄₀₀, PEG₅₀₀, PEG₆₀₀, PEG₇₀₀, PEG₈₀₀, PEG₀₀₀, PEG₁₀₀₀, PEG₁₁₀₀, PEG₁₂₀₀, PEG₁₃₀₀, PEG₁₄₀₀, PEG₁₅₀₀, PEG₁₆₀₀, PEG₁₇₀₀, PEG₁₈₀₀, PEG₁₉₀₀, PEG₂₀₀₀, PEG₂₁₀₀, PEG₂₂₀₀, PEG₂₃₀₀, PEG₂₄₀₀, PEG₂₅₀₀, PEG₁₆₀₀, PEG₁₇₀₀, PEG₁₈₀₀, PEG₁₉₀₀, PEG₂₀₀₀, PEG₂₁₀₀, PEG₂₂₀₀, PEG₂₃₀₀, PEG₂₄₀₀, PEG₂₅₀₀, PEG₂₆₀₀, PEG₂₇₀₀, PEG₂₈₀₀, PEG₂₉₀₀, or PEG₃₀₀₀. In one particular aspect, the PEG is PEG₄₀₀. In some aspects, the PEG is PEG₂₀₀₀.

In some aspects, a linker combination of the present disclosure can comprise several PEG linkers, e.g., a cleavable linker flanked by PEG, HEG, or TEG linkers.

In some aspects, the linker combination comprises (HEG)n and/or (TEG)n, wherein n is an integer between 1 and 50, and each unit is connected, e.g., via a phosphate ester linker, a phosphorothioate ester linkage, or a combination thereof.

In some aspects, the linker combination comprises a non-cleavable linker comprising a glycerol unit or a polyglycerol (PG) described by the formula ((R₃—O—(CH₂—CHOH—CH₂O)ₙ—) with R3 being hydrogen, methyl or ethyl, and n having a value from 3 to 200. In some aspects, n has a value from 3 to 20. In some aspects, n has a value from 10 to 30.

In some aspects, the PG linker is a diglycerol, triglycerol, tetraglycerol (TG), pentaglycerol, or a hexaglycerol (HG) linker.

In some aspects, the linker combination comprises (glycerol)n, and/or (HG)n and/or (TG)n, wherein n is an integer between 1 and 50, and each unit is connected, e.g., via a phosphate ester linker, a phosphorothioate ester linkage, or a combination thereof.

In some aspects, the linker combination comprises at least one aliphatic (alkyl) linker, e.g., propyl, butyl, hexyl, or C2-C12 alkyl, such as C2-C10 alkyl or C2-C6 alkyl.

In some aspects, different components of an EV (e.g., exosome) disclosed herein (e.g., an antigen, adjuvant, and/or additional moiety of interest (e.g., targeting moiety and/or immune modulator)) can be linker by a cleavable linker. The term cleavable linker refers to a linker comprising at least one linkage or chemical bond that can be broken or cleaved. As used herein, the term cleave refers to the breaking of one or more chemical bonds in a relatively large molecule in a manner that produces two or more relatively smaller molecules. Cleavage can be mediated, e.g., by a nuclease, peptidase, protease, phosphatase, oxidase, or reductase, for example, or by specific physicochemical conditions, e.g., redox environment, pH, presence of reactive oxygen species, or specific wavelengths of light.

In some aspects, the term “cleavable,” as used herein, refers, e.g., to rapidly degradable linkers, such as, e.g., phosphodiester and disulfides, while the term “non-cleavable” refers, e.g., to more stable linkages, such as, e.g., nuclease-resistant phosphorothioates.

In some aspects, the cleavable linker is a dinucleotide, trinucleotide or tetranucleotide linker, a disulfide, an imine, a silyl ether, carbonate, a thioketal, a val-cit dipeptide, Val-Ala dipeptide, Ala-Ala-Asn tripeptide, poly-arginine, phosphodiesters, acid-labile or any combination thereof.

In some aspects, the cleavable linker comprises valine-alanine-p-aminobenzylcarbamate or valine-citrulline-p-aminobenzylcarbamate, Ala-Ala-Asn-p-aminobenzylcarbamate.

In some aspects, the linker combination comprises a redox cleavable linker. As a non-limiting example, one type of cleavable linker is a redox cleavable linking group that is cleaved upon reduction or upon oxidation.

In some aspects, the redox cleavable linker contains a disulfide bond, i.e., it is a disulfide cleavable linker.

Redox cleavable linkers can be reduced, e.g., by intracellular mercaptans, oxidases, or reductases.

In some aspects, the linker combination can comprise a cleavable linker which can be cleaved by a reactive oxygen species (ROS), such as superoxide (Of) or hydrogen peroxide (H2O2), generated, e.g., by inflammation processes such as activated neutrophils. In some aspects, the ROS cleavable linker is a thioketal cleavable linker. See, e.g., U.S. Pat. No. 8,354,455 B2, which is herein incorporated by reference in its entirety.

In some aspects, the linker is an “acid labile linker” comprising an acid cleavable linking group, which is a linking group that is selectively cleaved under acidic conditions (pH<7).

As a non-limiting example, the acid cleavable linking group is cleaved in an acidic environment, e.g., about 6.0, 5.5, 5.0 or less. In some aspects, the pH is about 6.5 or less. In some aspects, the linker is cleaved by an agent such as an enzyme that can act as a general acid, e.g., a peptidase (which can be substrate specific) or a phosphatase. Within cells, certain low pH organelles, such as endosomes and lysosomes, can provide a cleaving environment to the acid cleavable linking group. Although the pH of human serum is 7.4, the average pH in cells is slightly lower, ranging from about 7.1 to 7.3. Endosomes also have an acidic pH, ranging from 5.5 to 6.0, and lysosomes are about 5.0 at an even more acidic pH. Accordingly, pH dependent cleavable linkers are sometimes called endosmotically labile linkers in the art.

In some aspects, the linker comprises a low pH-labile hydrazone bond, silyl ether, or carbonate. Such acid-labile bonds have been extensively used in the field of conjugates, e.g., antibody-drug conjugates. See, for example, Zhou et al., Biomacromolecules 2011, 12, 1460-7; Yuan et al., Acta Biomater. 2008, 4, 1024-37; Zhang et al., Acta Biomater. 2007, 6, 838-50; Yang et al., J. Pharmacol. Exp. Ther. 2007, 321, 462-8; Reddy et al., Cancer Chemother. Pharmacol 2006, 58, 229-36; Doronina et al., Nature Biotechnol. 2003, 21, 778-84.

Further examples can be found in U.S. Pat. Nos. 9,790,494 B2 and 8,137,695 B2, the contents of which are incorporated herein by reference in their entireties.

In some aspects, the linker combination can comprise a linker cleavable by intracellular or extracellular enzymes, e.g., proteases, esterases, nucleases, amidades, reductase. The range of enzymes that can cleave a specific linker in a linker combination depends on the specific bonds and chemical structure of the linker. Accordingly, peptidic linkers can be cleaved, e.g., by peptidases, linkers containing ester linkages can be cleaved, e.g., by esterases; linkers containing amide linkages can be cleaved, e.g., by amidades; linkers containing disulfide linkages can be cleaved, e.g., by reductases; etc.

In some aspects, the linker combination comprises a protease cleavable linker, i.e., a linker that can be cleaved by an endogenous protease. Only certain peptides are readily cleaved inside or outside cells. See, e.g., Trout et al., 79 Proc. Natl. Acad. Sci. USA, 626-629 (1982) and Umemoto et al. 43 Int. J. Cancer, 677-684 (1989). Cleavable linkers can contain cleavable sites composed of α-amino acid units and peptidic bonds, which chemically are amide bonds between the carboxylate of one amino acid and the amino group of a second amino acid. Other amide bonds, such as the bond between a carboxylate and the (3-amino acid group of lysine, are understood not to be peptidic bonds and are considered non-cleavable.

Some linkers are cleaved by esterases (“esterase cleavable linkers”). Only certain esters can be cleaved by esterases and amidases present inside or outside of cells. Esters are formed by the condensation of a carboxylic acid and an alcohol. Simple esters are esters produced with simple alcohols, such as aliphatic alcohols, and small cyclic and small aromatic alcohols. Examples of ester-based cleavable linking groups include, but are not limited to, esters of alkylene, alkenylene and alkynylene groups. The ester cleavable linking group has the general formula —C(O) O— or —OC(O)—.

In some aspects, a linker combination can includes a phosphate-based cleavable linking group is cleaved by an agent that degrades or hydrolyzes phosphate groups. An example of an agent that cleaves intracellular phosphate groups is an enzyme such as intracellular phosphatase, nuclease or phosphodiesterase.

In some aspects, the combination linker comprises a photoactivated cleavable linker, e.g., a nitrobenzyl linker or a linker comprising a nitrobenzyl reactive group.

In some aspects, the linker combination comprises a self-immolative linker In some aspects, the self-immolative linker in the EV (e.g., exosome) of the present disclosure undergoes 1,4 elimination after the enzymatic cleavage of the protease-cleavable linker. In some aspects, the self-immolative linker in the EV (e.g., exosome) of the present disclosure undergoes 1,6 elimination after the enzymatic cleavage of the protease-cleavable linker. In some aspects, the self-immolative linker is, e.g., a p-aminobenzyl (pAB) derivative, such as a p-aminobenzyl carbamate (pABC), a p-amino benzyl ether (PABE), a p-amino benzyl carbonate, or a combination thereof.

### Conjugated EVs (e.g., Exosomes)

Unlike antibodies, EVs (e.g., exosomes) can accommodate large numbers of molecules attached to their surface, e.g., on the order of thousands to tens of thousands of molecules per EV (e.g., exosome). EV (e.g., exosome)-drug conjugates thus represent a platform to deliver a high concentration of therapeutic compound to discrete cell types, while at the same time limiting overall systemic exposure to the compound, which in turn reduces off-target toxicity.

The present disclosure provide EVs, e.g., exosomes, that have been engineered by reacting a first molecular entity comprising a free thiol group with a second molecular entity comprising a maleimide group, wherein the maleimide moiety covalently links the first molecular entity with the second molecular entity via a maleimide moiety.

Non-limiting examples of biologically active molecules that can attached to an EV (e.g., exosome) via a maleimide moiety include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, or siRNA), morpholino, amino acids (e.g., amino acids comprising a detectable moiety or a toxin that disrupt translation), polypeptides (e.g., enzymes), lipids, carbohydrates, small molecules (e.g., small molecule drugs and toxins), antigens (e.g., vaccine antigens), adjuvants (e.g., vaccine adjuvants), etc.

In some aspects, an EV (e.g., exosome) of the present disclosure can comprise more than one type of biologically active molecule. In some aspects, biologically active molecules can be, e.g., small molecules such as cyclic dinucleotides, toxins such as auristatins (e.g., monoethyl auristatin E, MMAE), antibodies (e.g., naked antibodies or antibody-drug conjugates), STING agonists, tolerizing agents, antisense oligonucleotides, PROTACs, morpholinos, lysophosphatidic acid receptor antagonists (e.g., LPA1 antagonists) or any combinations thereof. In some aspects, an EV (e.g., exosome) of the present disclosure can comprise, e.g., a vaccine antigen and optionally a vaccine adjuvant. In some aspects, an EV (e.g., exosome) of the present disclosure can comprise a therapeutic payload (e.g., a STING or one payload disclosed below) and a targeting moiety and/or a tropism moiety.

EVs (e.g., exosomes) disclosed herein can comprise one or more affinity ligands that link or conjugate a molecule of interest (e.g., antigen, adjuvant, immune modulator, and/or targeting moiety) to the EVs (e.g., to the exterior surface or on the luminal surface) or to a target cell. In some aspects, an affinity ligand disclosed herein has one or more of the following properties: (i) derived from a synthetic library, (ii) sub-nanomolar affinity for a scaffold moiety (e.g., Scaffold X) with emphasis on slow off rate, (iii) binds epitope on membrane-distal IgV domain of a scaffold moiety (e.g., Scaffold X), (iv) free of disulfide linkages, (v) free of N-linked glycosylation sites, (vi) less than 20 amino acids in length, (vii) monomeric, (viii) electroneutral at physiological pH, (ix) hydrophilic, (x) resistant to protease digestion, (xi) amenable to expression in prokaryotic and eukaryotic hosts, (xii) can accommodate N- or C-terminus fusion, (xiii) nonimmunogenic, (xiv) can contain a tag for purification and/or separation, e.g., of an EV, and (xv) combinations thereof. As described herein, in some aspects, an affinity ligand disclosed herein can specifically bind (e.g., with high affinity) to a moiety expressed on the surface of an EV (e.g., exosome). In certain aspects, an affinity ligand specifically binds to a scaffold moiety expressed on the surface of an EV. In some aspects, an affinity ligand specifically binds to any moiety expressed on the surface of an EV (e.g., cholesterol). In some aspects, an affinity ligand disclosed herein can specifically bind (e.g., with high affinity) to a moiety expressed on a target cell. Non-limiting examples of such affinity ligands are provided throughout the present disclosure.

As described above, an affinity ligand useful for the present disclosure can be engineered to express one or more tags. In some aspects, such tags can be useful in the purification and/or separation of an agent that is conjugated to the affinity ligand. For example, in some aspects, an EV (e.g., exosome) comprises a scaffold moiety that is conjugated to an affinity ligand fusion, which comprises a molecule of interest (e.g., antigen, adjuvant, immune modulator, and/or targeting moiety) and a tag. In such aspects, the tag can be used to purify and/or separate the EV from a sample comprising the EV. In some aspects, a tag of an affinity ligand fusion described above is present between the affinity ligand and the molecule of interest. In some aspects, the tag of an affinity ligand fusion described above can be present at an end (e.g., N-terminus) of the molecule of interest, as long as the tag does not interfere with the activity of the molecule of interest. Any tags useful in the art for purifying and/or separating an agent from a sample can be used in the present disclosure. Non-limiting examples of such tags include polyhistidine tags, polyarginine tags, glutathione-S-transferase (GST), maltose binding protein (MBP), S-tag, influenza virus HA tag, thioredoxin, staphylococcal protein A tag, FLAG™ epitope, AviTag epitope (for subsequent biotinylation), c-myc epitope, and combinations thereof. See, e.g., U.S. Pat. No. 7,655,413, which is herein incorporated by reference in its entirety.

As described herein, in some aspects, a molecule of interest can be expressed on the surface of an EV (e.g., exosome) via a scaffold moiety. In some of these aspects, the molecule of interest can be linked or conjugated to the scaffold moiety via an affinity ligand. For instance, as described herein, in certain aspects, an affinity ligand can be fused to a molecule of interest (e.g., antigen, adjuvant, immune modulator, and/or targeting moiety), and then the molecule of interest can be conjugated to a moiety expressed on the surface of an EV (e.g., scaffold moiety) via the affinity ligand. In some aspects, the affinity ligand increases the binding of the molecule of interest (e.g., antigen, adjuvant, immune modulator, and/or targeting moiety) to the moiety on the EV (e.g., scaffold moiety). In certain aspects, the binding of the molecule of interest to the moiety on the EV (e.g., scaffold moiety) is increased by at least about one-fold, at least about two-fold, at least about three-fold, at least about four-fold, at least about five-fold, at least about six-fold, at least about seven-fold, at least about eight-fold, at least about nine-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, at least about 10,000-fold or more, compared to a reference (e.g., binding of the molecule of interest to the moiety on the EV (e.g., scaffold moiety) without the use of an affinity ligand).

In some aspects, an affinity ligand that can be used with the present disclosure comprises a linear peptide. In certain aspects, an affinity ligand comprises at least about two, at least about three, at least about four, at least about five, at least about seven, at least about eight, at least about nine, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

Not to be bound by any one theory, using an affinity ligand (e.g., disclosed herein) to link or conjugate a molecule of interest (e.g., antigen, adjuvant, immune modulator, and/or targeting moiety) to a moiety expressed on the surface of an EV (e.g., scaffold moiety) can improve one or more properties of the EV (e.g., exosome) disclosed herein. For instance, in some aspects, by increasing the binding of a molecule of interest to a moiety on the EVs (e.g., scaffold moiety), an affinity ligand disclosed herein can allow for increased expression of the molecule of interest on the surface of an EV (e.g., exterior surface). Accordingly, in certain aspects, a fusion protein comprising (i) a molecule of interest (e.g., antigen, adjuvant, immune modulator, and/or targeting moiety), (ii) an affinity ligand, and (iii) a scaffold moiety is present in the EV (e.g., exterior surface) at a higher density compared to a reference (e.g., corresponding fusion protein without the affinity ligand). In some aspects, the density of the fusion protein on the surface of the exosome is increased by at least about one-fold, at least about two-fold, at least about three-fold, at least about four-fold, at least about five-fold, at least about six-fold, at least about seven-fold, at least about eight-fold, at least about nine-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, at least about 10,000-fold or more, compared to the reference.

In some aspects, an improved binding of a molecule of interest (e.g., antigen, adjuvant, immune modulator, and/or targeting moiety) to a moiety expressed on the surface of an EV (e.g., scaffold moiety) can reduce the time required to produce an EV (e.g., exosome) disclosed herein. Accordingly, in some aspects, an affinity ligand disclosed herein can reduce the time required for producing an engineered EV (e.g., exosome) disclosed herein (e.g., comprising a molecule of interest and a scaffold moiety). In certain aspects, the time required to produce an engineered EV (e.g., exosome) is reduced by at least about 5%, at least about 10%, at least about 20%, at least about 30%, at least about 40%, at least about 50%, at least about 60%, at least about 70%, at least about 80%, or at least about 90% or more, compared to a reference (e.g., time required to produce the corresponding EV without the affinity ligand).

In some aspects, an affinity ligand useful for the present disclosure comprises a cleavage site, such as a protease (e.g., thrombin) cleavage site.

A non-limiting example of an EV (e.g., exosome) comprising an affinity ligand is described below. It will be apparent to those skilled in the art that an affinity ligand disclosed herein can be used in combination with other EVs (e.g., exosomes) disclosed herein.

In some aspects, an EV (e.g., exosome) comprises (i) an antigen, (ii) a scaffold moiety, and (iii) an affinity ligand, wherein the affinity ligand is used to link or conjugate the antigen to the scaffold moiety. In some aspects, to produce such EVs, the affinity ligand is fused to an antigen (e.g., spike S protein), and then the antigen-affinity ligand fusion is linked or conjugated to the scaffold moiety via the affinity ligand. In certain aspects, the antigen comprises a receptor-binding domain (RBD) of a spike (S) protein of a coronavirus disclosed herein (e.g., COVID-19). In certain aspects, the scaffold moiety comprises a Scaffold X. Accordingly, in some aspects, an EV (e.g., exosome) comprises (i) a RBD of a coronavirus S protein, (ii) a Scaffold X, and (iii) an affinity ligand, wherein the affinity ligand is used to link or conjugate the RBD of a coronavirus S protein to the Scaffold X. In some aspects, the affinity ligand can be used to link or conjugate a fragment of the RBD of a coronavirus S protein. In certain aspects, a fragment of the RBD of a coronavirus S protein that can be linked or conjugated to a Scaffold X using an affinity ligand disclosed herein is less than about 100 amino acids in length (e.g., less than about 90 amino acids, less than about 80 amino acids, less than about 70 amino acids, less than about 60 amino acids, less than about 50 amino acids, less than about 40 amino acids, less than about 30 amino acids, less than about 20 amino acids, less than about 10 amino acids, or more).

### Producer Cell for Production of Engineered EVs, e.g., Exosomes

EVs, e.g., exosomes, of the present disclosure can be produced from a cell grown in vitro or a body fluid of a subject. When exosomes are produced from in vitro cell culture, various producer cells, e.g., HEK293 cells, CHO cells, and MSCs, can be used. In certain aspects, a producer cell is not a naturally-existing dendritic cell, macrophage, B cell, mast cell, neutrophil, Kupffer-Browicz cell, cell derived from any of these cells, or any combination thereof (i.e., non-naturally existing producer cell). As used herein, the term “non-naturally existing producer cell” refers to a producer cell (e.g., dendritic cell, macrophage, B cell, mast cell, neutrophil, Kupffer-Browicz cell, cell derived from any of these cells, or any combination thereof) that has been modified, such that the producer cell differs in structure and/or function compared to the naturally-existing counterpart. As described further below, in some aspects, the non-naturally existing producer cell has been modified to express one or more payloads disclosed herein (e.g., antigen, immune modulator, and/or adjuvant. In certain aspects, the non-naturally existing producer cell has been modified to express one or more targeting moieties disclosed herein. In some aspects, the non-naturally existing producer cell has been modified to express one or more scaffold moieties disclosed herein (e.g., Scaffold X and/or Scaffold Y).

The producer cell can be genetically modified to comprise one or more exogenous sequences (e.g., encoding an antigen, adjuvant, immune modulator, and/or targeting moiety) to produce the EVs (e.g., exosomes) described herein. The genetically-modified producer cell can contain the exogenous sequence by transient or stable transformation. The exogenous sequence can be transformed as a plasmid. The exogenous sequences can be stably integrated into a genomic sequence of the producer cell, at a targeted site or in a random site. In some aspects, a stable cell line is generated for production of lumen-engineered EVs (e.g., exosomes).

### Pharmaceutical Compositions

Provided herein are pharmaceutical compositions comprising an EV, e.g., exosome, of the present disclosure having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of extracellular vesicles. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

In some aspects, a pharmaceutical composition comprises one or more therapeutic agents and an exosome described herein. In certain aspects, the EVs, e.g., exosomes, are co-administered with of one or more additional therapeutic agents, in a pharmaceutically acceptable carrier. In some aspects, the pharmaceutical composition comprising the EV, e.g., exosome is administered prior to administration of the additional therapeutic agents. In some aspects, the pharmaceutical composition comprising the EV, e.g., exosome is administered after the administration of the additional therapeutic agents. In further aspects, the pharmaceutical composition comprising the EV, e.g., exosome is administered concurrently with the additional therapeutic agents.

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the extracellular vesicles described herein, use thereof in the compositions is contemplated. Supplementary therapeutic agents can also be incorporated into the compositions. Typically, a pharmaceutical composition is formulated to be compatible with its intended route of administration. The EVs, e.g., exosomes, can be administered by parenteral, topical, intravenous, oral, subcutaneous, intra-arterial, intradermal, transdermal, rectal, intracranial, intraperitoneal, intranasal, intrathecal, intramuscular route or as inhalants. In certain aspects, the pharmaceutical composition comprising exosomes is administered intravenously, e.g. by injection. The EVs, e.g., exosomes, can optionally be administered in combination with other therapeutic agents that are at least partly effective in treating the disease, disorder or condition for which the EVs, e.g., exosomes, are intended.

Solutions or suspensions can include the following components: a sterile diluent such as water, saline solution, fixed oils, polyethylene glycols, glycerin, propylene glycol or other synthetic solvents; antibacterial compounds such as benzyl alcohol or methyl parabens; antioxidants such as ascorbic acid or sodium bisulfate; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as mannitol, sorbitol, and sodium chloride can be added to the composition. Prolonged absorption of the injectable compositions can be brought about by including in the composition a compound which delays absorption, e.g., aluminum monostearate and gelatin.

Sterile injectable solutions can be prepared by incorporating the EVs, e.g., exosomes, in an effective amount and in an appropriate solvent with one or a combination of ingredients enumerated herein, as desired. Generally, dispersions are prepared by incorporating the EVs, e.g., exosomes, into a sterile vehicle that contains a basic dispersion medium and any desired other ingredients. In the case of sterile powders for the preparation of sterile injectable solutions, methods of preparation are vacuum drying and freeze-drying that yields a powder of the active ingredient plus any additional desired ingredient from a previously sterile-filtered solution thereof. The EVs, e.g., exosomes, can be administered in the form of a depot injection or implant preparation which can be formulated in such a manner to permit a sustained or pulsatile release of the EV, e.g., exosomes.

Systemic administration of compositions comprising exosomes can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects, the pharmaceutical composition comprising exosomes is administered intravenously into a subject that would benefit from the pharmaceutical composition. In some aspects, the composition is administered to the lymphatic system, e.g., by intralymphatic injection or by intranodal injection (see e.g., Senti et al., PNAS 105(46): 17908 (2008)), or by intramuscular injection, by intrathecal administration, by subcutaneous administration, by direct injection into the thymus, or into the liver.

In certain aspects, the pharmaceutical composition comprising exosomes is administered as a liquid suspension. In certain aspects, the pharmaceutical composition is administered as a formulation that is capable of forming a depot following administration. In certain preferred aspects, the depot slowly releases the EVs, e.g., exosomes, into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein comprise the EVs, e.g., exosomes, described herein and optionally a pharmaceutically active or therapeutic agent. The therapeutic agent can be a biological agent, a small molecule agent, or a nucleic acid agent.

Dosage forms are provided that comprise a pharmaceutical composition comprising the EVs, e.g., exosomes, described herein. In some aspects, the dosage form is formulated as a liquid suspension for intravenous injection.

In certain aspects, the preparation of exosomes is subjected to radiation, e.g., X rays, gamma rays, beta particles, alpha particles, neutrons, protons, elemental nuclei, UV rays in order to damage residual replication-competent nucleic acids.

In certain aspects, the preparation of exosomes is subjected to gamma irradiation using an irradiation dose of more than 1, 5, 10, 15, 20, 25, 30, 35, 40, 50, 60, 70, 80, 90, 100, or more than 100 kGy.

In certain aspects, the preparation of exosomes is subjected to X-ray irradiation using an irradiation dose of more than 0.1, 0.5, 1, 5, 10, 15, 20, 25, 30, 35, 40, 50, 60, 70, 80, 90, 100, 200, 300, 400, 500, 600, 700, 800, 900, 1000, 2000, 3000, 4000, 5000, 6000, 7000, 8000, 9000, 10000, or greater than 10000 mSv.

### Kits

Also provided herein are kits comprising one or more EVs (e.g., exosomes) described herein. In some aspects, provided herein is a kit comprising one or more containers filled with one or more of the ingredients of the pharmaceutical compositions described herein. In certain aspects, the kit comprises a first container and a second container, wherein the first container comprises a base EV (e.g., exosome) described herein (e.g., comprising an adjuvant), and the second container comprises an antigen of interest (or any additional moieties of interest, e.g., additional adjuvant, targeting moiety, or immune modulator), and optional an instruction for use. As is apparent from the disclosure, in some aspects, combining the first and second container results in the antigen of interest (or any additional moieties of interest, e.g., additional adjuvant, targeting moiety, or immune modulator) to link to a surface of the EV.

### Vaccines

Also provided herein are vaccines comprising one or more EVs (e.g., exosomes) described herein. As described in the present disclosure, such vaccines can be rapidly produced and manufactured using the methods disclosed herein. Such vaccines can also be tailored to a specific geographical region and/or to a particular individual (or subset of individuals). Accordingly, in certain aspects, the vaccines provided herein are regionalized vaccines and/or individualized vaccines.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); Crooke, Antisense drug Technology: Principles, Strategies and Applications, 2^{nd} Ed. CRC Press (2007) and in Ausubel et al. (1989) Current Protocols in Molecular Biology (John Wiley and Sons, Baltimore, Md.).

The following examples are offered by way of illustration and not by way of limitation.

### EXAMPLES

### Example 1: Generation of EV-Based Vaccines

To generate the EV-based vaccines described herein, EVs (e.g., exosomes) will be isolated from producer cells and stored in a separate container (independent of the producer cells) (i.e., referred to herein as base EVs). In some aspects, the producer cells will be modified to express one or more moieties of interest (e.g., adjuvant, targeting moiety, and/or immune modulator), such that the isolated EVs will comprise the one or more moieties of interest. Then, an antigen of interest (e.g., comprising an epitope derived from the S2 coronavirus protein) will be linked to the exterior and/or luminal surface of the base EVs (e.g., exosomes). The antigen of interest will be linked to the surface of the EV using the various approaches described herein, e.g., via a scaffold moiety, an anchoring moiety, affinity agent, chemical conjugation, cell penetrating peptide (CPP), split intein, SpyTag/SpyCatcher, ALFA-tag, Streptavidin/Avitag, Sortase, SNAP-tag, ProA/Fc-binding peptide, or any combinations thereof.

In some aspects, the constructed EV-based vaccines will be tested in an animal model to assess the ability of the vaccines to prevent and/or treat a disease or disorder.

### Example 2: Construction of EVs (e.g., Exosomes) Comprising Acceptor Domain Fused to PTGFRN

To generate the EV-based vaccines described herein, several different “plug-and-play” or “clip-on” strategies were analyzed for their ability to quickly and efficiently attach a molecule to the surface of EVs (e.g., exosomes).

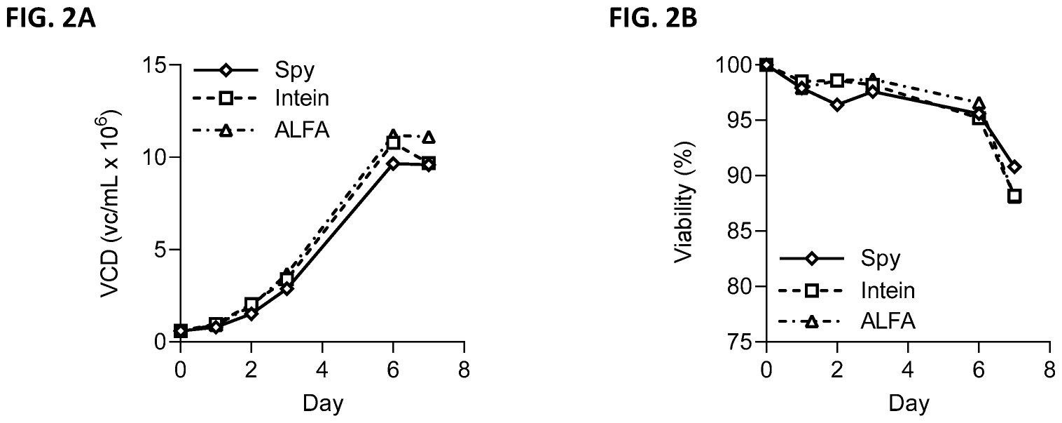

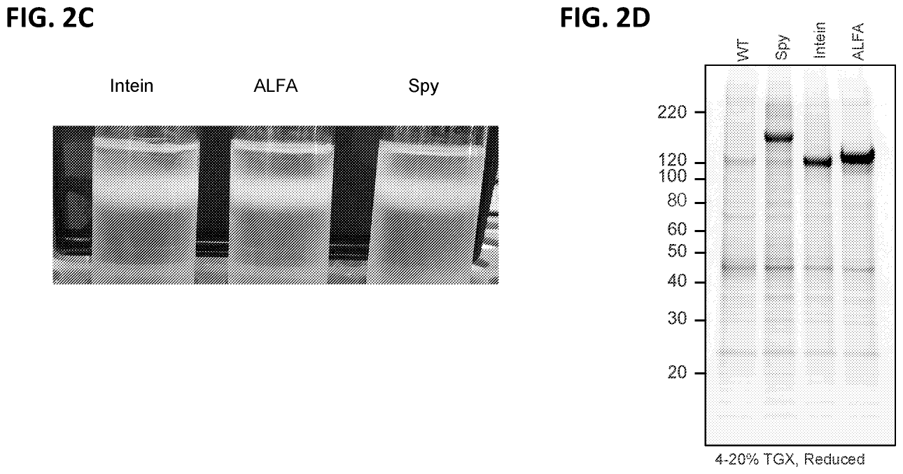

Briefly, HEK293 cells were transfected with constructs encoding an acceptor domain fused to the N-terminus of PTGFRN. Three different acceptor domains were analyzed: (1) SpyCatcher, (2) CfaC, and (3) ALFANb. As shown in FIGS. 2A and 2B, all of the PTGFRN fusion protein were stably expressed for at least a week post-transfection. Then EVs (e.g., exosomes) comprising the PTGFRN fusion proteins were successfully produced and purified from the producer cells (see FIGS. 2C and 2D).

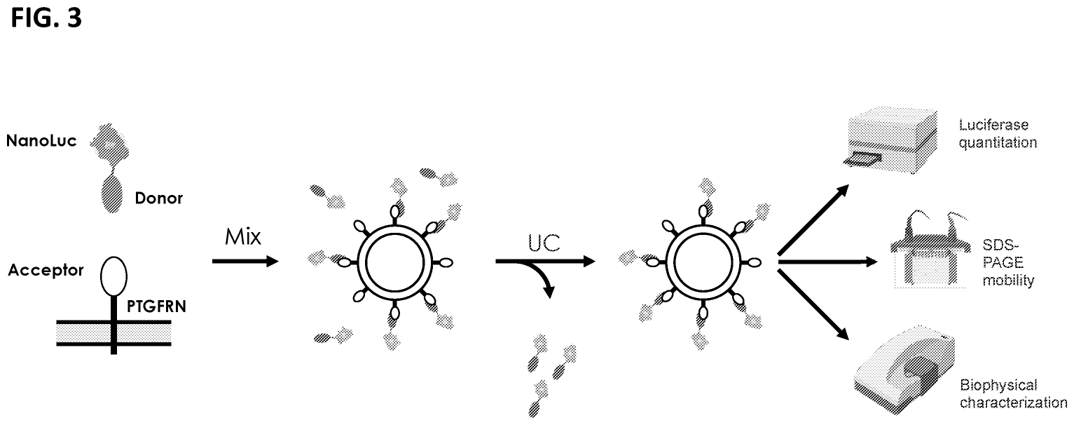

As described herein and further illustrated in FIG. 3, the EVs (e.g., exosomes) produced here could be useful as “base” EVs to which a moiety of interest (e.g., antigen) could rapidly be attached.

### Example 3: Construction of EVs (e.g., Exosomes) Comprising NanoLuc Luciferase Fused to ALFAtag

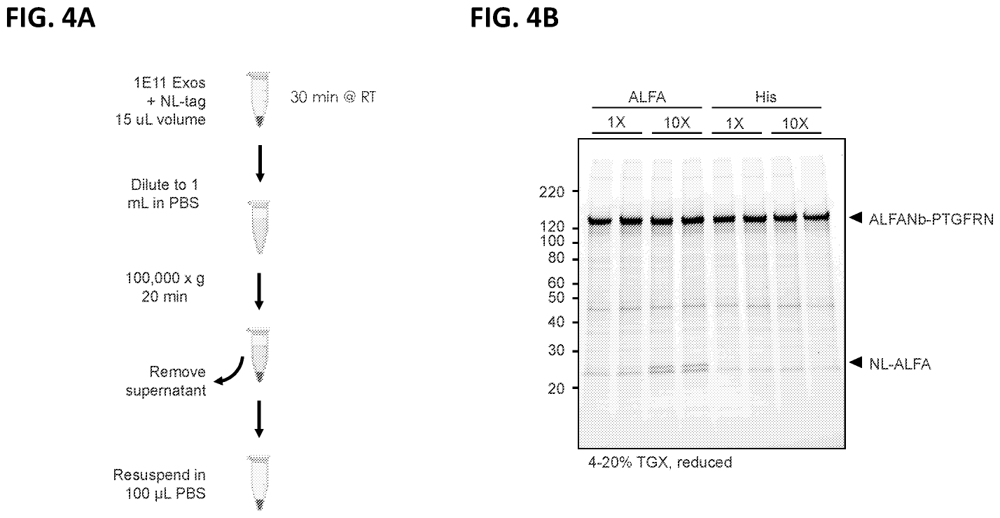

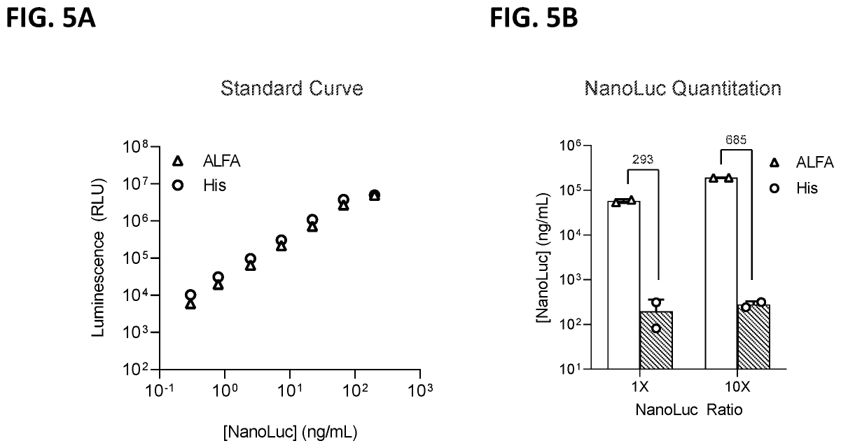

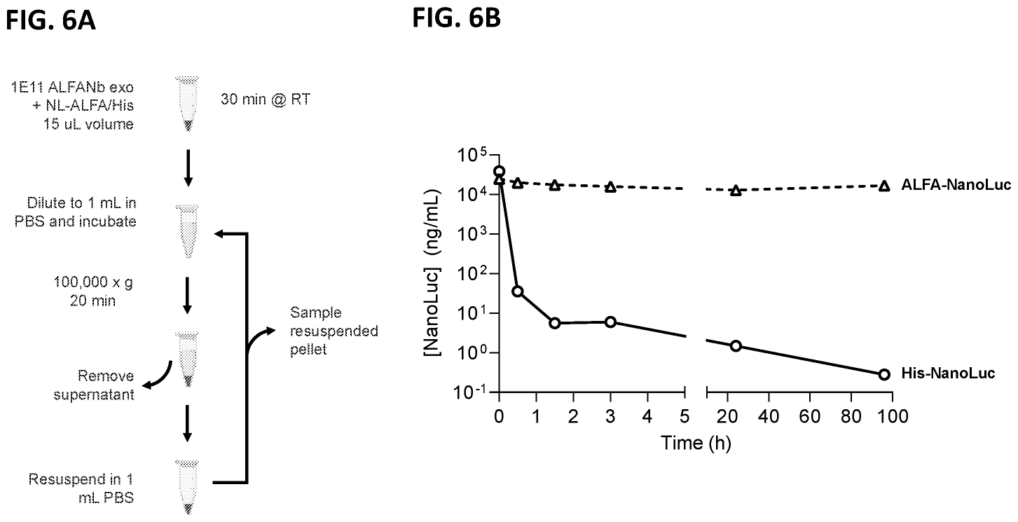

To further address the capability of the EV-based vaccines described herein, EVs (e.g., exosomes) overexpressing the ALFANb fused to PTGFRN were functionalized with solubly expressed NanoLuc fused to the ALFAtag peptide. The ALFAtag is capable of interacting with the ALFANb to form a stable, non-covalent interaction. FIG. 4A provides the general methods used. And, as shown FIG. 4B, SDS-PAGE analysis confirmed that the NanoLuc-ALFAtag was able to associate with the ALFANb-PTGFRN protein expressed on the exosome surface, at 1× or 10× molar excess of NanoLuc-ALFAtag. As shown in FIGS. 5A and 5B, compared to the control (i.e., exosomes mixed with NanoLuc fused to poly-histidine tag), loading of the NanoLuc-ALFAtag was significantly higher on the exosomes, indicating that the loading was due to the specific interaction between the ALFAtag and ALFANb-PTGFRN fusion on the exosomes. And, as shown in FIGS. 6A and 6B, the interaction between the ALFAtag and ALFANb-PTGFRN fusion remained stable for an extended period of time under physiologic conditions.

### Example 4: Construction of EVs (e.g., Exosomes) Comprising NanoLuc Luciferase Fused to Cfa Split Intein

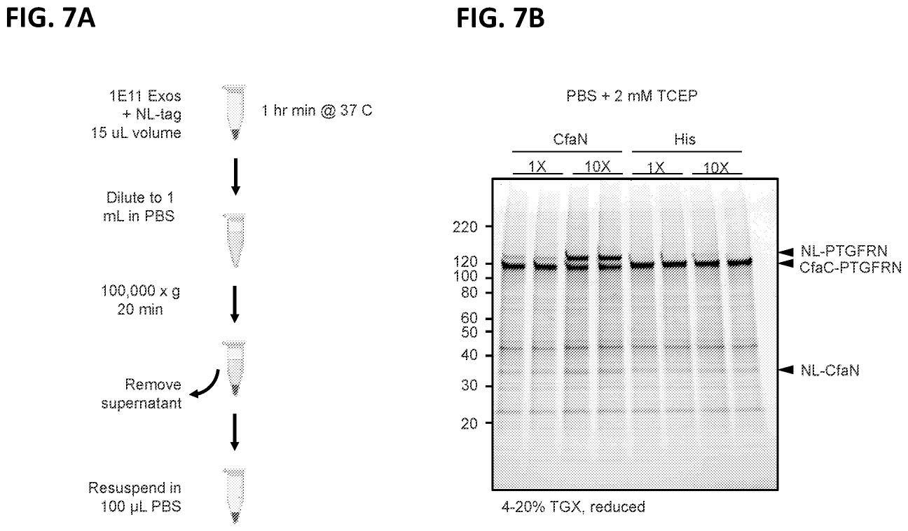

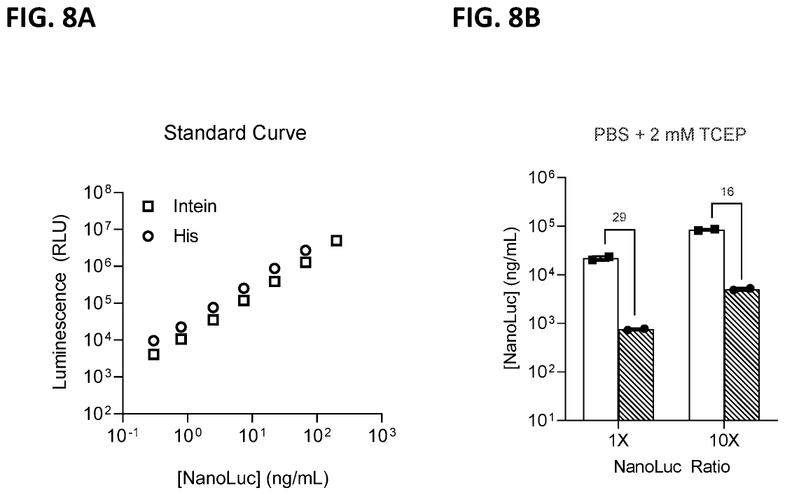

To confirm the results provided above, EVs overexpressing CfaC fused to PTGFRN were functionalized with solubly expressed NanoLuc fused to CfaN. Again, NanoLuc fused to a poly-histidine tag was used as a control. The general methods used are provided in FIG. 7A. Since intein trans-splicing results in covalent attachment of NanoLuc, a clear shift in molecular weight was visible by SDS-PAGE (NL-PTGFRN) (see FIG. 7B). The successful attachment of the NanoLuc was further confirmed when the expression of NanoLuc was measured on the exosomes. As shown in FIGS. 8A and 8B, compared to exosomes that were mixed with the NanoLuc-histidine control, exosomes that were mixed with the NanoLuc-CfaN had significantly higher NanoLuc expression.

### Example 5 Additional Construction of Modular EVs

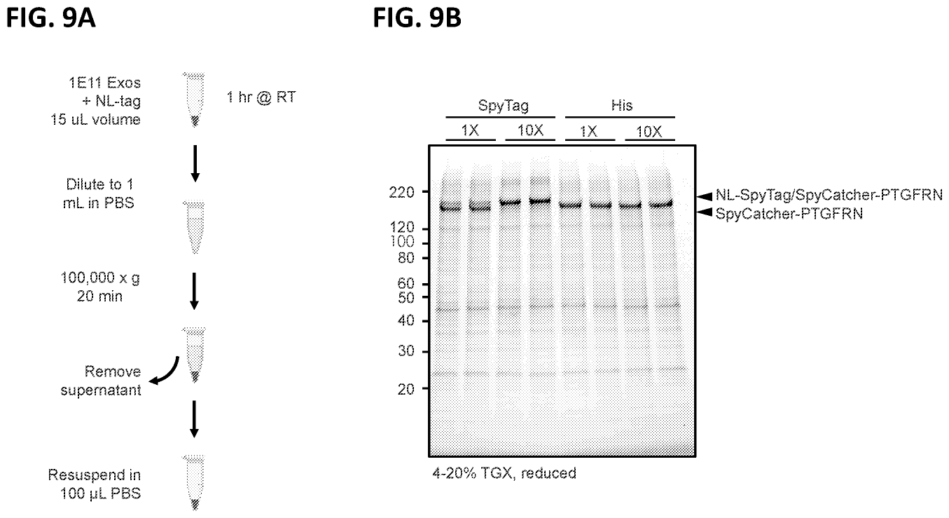

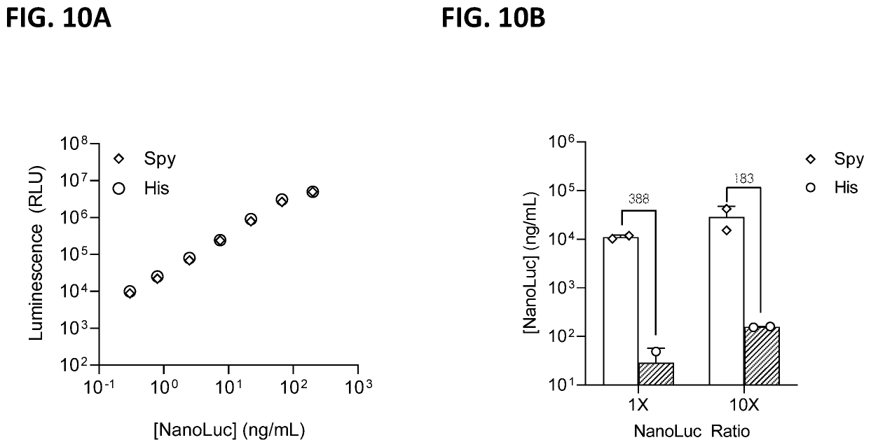

Further to the examples provided above, isolated EVs overexpressing SpyCatcher fused to PTGFRN were functionalized with solubly expressed NanoLuc fused to SpyTag, as described in FIG. 9A. The SDS-PAGE provided in FIG. 9B confirms the loading of NanoLuc-SpyTag onto SpyCatcher-PTGFRN overexpressing exosomes. Since SpyCatcher/SpyTag forms a spontaneous isopeptide bond, covalent attachment of NanoLuc-SpyTag resulted in a clear shift in molecular weight is visible by SDS-PAGE (NL-SpyTag/SpyCatcher-PTGFRN) (see FIG. 9B). FIGS. 10A and 10B confirm the results quantitatively.

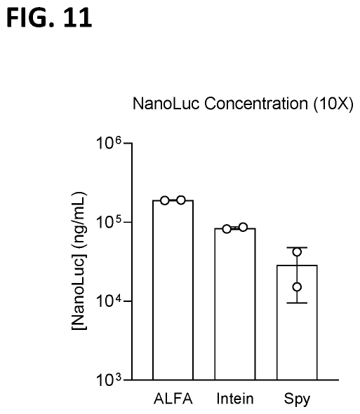

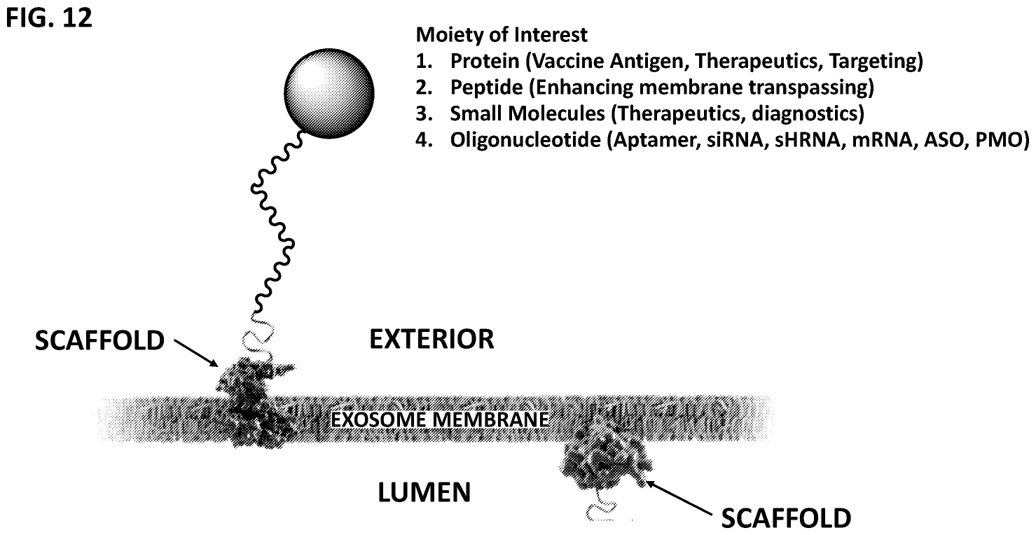

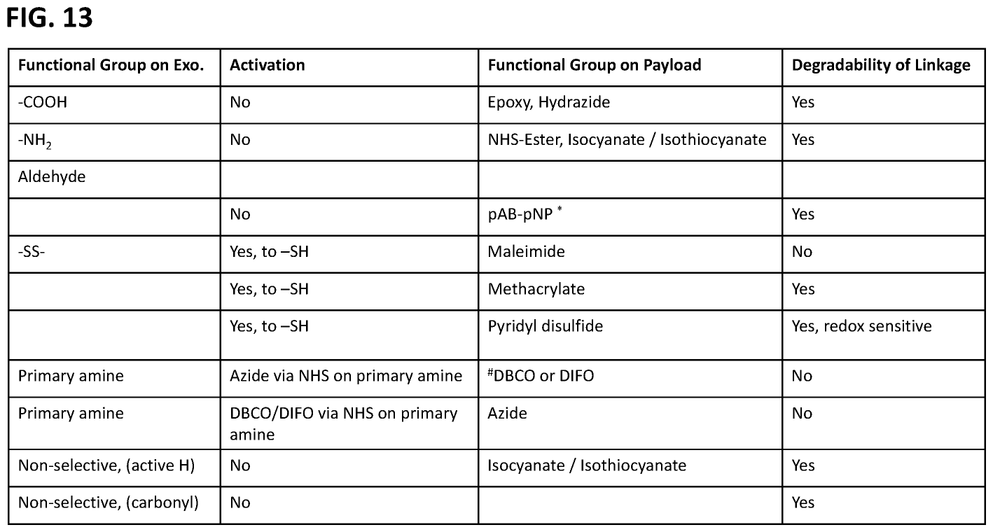

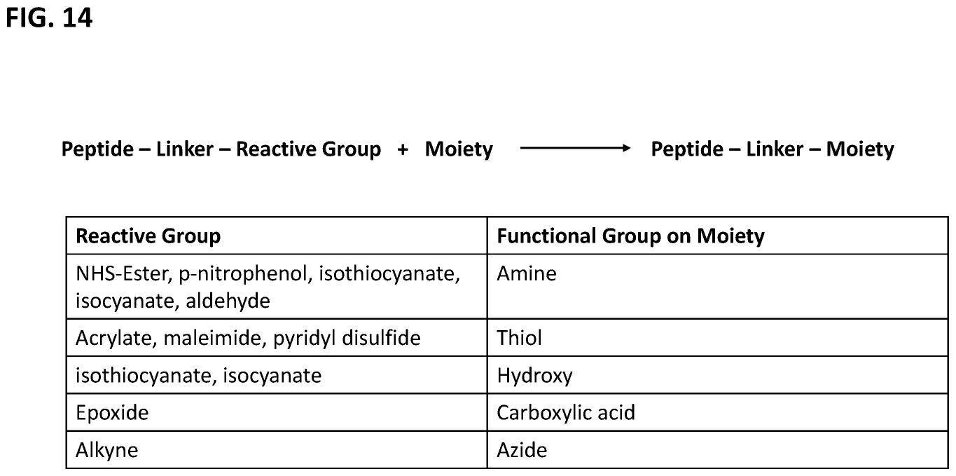

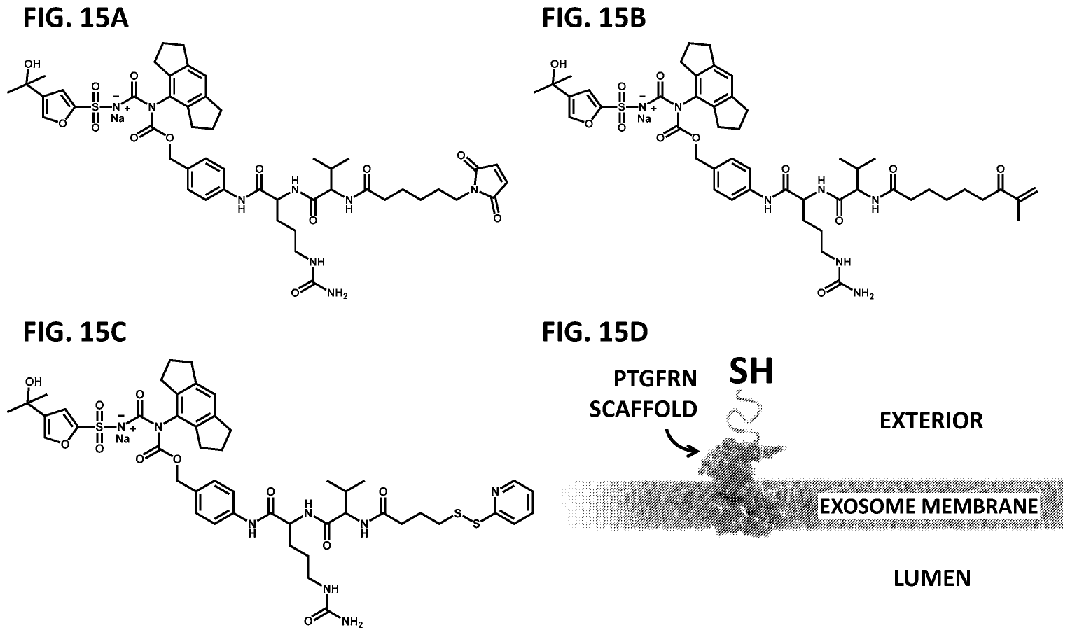

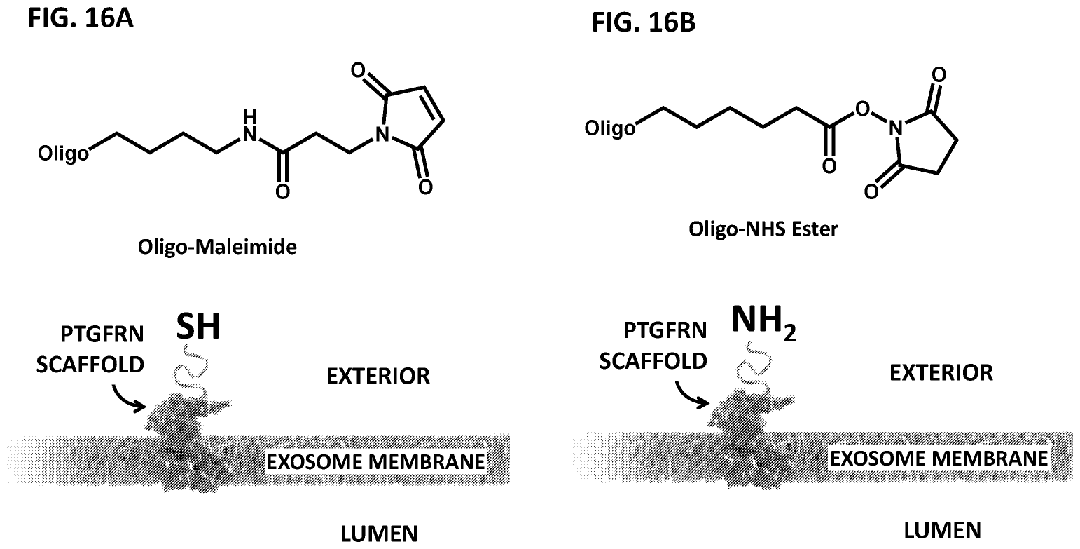

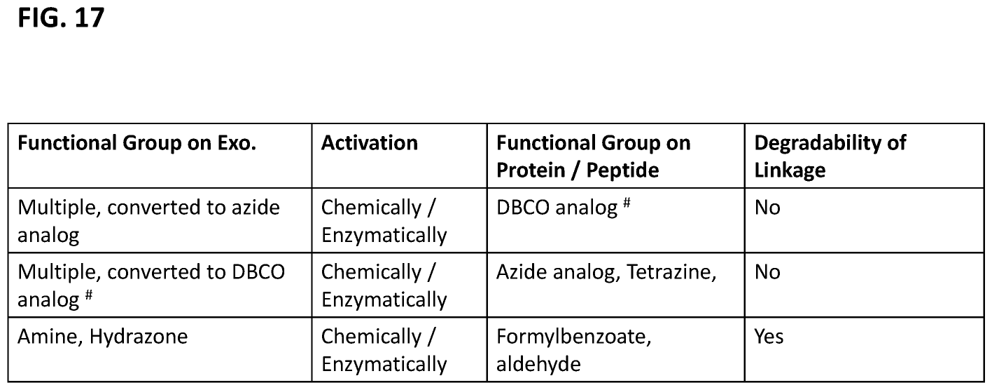

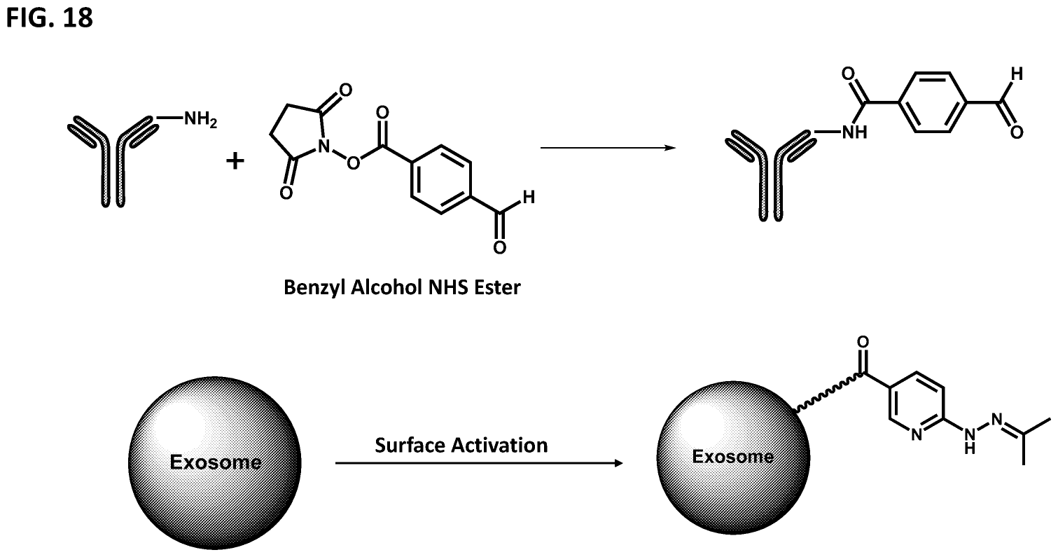

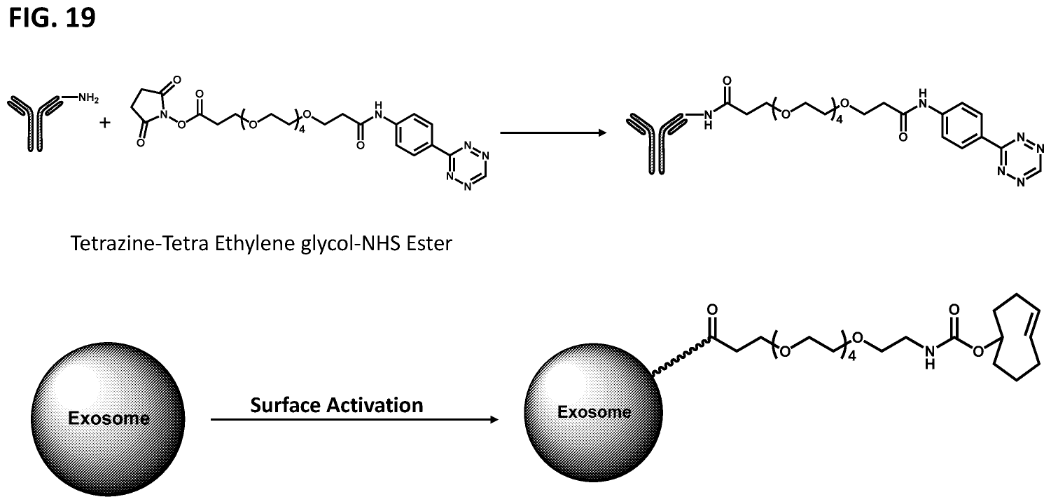

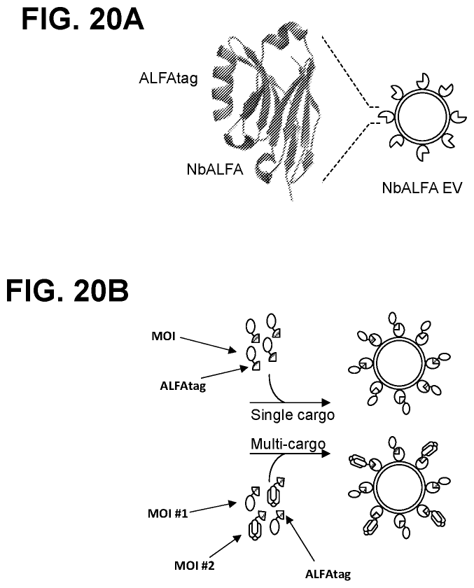

As shown in FIG. 11, similar results were observed with the earlier discussed acceptor domains (i.e., ALFANb, CfaC, and SpyCatcher).

Collectively, the above results confirm that the EVs (e.g., exosomes) described herein could be a useful vaccine platform for rapidly treating a wide range of diseases and disorders.

### Example 6: Analysis of the Loading of Multiple Payloads in EVs

Next, the ability of the EVs described herein to comprise multiple payloads (e.g., antigen, adjuvant, immunomodulatory, and/or targeting moiety) was assessed. Briefly, NANOLUC™ luciferase (Nluc) fused to the ALFAtag peptide (10 μg) (Nluc-ALFAtag) and molar equivalent of mouse IL-12 fused to ALFAtag peptide (mIL12-ALFAtag) were mixed with the following EVs individually or simultaneously: (1) native EVs; or (2) engineered-EVs overexpressing ALFA-specific nanobody (NbALFA) fused to PTGFRN (NbALFA-EVs). The mixture was incubated for 30 minutes at room temperature. Then, unbound Nluc-ALFAtag and/or mIL12-ALFAtag was removed by ultracentrifugation (20 minutes at 100,000×g). The EV pellets were resuspended in PBS and analyzed by SDS-PAGE and Western blot.

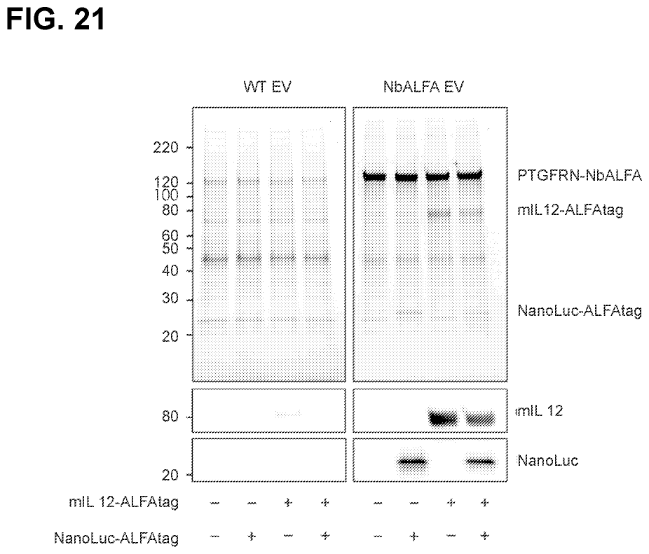

As shown in FIG. 21, in native EVs, no meaningful loading was observed for either Nluc-ALFAtag or mIL12-ALFAtag. However, in the NbALFA-EVs, significant loading of Nluc-ALFAtag and mIL12-ALFAtag was observed, as measured by both Western blot and SDS-PAGE. Similar results were observed whether Nluc-ALFAtag and mIL12-ALFAtag were loaded individually or simultaneously.

The above results further demonstrate that the EVs of the present disclosure can be readily modified to simultaneously comprise multiple payloads. As described herein, such EVs can be useful in treating various diseases and disorders, such as those disclosed herein.

### Example 7: Construction of EVs Comprising Coronavirus Antigen

Prior to the use of the plug-and-play system described herein to engineer EVs comprising a coronavirus antigen, EVs comprising the RBD of a coronavirus spike protein fused to the N-terminus of PTGFRN on the exterior surface of the EV was first constructed (“exoRBD”). Then, the immunogenicity of the engineered EVs was assessed in vivo. Briefly, wild-type C57BL/6 mice were immunized subcutaneously with one of the following: (i) exoRBD alone (at three different doses: 8 μg, 2.6 μg, and 1.3 μg); (ii) recombinant RBD (“rRBD”) (at two different doses: 8 μg and 2.6 μg); and (iii) control EVs (i.e., comprising PTGFRN but not RBD) (“exosomes”). Each of the animals received three total doses (two weeks apart between doses). Sera was collected at various time points, and both the total anti-RBD IgG antibody levels and neutralization activity were assessed. Anti-RBD antibody titers were measured using ELISA. Neutralization antibody activity was measured using a competitive ELISA assay, which was designed to measure sera antibody inhibition of recombinant RBD binding to recombinant human ACE2 protein. Tan et al., Nat Biotechnol 38(9): 1073-1078 (September 2020).

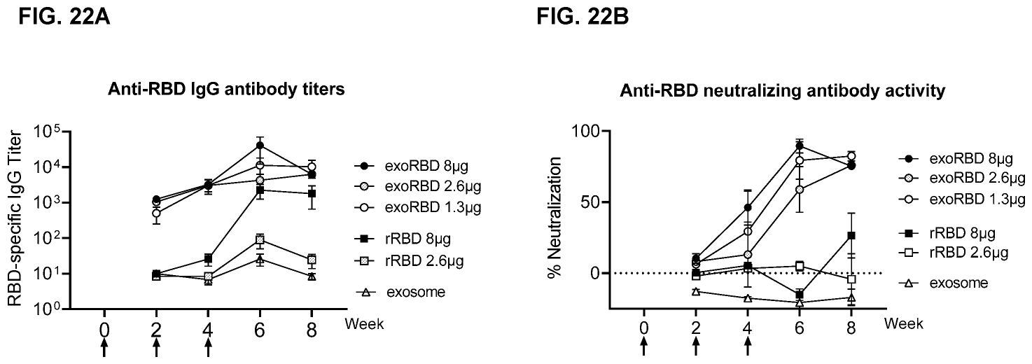

As shown in FIG. 22A, animals that were immunized with exoRBD (even at the lower dose of 1.3 μg) had significant greater amounts of anti-RBD IgG antibodies in the sera compared to animals immunized with rRBD or the control EVs. Similar increase in neutralization activity was also assessed (see FIG. 22B).

Next, to assess whether the addition of an adjuvant can further enhance the above-described therapeutic effect, some of the exoRBD were further loaded with a STING agonist in the lumen of the EV (“exoRBD+STING”). Then, wild-type C57BL/6 mice were immunized subcutaneously with either (i) exoRBD+STING or (ii) exoRBD alone. The animals received two total doses (2.6 μg/dose, two weeks part between doses). And, again, sera was collected, and both the total anti-RBD IgG antibody levels and neutralization activity were assessed.

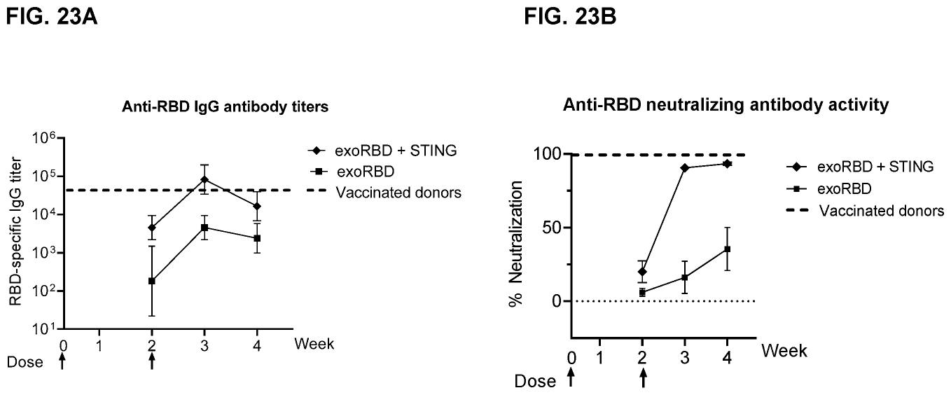

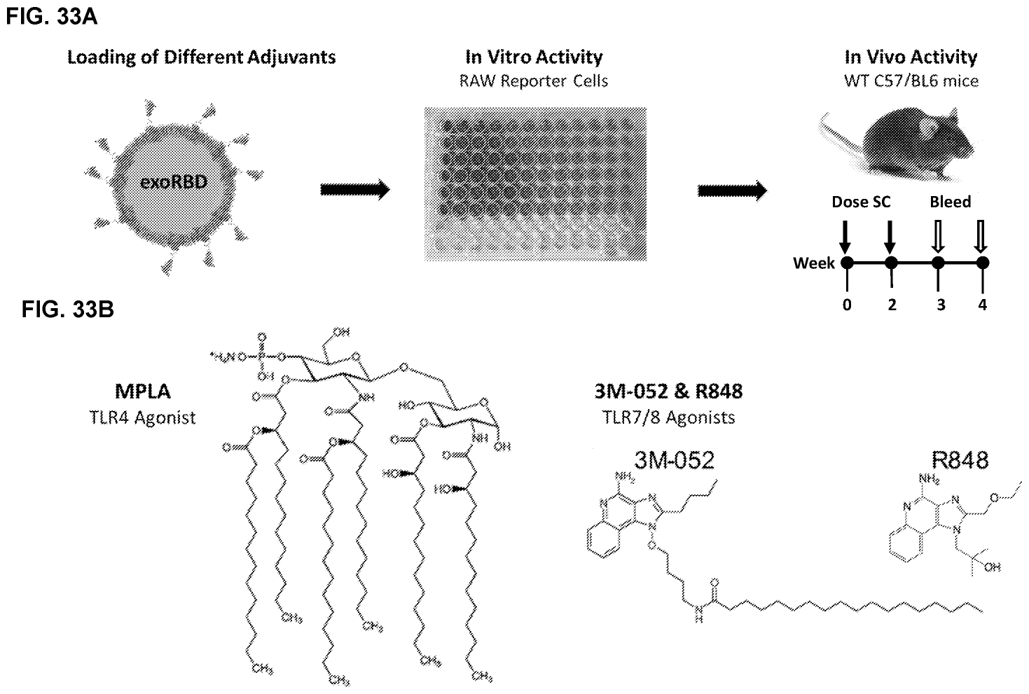

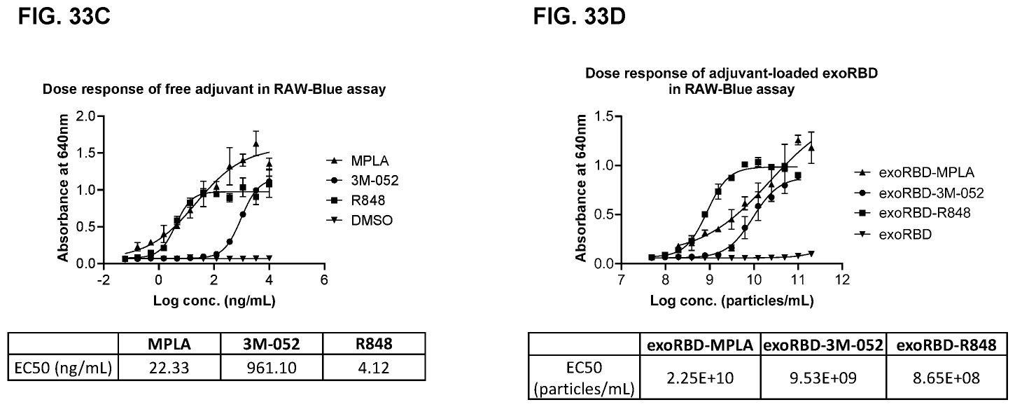

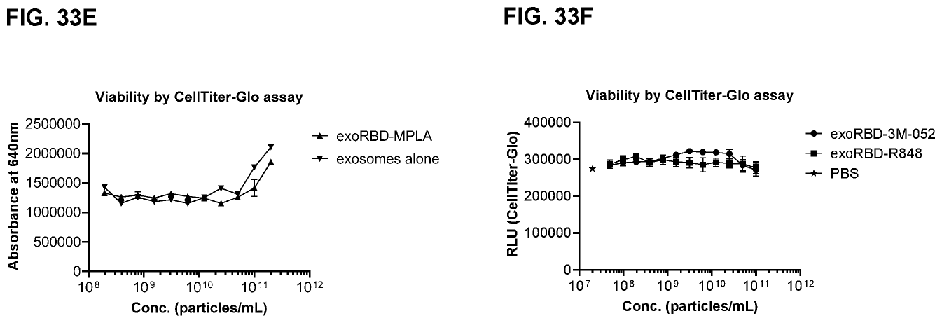

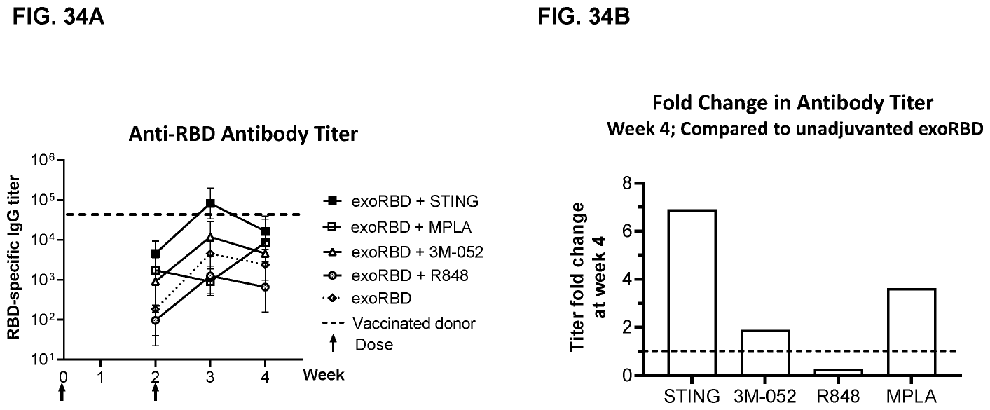

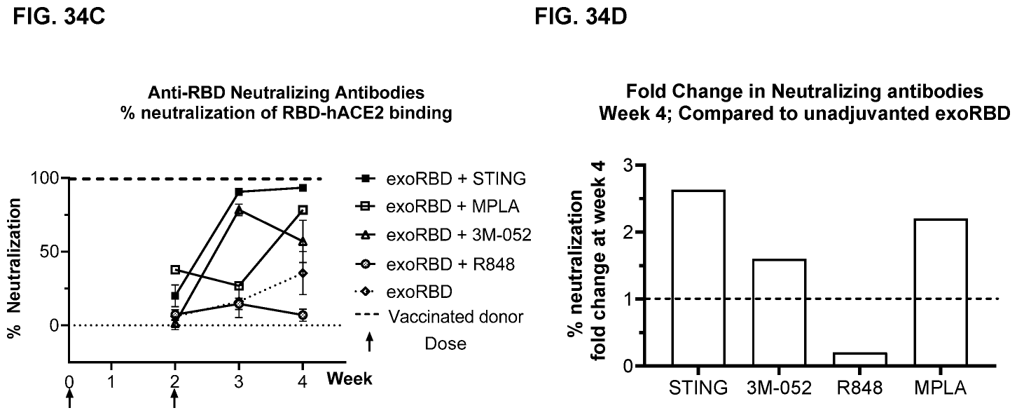

As shown in FIGS. 23A and 23B, the loading of the STING agonist significantly increased both the total anti-RBD IgG antibody levels and neutralization activity. The total antibody levels and neutralization activity in animals immunized with exoRBD+STING were comparable to corresponding levels observed in the sera from human subjects who received two doses of a COVID19 mRNA vaccine (see dark dashed lines in FIGS. 23A and 23B). Similar results were observed with other adjuvants. For instance, as shown in FIGS. 33A-33F and 34A-34D, exoRBD loaded with MPLA (TLR4 agonist), 3M-052 (TLR7/8 agonist), or R848 (TLR7/8 agonist) also induced significantly higher anti-RBD immune responses compared to exoRBD treatment alone.

Next, to further compare the anti-RBD IgG antibody activity observed in the above mice to human subjects, wild-type C57BL/6 mice were again immunized subcutaneously with one of the following: (i) PBS alone (“1-PBS”); (ii) a mixture of recombinant RBD (2.6 μg), soluble STING agonist (i.e., not loaded in an EV), and control EV (comprising PTGFRN alone; “PrX-exo”) (“rRBD+STING+PrX-exo”); and (iii) exoRBD (2.6 μg) loaded with STING agonist in the lumen of the EV (“exoRBD+STING”). The animals received two total doses (two weeks apart between doses). Then, sera were collected from the immunized animals, and neutralizing antibody activity was compared to sera from human subjects vaccinated twice with a COVID19 mRNA vaccine. The ability of the anti-RBD IgG antibodies from immunized mice and vaccinated human subjects to bind different recombinant RBD variants of concern (VoC) was also assessed using ELISA.

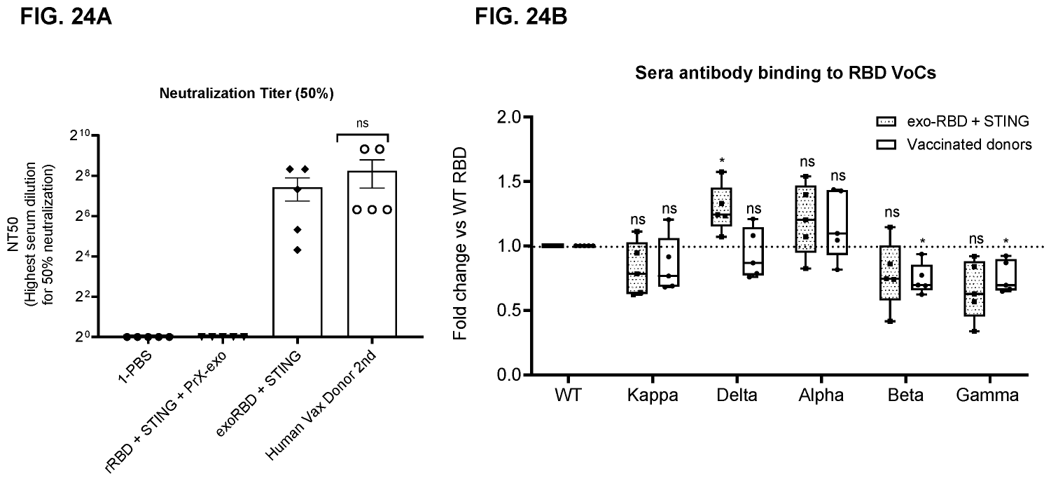

As shown in FIG. 24A and in agreement with the earlier data, no significant difference was observed in neutralization activity of anti-RBD IgG antibodies from mice immunized with exoRBD+STING and the vaccinated human subjects. In contrast, animals from group (ii) (i.e., rRBD+STING+PrX-exo) failed to elicit detectable neutralizing antibodies, suggesting that the immunogenicity of exoRBD requires the attachment of the RBD antigen to the EVs. In addition, the anti-RBD IgG antibodies from mice immunized with exoRBD+STING were also able to bind to all RBD VoCs as efficiently as the anti-RBD IgG antibodies from the vaccinated human subjects.

Next, to assess whether the above engineered EVs comprising the coronavirus RBD antigen have any effect on antibody isotype switching and germinal center formation, wild-type C57BL/6 mice were immunized subcutaneously with one of the following: (i) PBS alone (“PBS”), (ii) recombinant RBD alone (“rRBD”), (iii) recombinant RBD in combination with soluble STING agonist (“rRBD+STING”), (iv) EVs comprising the RBD region of coronavirus spike protein (fused to the N-terminus of PTGFRN) (“exoRBD”), and (v) exoRBD loaded with a STING agonist in the lumen of the EV (“exoRBD+STING”). Again, the animals received two total doses (two weeks apart between doses). Then, sera were collected from the animals at various time points, and the presence of different anti-RBD IgG isotypes was assessed using ELISA. Draining lymph nodes (cervical lymph nodes) were also collected after the animals were sacrificed, and flow cytometry was used to assess both germinal center B cell formation (defined as CD38⁻GL-7⁺) and plasma cell formation (defined as CD38⁺IgD⁻).

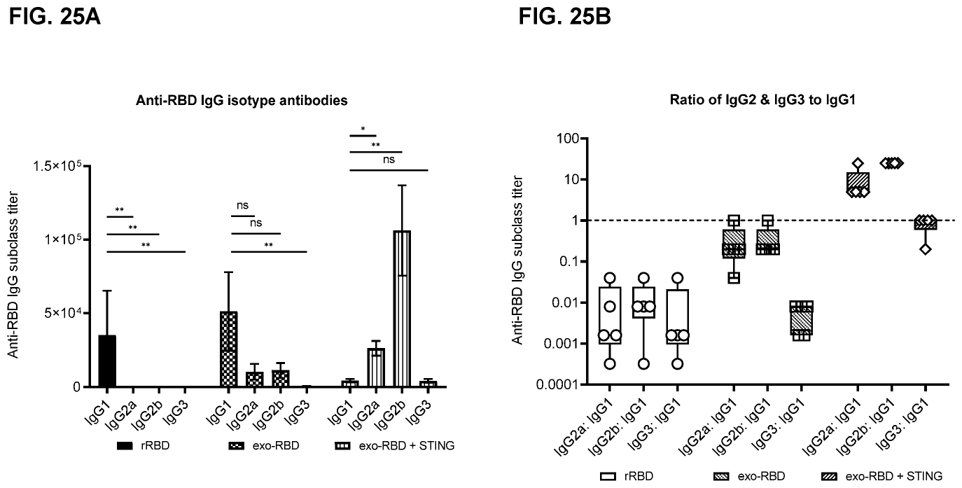

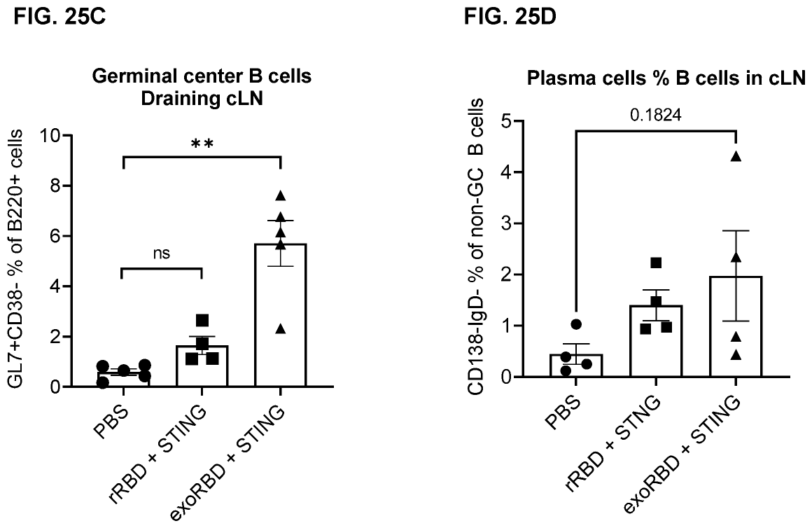

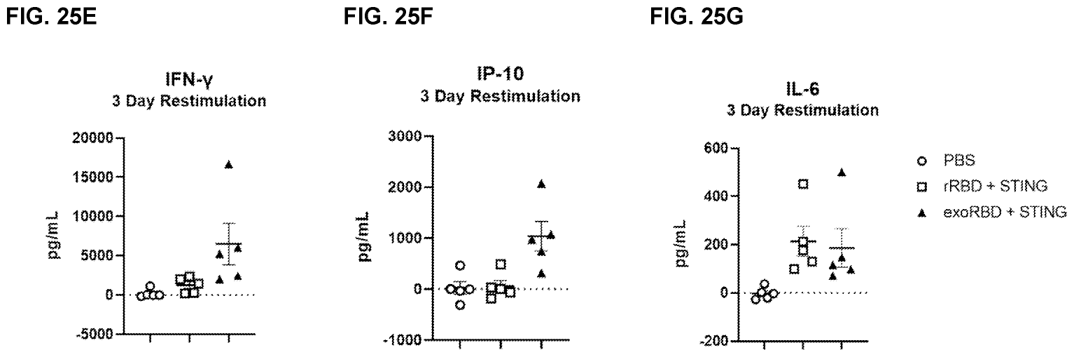

As shown in FIGS. 25A and 25B, in the animals that were immunized with exoRBD+STING, there was a shift towards a more Th1-like antibody response (as evidenced by the increase in IgG2a and IgG2b, and a decrease in IgG1). In contrast, in animals immunized with rRBD, the observed antibody response was largely Th2-like (predominantly IgG1 antibody only). Compared to the other treatment groups, animals immunized with exoRBD+STING also had significantly enhanced germinal center B cell formation (see FIG. 25C) as well as an increased trend in plasma cell formation (see FIG. 25D).

Lastly, to assess any effect on T cell immune response, wild-type C57BL/6 mice were immunized subcutaneously with either (i) recombinant RBD in combination with soluble STING agonist (“rRBD+STING”) or (ii) exoRBD loaded with a STING agonist in the lumen of the EV (“exoRBD+STING”). Control animals received PBS alone. As in the studies described above, the animals received total of two doses (two weeks apart between doses). Two weeks after the 2^{nd} dose, animals were sacrificed and the spleen harvested. Then, the splenic T cells from the animals were restimulated in vitro with overlapping RBD peptides for 3 days, and IFN-γ, IP-10, and IL-6 production were assessed using AlphaLISA.

As shown in FIGS. 25E-25G, upon antigen stimulation, T cells from animals that were immunized with exoRBD+STING produced robust amounts of all three cytokines (IFN-γ, IP-10, and IL-6) compared to the PBS control group. In contrast, T cells from animals that were immunized with rRBD+STING only produced IL-6 upon antigen stimulation.

Collectively, the above results demonstrate that the exoRBD (i.e., EV modified to comprise the RBD of a coronavirus spike protein fused to the N-terminus of PTGFRN on the exterior surface of the EV) is highly immunogenic (e.g., can induce strong humoral and cellular immune response). And, when loaded with a STING agonist, the exoRBD can induce anti-RBD antibody responses that are comparable to that observed in human subjects vaccinated with two doses of a COVID19 mRNA.

### Example 8: Construction of EVs Comprising RBD Using the ALFA Plug-and-Play System

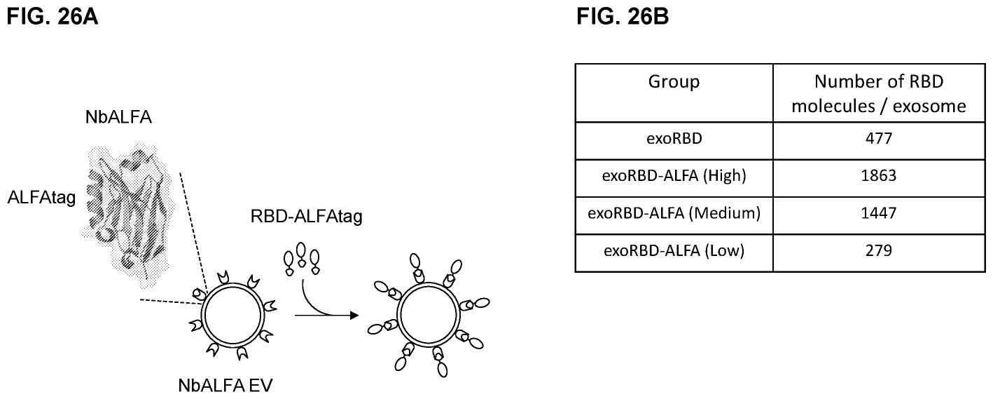

As further demonstration that the EVs described herein can be used to treat a coronavirus, the ALFA plug-and-play system described herein was used to modify the EVs to comprise the RBD protein of a coronavirus spike protein. Briefly, EVs were modified to comprise ALFA nanobody (NbALFA) acceptors on the exterior surface of the EVs (fused to the N-terminus of PTGFRN). The RBD was expressed and purified using conventional transient transfection and column chromatography, respectively, and were fused to the ALFAtag. FIG. 26A. ALFA-tagged RBD (at three different concentrations: high (8.73 ug RBD/1e11 exosomes), medium (6.8 ug RBD/1e11 exosomes), and low (1.3 ug RBD/1e11 exosomes) was conjugated to the NbALFA acceptors on the EVs by mixing at room temperature for 30 minutes. Unbound ALFA-tagged RBD was removed by ultracentrifugation or size exclusion chromatography, resulting in stable association of the ALFA-tagged RBD on the surface of NbALFA comprising EVs (“exoRBD-ALFA”). To quantify the surface loading of the ALFA-tagged RBD molecules, an ELISA specific for SARS CoV-2 RBD was used.

As shown in FIG. 26B, compared to exoRBD (described in Example 7), at both the high and medium concentrations, there was significantly greater number of RBD molecules associated with the exterior surface of the EVs using the ALFA-tag system. Additionally, the ALFA tag system enabled a titratable, dose dependent surface loading onto EVs.

Next, to assess the immunogenicity of the above described exoRBD-ALFA, wild-type C57BL/6 mice were immunized subcutaneously with one of the following: (i) PBS alone, (ii) exoRBD-ALFA (surface loaded with one of the following concentrations of the ALFA-tagged RBD: 24.1 μg, 10.9 μg, and 1.9 μg), (iii) recombinant RBD alone (“rRBD”) (at two different doses: 8 μg and 2.6 μg), (iv) rRBD in combination with soluble STING agonist (“rRBD+STING”), and (v) control EVs (i.e., comprising PTGFRN but not RBD) (“exosomes”). The animals received three total doses (two weeks apart between doses). Sera were collected from the animals at various time points post immunization, and both the total anti-RBD IgG antibody levels and neutralization activity were assessed (as described in Example 7).

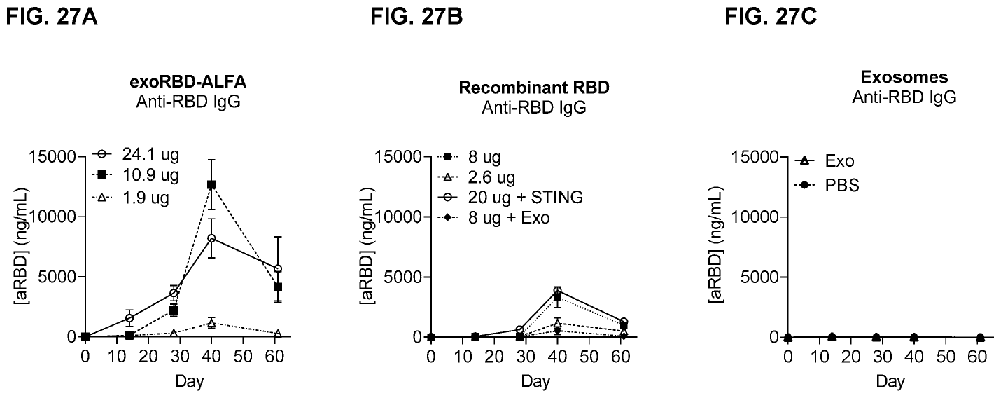

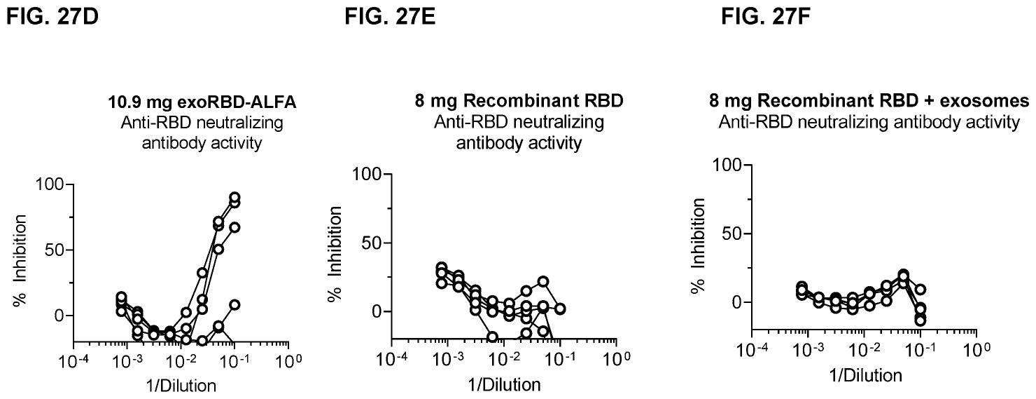

As shown in FIGS. 27A, 27B, and 27C, compared to the other treatment groups (including the group in which the animals were immunized with both rRBD and a STING agonist as an adjuvant), animals immunized with exoRBD-ALFA (at both 24.1 μg and 10.9 μg) had the greatest anti-RBD IgG antibody levels at the time points assessed. Similar results were also observed for neutralization activity (see FIGS. 27D, 27E, and 27F).

The above results demonstrate the therapeutic capability of the exoRBD-ALFA in treating a coronavirus infection, such as COVID-19. The results also further confirm the importance of the RBD antigen being attached to the exterior surface of the EVs to elicit an immune response.

### Example 9: In Vitro Analysis of EVs Comprising ALFA-Tagged CD40L

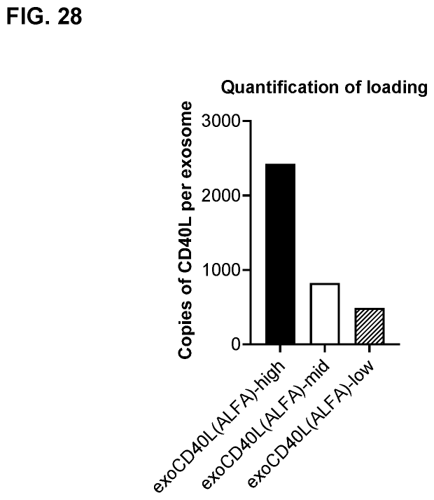

To further assess the capability of the plug-and-play systems described herein, EVs modified to comprise ALFA nanobody (NbALFA) acceptors on the exterior surface (see Example 8) were mixed with three different concentrations (high, medium, or low) of ALFA-tagged CD40L protein at room temperature for 30 minutes. Any unbound ALFA-tagged CD40L was removed by ultracentrifugation or size exclusion chromatography. The resulting EVs comprising ALFA-tagged CD40L conjugated to the NbALFA on the exterior surface of the EV (“exoCD40L(ALFA)”) were assessed using an ELISA assay and the copies of the trimeric CD40L protein per EV was quantified. As shown in FIG. 28 and similar to the results described in Example 8, the ALFA-tag system described herein allowed for the successful loading (in a dose dependent manner) of the CD40L protein on the exterior surface of the EVs.

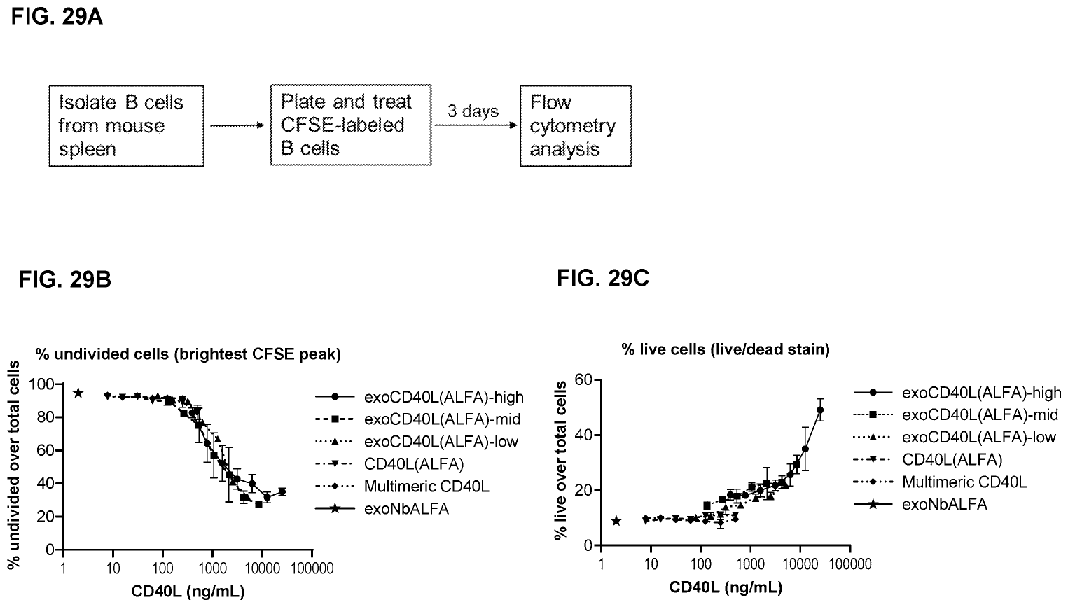

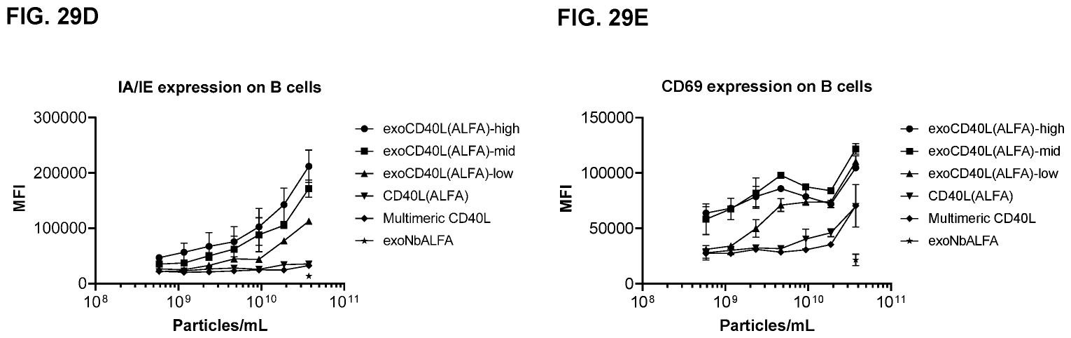

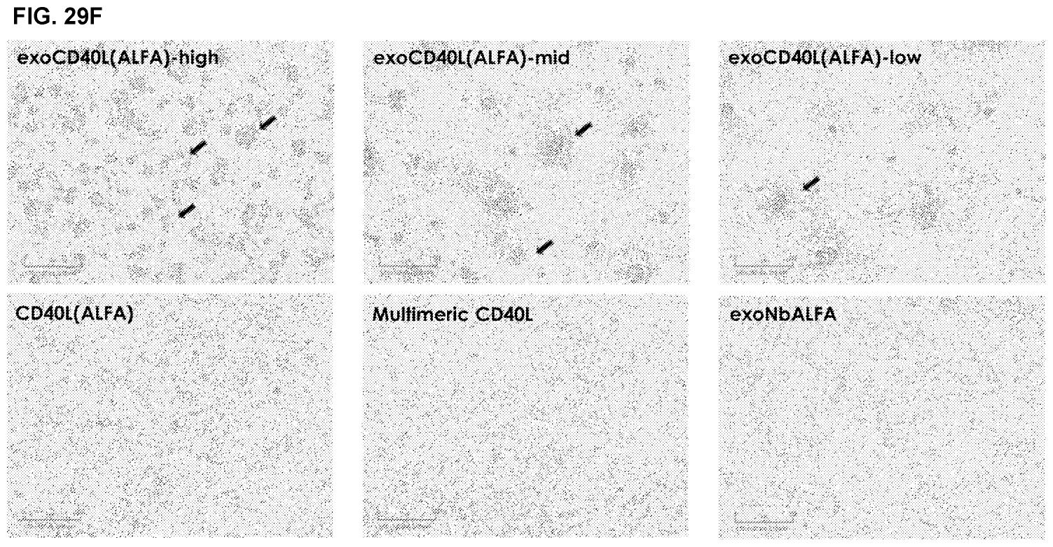

To begin assessing the activity of the exoCD40L(ALFA) described above, mouse splenic B cells were isolated using magnetic beads (negative selection) and labeled with CFSE (FIG. 29A). Then, the cells were plated in 96-well plates and treated with one of the following: (i) exoCD40L(ALFA)-high (i.e., loaded with high concentration of ALFA-tagged CD40L), (ii) exoCD40L(ALFA)-mid (i.e., loaded with medium concentration of ALFA-tagged CD40L), (iii) exoCD40L(ALFA)-low (i.e., loaded with low concentration of ALFA-tagged CD40L), (iv) soluble ALFA-tagged CD40L protein (i.e., not fused to the NbALFA on the exterior surface of the EVs), (v) a multimeric CD40L protein alone, and (vi) EVs modified to comprise NbALFA but not fused to the ALFA-tagged CD40L protein. Approximately three days later, cells were harvested and analyzed by flow cytometry.

As shown in FIGS. 29B and 29C, there was a comparable dose-dependent increase in both proliferation and survival of cells from all the treatment groups. However, only exoCD40L(ALFA) (at all three concentrations) was able to induce the activation of the B cells, as evidenced by the dose-dependent increase in the expression of both MHC class II (FIG. 29D) and CD69 (FIG. 29E). Additionally, B cell clusters (indicative of proliferation and activation) were only observed (with IncuCyte Zoon) when the cells were treated with exoCD40L(ALFA) (at all three concentrations).

The above results demonstrate that the plug-and-play system described herein (e.g., ALFA-tag) are effective in loading a multimeric CD40L protein on the surface of EVs. The results further demonstrate that such EVs are functionally active and can induce the activation of B cells in vitro.

### Example 10: In Vivo Analysis of EVs Comprising ALFA-Tagged CD40L

To assess whether the exoCD40L(ALFA) described in Example 9 are also functional in vivo, wild-type mice received an intraperitoneal administration of one of the following: (i) EVs modified to comprise NbALFA but not fused to the ALFA-tagged CD40L protein (“exo-Nb ALFA”); (ii) EVs modified to comprise NbALFA and loaded with ALFA-tagged CD40L protein (“exo-Nb ALFA+mCD40L(ALFA)”); (iii) soluble ALFA-tagged CD40L protein (i.e., not fused to the NbALFA on the exterior surface of the EVs) (“mCD40L(ALFA)”); (iv) agonistic anti-CD40 antibody (100 μg); and (v) PBS alone. Then, three days later, the animals were sacrificed and splenocytes were isolated and assessed using flow cytometry. Peritoneal lavage was also performed, and the collected cells were also analyzed using flow cytometry.

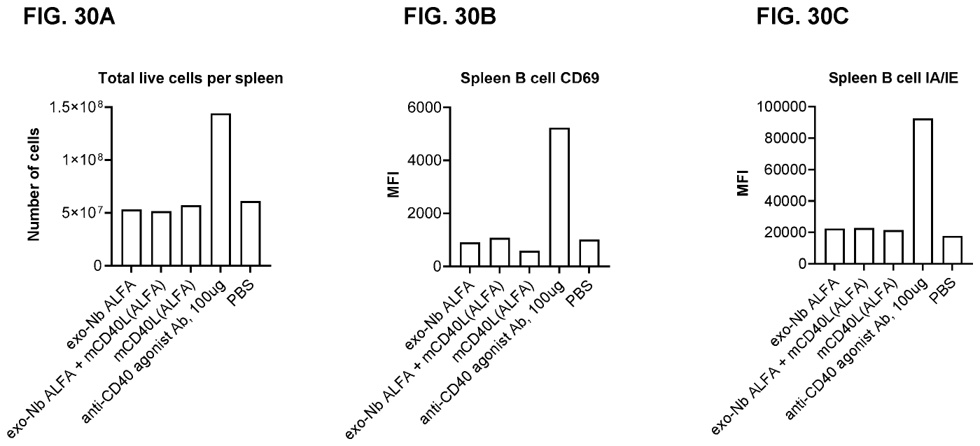

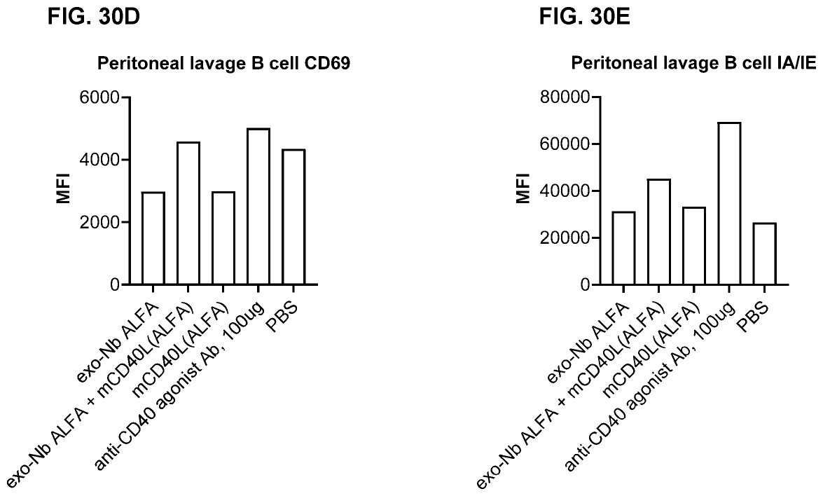

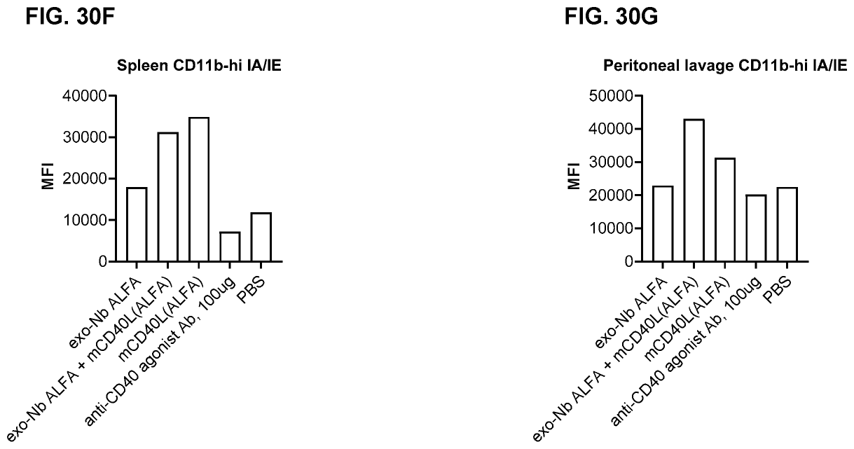

As shown in FIG. 30A, in animals treated with the agonistic anti-CD40 antibody, splenomegaly had occurred, as evidenced by the high total live cell count in the spleen. There was no evidence of splenomegaly in the animals from the other treatment groups. Similarly, in animals treated with the agonistic anti-CD40 antibody, there was increased expression of CD69 (FIGS. 30B and 30D) and MHC class II (FIGS. 30C and 30E) in B cells from both the spleen and peritoneal lavage. In contrast, an increased expression of the activation markers was only observed locally in the peritoneal lavage in mice treated with exoCD40L(ALFA).

To further characterize the above difference between exoCD40L(ALFA) and agonistic anti-CD40L antibody treatments, myeloid cells (CD45⁺ CD19⁻ CD11b^{hi}) from both the spleen and peritoneal lavage were assessed for the animals of the different treatment groups. In mice treated with the agonistic anti-CD40L antibody, activation of the myeloid cells (as evidenced by MHC class II expression) was only observed in cells from the peritoneal lavage (see FIGS. 30F and 30G). In contrast, comparable activation of myeloid cells from both the spleen and peritoneal lavage was observed in the animals from the other treatment groups.

The above results further demonstrate the ability of the exoCD40L(ALFA) to activate B cells in a site-specific manner, suggesting that at least compared to treatments such as agonistic anti-CD40L antibodies, the exoCD40L(ALFA) could be associated with less systemic side effects.

### Example 11: Analysis of EVs Comprising ALFA-Tagged IL-12

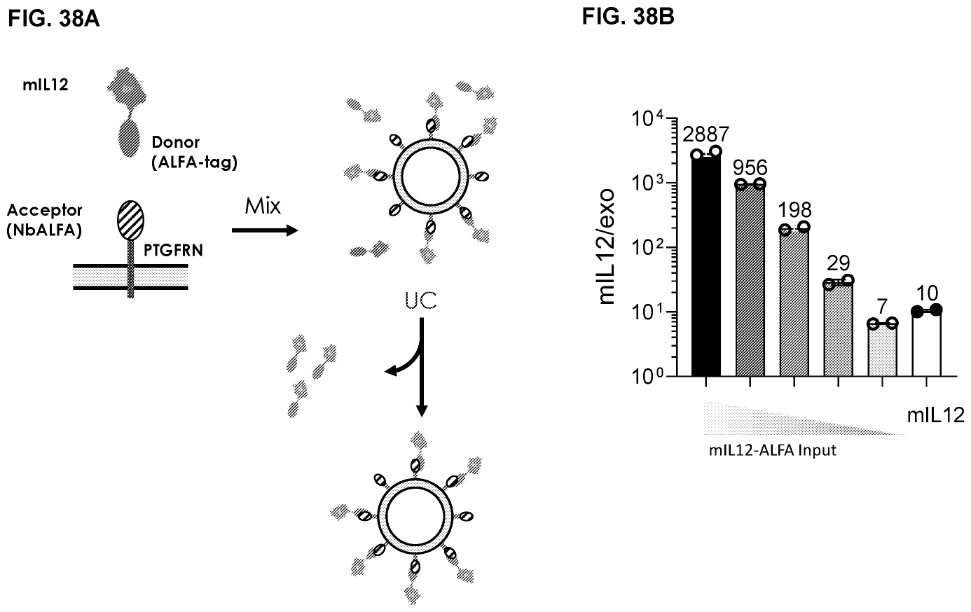

To further assess the different types of moieties that can be attached to EVs using the plug-and-play system described herein, EVs modified to comprise ALFA nanobody (NbALFA) acceptors on the exterior surface (see Example 8) were mixed with ALFA-tagged IL-12 protein at room temperature for 30 minutes. The ALFA-tagged IL-12 protein were added to the mixture at five different concentrations: (1) 34 μg, (2) 11 μg, (3) 2.3 μg, (4) 0.33 μg, and (5) 0.08 μg. Any unbound ALFA-tagged IL-12 was removed by ultracentrifugation (FIG. 38A). The resulting EVs comprising ALFA-tagged IL-12 conjugated to the NbALFA on the exterior surface of the EV (also referred to herein as “NbALFA-mIL-12 exo” or “NbALFA exo+mIL12-ALFA”) were assessed using an ELISA to quantify the number of IL-12 molecules displayed on the surface of the EVs.

As shown in FIG. 38B, the ALFA-tagged IL-12 proteins were successfully loaded onto the surface of the EVs in a dose-dependent manner. Compared to the control (i.e., IL-12 protein genetically fused to PTGFRN on the exterior surface of the EVs), approximately 290-fold more IL-12 protein was able to be loaded onto the surface of the EVs using the ALFA-tag system described herein.

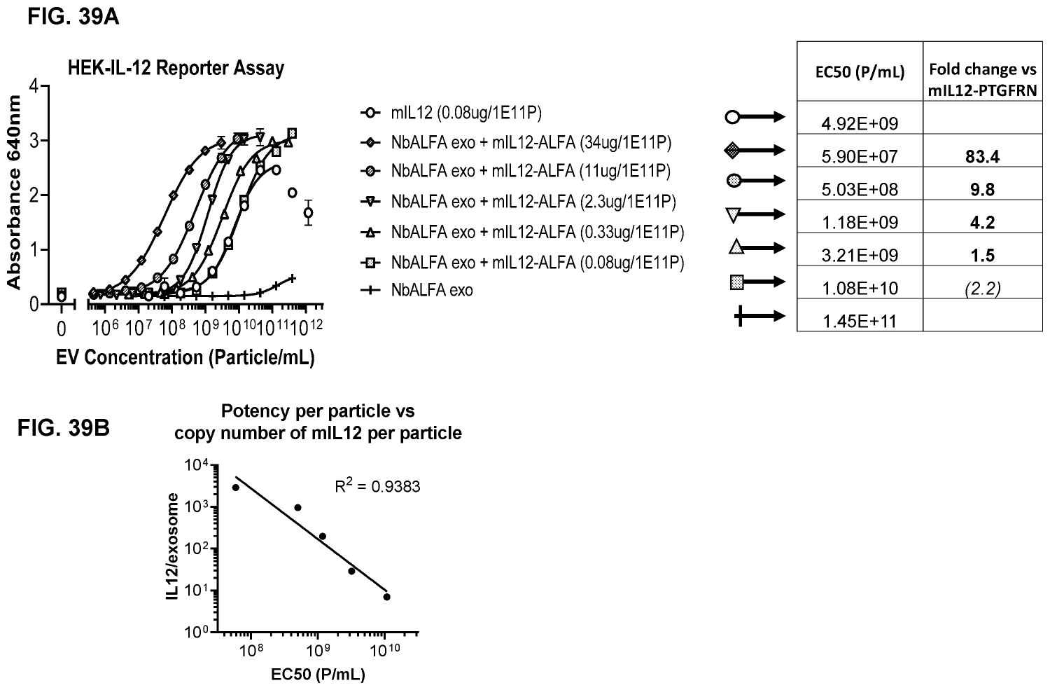

Next, the potency of the above EVs loaded with different concentrations of the ALFA-tagged IL-12 was assessed by performing a 10-point dose titration in an IL-12 reporter cell line (InVivoGen) measuring STAT4-mediated transcriptional activity via secreted embryonic alkaline phosphatase (SEAP) expression measured at absorbance 640 nm. As shown in FIG. 39A, except for the EVs loaded with the lowest concentration of ALFA-tagged IL-12 (i.e., 0.08 μg), all the NbALFA-mIL-12 exo produced showed higher potency compared to the control EVs (i.e., comprising IL-12 genetically fused to PTGFRN). And, as shown in FIG. 39B, there was excellent correlation between potency per EVs and the number of IL-12 molecules attached to the surface of the EVs, suggesting a dose-dependent increase in potency.

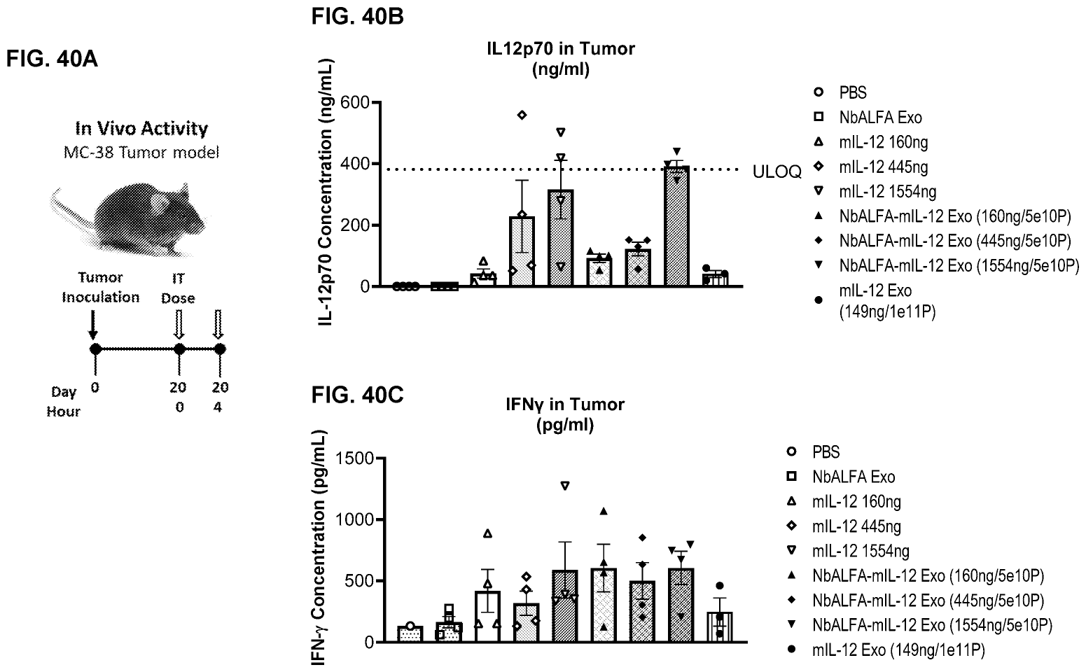

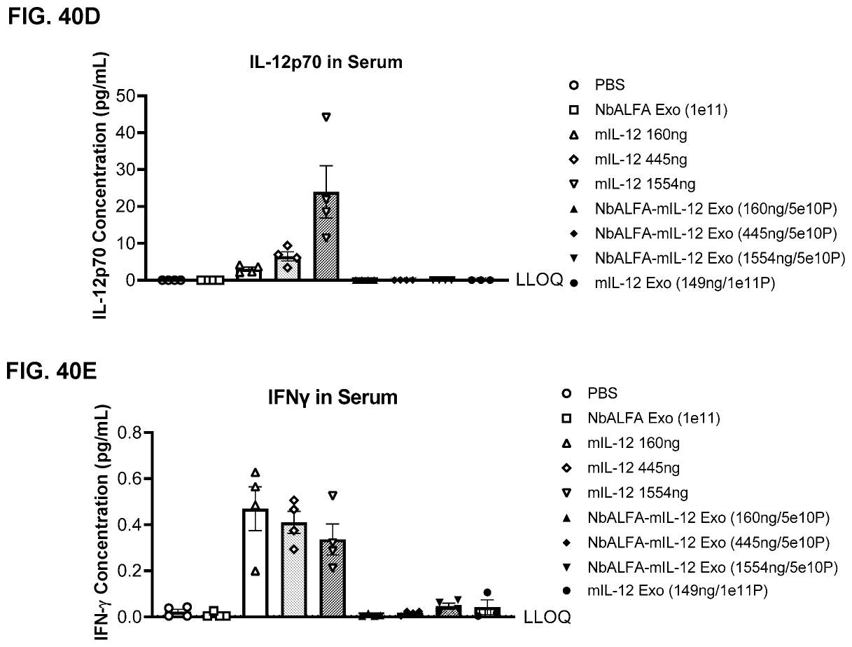

Next, to assess in vivo activity, a mouse MC-38 tumor model was used. FIG. 40A. Briefly, MC-38 tumor-bearing mice were immunized intratumorally with one of the following: (i) PBS only; (ii) recombinant IL-12 protein (at three different concentrations: 160 ng, 445 ng, or 1,554 ng) (“mIL-12”); (iii) NbALFA-mIL-12 exo (at three different concentrations: 160 ng/5×10¹⁰ particles, 445 ng/5×10¹⁰ particles, or 1,554 ng/5×10¹⁰ particles); (iv) control EV #1 (i.e., comprising NbALFA conjugated to PTGFRN but not IL-12) (“NbALFA Exo”); and (v) control EV #2 (i.e., comprising IL-12 genetically fused to the N-terminus of PTGFRN) (“mIL-12 Exo”). Then, four hours after administration, tumors were excised from the animals, and IL-12p70 and IFN-γ levels were quantified. Sera were also collected from the animals for IL-12p70 and IFN-γ quantification.

As shown in FIG. 40B, similar amounts of IL-12p70 were present in tumors injected with either NbALFA-mIL-12 exo or mIL-12 in a dose dependent manner, demonstrating that EVs loaded with mIL-12 ALFA tag protein are stable in vivo. Similarly, at the same dose, NbALFA-mIL-12 exo induced similar amounts of IFN-γ in the tumors compared to mIL-12. FIG. 40C. In contrast, in the tumors of animals treated with mIL-12 Exo, there was generally much reduced levels of both IL-12p70 and IFN-γ.

While cytokine levels were similar in the tumors, there was a stark difference when the cytokine levels were assessed in the serum. As shown in FIGS. 40D and 40E, in animals that received mIL-12 intratumorally, significant amounts of both IL-12p70 and IFN-γ were observed in the sera. In contrast, in animals that received NbALFA-mIL-12 exo, IL-12p70 and IFN-γ were only detectable at the site of injection (i.e., in the tumors and not in the serum).

The above results further confirm the therapeutic potential of the plug-and-play system described herein. More specifically, the above results suggests that ALFA-tagged mIL-12 can be loaded onto EVs in high copy number and is biologically active both in vitro and in vivo. The results also show that the ALFA-tagged mIL-12 EVs display retained pharmacology at the site of administration and do not distribute systemically when administered locally.

### Example 12: Analysis of EVs Comprising Both ALFA-Tagged CD40L and ALFA-Tagged RBD

As demonstrated at least in Example 6, the plug-and-play systems described herein can be used to load multiple payloads onto the surface of EVs. To assess whether such EVs are also functional, EVs modified to comprise ALFA nanobody (NbALFA) acceptors on the exterior surface were mixed with both ALFA-tagged RBD and ALFA-tagged CD40L protein to produce EVs comprising both ALFA-tagged CD40L and ALFA-tagged RBD conjugated to NbALFA acceptors on the exterior surface of the EV (“exo-ALFA-RBD&CD40L dual”). Then, wild-type mice were immunized subcutaneously with one of the following: (i) exoRBD-ALFA; (ii) combination of exoRBD-ALFA and exoCD40L-ALFA; (iii) exo-ALFA-RBD&CD40L dual; (iv) exoCD40L-ALFA in combination with an agonistic anti-CD40 antibody (“exo-ALFA-RBD+anti-CD40 Ab”); and (v) PBS alone. The animals received two doses (two weeks between doses). At day 28 post initial immunization, sera were collected from the animals and both anti-RBD IgG titer and neutralizing activity (i.e., capable of blocking the interaction between RBD and hACE2 proteins). The spleen was also harvested for splenic germinal center formation.

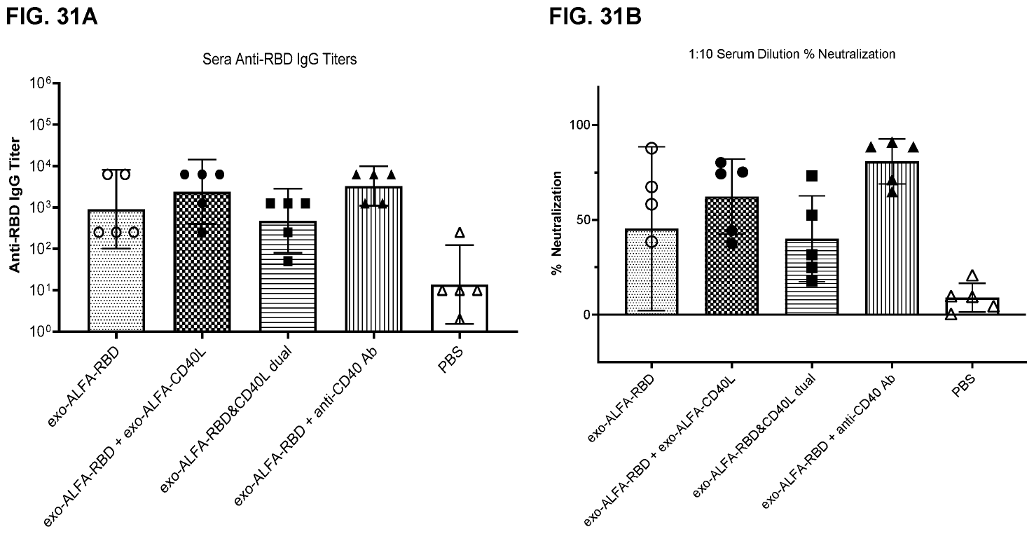

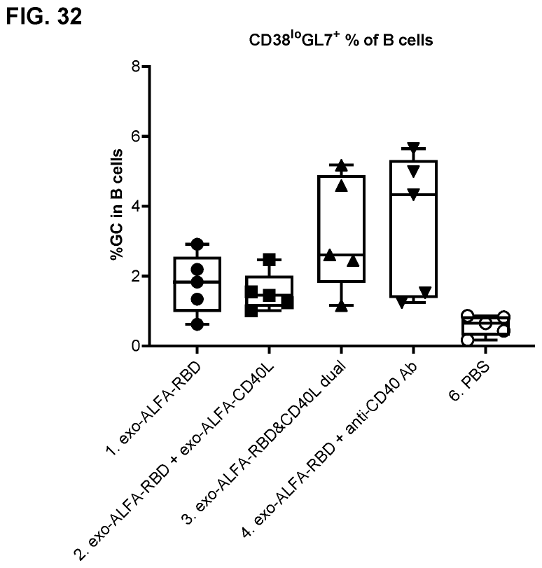

As shown in FIGS. 31A and 31B, except for animals treated with PBS alone, comparable anti-RBD IgG antibody levels and neutralization activity were observed in animals from the different treatment groups. Splenic germinal center formation was also observed in animals treated with EVs loaded with CD40L (see FIG. 32).

Collectively, the above results further confirm that the plug-and-play systems described herein can be useful in loading multiple payloads (e.g., RBD and CD40L) onto the surface of EVs and that each payload retains biological activity.

### Example 13: Analysis of the Effect of Different Dosing Regimen on EV-Mediated Immune Response

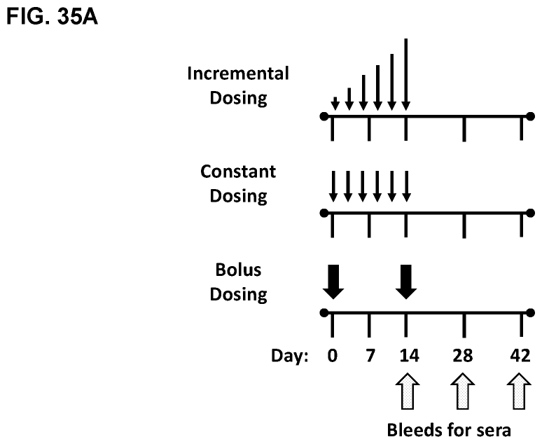

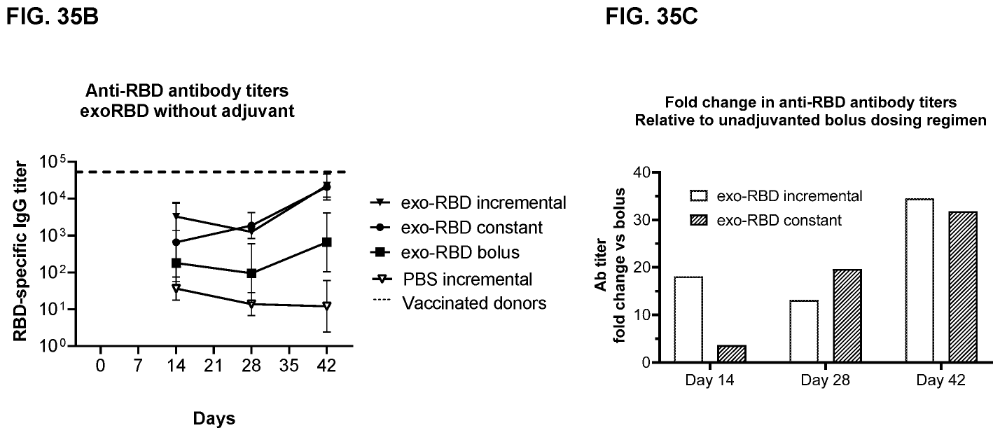

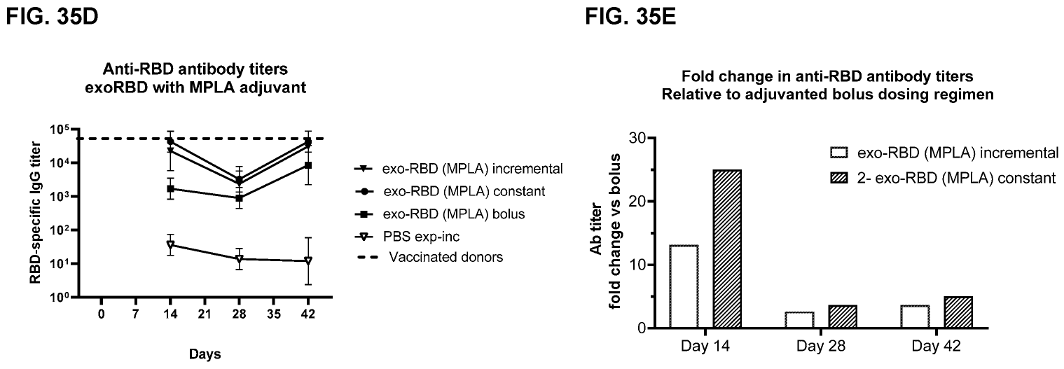

To assess whether above-described EV-mediated immune responses are affected by dosing regimen, wild-type C57BL/6 mice were immunized subcutaneously with either exoRBD or exoRBD-MPLA (i.e., exoRBD loaded with MPLA adjuvant) using one of the following dosing regimen (see FIG. 35A): (1) incremental dosing (cumulative dose administered was given as 6 doses with each dose being greater (double) than the previous dose); (2) constant dosing (cumulative dose administered was also given as 6 doses, but each dose was the same as the previous dose); and (3) bolus dosing (cumulative dose was given as 2 doses). Sera were collected from the animals at days 14, 28 and 42 post immunization, and analyzed for both anti-RBD IgG antibody titers and neutralization activity (see, e.g., Example 7). At day 42, the animals were also sacrificed and the draining lymph nodes (cervical lymph nodes) were harvested to assess germinal B cell formation.

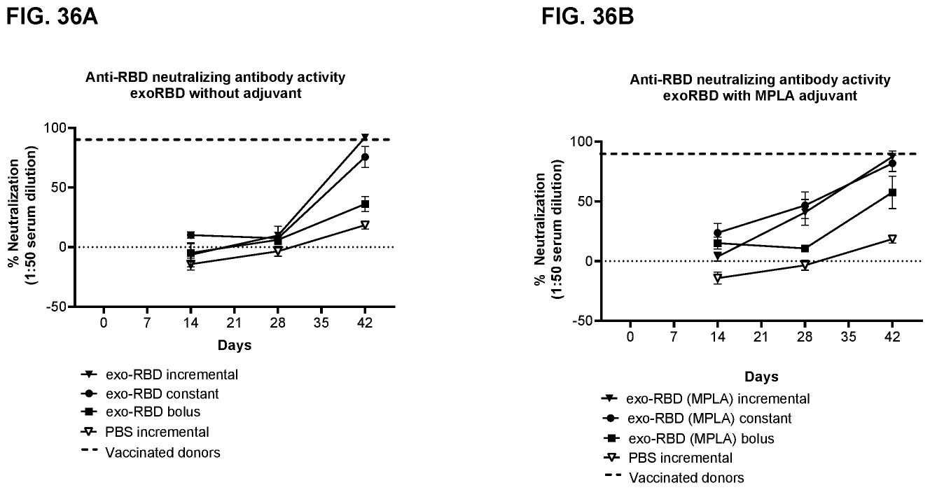

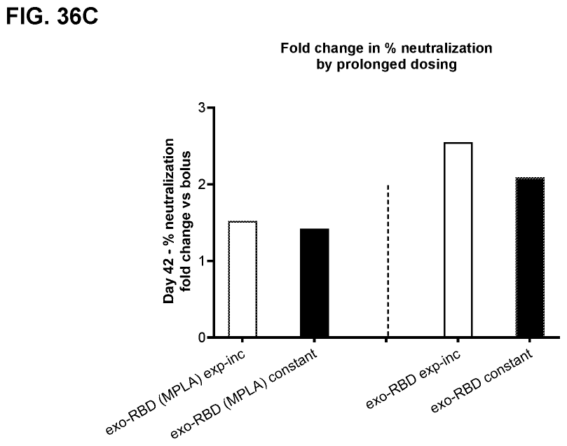

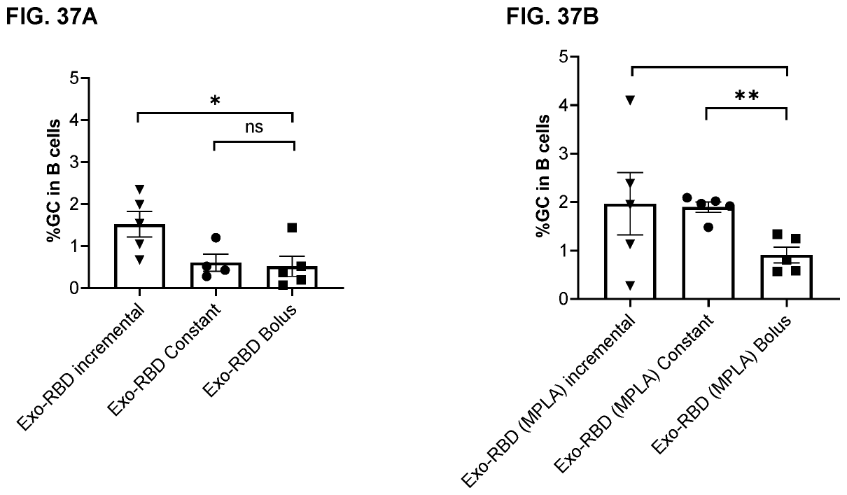

As shown in FIGS. 35B-35E, both incremental and constant dosing regimens significantly enhanced RBD immunogenicity compared to bolus dosing for both adjuvanted (exoRBD-MPLA) and unadjuvanted (exoRBD) EVs. The relative enhancement of RBD immunogenicity was greater in animals treated with exoRBD. Moreover, incremental and constant dosing regimens (for both exoRBD and exoRBD-MPLA) induced anti-RBD antibody titers that were similar to that observed in human subjects who had received 2 doses of a COVID19 mRNA vaccine (dashed lines). However, constant and incremental dosing of the MPLA adjuvanted vaccine accelerated the induction of anti-RBD titers, such that they approach mRNA vaccinated subjects 2 weeks after the first dose. Similar results were observed for neutralization activity (see FIGS. 36A-36C). As for germinal center (GC) B cell formation, incremental dosing significantly increased GC B cell formation when unadjuvanted exoRBD was dosed while constant dosing significantly increased GC B cell formation when exoRBD and MPLA was dosed.

The above results demonstrate that the dosing regimen can have an effect on EV-mediated immune responses. In particular, the results show that incremental and constant dosing regimens can enhance immune response, even in the absence of an adjuvant, compared to traditional bolus dosing regimens. Therefore, such dosing regimens have utility for enhancing exoVACC vaccine (e.g., constructed using the play-and-plug system described herein) efficacy especially in the absence of a co-loaded adjuvant.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific aspects have been illustrated and described, the above specification is not restrictive. It will be appreciated that various changes can be made without departing from the spirit and scope of the disclosure(s). Many variations will become apparent to those skilled in the art upon review of this specification.

1. A method of manufacturing a vaccine for a disease or disorder comprising, preparing an extracellular vesicle (EV) for the vaccine, comprising loading an antigen to the EV that has been isolated from a producer cell, wherein the disease or disorder comprises an infectious disease.

2-3. (canceled)

4. The method of claim 1, wherein the antigen is: (i) linked to the exterior surface of the EV, (ii) linked to the luminal surface of the EV, (iii) in the lumen of the EV, or (iv) any combination thereof, wherein after the loading of the antigen, the EV is capable of inducing a T-cell immune response, a B-cell immune response, a NK response, or a combination thereof, wherein the antigen comprises a single epitope of an antigen, or a concatemer of multiple epitopes of an antigen.

5. The method of claim 1, to wherein the EV further comprises an adjuvant, wherein the adjuvant comprises a non-toxic mutant of diphtheria toxin (e.g., CRM-197), peptide adjuvant (e.g., muramyl dipeptide (MDP)), STING agonist, TLR agonist, universal T cell helper peptide (e.g., PADRE), or any combinations thereof.

6-10. (canceled)

11. The method of claim 5, wherein the antigen and/or the adjuvant is linked to the exterior surface and/or the luminal surface of the EV by an anchoring moiety, affinity agent, chemical conjugation, cell penetrating peptide (CPP), split intein, SpyTag/SpyCatcher, ALFA-tag, Streptavidin/Avitag, Sortase, SNAP-tag, ProA/Fc-binding peptide, or any combinations thereof.

12. The method of claim 11, wherein the anchoring moiety comprises a sterol (e.g., cholesterol), GM1, lipid (e.g., fatty acid (e.g., palmitate), ionizable lipid, glycerophospholipid, sphingolipid), vitamin (e.g., tocopherol (e.g., vitamin E), vitamin A, vitamin D, vitamin K), alkyl chain, aromatic ring, small molecule, peptide, including any derivatives thereof, or any combination thereof, wherein the anchoring moiety is attached to the antigen and/or adjuvant by a linker, wherein the linker comprises:
(i) a polypeptide linker, non-polypeptide linker, or both;
(ii) a hydrophilic linker (e.g., PEG and derivatives);
(iii) a hydrophobic linker (e.g., alkyl chain, hexamethylene);
(iv) a cleavable linker (e.g., phosphodiesters, valine citrulline, disulfide bond, acid-labile); or
(v) any combination of (i) to (iv).

13-17. (canceled)

18. The method of claim 11, wherein the chemical conjugation comprises a maleimide moiety, copper-free, biorthogonal click chemistry (e.g., azide/strained alkyne (DIFO, dibenzocyclooctyne (DBCO), bicyclononyne (BCN)), metal-catalyzed click chemistry (e.g., CuAAC, RuAAC), or any combination thereof.

19. The method of claim 1, wherein the antigen comprises a peptide, wherein the peptide:
(i) comprises a natural peptide, synthetic peptide, or both;
(ii) comprises a lysine, wherein the lysine is a N-terminal lysine;
(iii) comprises unnatural amino acids with side chains that allow for the binding of an azide, strained alkynes, maleimide, pentafluorophenyl (PFP) esters, or NHS;
(iv) is less than about 150 amino acids in length, less than about 140 amino acids in length, less than about 130 amino acids in length, less than about 120 amino acids in length, less than about 110 amino acids in length, less than about 100 amino acids in length, less than about 90 amino acids in length, less than about 80 amino acids in length, less than about 70 amino acids in length, less than about 60 amino acids in length, less than about 50 amino acids in length, less than about 40 amino acids in length, less than about 30 amino acids in length, less than about 20 amino acids in length, or less than about 10 amino acids in length;
(v) comprises a linear epitope of the protein from which it is derived, a conformational epitope, or both; or
(vi) any combination of (i) to (v).

20-33. (canceled)

34. The method of claim 1, wherein the antigen is derived from a virus causing a pandemic, wherein the virus is a coronavirus, an influenza virus, an Ebola virus, a Chikungunya virus (CHIKV), a Crimean-Congo hemorrhagic fever (CCGF) virus, a Hendra virus, a Lassa virus, a Marburg virus, a monkeypox virus, a Nipah virus, a Hendra virus, a Rift Valley fever (RVF) virus, a Variola virus, a yellow fever virus, a Zika virus, a measles virus, a human immunodeficiency virus (HIV), a hepatitis C virus (HCV), a dengue fever virus (DENV), a parvovirus (e.g., B19 virus), a norovirus, a respiratory syncytial virus (RSV), a lentivirus, an adenovirus, a flavivirus, a filovirus, an alphavirus (e.g., a rhinovirus), a human papillomavirus (HPV), Eastern equine encephalitis (EEE), West Nile virus, Epstein Barr virus (EBV), Cytomegalovirus (CMV), Hepatitis B virus (HBV), John Cunningham virus (JCV), Japanese and tick-borne encephalitis, encephalitic equine viruses, Human Metapneumovirus (hMPV), rabies, or any combination thereof.

35-38. (canceled)

39. The method of claim 1, wherein the antigen:
(i) is a universal SARS coronavirus antigen;
(ii) comprises a CD8+ T cell epitope selected from LITGRLQSL (SEQ ID NO: 423), FIAGLIAIV (SEQ ID NO: 425), RLNEVAKNL (SEQ ID NO: 439), VVFLHVTYV (SEQ ID NO: 427), VLNDILSRL (SEQ ID NO: 429), ALNTLVKQL (SEQ ID NO: 445, NLNESLIDL (SEQ ID NO: 431), MEVTPSGTWL (SEQ ID NO: 451), GMSRIGMEV (SEQ ID NO: 452), ILLNKHIDA (SEQ ID NO: 453), ALNTPKDHI (SEQ ID NO: 454), LALLLLDRL (SEQ ID NO: 457), LLLDRLNQL (SEQ ID NO: 458, LQLPQGTTL (SEQ ID NO: 460), RLNQLESKV (SEQ ID NO: 448), or any combination thereof;
(iii) comprises a CD8+ T cell epitope selected from FIAGLIAIV (SEQ ID NO: 425), LITGRLQSL (SEQ ID NO: 423), RLNEVAKNL (SEQ ID NO: 439), NLNESLIDL (SEQ ID NO: 431), ALNTLVKQL (SEQ ID NO: 445), or any combination thereof;
(iv) comprises a CD4+ T cell epitope having the amino acid sequence AKFVAAWTLKAAA (SEQ ID NO: 382);
(v) comprises a B cell epitope selected from TESNKKFLPFQQFGRDIA (SEQ ID NO: 582), PSKPSKRSFIEDLLFNKV (SEQ ID NO: 583), or both;
(vi) comprises both T cell and B cell epitopes;
(vii) comprises the amino acid sequence VLYENQKLIANQFNSAIGKIQDSLSSTASALGKLQDVVNQNAQALNTLVKQL SSNFGAISS (SEQ ID NO: 584), GAISSVLNDILSRLDKVEAEVQIDRLITGRLQSLQTYVT (SEQ ID NO: 585), or
both; or
(viii) any combination of (i) to (vii).

40-45. (canceled)

46. The method of claim 1, wherein the loading of the antigen and/or adjuvant to the EV occurs at least about 1 day, at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 2 years, at least about 3 years, at least about 4 years, at least about 5 years, at least 6 years, at least 7 years, at least 8 years, at least 9 years, at least 10 years, at least 15 years, at least 20 years, at least 25 years or more after isolating the EV from the producer cell.

47. The method of claim 1, wherein the loading efficiency of the antigen and/or adjuvant to the EVs is increased compared to a reference loading efficiency (e.g., loading efficiency of the antigen and/or adjuvant without the use of an anchoring moiety, affinity agent, chemical conjugation, cell penetrating peptide (CPP), split intein, SpyTag/SpyCatcher, ALFA-tag, Streptavidin/Avitag, Sortase, SNAP-tag, ProA/Fc-binding peptide, or any combinations thereof), wherein the loading efficiency is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, at least about 2,000-fold, at least about 3,000-fold, at least about 4,000-fold, at least about 5,000-fold, at least about 6,000-fold, at least about 7,000-fold, at least about 8,000-fold, at least about 9,000-fold, at least about 10,000-fold or more, compared to the reference loading efficiency.

48. (canceled)

49. The method of claim 1, wherein the time required for manufacturing the vaccine (“manufacturing time”) is reduced compared to a reference manufacturing time (e.g., manufacturing time of a method wherein the loading of the antigen occurs by introducing the antigen into the producer cell, or manufacturing time of a method for producing a vaccine that does not comprise an EV, such as a traditional peptide vaccine), wherein the manufacturing time is reduced by at least about 10%, at least about 20%, at least about 30%, at least about 40%, at least about 50%, at least about 60%, at least about 70%, at least about 80%, or at least about 90% or more, compared to the reference manufacturing time; and is less than about 12 months, less than about 11 months, less than about 10 months, less than about 9 months, less than about 8 months, less than about 7 months, less than about 6 months, less than about 5 months, less than about 4 months, less than about 3 months, less than about 2 months, or less than about 1 month.

50-52. (canceled)

53. The method of claim 1, wherein the EV further comprises a first scaffold moiety, wherein the antigen is linked to the first scaffold moiety on the exterior surface of the base EV.

54. The method of claim 53, wherein the EV comprises the first scaffold moiety prior to the loading of the antigen to the EV.

55-56. (canceled)

57. The method of claim 53, wherein the EV further comprises a second scaffold moiety.

58. The method of claim 57, wherein the EV comprises the second scaffold moiety prior to the loading of the antigen to the EV, wherein the antigen is linked to the first scaffold moiety, and the adjuvant is linked to the second scaffold moiety, wherein the first scaffold moiety and the second scaffold moiety are the same or different, wherein the antigen is linked to a first scaffold moiety on the exterior surface of the base EV, and the adjuvant is:
(i) linked to a second scaffold moiety on the exterior surface of the base EV;
(ii) linked to a second scaffold moiety on the luminal surface of the base EV;
(iii) in the lumen of the EV; or
(iv) any combination of (i), (ii), and (iii).

59-61. (canceled)

62. The method of claim 58, wherein the first or second scaffold moiety is a Scaffold X comprising a prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), or any combination thereof.

63. The method of claim 58, wherein the first or second scaffold moiety is a Scaffold Y comprising myristoylated alanine rich Protein Kinase C substrate (the MARCKS protein); myristoylated alanine rich Protein Kinase C substrate like 1 (the MARCKSL1 protein); brain acid soluble protein 1 (the BASP1 protein), or any combination thereof.

64-70. (canceled)

71. The method of claim 1, wherein the EV further comprises a targeting moiety, wherein the targeting moiety is loaded before, after, or together with the loading of the antigen.

72-81. (canceled)

82. A method of treating a disease or disorder in a subject in need thereof, comprising administering the EV of claim 1 to the subject.

83-84. (canceled)
